SEQUENCE LISTING (344 sequences)

SEQ ID NO: 1 - DNA, 137001 bp, Homo sapiens
caaaaattagccgggagtggtggcatatgcctgtaatcccagtagctgggaggctgagacaggaaaatcgcttgaacccgggaaacaggttgcagtgagccgagatcgtgccactgcactccagcctgggcaacagagcgagactccatctcaaaaaaacaaaacaaacacacacaaaaaaccaaaaataaataaataaaatgatcacttctgaatactgatctaactaggggttgcagggtgggctgatatagggagaaactggagagcaaggagatcactaaggtccctacatgtccagaaccaagatagaggtcttgaactaggatggtggcagttagaacaacaacaacaaaaagtcaattccaggctgagtgcagtggctcatgcttgtaatcccaacgctttgggaggctgaggtgggagttagaaagcagcctgggcaacactgcaagacctcctctctaaaaaaaaaaaaaaaaaaaagttagccaggtgtggtggtgcccacctgtagtcccagcaactcagaaggctgaggtgggaagattgcttgagccccaggagttcaagcttgccgtgagctacgattgtgccactgcactccagcctgagcaagaccttgtctccaaaaaaaggtcaattccactgacttttctaaggtgtacaccatcaaggggcagctccatctccaggccattggctcatgagacattctgtagtcagaaggctagggcagattgctttgagcaagcccccatggtggttctcactcctacttctttgggtatatgcccctctgtttaaaaataaagttaatatgcatttaaaaaaaaaaaggagaaaaaggtcagttccagaaactgtgtgaataaagcattttacttgctttttctattaatctataacatatgttgattttttaaaaagaatataagagctatgcaaattggagcttcaagacaacttcccatctccctaggaggagatggctgccctaaacccccctacatagaaatcatcccactgcttgggcttaaacttgatgttggggaaatgaaaaatccaagctaaggccgaagcctggggcctgggcgaccagcagaatgaggaccactggtcagtttcaggctgaggtgcgtcttccaggggacaatctctagctggcccttaaacattcagacttcaagctctatttacagcataaaggtgtttcaaaagacgtgatacaaataactgcaaatgctctgcgatgtgttaagcactgtttgaaattcgtctaatttaagatttttttttctgacgtaacggttagattcacgtttctttttttttaagtacagttctactgtattgtaactgagttagcttgctttaagccgatttgttaaggaaaggattcaccttggtcagtaacaaaaaaggtgggaaaaaagcaaggagaaaggaagcagcctgggggaaagagaccttagccaggggggcggtttcgggactacgaagggtcggggcggacggactcgagggccggccacgtggaaggccgctcaggacttctgtaggagaggacaccgccccaggctgactgaaagtaaagggcagcggacccagcggcggagccactggccttgccccgaccccgcatggcccgaaggaggacacccacccccacaacgacacaaagactccaactacaggaggtggagaaagcgcgtgcgccacggaacgcgcgtgcgcgctgcggtcagcgccgcggcctgaggcgtagcgggagggggaccgcgaaagggcagcgccgagaggaacgagccgggagacgccggacggccgagcggcagggcgctcgcgcgcgcccactagtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtaagcgccgcggctccgaaatctgcctcgccgtccgcctctgtgcacccctgcgccgccgcccctcgccctccctctccgcagactggggcttcgtgcgccgggcatcggtcggggccaccgcagggcccctccctgcctcccctgctcgggggctggggccagggcggcctggaaagggacctgagcaagggatgcacgcacgcgtgagtgcgcgcgtgtgtgtgtgctggagggtcttcaccaccagattcgcgcagaccccaggtggaggctgtgccggcagggtggggcgcggcggcggtgacttgggggagggggctgcccttcactctcgactgcagccttttgccgcaatgggcgtgtgtgtgtgtgtgtgtgtgtgtgtgtgtgtgtgtgtgtgtgtggaggggtccgataacgacccccgaaaccgaatctgaaatccgctgtccctgccgctgttcgccatcagctctaagaaagacgtggatcgggttctagaaaagatgactccctgcacgcccctccctgcacctcccgagcagtgattccgacagggccttcactgcccctgattttaggcgggggccggccccctccccttttcctccttcagaaacccgtaggggacatttgggggctgggagaaatcgaggagatggggaggggtccacgcgctgtcactttagttgcccttccccctgcgcacgcctggcacagagacgcgagcagcgccgtgcctgagaacagtgcgcggatcccactgtgcacgctcgcaaaggcagggttcacctggcctggcgatgtggacggactcggcggccgctggtccccgttcgcgggcacgcacagccgcagccacgcacggatgggcgcggggctgcaggtgcatctcggggcggatttctttctcagcgctcggagcgcagggcgcccggcgtgtgcgctccctgccggaggcgcggggctggcgcgcagggctcgcccctcactgcggcagtgggtgtggaccctggtgggcgaggaagggggaggataggctgtgcctcctcccactcccgcccccagcccccctttttttccccctcggaacgcgaggtgccatcttttttcggcgtgtcacgtctttacggtgccatgccaaaccgggtggccgggcttcataggacagggcggggcctggcattaaagggagggggacaatcagcgctgaaatcttggcgttttgctgctgcgggcgtgagcactgggggcgttcgcccagcaccttcttcgggggctctttgctttgtctgtagaggttacgtgatctgcgctcccagccctggtttctggcttttattctgagggtgttcagtcaacctcccccctacgcccatgcgcctctctttcctttttcgctcctcatttccgagcccattgttggatctcgaggcttgctgggttcgatgaactcgagtcaaccccccgacccccggcacgcatggaacgggcgtgaccgcgcgcagcctcgtctcggagtctgccggcgccgggaagcttctgaagggatgggattcgagtctccgtgcgcgctgcgggcggcggcagagggatctcgcccctccctacaccccaagtgtcctgagggccacgccacaccaggttgcccagcgagggacgctggctacccatccggggatgggtggggagccctggcggggcctctccggctttacgccctgttgcttcgcctggccggagaatgtgaggaaggggcataaggttactggtgcttcggccacacccatctttctgagcccactggactgggcgcagaggggggattgccatggaaaccacaggtgtccggagaggggatcttggggctggcctcaccccttccctgcggagattggggaccctggggtagggggagccgcgcccagtcggcctcctggaggacacgggaggaagccccgaacccccgcgcctgaggctgtttctgattggcccctggaggccgcagacacgcagataggcggccctgggtgtatttttattaatattatgtccgtactgattaatattatttatcttaaataaatttcacccgtgtccaagttcaccgcgcccccaaaaccgagtctggggcggcagggggaactcctggccaacgaatccatgcctcgccctcctgtgatgaacctggtacgcacggttttctggttaattctatcgctgaaaactggtgcggggggcgcacttctgagacggaagagcatctaggagctgaatcctccacgcgggtcgcccaggttgatctgaatttctggggaatggcttggctgcccgcccgggaccaggccgaccctccttgacggtggcgtagagggctggagcctgggtactgcgaggctcctcgcatggctgggcccgccgcgaggggttgcagagcggctcagggatcgattcaagcatcgtctctcctccctcgcccccagacagagctgggcgcggggttccccttccagatggagcgagggtctcggggtggccccggaaaaggggagcccgcggccacggctacgtattgccatctcgcgagcagagatgtcacctcctgcctttggaggaaagggagcccggtggggatgagcgcatttagcccaatgctgggaacaaagcgcactccgcgcttctgcgatttcgctccattttgaaatgtgttggcgctttggtggggccgctgcggtgggcaaggccgggggcgctgttaatggaggaacctcagggggacggtccttcgtaggaaactctatcctggctctgcgcgcgctttaaggaaatggcttccctccaggacctcgagggatgcagcttttgcgcggatgacggtggggtgctgaaccagccggtgcgcctctggaaatgtctgggcacggatcctggggccatcgacgactcctccccattcccagcaggcgggagctcttacattccgagcgagtgacccctctcaccctctggcgctcacacacctgtaactccaaacctccgtctcagaatggtccaggctggaagggatgatgggggctccgacagcgactgcctagctcacccctctgcgtgctcaggctccaggctcagcaggaccaatttgagttctatctgatccccctcggccccttaactgacccatcctacaggagacagggaaatgtctttcctaccgcggttgattctggggtgtcattttgtgttttgtgatggctgcttatatttactgtataagcattgtatttactgtataagcattgtattataattactgtataagctgcttatatttactgtataagcatctccaaatcctccctctacgtaaacaaattaatggataaacagataagtgtatcccctgcccccacccctgctacgcaggtccggagtgactcttgaagctcatacattccttggccaagtttgcttctctaacagatgtttatatagcaataacctggcttggctcttgggttcacctttggacgatttggggaaggggcttgttggctttgctgggttttggatgagtgacagtccatgactgttcctgctggaagggcgtgacttttaagtggtttctaatatcaggcattgctcctccgacaggaacaaaagaaatggatactgcccataaattgttagaaaacttagaatcgctttgattgaggaaaggttagatttattccggttggaaaaagtggcctttctattaaacgtgccctttgaccctcatgcccttggaggtcggtgccagcctggagatgggataagattgtggttttccttctgcctttttaacatctgttgttacagtccatttgttgaaaatttaaagaaactgttttattccactttccctcagcatttatgtgtgtggtttcagtagctctgtggctatatgtacgaacacgtgttatttttccaattggacatgtgataattttccaactggaccttgccttctattgatgtatttatttagcatcttccttactccctccttgaaaaagaatcactcaaaaacaaataaaaacagccgtaggggcctaatacagtgctagacatacaagaggtattcggtccataccaaatggattttatccatgaaggataaatggggaaatacagtgggaagcaggtgggaaactgcgtttgactctgctctttcctccaccaccactttcctcatcaccgtgttcagagacccccaaagccccctcacactcccagaaacacccccctggccactcctaacttgccatgcccaggagttaggtgcttccactagtgacatggagctggcgtttggggggcacctcagcaggtgacgggaagagaagaccccagcctcaccagctgggctgcagcagggagaggagtcctcatgttccagcagggactctcagctgttttcctgtaaaaccatggttctcaactgggggccactgagatgtctagagagatgtttttgttttcacaactcggggagggtgctactgacatcttgtgggtagaggccaggaatgctgttaaacatcctacaaggaaggcacaggacagtctcctacatcaaaatatgacccagtcccaatgtcaccactgctggggttgacactggcactgctatcttaattacattcattgagtgtcttttaggaggccctattctaagtgcttgctaagattatctcatttaatcctcacaacacttccgctatgtagcaggtgctgttattatctccgtgatggggaaactgaagcacagagagggttagtaacttgctaaaggtcacagagccagtgggtggtggagctggttgcctgacactagttccctcccctctcagccacatgtgggtttacttggccattgtggactagtctgggaacccagatatgatctataacattgacccagtagaatattgattccaaaaccactgtctcacaaatgaatttttacaagagtctgtaatcggagcatgacccagaataaggttagggagatgtggagttaaagctctcaatttcttatctggccccgacacagagagcaaggcatttcactctacattggtgctctgtttataaaacaaagagcaaatatctcttcctaaggtccttaaacctcttcccccaatccagggtttctggactgctctgccatatgacggggcagctggtttgattgacccagggaaggctggaaatcaagactgggggatcaagacgtagattcagtgtggccaaggtcaagtctctgaggtttagggacatcagatccccagcttaggttctgtacctcggcaaggtgaaagcgttggcgcccactgatgaggcctgctctgagattgtgggtgtgggttgagttgggtgggcataggcaagtcctcttgtaagaatcttttggcaaagatgggcctgggaggcttttctcacttcctggggcccaggctttgcaataagtattccattatactgtggtaccttggggctacctgagaatcctctgtctcgcccctgttgccttgccaaagagtttgctgtccaagaattcctttcctgtctccaggtgccatgctcctgccacctctgccaggttccctgcctgcccagatggctcccaactgagtgtgaggaggaatttgagacaggttttgagctttctgggttctccagttaggaaactttctgtaagcatgcagatagaatgggcttcagcaaaatacaaactcgaacaacttccatgtatagtcccttaattttctttgcttttttcatatttcatcaggctccatgctgagcccaatcagggacccgatagaaatccaaacaccatgtcagcgagtccccaagaaatgcattttgtgccaaggctattcaaggaaggtttgggagcagctcaagggcagacactgttaccctcccccaggtccccagtgcagggcagtgttctgcatgtggaggcagtttggcctaatggttaaggaggtaggctctgatcgggcctcctgggcacaaatcccagctccctgctcactgtgagacctaagccatattgtttagctgcttggagagttttttgtcatccacaacttggagtatgatggtacctgtctcacgggttgccatggggttcacacaagctaacccggtactcactagggccaagcacatagtaactgctcagtaaatggcatcatcggcggtgtcctgtggatgagtgcttgtgattggctgaatgaccagaggggtctaaagatcctggtgatggaatcagttgtacagataaattgttacactgagtagggatcaagataggaaaagtcggcaactacccagctcccctgcaccaaactgggcagaagtggatcctctgaaaattgcacacacccatgtttaaatgtacacacagaactcttgccacaggcaagcggagatttgtcatctgctgtccctgcctcatcttcttcctgaaatccactccatgccaggaataaactgcatgctctccaccagcccaaactgacctgccttcccgccagccatcccgggcagggtgacctggcttagtacatcgggttcagagatctttccagtttactcgttgaataaaaagtgagggctgatcgagaaagtaatggcagtcagggaaggcgaaggaggtaaagaagagattttacaaatgaagtaattcaacagagtgctgacattggtaaactggcaaacagatttcagggtggttggttgagagtagagtagaaaaggattaaataaagcaaacttgtggtgtactgaatcttaggaattccatgtatccaataagtatagtcatttatgaattaataaattcggcctaagaagccttcttatcgcttaaatcaagactaagtaacaatatatcagttttaaaaagtcattatatcagaaaatcatttaaatgatacacatagatttccaagattttactttaaccgaaactatataaatgtgaatttgttcacccatcttttgacacagggctcaggtcttctcttggtgtctggatcagccagttgaaatttcttgtctgttttgcctatgccacattaataatgcactgtctgggtcctccgatttcagtttggattttgggtttacattgtggagtcatctgaatgcagaatccttcagggattttacttttttttttttttttcatggtctttaccatcccatttgatagtaaatattactcacctttatgaagtctttccaaaacattcaactaaattttcttaaaatcattgaatgatttgaagagcttattcctcagcacttttactccatcagcttgcaccttattttttaatctttttttgagacggagtctcgctctatcgcccaggcttaagtgcaatggcgcgatcttggctcactgcgacctccacctcctgggttcaagcaattccgcctcagcctccgccgtagccgggactacaggtacacaccataatgctcggctgatttttgtatttttgtagggatggggtatcgccatgttggccaggctggtcccgaacttctgacccaagtgatccacccacctcggcctcccaaagtgctgggattacaggtgtgagccaccgcgcccggccagcttgcaccttatttaggatatgtgattattatagcaagtctggtgtacatacaagattttgaatgggcacagatgacctttagtaagtgcttggctgtgataagaggcagtcctgactgcagatcaggctgtgtggaccccagccttgcatgtttacagaccttcatgtcttattcttacagggtatcagaagaacacctactggggaaacttataaattagtaaaaggtgggcattctccccgcccatcttctgtctgtctgccaggactagcacagcactttgaagtcattcacatagaatcccaacttaagagggtaaaatcctcctcaacagactgaaaataagtttaaattccctttgctatattaactcccctgaggaaagagtcttagatcaatgtccaacactaaaaacagttttaaatcagcaagtgagaattaaatctgaagcaattgataataatgtttcattcattcctctcctttggccccgtccaccctactgctaaatccaggcatcaaagagaagagggacataattatctctagtcccagctgctggttttccttccagcctatggcccagttttctgttttactgagaaggctggtgatgttatcttgggatctaagtctgcagtttcaccacaaaaagtccagggatgcactttcatgcttgtgtcctcctccctgggatagcaaggatattagaagacccctggctctgtaattgcttgtcatgtgctctacagacgccacagaatgccaagaacgaagtgctgggaaggacaaattcatggaaccgtgggacggtgctcctcccccagcgtaaaggacagctcctcctcctgaattggagccagcgttctaaatcatgtgtcaacagagttgtcctggatcggatccagttctgccattgatttgcaggtcatttcagtggtacctgtttccagttgttcttaattgaacagtggcaccaaactattgtcttgcctcatccccctcccatggcctgtcccccaaaaagagacttcttgggtaattaatcagggcaacatcaggcagtctgggcgcggtggctcacgcctgtaatcccagcactttgggaggccgaggcgggcagatcatgaggttaggagattgagaccatcctggctttgtgaaaccccgtctctactaaaaatacaaaaaattagccgggcgtggtggcgggcgcctgtagtcccagctactcgagaggctgaggcaggggaatggcgtgaacccgggaggtggaggttgcagtgagccgagatcgcaccactgcactctagcctgggcgacagagctagacttcttctcaaaaaaaaaaaaaaaaaggaatctctttggttttatatatttttttttatatatataatatatattaaaatataatatatatatttatataatataatatataaatatattatatattatatatttttatatattatatattatatatattatatattatatatttatatatttatatattatatatatttatatattatatatttatatatattatatatttatatataatatatattatatattatatattatatattatatattatatatttatatatattatatattatatatattatatattatatatttatatattatatatttatatatattatatattatatattatatatttatatattatatatttatatattatatatatttatatatattatatattatatattatatatgtatatattatatatgttatatattatatatatttatatatataatatattgtatatattatatatctaatatattatatatattatatatattatatattataatatatattatatattatatatatttttatatatataatatgtataatatataatatatataaaaacatatataatatatattatatattatatatatattatatatattatatatattaaatatattttatatatattatatatattatatatattaaatatattttatatatattatatatatatacacatatatatatataaatgaggccaggctcggtggctcacacttgtaatcccagcactgtgggaggatcacttgaagccaggagtctgagactagcctgggcaacaaaacaagatcctgtctctacaaaaggaaactgtaaaaattagctgggcatgatggcatgtgtctgtagccctagctacttgggaggccgaagcaggaggatcgcttgagcccaggagttcaaggctacagtgagctatgattgtcccatagcactccagcctgggtaacacagcaaggccctgtctctaaactttttttttttaattctatttatatttacatgtatttaaatgtgaatattcactacctatttgttgcatgcctgcattttttatactgggcttgccaaaaacccgaacagctttctactttgacaatgtatcagaatttaaatcagcaatatgttaataagccaagcaaaggttatatatgcaaataaaactgttgtctataacctcctgttacactggggcacagcaaaagtcatggtgtagtcgcatgtgaacctgtccctttcatagctgctcattgccaggaaacatcaggaatagccatttggaagagtcatcagccctcccaccatccgttttctgtcttgtcttttccctatgagcaggggaaattccacgctggccccaatccccagtgcagcggctcagcctctgcctctgctgctggtccccatgaggccagcttagaaacggaggattttgcagaacatccctaaatccgcttgaataatgaagtgatcattcataaactcacctgaaccttattaaaacctatttaatatttttcctggataatcctatagggataacttgcctcctgggcttctctccaccgggttcagttcttcctttagtggtgaagttcctcccttcttagcatctcaactgtgcctgagaaaaggccagtggcggctgcactctgttccctgtggagtgttaataaagactgaataaattgaaataaatccctttcaatgtcattaagtgctataaataatcatgaaccaatgttcgatggctgatgagaaatgcaagaaaaaatttttaatcagtaggattcataagttgacaatctgggccaagttaaaaaaaataaaaataaaaagacttttaaaaagatcttatcgtttgttaccagtaagactgaattccagaagcaagctactccctcatttgtgggcccctgttatcactggctgcttagggttgccaagccctgaattcatttgtcaactaagagatttttggccaagattaagatttcccatgcctccatatttccatctgagaaatggagattatactgtcttccccctcagaatggatgataatgtggtctctcttctgttcgcatagtcatagaactgaaataaaacaacttaagagaattcctttgagcttctcagaagtgctgcagggctgggggatgcctcccaggagccgcagtcaggtgctgatctgaagtctttggtgggctgactttagcctgacctgaaatagtatagctgctgccacctggctcccttagcgtcagtcagacggtgcagctggttcctaggggtgagggctgagccagcagggtccgtgcccaggagggatgcatgggtggccacagcccagcctgcactgatcttgtctgtccccttctttggaaggaaggagccccaaaccagggtgcaagacagtgggtgggggtgccttgagcatgacctcaagtgatttccagcccctgccagtgctgacttctctggggaagggctgggacttccttctgggctcaagtcacgacccttggatggaatttcctgggagcttttctgttttttctggagttttcagttttttcctaaccagacagggacttggtacagaatctcatattctaattatgcctaggagcagcctctccccaccactcacagtgtttagcatgtgacaggaatcgattaaggcatgagtgattaaattaaagccaggcattgacttggatggtgtaatattctgacatctgtttggtgtcaaaggcacggggcaggcgcgttaattgaactgcttgcacctggcatttgaattgagccagagcggggctaaagtcagtttgccttcaccctgtaaatggagggtttctccggagcgtggatggtgggaggtatttcagggtgtatgcataacccccaccctgacaatggcccatctcttctccagcgtggccaggtttgagtgccagtcctgggtgtccagtggccccatagccttgcgttttagtaaaatgctgcccccattaccacctggtctgtgcacttcggtcactggaatttgccatcttccagtcccgaatgtggcaagccatggagccttaagctcttctccctccacatcctggaacagacccgccagtttcttccaggcattgcctcagtttgcccctctgtttccagtcacactctcaccagcgataaaatgattttagaccttatcatctcaccctcggatccttatggaaacaataatgagttgttccctgtttcaattccaaaattcatatccaatccgttttgcatgccattgccaaattcctcccagagcaaccccgtcacctgccctggccctctccaagtgtggtcctgccatgggcatcgcctgctaagccaagctggcctcgagctgcctgcccgggtccccacaccttggctcacctccctgcccagtcccgcctcctgccagcctgccctgtggctccttcatagatgccgtgctctttctgccccttgctcacccatggcagccttgcccctctctccctgccccaccccctatttaaattgacctgaccttcctcagtgtccatcttccccgaagctttccccagccttggcactcaaggtccagaggctacgcgtttcctctcacctgtggcagcgccgtgctccccagtgcctcacagtttccttcttgcccccgcttcctgtgtaggactcatctgcccacaggttgcacgtcctgtgagggcaaggactgtgtcttatgtgactttccttctccagtcacagagctgggcacatagatagctcaaaaccctctttattaacacagttggatgttgagaaatcaaacaggccaatgtcaaatgagctctccttatttaaatcaagtcagttctccacctcctagcactcagttccagtactctatatacatggaaataataaaaaacacatttcctttgaaacattctataatcgttcctttgccctacttcagaccaacttaacgcactccccattggtccaaatgagttttgctatacgaagatgctgataataatagcagcagtggattattctgctaaaaccattgcctcgttaatcctcagtcccgaggtggggattattatcctcattttgcagagaagcaaactgagactcagagatttcacagctggggagggagccagctcatccctctgtccaggcccaagctctctcccgcttgccttcctgcctctgcaacctcagagcatcccccatctggttctactgcctgtgctagtcgtgcaggagccaaaagacacgtctttagtgctaaggactggagaagccatgccctccagcctctgtgaatgggtcatatgtaacatgagcctggagaaattatttgaaaccaaaggcaagcctctaaaccaggctgctgcttcatggcgccggtgacggcagaaccaaatttagtgctgtgggcaggtccacacttatcaaatagagaagctcatttttcttccggctcacatcaagcatgaaaaatgttcacacataccccccacacacacatgctttccggaggggtccatgtggctagaggctggaagatgtggatgagaggagcctggcaggtaagcccagggaagatgacattcagcttcccagacagcatctacagggagaaatttaattaaaagtggggcggtttccctgagcaaggcagacaaagtcagccctctactgttaagaaaaagggtcacagtgagaggggaggtgaggagactgagtctgtattttctagtctgttgggctacactacctgatcccccttcctcaaaaatccactttactttccccatgtctacaccaatgtggttcacactctgggaccaggaaaagggggagtgatggggaacagagaagggaggagctcacacagctgaggctggggttatgcatatcgaattacttagaatttgcaacctcacagggtactttcatggcgttgaaatacacttcccacagccaccctccctctaactaaaagcaagagtcatttctcagttctggtcttgcctcccacgttctcctccacatttaagaaaatccaccagctacaaagtgaagataccatatgtgatatcccaccctagtttctgttttatcagggtttggagcaggtggagcaggcagagggatcatttcagcctataaattgtattaagggtgagtactgagtcattcttcaagaaaagttttagaagcatccaaaactgaagggtggagccacctggagacagtatcatcagtcctggccccgagcatggcctgcataggcccccatggatcccagcgggagctgcagagtgcgggcaccttggcacacagccctgagtgcaaaattaggagctgggcagagggcatctctctgtcgccattgggcagcccagggcacactggtcatagccttagaccacgaacaccctgtgcccgggggacagatgcaaccagtgtgccctgggctgcccaatggcaacagagagatcgacacctggaccccatgtcacggggactccactactaaggctcctaagactgccaccttccagtgggataagccctgcctcctactgggcccacaatgtgcagagaacacttgggactacctggctttctggatacacaaatattgatccaatctggactaattagaaggtcagtcccaataacaaatcgaagtcagctgggcgtgatggctcactcctataatcccagcactttgggaggctgaggtgggcagatcatttgaagccagaagttcaagaccagcctgggcaacatagcaaaaccctgtctctactaaaaatacaaataattaggctgggtgtggtggctcatgcctgtaatcccaacagtttgggaggctgaggcaggtggtcacctgaggtcaggagtttgagaccagcctggccaacagggtgaaaccccgtgtctactaaaaacataaaaattagccaagcatgatggcatgtgcctataatcctggctactagggaggctgagacaggagagaatcgcttgaatccaggaggtggttgcagtgagctgagatggtgccactgcactccagcctggttgacagagcaagactctgtctcaaaaaaaaaaaaaaaaaaaaaaaaagccatgcctggtggagcactacgtgtaatctcagctatttgggaggctgaggcacgagaatcacttgaacctgggaggcagtggttgcagtgagctgagatcgcgccactgcactccagcctgggcgacagagtgagtgagactccatttcaaaaaaataataaatctgagtcactttaatattgttatttggatgtcaacctctaggtgtttgagacaggagagtgatatgggggcactggaaacacacaggcacggggtgtcctcacacttgggtagcccacacgatgtgatttcagggtgctgggaggtccccccactccccaaattactaacaagtggatagtactttacagtttatatgatctcatttgattcttaacatgagcctgtgagtgaaaaattccttcccctcttctacagattaggacgttgagattcagggaggttcagagggattcagggaagtcaagtggcacctggagtcccgtggctaatttgaggccggtaggggattcgaacccaggatttgtgcttcttatgcctgggcttctgctccctggggcatggtcttccccctagctttcccattcactgctttagcctaggggtcctaccctttattaaactgccagtgcctcactgcttttctcccccaaagacaaaaaaaaagtgtttttgcttttgttttgtttttcatgggcagagacctggaatttcagcttgagaatttgtgccatatgataaataaatcaacagatggctttttccttaaaaaaaaaaaaaaaaaaaactaagatgtatttgcagtgaggcataatttgtaccaaaaagtgctcaccacactgtagtcatgggggcaggaggcagccgcgggtgaagggagaaatcttggagtccaggcagcccccttctgggctgaactggggagctgggggtgctgccagccctgccaggttctcctaggaggcggcagctcatatggctgtgggaggaggcagagggagcctcatatgcacccacatttccagggatctagaagacagaaggaggaaaaccaccatcatgttaaagcagacagttaggtaacacatcctgtaatacaagttattttttccacatctaaaggctaaaaatagttgttagaatttaaagataattggtaaatgagtttctatccttctagtttcacatcaaatggaatcatgctgccttcacatcactagtgcccgttatttgtgtttaatttccacaatgttgtctaattccactctttgggcttccccagggatccagcctccctcactcgcccatcgcagggagatgctttattcatctttgtgtcttctgtgccgggcatagcgcatggcacagaataagcactcagtaattgattcacgagtgaataaatggatgagtgggtgagttcaatattgactacaaaaaccctaaggccacactggtgagtggctgcgcctgtagtcccagctgctggggaatctgaggcaggaggatctcttgagcccaggagtttgaaactagcctgggcgatatagcgagaacctgtctcaaatgacaaaaacagggccaggtgcagtggctcacgcctggaatcccagcactttaggaggccaagatgggaggatcacttgaggccaggagtccgagaccagcctgggcaacatagggagaccctgtctctacaaaaaattttttaaaaattagctgggcatggcggtgtgcgcttgtagtcccagctactcaggaggctgaggcaggaggatcacttgagcccaggaaattgaggctgcagcgagccatgatggcaccactgcactgcagcctgggcgtcagaacgagacctgctctcaaaaaaacaaacaaacaacaaaaaaaaaggctttcttaaagagacttgagaacagaaaggggaacagatacataacttatatatttatttgttcatctttccaccttcctggagggtggaggggaacaggtctgtatttggagttttgaatgctaaaagtgggaatacatgtactgtttgccatgatctgttcaaaagttaagccaaatgccttagattctcctgaaaactggaatgccactgtaaactataagccccacttcaaagataaaagatcttgatgaacagggctgggtctgtggactgggcctctccccaccacacaaggaagggtggtgccagttgaaggaaaatcacttaaatccttgctgtctcctaataaggtgtggtcccaggtagggctgtcagaattagcaaattaaaacacagggcatctgtgaaaattagaatttcagataacaacaaataattggcataggctgcataatgtccctcaaagatatcaggtcctaatctccagaacctgtaaatgtgatcttatttggaaaaggggtctttgtagatgtggttaaattaaggattttgagatggggggattatcctgtattatctaggtaggtcctaaatgcagtcacactcatccttgtaagaggaaggaagagagagatggaaaacacagaagagaagacaatgtggtgatggaggcagagattggagtgaggtggccacaagccaaggactgctggcagctaccagcagccagaaaagtccaggaaccaattctctcttggagctccagagggagtgtggccctgctgacaccttagcttcaacctagtgatcctgattttggactttggccttcagaagtgtgagggaatgaatatctgttgttttaagccaccaagtttatggtcatttcctacagcagccacaggaatcaaaaacagtaagtatgtcccatgcaatgtttgtgacacacaccaaaaatattacttgttgttcacctgaaattcaaatttaactgggtctcctgtattttatttggccaacctagttcccaggcccaaagaaagaggcttttgaaatttgcaagaaagctggttggagctgtcagaaagtggactttgtaaacacagtaccaccgaaccaatttgaactgtactacctctagacaaaagagagggcagtcagacagttgttcgtgatttcttctttcaacagtcatttgagcacttactacaaaacagaagctatgtgtaagggtggaggcgttagctgttaatcaggacctccaggctaagtttctgtattagtccgttttcacgctgctgataaagacatacccgagactggggaatttacaaaagaaagaggtttaattggacttacagttccaagtggctggggaagcctcacaatcatggcagaaggcaaggaggagcaagccacatcttacatggatggcagcagacagacagggagagagagcttgtgcaggggaactcctctttttaaaaccatcagatctcgttagacttattcactatcaagagaacagcacagaaaagacctgcccccatgattcagttacttcccaccagatccctcccacaacatgtgggaattcaagatgagatttgttaccatatcagttaccaacccttccagataaatcacgtgaaatatcgccattaacagagtgagctcaggtggttcttcagtgcatttctgatacctgaaccttccctgggaatttcacagaccatcaggctctccaccctttgatagcaggatagcagggcccaggttctgcaggaggagatgttaccacaggcctgaaagggagggaggggcagatgctacaggaagatgctggctctggattcgctggaggagctttcaagggaagtagatacacactgtctccatcatttcatgtccatcacactctaaaatgctttggacaagaagcaaatgttaaagacaaatgtggcccattttcctgtacaaagagggctgctcccatgccaggctattggcactggtgggcatgaggcttctctgctgccctggccggggggttctctcactcaccattggctctctgacacctggagagaccaccacccttgggctttcatgatgctcacagaatccacactgttggagctttaaggagcctggatcaactggaacaggcagggagtactaggacagcccagcattgccccaaaatatccaggcctgataaaagagaaaaacaggtagctcacaggaaaaggataaaaaaaggaggagggatttaacatgaaaaggtgcttgatctccctcataataaaaagactgctgattccatccaggcaagtgacagaaaaaaaaaatttaatttaaaaagactgctgataaaaccacagcgagacactgctgctcagggatctgagggtgtgggcagccaggctgccacgcatcatgggtcggagaggaagaccacacccctggagcagagggcggctgatctgtcagatgccctttgacagcacctcagcttccaagaattaaccctttctatgtgagcagaggcatccatggggggacacactggtgaatcatctgttatgtagaagtctggaaaacatcaggatggaactggtgaaataagtgtggcctctgacggaatggagcggtccgtctgcactgctgcgggtgcccctcagatcctgtgggtcagtgagaaaagcagtgaggaacaaggcaggtactgtgtactgtcctctgcgtgcaaggaaggccagcgcatgcaacagagtccacacagacatagcctaactctggaaggaagaatgagaatgcagtttcagtggtggcctctggtggggagaaactgggtgaagggagatgtcatttccatttctctactattaattttgtattaccatgcttaaatgttactttttacctttttttttttttttgagacagggtctctctctgttgcccaggcaggagtgcagtggtacaatcatggttcactgcagcctgaacctcccaggctcaagcaatcctcccacctcagcctcctgagtagctgggactataggcacgcataccaccgtgcccagctattttttttaatcaagatggagtttttctatgttgcccaggctggtctcaagctcctggactcaagcaatcctcctgcctcagcctcccaaagggctgagattaaaacgtgagtcaccctgcccagccaattgctttttaaaaaagattaaatgcatgtatacgctcaggcatcagcacacttggaaaggatgaaaatatccggaagaagggttcttttaaaaggctcctcaagtgatgctggcaggcatgacgaatgtccctggtcacaaaagctctgatctggcctaaccctgtcatgttagagactggagtgcgtgtgtgtgcgcgcaaagtgtggggggatgggggtgagtgtgtgtggtgtgtaagcatgagtgtgtatgtgtgtggtgtgggggtgtgtgctgtgtgagcgtgtgtgagtctgtgtgtgtagtgtgtgtgtgaagtatgtggtgtgtatgtgtgacgtgaggtgtgtgtggtgtgtgagttgtgtatggtgtgtgcatgagcatgtgtgtgggcatgtgatgtgtgtgtggtgtgtaagcatgtgtgagtgtgtatgtttgagcatgtgtggtgtgttgtgatatgtgtgtggtgtgtgagcatgtgtgtgtgatgtgtctgtgtgtggtgtgtgtgagcatgtgtgttgtgtgtgtggtgcatgtgtgtggcgtgtgagcgtgtgtgtgcattgtgtctgtgagcatgtgtgagtgtgtgtgtgttcagcatatataaggcatgtaactgaacacagcactttagagggctctcctggagtcagagggggtgggtaggaggagaagggaggtgggctagtgtgctgaagtatctactccttgtcatagtctgtgacaacccagactagcccatgagccaccctgttccctgcatttccaatgagacctcggtggacatgttccctgaggtgaggctgactgatgtcatttgacgatcttgatgccaaatccttttatatcaaaaacaaccagaacactctcttttctcttagtgctttcacccagatgaccacatttcatcctcccagccactctgggccaggtggcactgctggtttgaaagggaggtctcccctggagtaacttccgtgggcggattcacaccctgcccacagtcctgtcccagtcagcccaccatggtggtctccggttcctccagaattcccgcttttcagctcatccccacattcccggagggactgagagcgcagccccagggccctgctctttgggggccgtctctacacccagagaagcagcaaggcattcctaggtttctctttcagatgcagaacttcagtgttcagagatgttcccactggtcctgagagggctcagttcagctttaatgactgcgctgttgcgtgtgctctgcagagggcgggtggcccagcgtggctgactgcagttttcctgacgtggagcccgagcctgccccgctgtttattaattaaggatcactctgcttgcagaaccctgaactccccagaactgtgaggtgggagaaccccgagaggccacctggccccacttcccacctgctgcccaaaccccctctctgccttcctgacagtcaccccaactcccagtgatccccatcaaccatctgacaaggggactgagagggaagagaaaggaggggcccaaagaggaaggtaaaactgtcgggaacagcccccaaatgtgtgacagccttcagtggagttgcccactttcccttttctcctccctgcaggacctcccttctccccagtcctccccaacttctgaggttacattgagaaaagtctgcagagaggtgccagcatcacaaggtgttaaggaccacgagtttggcattttaacagatgccagagccacttgagaaatgtggtaactaagcccagagaggtacagttaacctccccagagtcacacagcaggttcatggcaaagctggactagcacaggtgtccttcccctgcagatccccttctgtgccccacatcacctccctccagtgtctgggccacctggagatgggccctcagactcacccggccagaggtgccatctcatgggagaggtctggccaggaagcatcgatatttgagatcccaagaaatgaagacttggcctgtcagatgacagacttcggtcatgggaacacgtgatctgttttacacatgcgtcccctcagcagcagctttccagaacattcccactttcttctgtagtgagaagaactctttccctgcagcctcctgcccaactcctccttcagtgtctttgcttcagtgtctttgataaaccattctgctttgcagagtgcgagctctgccttgcagggttcgcatctgcctgtgctgagtaaccaacgctaaggtcgagtggtcggtcacctctcataagagctagggttgtctcatgctgatgactaggacttgccctcaaggagaaaaataaatcaaaacaaaagcaaaaacagcaaacatgcatctcttaaagaaggctctgagtccaggtaaatttccttccactgaagcagccaggctgaattcgaattatctttgcccctgcttaaaaactaatgcaaattttcctagagaatatccactaattcctggagggggcatgggcattcctgatgcccatgagaggaccatttgctcttccctcagtatgctaaataacagaagcgacatttgttgctggaaagtatcagtgaagttaataaggtttttcttgcccagggtgagggaacagttcccaatgacaaatgctgtatgggaaggggctgtagaactgccagcccctttggtccatccgtaaagtgaactctgtggatcctggaggattccagcgtctttttttttttttctttttttttaagacagagccttgctgtcacccaggctggagtgcagtggcacgatctcagttcactgcaacctccgcctcccgggttcaagcgattctcatgtctcggcctcccgagcagcaagactacaggtgcgcaccaccatgcccgactaatttttgtattattagtagagacgggggtttcactctgttggccaggctggtctcaaactcctgacctcaggtgatccacccgcctcagcctcccaaagtgctgggattacaggcatgagccaccatgcccagccagcatctttcatttttctgtctgctttggccctttcctctctcactgtcttccttttccatttccaaagtcagtccatctcactattagcacaaaaactgctagagcgcttgtcattggtcatctctccctgcacctggctggtctgttcttggccactgaagcgtttcccccagctgttgctttaatcattttattgttattatgccttacttaagaaatggatatgagatgcatttacctgtctcttcctgccactctgcagagccagtaagatgtggtggaaagggcccaggctttggaggagggctggctggggttggatcttggctgccccctactagctgtgtgaccttgggtaagtagctggacctctctgagcctggttcggaatcatagcacctctctttcagggctgctgtaaggaatagcagtggtgtgtataaagcagagcgcacagccagcaactggcccctagccacactgctgagcacctactgtgataagctgccattgtggtgtgtgaagcaaaggggaaacatgcctgctgtagtgagcttcctgtagggcaggttgtagaaccagaggtgggttccaaggttacaaagggactcttagtgtattagtctgttctcacattactataaagacctacctgagactggatcatttataaagaaaagaggtttaattggctcacattggctgggtgcggtggctcacgcctgtaatcccagcattttgggaggccaaggccggcggatcacttgaggtcaggaatttgagaccagcctggccaacatggtgaaaccctgtctcttctaaaataaaatacaaaaattagctggccatggtggtgtgcgcctggaatcccagctactcaggaggctgaggtggaagaattgcttgagcccgggaggtggaggttgcagtgagccaagatcgccccactgcactctagcctgggcagcagactgagactctgtctcaataaaaaaaaaaaaaaagaaaagaaaaagaattgcaagaaataaattattgtttatgagctatatggtctgtggtaccttgttgtgggactgggagtcttggcgtctccctgaccctgcctgttgctgcagcaccgctcagccctgcctgctccctacctgcctcccctcggcctctcctgcctccaccgggcccctggtgcctcctctagagacagtcctcctgggaccgattgtgttctcacttacacgaggcatccaggactacagataaccagaggaaggggcgccccccccgcctgccctcctccctggcatcctcacgctgcagaggtcagagcctcatcccagccccttacctgcccctactctgtggagaaccgtggtcagttcgccaggccggatccacgaacggccttgtggaagatggtgagctcacacccagagctggctccgatgaccctgtctcctttacatgtttctaccttcccctccctaccttcccccactgctgggcgcagagtggaggcagatgaggtttaaagctcagaagggcttaaacgggttggggcgcagtggctcatgcctgtaatcccggcactttgggaggccaaggcagaggatcacttgagcccaggagttcgagaccaacctgagcaacatagtgagaccgcgtctctacaaaaaataaaataaataaaattagctttgcagggtggcatgcacctgcagtccctgctactcagaaggctgaggtgggaggatcgcttgtgcccaggagtttgaggctgcagtgagctatgctggcaccacagcactccagcctgagtaacagaatgagatcctgtctcaaaacaaacaaacaaacaaacaaaagaaggcttaaagggggctccaggtgggcttggcagcacaaagctatgaagttctatcttagacacaagttctgttactgggcctttgcaggctggcctgggtacctggctgccatagacagggaaccttccagatgagctgcaggcgtggagcacaggagccagggtgctcttcctgggctctgtccacaggcagaacgtacacagtctttgtacacgtccggcggctctggtgcctatttttgtttgtgtttttcttttgtttggggggatggatttggtttcccccgagccctctgtcctcctgtcacctggctggtgctcggcaatgttgaccagctgcctggctggagttggcagtggctaaggctgtgacagctaacatgttcctgagtcctctcatttcttcaccataatgccctgttgagtttgcagatactgtctctgtttttatctcccggggaaactgaggctcagagtggctaggccaccttcccatggtccctcagctcatgagggccacacagggcattgcggtggccttctcctcagccttgaccctccggccccagcattgctgcctcaaggggtctcctctgctgagccgtgcaccttctgcctggcagctccaactctgtggctgtgttcagtggctcagcactgccccttgaccctccctggccttctgcggatgccagactggagcactctgacaaggtctggggtggttgtatgggtcctgtgacctctatacacctcccagtgcctgggaatcctgcagatacaccctccttagccgtccctaaccatagaggacatttctgaggtccccgagagagtggggcacccctgcaggatccaactgctgggcccaggaaggatagcagcagcatgaggggttccattagccacaaactcacggcatggaaccttcacccacctcgcccctcatctgctgtttagcacctggcacgccgtgtatacttactgattattacattttaatggcaaattatagtggcaaacgtatgcatctttgcacaattgttgtacagcatgatgaacaagtcattaatagtaaagaataaatgtgaaagtgagaaaaatctgactgccaaagtttttactccttccttccctccccagacttttaaatgaaagtttagggataatcccttagttgtcctgctagtaggacttgcaattaaaagaattgggccaagaacacttctacgcttctccttttaggtttgggtgtaaattcggggtatttctcactgatgaaagcctggtgcagggcagaccgtgggaagctttcatttccggaatggaccatcaacatcccttggagaagaattctcttctccagacccagacctggtgtcctggcacccattgggcaagtgggtcctagaagacaaacctggtcagagcctggaggctgcttagcattccccacgcacattagcagctcggagagctcaggaagccgcagcccctccttgcctcaccagcctggatcaggacagcatcccctggaagacacacagggcctggcctctgattacccagcctggagggaaagctcaatcgagcatcatgtcacccggtgcccccatgcagggtggcactggtgagacccccaagccaatgataccacctcacaggagtgcaggcccattgtggccagatcatcttgacttttcaagataaatcagaaatcgtatttccatgagatatccctatttgcaagtgatggtgactaaattagaagtttttgaatattgtaacatgttcgtaggctgtttgtctggtttaaactctatctggaggaattcaagctagacttcaggaataacttcttgaggcaaggattttgagaccttagggaaagaaggacgtcttgggggtattctgactgttgtcctcctggaagggaagaacagagaactagaagactgcccttagcgaagttcaaagcacctaagcccgggaccctcagcaagtgttcttgagtcacagattctccctgaggcgcctctttctggctccatagaatggctgattctgtaactcggtgagtttgctttttttttttcctccatcacccaggctggagtgcagtgaagctggagtgccgtggagcgatcactgcaacctctgtctcccaggttcaagcaattctccttcctcagcctcccaagtagctgggattacaagcatgcagcaccacacctggctaatttttgtgtttttaatagagacggcccgaagtgctaggattacaggcatgagccaccgcggccagccataactctgtgactcttgttacaaaggccttatattttgctctttgagggtggttttggtttgatgcctgttggttgccatcttttaactagggatgttttatcaaaatgcccagccaaagtgtccaaacaaattataccttaaagtttgaaaatgtctggcacttctaattcaatgcctgttgtgccaggcactgggctgctgaggaactgagtcccgtccctgcaggctagctagagaacacacacacacacacacacacacacacacacagagtggtcttacaagtcagttttatattctacctatatgcaataaaggtattattatgttgaggtgccttgatataaaaatttttcttaaaggagaggatgcctaaaacaggcattacctgaaacctcctctctccagcattggttgtcttctgtcatgactcagggttttcactgagaatgggatggaaatgtggtctaaagatagggccaatgttgggactggatcccctctgggaagtcagaccaggctagggcaggtccttgaagccatcaggaaaagcctctggagccagaaacaaaacaaaaaaaaaatggtgttaactaaactcagtctcaaatcctgaataggactcaagtcaagcaaaataattaaaggagttagcaaagggcaagtcagagagaccgagcaacaccaatgtcttccgggagccctgtggcgagtgacagagcctggactctggagtagaactcatcttgtgtcttcttctgccactcgttagctgggtgaccttgagccaagccccttaacctcttggaccctatgttcttatctctaagtaggggctggtaatatcttcccctttgaggaatgccctctaaggggtgttgtgaagattcggtaaggtggcaggggtaggactcctggccagaaacaggcacataataaatgctaagtctctccttctctccacctgctggatgctgtagatactaaggatttcgatgtgaatgagacaaaacccctgccttccaggagcctttgagaatcagagaactagacccatttccagaacaaggggatgcagggtctggataaagttttggggatcaatagagcagagggctcccagaggatcccatagggttgactcctaactcaagggcatgagacaacccccaggaagggcaccctggaaggggtccggctgtccctgatttacttgtgggcactgggggaatgcccggagccatccagccctcagggctctgtgtgattctgggttcctcccataaaagataatcagattctttcacgttaatgtctttctccacctcattgcacatcatgcagctattcattgactcagcaagtatcagctttgcatgcgaccttggcctacccactttagcttttagtaatagctcccttcttgaataatacaaccagtggggaaacagaacctaactcttacctctgggaggcttatttgctttgagaacatatgtcctgcagttttgttcatatggcagtgaagtttcgtgcacacactctagagccaggcagcctgggttcaaagcgcagctctgccaggtcctaactgcatgaatttgggcaagtcgctcaacctctccatgcctgagtttcctcatctgtaagattggagcaatggtaatacctgctttttagggttgagaagagaattaaatgaattaagatgggtaaagtgcttagagtggagctttgcaagtagtaagtgctatgtaagtgttcgatttaaaatgaaagacccttaaatacattctttgttcatttcacaagcccttcatttcacaaccttacatttcacaaccaagctctgtctcccctggaatccagccataactctgctcacaagtgtgagacaggccccagcagagctgcacgaagaggagagaaggcagccccccagactcccaaccccctgtccaagatggcaaaaccagaacacagcctctgtaccaccccagcaggtattcagaatctgcaatctccaaagcccacttcaattgtaaatgtagagccacgtgcgctttaagtcacctgtcactctggaggctcttttgctcagttcctcaccattagcagggatgacagggagtgcaggagtgcggtcgactcccagatattggagagcgctgggctagctgcccattctcccggcctccactcctctttgctgtccagccatcacttgctctttgaaggcaaacaaaacagaaaacagtgccaaaagtatgggaagaaagccagcttctcccctggggtgcctgtgatgccatgcccaccctccctgaccacgcagcccctgtggaccctcagggccccaagcccccatttccatcacatgcgtacacccatgtgtgtccatagccgcccatctcagtcaataaggctgctcctgcccacttggaatagtggtgacaaccaggagtggcttatgggaactatcccaatggcctgacagcatgtccgctgcaaaccgctgaggtaggacactgccctcatgtctagctgatcagcaagaggcgcagttgctttcttaggtaacattgctgctgtgtcctggccattgctggggggtggcacttaatctacaccagatttttccctcctgtatcttccaagctgcttggatcttggtgctgaattaggttggactttgtcttgtggggaagggaggactatagaccctcaacgtaagcaatggtcagactattctaagaaaactcgccgaattaaagcatgaggtaaatttagttctgacttctgtccaccccactgccactgtccccttttatcccatgatcccttgcttttcttttcctcctctctccctatctcttgtgtttgacgcatgataggaattcagaaatatatgtttgtggatttgtttattcacgtagcaaaccatttcttgagtgcctaccatgggccaggtagaatgggcggccccgggctgcagtggtttcttcagcccctctccagggtttacactgtgcaagacggtttgtgatgggtcctcccatcgaggaccacactcttctttctctgtgccccttggtcctcagtctctgaccccacttcaaaggcagcattcactcagggaagctcccatacaatgctagtcagagtaaaagtttggacaaattgccaggaagcagcttgtcagtatgcataaacagcctttaaaatattactactctttgacccagaatttcacttctaggaatctgtcctaaggaagtagtcacatgcaaaagatttatgtaccaagatgttcatcaaagtgttgttttataacaggaagtctcagaagctggataaatatccaacctctggaaatggttagatagaatagtatgtagccattagaaaattatgtctatggggtttaaaatgtcatgggaaaacacttctgacataaaagagcatgagaactgtatatttagcataatcttaactatgttttagaatgcacaggaaaaaaatgtacaaacatattcatagtgatgtctctggtggtaggattatgatcagtaagtacttctgtctcttcatattttcctgtatttgataatacatgcatatgttgtttttaaaataagaaaaattttaagtttaaaattggagctgaaaagtgtttttaggtcaggcgaggtggctcacacctgtaatagcaccactttgggaggctgaggcagtcagatcacttgagcccaggagttcgagaccagcctggccaacatggtgaaaccccatctctactaaaaataaaaaaattagccatgtgtggtggcacacatctgtaatcccagctacttgggaggctgaggcatgagaattgcttgaacccaggaggtggaggttgcagtgagccaagatcgtgccactgcactctagtctgggcaacagagtaagactctatgtcaaagaaaaaaaaaaaagaaaagcctttttaaacagtagcagacataactatataatccttactaagctgtcggtcaaatttttatttatatatttattttattcatttattatttttagacagggtctcactctgttgcccaggctggagtacagtggcgtgatcatggctctcttcaaacttgacctcccgggctcaagtgatcctcccatcttagcctcccaagtagatgggaccacaggtgcataccaccacacctggctaattttttttattttttatttttagagatggtgtttactatgttgcccaggctagtctcaaactcctgggctcaagctatcctcccacctcggcctcccgaagtgctggggttaccagcatgagccactgtacccagccctcaaatttttaaaaatctataagagacattattggacaattagagaaattcacatatggacttataatagtatcagagtgtgtggtgtgatggttctggagggaatggactttttctttggagacaggcttttctatgcccacccttttatcttgctaacttatcatcatccaggttccagcagaaacattacttcccccaggaaatttcttaagggtgcagtatcatgatgtctgcagcaaattctcaaatagctcaggaaaaaagtacgtgtgtggtatgagtgtgtgtatgtatgtgtgtatatatatacacatatatacacatatatatacatatatgtgtatatatatacatatatgtgtatatatatacacacacatacacatatatatacacacacacatacatacatgtatttttatataattatatatgcagagagtgcaaatgttgccaagttaaagattggtgagtctaggtgaagggaatatggtatttattgtattatttgtgcaacttttcttaagtttgaaaattttcaaaacaaaaaattggaggaagaaggcatgccagtctaccccaagccctccattggaatgctgaaaatctaaacaatgtgatttggcaatttcatttcttttctgttgtgggccagtagtccttagatgttggggaagggggtagtcgctgaggtgtggttgacttaggatggaagaagcagaagtcaagactcccagggtcaaagtggtttgctctgctgacccaagtgtgggaggcccagagtcagcgtttcaggtgtgctaattcagcatggttctattcacggccaaagtccaccctgggcacctctctggcagcaatcttgggtgactctactaaggccaggcctccatgaccctatgtctggatcccatatctccacctctcccactgtctcaggaacggtgcttagctttttcttttccctctcctgtcttctttgccagcatgtagaaagtttaaataattcccctctttacaacaaaacaaaacatacccccttcagtcaaccaccctagctctcttctccttttcccagccagatttttttaaaagcatcctaggccaggcgcggtgactcacgcctgtaattccagcactttgggaggccaaggtgggtggatcacaaggtcaggagatcgagaccatcctggctaacatggtgaaaccccatctctactaaaaatacaaaaaagtagccgggagtggtggcaggtgcctgtagtcccagctactcgggaggctgaggcaggagaatggcgtgaacctggtaggcggaggttgcagtgagccgagatggcgccactgcactccagcctgggtgacagagtgagactccgtctcaggaaaaaaaaaaaaaaaaaaaaaaaaagcatcctcagcactttggcaactccatctcctcccaacatgtccctgttactggaatccagccaggactcagccccgatctttctactctaaccagttgtctcagttaacaaggacaggtttatgctgcagtgacaaacaagatcccaaattcttgtggcttcacacatctggcaccacctcatcttccagccttaggagtcatcttttagttccttgaaaactctttacagttttctgttggggccttgtcatatactattcccctggaatgttctttcctatcccctccctttcaccttgctaacttgtgcccatccttcaggtctcagcagaaacatcacttccttggggaagttttctccaacacccacactacacaggtgtcccatctacactcctatgactttgtggtacttgtctcacttcattttccactgccttccccacaaggcacctgcacaagggcaaggaccgtaccactgtacctatgtcactcattgctgtggtcacctgcactctggctgcctaccttaactacacattagaatcacctgaggagcttttaaagccacaatgcaagactccaccctaggccaattggatccaaatccctggggtagggccagacatcagtggagttatatatacatatatatattttgtttgtttgtttgtttgttttttgagacagagttttgctctgtcacccaggctggagtgcagtggcgcgatcttggctcactgcaagctccgcctctcgggttcacaccattctcctgcctcagcctcctgagtggctggaactacaagtgctcgccaccacgcccagctaatttttttgtgtttttagtagagatggggtttcaccgtgttagccaggatggtctcgatctcctgacctcatgatctgcctgcctcatcagcctcccagagtgctgggattacaggcatgagccactgcacccggccatcagtggatatatttttaaagcactgcagagaattctgttgcatcagcttgagaaccactgatctgccttgtgcttcacatttaaaacttttttttaatgaataaataaaccccaaaaaattaatctccctaagcctccctagaagataggatggtaaggatattttcctaggtaaaaatatgttaatttcatatttcatgaaatttcatgtttcatttcaatcaagctctgtcatacaccttacatggggcaagcccagtgcctgggcagggtgtaattatactcattacacaggcaaggaaaagtcacattaggtgatggagcacaaataggcagttaatggtttcagggctagttaggatatgtttgtctttcaattgcaagtaatagaagcccaaagaaattggttatttatataatataattgattggttcccaaatttgaaaaattcaggaatagacccagcttaggtacagctggatccagtcactcaaacaatgtcacaaagaaccctttgacaggaatgtatcctgtgttgactctactttgctctgagtagtctttccccaggtgatgataaaaatggtcatcatcgccaggcttgtgtcctgtttagtaggaatatacaagaagagctcagtaaatgctggccccaccactaagcaaaaacaaaacttttgttgttgttattgttgttttaaataacagcttagacctttcttctttccttgttattctctttcatctgtaatccagttttctacttctgaagtatagaatgttctgatgatttattcttcattacccacaacttgcacatgtttatttaaaaatgccaggattgcctggccgttgtgtgctgttaacctttgtttgctgttagtggatccctgaagttcaggctcccaggggagcagataatgggtatccagttcctgcaatatccaccctctggcaagccaagttccttcctgggtaaggttttgcctacctgcattcctagggaagtttctgggcctgaccaccaagccagctctgagaaggggtgcataagccccaccatgctttggctctgtccctatagaatattttatgttgttactgaaaactaaaggaagatgggtgcggtggctcatgcctgtaatcccagcactttgggaggccaagacagattgatcactcgatgccaggagttcaagaccagcctggccaacatggtgaaaccttgtctctacaaaaacaaaacaaaacaaaaattagccgggtatggtggcatgcacctgtggtaccagctactcaagaggctgaggcacaagaatctcttgaacctgggaggtagaggttgcagtgagccgagatcgcactactgcattccagcctgggtgacagagcaagattctgtctccaaaaaaaaaaaaaaaaagaaaaggaaagctaaaggagagagactaaaatgatatcaggttcctggagaacaaacagacatgattttgcttcatggcaggacagccggaagaagtgggattatatcctcacattacaaataagaaaactgagactcagaatggttaagtcacttgtcccaggccacacagccagtaaattacagaaacagaatttgaacccaaatcttccagctccaaagcttgtgttcttttcactacctcctgcttaattttttaatttctaagattagacccttcatctatccatgacacctgcctgtcatcccctgaaaaaaggtgaacgccgttcagaaatttttctagcctgagctcactcccagttcacttatttttgctttgtcatggctgcccagtccccacttgtagaccaggaataggtcatggctgcggggactacacgctgtcgctgctgcaagggccggcctctgtttccggggctgagtgggggccagacctgccaggagcaccatcttctgtgggtcctgcctggatgtcacatcccggccccaagaagtcactgcaaaccttcgtattattgagcttcacatcctagaatttgctgtcactgtggctgctgcatgaagttgtcctgagagaaacgggcattgtcattaacagggaaattgatggtctgggggaaaagtcatcctcattctcttgcagatctatgggtgattgagactggctgatgttgaaggggtttctcagccatcgtgtgccatgttatggaacagtggtgtagccagccatttgacacccagcgctgacctttgtttaacaacctcacctatatatgacaaaatgattgtcagaaataatcgtgtaatgaaatgactgtaataatggccagaaaagaaacgcagatagtaaaatgtttctcttgttgaactctgtacatataattgcaccaggatttttttcaaataaaaagtaaatattatactacaaaaaagggaaaaagcacaagcatttattaaatagctttctatatctttctgagttttgatcctttgattgcagactgatgtaatattttatgtaaatcattgcttggttactaagtgaactttaagaaaagtgagacgtctgcagaagttgcccataatttagcagctactgtattgtaccattgatgtacggctttattttcttgattaattatttaaacaatataattcacaattttaaaataataaatttccacttaaaatggtatttaaactcagcaaaatatatcatctatgagtaaaatttgtatttaccaagcaaaaatattacagtttgtggttcacatgctgtctcactgttttaaattttaaatacaaaaactccaagtaggctgggtgtggtggctcacacctgtaatcccagtactttgggaggctgaggcaggcatatcgcttgagttcaggagttcaagatttgcctgggcaacatagtgagatcctgtctctactgaaaacaattagctgggtgtggtggcacatgcctgcggtcccagctactcaggaggctgagataggaggatcacttgaaccctgggggacagaggttgcagtgaggcaagattgcaccactgcactccagcctgggtgacagattgagaccctgtctcaaaaaaagaaaaaaaaaaaagaaacacaaaaactccaggtggtcgcacagaatgacaggactgaagtaacttagctccaatttctgtcttcataatcactgtcctaccattgtctgtgcttagaatctacttgcttaatgcaggaacatgtgttctcacagagatggaaaatgcaaatggcgccagaagcaagctggaaattctgaaccattaagaatttactctctgccaggcacggtggctcacgcctgtaatcccaggactttgggaggctgaggcaggcagatcatctgaggtcaggagttcaagaccagcctggccaacatggtgaaacttcatctctacaaaaatacaaaaattagccaggcatgatggtgggtgcctgtaatcccagctactcgggaggctgaggcaggagaatcgcttgcacctgagaggtggaggttgcagtgagccgagatctatctgcaccattgcacttcagcctgggagacagagtaagactccatctcaaaaaaaaaaaaaaaaaaaaagaacttactctcaaaataaatacgtgtggctgactccacatatggtagggccaactgtataactagaagttctccaaataacttctgtggagaaaaaaaagtttattaaaggttaacttttttaaagtgctaactagaaccttactaacactgagatcgcaccaattgtttataacttagacagggccgggtgcagtggctcatgcctataatcccaacactttgggaggccgaggcaggtggatcacttgatgtcaggagttcgagaccagcctaaccaacatgatgaaaccccatctctactaaaaatacaaaaattagccaggcacggtggtacacgcctgtaatcccagctactggggagggtgaggcaggagaatctcttgaacccaggaggcggagattgcagtgggccaagatcgcaccattgcactctagccccagcaacaagagtgaaactctgtttcaaacaaacaaacaaaaaaaaaaacctcttggaccaggaaaatattttttaagggaggagtattttatcactggcattgtttaggattgcaggcacatgatgctaatgaaaagcagactaactattagttggttttattactgtttttgaactctctctctcccttttttttttttttgagacagagtctctctctctgtcacccaggctggaatgcagtgactgcagtctcagctcactacatcctctgcctcctcagttcaagtgattctcgtgcctcagcctcccgagtagctgggattacagggcaccacaccaggctaagtttttgtatttttagtagaggcagggtttcaccatgttgcccaggctggtctcaaactcctggcctcaagcgatctgcccatcttgacctcccaaagtgttgggattacaggcgtgagccaccgtgcctagccctgtttttgaactctctagagacagtccagccccttattacttgtcctgaggcagctgctcccttcacctggccccccgcattgtgttccggacccttgtcctggtggtgctaaagaatatctctgtcgatcctttggggactggggaaactgaggcccagtgccacgcgatgccatttgttcagggaagattaggtcatctgctaggtccccagtcacttgaccttcttcccagacaggaagaagctgctctgggtctctcagtgctccacgtgtctttgcacattgaaatgttttctgatttttttttttttttttttgctgttacatttacttttaaaaaataacaagcaataaaatgttacatttgagaaggttgaaatgagaattgatttgagttaaattctagcagatttttcttagaagaatgatatcatcatctccagctacctgcaattgatctactctgaattaagaaagagacttccatttgttgtttatattttgcactcttgatgtgtttctttaaattatggtcatgggccaggtgtaggagctcacacctgtaatcccagcaccttgggactctgaggagggaggatcactggaggccaggagttcaagacctcgtctgtacagtaaattttaaaaattagccaggcatggtagcattcacctgtagtcttagctacttgggaggctgagatgggaggattgcttgagccagaactttgaggctacagtgagttattttcacgccactgccctctagcctggctgacagagcaagacctgcctcaaaaaaataagtaaaaaataaattaaatttcaatcattagcagtcattaggatatttaaatacagtatgttgaatcaaagttacgcatgtgtgtatttttttttccagagagttgtttatcatgtgggttttaatttaactttaaaaaaatgttggctggacagttgcccaaatggtatcatcagccatttggttgagaacgtatgtcctgcgggctcctctgtcactggagttttgctagctgacagccactggctagttagagactgcagtcagcacagatgcaggcgtggacttgcgcacgtaaccatgtcaatgcaaagccatcacttcttaaaaattctgaaccctgctgtctgagatggtggtgcagcggatagaactctgctctaagaggcagtagctaattccatgtcttctttgcccttgactagctgagtgactttgcacatggggcttgcctctctgttgccttgtctgcaaagtggaatcatcttttccttgctagacagaaggtggaccctggacctatggcctttttgagtttcccccccgcttcttagaaggacctctgatcctactgagtttaatacccacgggttaataattgggaaaagcaaaggaagcgcttctgtttaggtaattatatgcatgtttttgtctttttctggctggaaagatatccaagccactgggaaggtccgtggctacccagggtagccctctctggggagggctgctatatccaagagcccctcatgagaatttgaaaatcgaccatggtagggcctgctgacttttgacagctaatggtgtgctgagaattgtccctccaaagatgcctttccattccctcgggagagtctgggcagcccctactgggggctgggatgctggctcttccctcagcctccaccccaactgctctcttccctcctcccctccccagccccctaatttctctcacaaggctttgttctgcagcaacctttcctaatgcagtcctggcctcttcgcagcttcattacataaccttccgtggactcctggtccaaggatcaccccagaaagccagtcagaggtaggcacgcagctggggtccatttacttaccttccccaccccctcggaactcagaggtggtgcaggaatttggactccaagaattaacagctccaccaccatcaccagagccaaaactcaggatgcatgtgcttcatctgctgcttatttccagctgagagccagtggtgccatggttccttagggagccggtcccctgatgccggctcctggccccaaatctctctgatccgggctcttccagaatgtcttgtctccaccatcgcctttgaccaatggtgtccctttgcctggtaatgtcccctttgcctgatgatggccctgtcactcctctctttagcacagaggaggctgtttcatcccttcaagcctgccctcccttcaagtcttagctcaagttcaccttctccgcagagccttctccaatcttcttgactacgtctcctctcagctccagcaacctctgtctctggcactgattccttacttagctaagagaatcacagacacttggggctcaggacaatctgctttctctcttcttacccatggccttggactgtgtgtacctctttgtctccactcccaaacccaacccccagagggcagagagcatgttgtctgtccctttgctcagcatgaagccatgcgtgtggtagatcggcagagttccataacttgtgttgaccgaggggtcactttgctctgaaattacccctgtgtccttcagtatttgcacagatagcttcctggccagaccgaatatatccaagggcatggcccacctctgctcctgtttccaggtccctggtgggggttagttcatgccttcctcataatctgcccactggcctggtcctcaaggtcttcccaactgctcagccagagttgagaaaatgggtcgctccatcctgtttgtgtcgttctctccttcctggcccactctcctgcccacaggtatccaggggctgcctgtagcattagaggacatacatgcacatgcgtgggcatgggacactcacgtagcctccaagcacagcatcaataatgcattctgtgctttatagcatggaaagctgctctaaactttattacacagtggacatgtctgaagcagctcccaaatccacccctgagtgtgttggaattggcaagcctatcacttgggagtctagtttttttgttcgttaataatagatgcttcctgtggccccagcttggcaattttgatttaaagtgatcttaactgaagagactaatggacgggtctgaatttgtgccttttaagcacaaagtattgctcttaattaactggattctatcctttgagcaggcagaggccttcccccaagggcgtcattaacgatccacatctggacatcttccaaagccttcttctgtttcaggccaaccgcaggtgtgttcctgaacacccaggaggctatgagagccacatatgcctcccaaatacacacagtgtgcatgcccagggacatagagcagtgtgcaaagtcccattccatctctctccacctgggagaggatggctcttctgtctgattcatggctcaaagtggtaaaggagctccccactccccgtcccacgcctactcagagtctgcaaatatgtatgcgatatgagagctcgtcagttagctgtcttcagtgtggcgcacatttgaggagtctgactcccctccagcacaggccaatgtgcactgctctcctatctttgtacccccactgttgcactgtgcagaggttggagccatagaagtaccagagctgtgaaaggagaggccccctctcacctctgccctggtctccatccccactttctctaggaagctagtaggtgctgacaggggagagaagggaggggaggggtccagaaacagtggctcatgcctgcaatcctagcactttgggaggctgaggcaggaggatcatttgaggtcaggagtttgagaccagcctgggcaatgtagcaagaccctatctctacaaaaagaaaaaatgtaattagctgggtgtggtggtgggcacctgtagtcctagctacttgggaggatgaggtgggaggattgcttgagcccaagagtttgaggttacagtaagctgtgattgcaccactgcactccagcctgggcaacagagctgagaccctatctcaaaaaaagaaaaaaaaaaagaaaggagagagagagaaagaaaagaaaagaaaaaaaaaaaagaagggaagggaaagcccagaagagtgtggggagaggaggcggccgtcattctggggccctcagtgtgcacaaccagataacacatgctctgtgggcttttgtaccattttgcttgagcataaagaaaggaaggctgcccctaaatagaaagcactctggaggcaaacaaatctgactccaatcctggccctgccactttcccagctgaggacttagacaagcaccctagcctcttggacattctcagagccatctgctgcaagtgggtgctgccatacccaccttactgggcaggcttgggggaccaagggtggtaaatggctcagtctttcatgatgcggccacacagcaggtgcgccatccaggtccatttctttccttcctttcccccaaatcaagttgtcattaaagtactagtccacattaatgaaatcaactgtattaattttctatttgctgctataataaatcatcagaaatttagtggcttaaaccaacacaaatgtattaccttacagttctggaggccagaagccctccataggtgtcactgggctgaaatcaaggttttggcaaggttgcggtcctttctggagggtccaggggagaatccattttcttcctttttccagcttctaaaggtttcatgcattccttggctcatgatcttctatagctatagtcagaaaaattttccatcaatcatcttcaaagccagcaatggcaggatgagtcctcacatcaccttgctctgacaccagttctctgcctccctcttccacatgtcaggaccctcatgattactttgggctcactctgataatctgggatgatctctctattttagagtcagctgactgggaaccttaattccatctacaaccccaattcctctttgccatgtacagtgacatattcacaggttctggggattaggacgagcctgtctctgaaaggctactttacatgaaaattcatttttttaattaagatttttttttcctcttgagacaaggtctcactctatggttcaggctggagtgcagtggtatgatcacagctcactgcagcctcgacgtctctgggctcaggtgatcctcccacctcagcttccctagtagctggaactacaggggtgagcccccatgcccagctaatttttttttttttttttttttgagacagagtctcactcagtcacccaggctggtgtgcagtggtgcaatctcagctcacagcaacctccgcctcctgggttcaagtgattcttgtgcctcagcctcccaaggagctgggactacaggtgtgcaccaccacgcccgactaatttttgtatttttagtaaagatggggtttcaccatgttggccaggctggtctcaaactcctgatctcaagtgatccaccaacctcagcctctcaaagtgctgggattacaggtgtaagccaacatgcccggccccagctaatttttaaatattttttttgtagagatggggttttaccattttgtctaggctggtcttgaactcctgggctcaagcaaacctcccaccttggtctcccaaagtgctgggattacagcatgagccactgcactcggccttaagagaagatttaataattaatactttacaacaagatctggaagaggtgggatgagtaactaaatgaggatacaagtaacccgggtcatatttgctaatacccttggtcacattgaacttgatatcttatcagattttcctaatcagctcctttagcagcagtgttgcagcatcttatctcattttgttttttgtttttttgcctagcacatgcctgtaaatcactggattgaggtgtttagatgtttgttgtcctttggatgcttcttataaatccatatttcatggctccctggaaagtgctatgcaaatgataagctgcaaggatggaaaggaaattgcagtgctcctgaattgtaaatgggcttttacgaggaggtttctaattactcgctctttctcttgaactgaggagttgaagtgtaggtggcagatccataacagataatcatgtgtgtgatgtgacttcagcctgagcgtcgaggaccaagtcacagagcaggaacagccactctccagtgtccttggggctacgtctgaggagaacctgggatttcatatatgacctgcactggctggggggctctcttgacgtaacgtgttccctctgagcatgttacagattctgacattcttatgttccttctgtggagagacatgtacttagtgacctaactcactttagcatatttttgctcatcgtttgtgtagcttaaaggaatcagataattaccccctccccactactttcggaagcacaaatgcaatgccctagaattgtactggggactcaaaaagaaaagagagtagtaaaatctattaaaggggacaaagacagcctatatactacaagctttctatttttatggcagagaatgccattttctaagtaaacagagaactgcatttgacctgcaatatcaaatgcatggatttgatgctttggaaagcaactgttttctgcgttaatctgggtgtcttccgtgaaatgtcctcctgcctttggcttaaacactagctttgtctacagccattccatcctgaacctgcccaatcttgtctgaatcctggtttcaccactgacaagctgtgtgtccttgggcaagttacttcacctgtctgtgcttcagagtcctcatctgtgagttggggaatctggacagaatctaccccatagggcgtagtgaggatgtgttgaattatcccaagtggctacacagagtaagcactcaaatgatgtcatcgttgtcatgattgctgttaccagagcctagagttcattctgatactcgagtctgtggcccatccagcccaggtaaggaatagttggaggagttgggcatgttcagcttgaagaggagacgacaggggatatgggatagttgaatctgtgaagggccccctgggatgaagaactggcatgttctgtgtggctccagggcactgagcaggacccatttgccaaagtctcagggacacagtttctagctatagacagaaaaattttctgtcactcagaggatgaaaatagaatgagcccccttaagaggtaatgagctccctgtcattggaaggattccagaagagctaggtaaccactttaggtgctatcaaggggcttttttctttaaagtcctttccaaaagcttctgagattgcataaacaataggaagccatcttggtgctttaacacaaactctccccagtgatgagggttgagccaaagccagattggcaagcagagaggagacttgtgtacaaggagttcctcgagtcaattgctttttccttgttctagccagccagagggctcctgttggaaaacaggagaccggagaggctgaggcctgaccaaaccagcttctgcaggccagctgggaggccacaactcctacctacgggaaaactgaagggcatctctatttttagattagcaaaagaaaataaatttaagtttgagtctcctttgcaacttttaaaagacatctttattgagatgatcattcacattctataaaattcccccactttgagttacaattcagtggttttagtcttccttgatgattttgatggtcttttcttaaggctcttggaagacccagaagcctctcagacacaggtgggtgtggagggcgtagcacagaggcagacttctcatttcctgggtctcccctttaatgactctcagagacccctccttccccctgcccctggcttctaccccaggggtgtagagttttgccattttccaagcagaacttcatttcctcttctgtgtctacactctttgtgcttctttcttgccagctttttctcctttgcccgcccttccttccttccttccctccctccctccttccctccttccctctttccctccttccccccttccacccttccccccttccccccttccctccttccttccttccctccttccttccttccttcctgccttccttccttcctgccttccttccttcctgccttccttccttccttccttccttccttccttccttcctggtatgtgactaatttctgtttcaggacataaatgttgtccaggctgttctttggtctttctgttggataatggacatttggcattgagagaggctgctttttctgaaatcatgttcttggggcccagaacctaggtgtgtgcttctgactttgttttcttcctgatccaaattctgatatgtccatttaaattgatctagacccacagggcactgtgggacagatcctcagtggaacatgactctgtaacgagagcattttgttttgtcaaaatgagaacatattattgcctttcatctgattgtaaacataatacatgtttataaaacagtataatgagacaaaaatgtagacactaataagggaaaatctccctaattgtatttctcttcacagagaaagcccctgttgggcatatatactctagtttgtttatttgtttgactacacatatatgtattcttttcttatgtataaaaattctgaacatgcacatttctgcaactactgttttcacttgatgatgcatggacctctctagagtgtacgtttcttcttccttacaaagcagttggcttcgcccagggtacaccaggacacggttttggctctgtccccagggtgtcacgggaccaggggatgatctcacagggtctgccatctgccctgcctggccggaggctgcatcgagagggccaaggggcaccacgtgtcgtgggtactgtcaaacaagagccttcagagccttccacagtctttcttttgcttcccagcattgcttccccgctggtggactctgaatctagaactagctccaggcgcctctccaaattcagacgggagctggggcactattataatgcaaatctaggcaaagccctcccaataccaggatccagaatggggtggggccctttgccctgaaaagctgtttagtttgaaaatacaaacaggagacagaaaagtttggctaaattaatggataaagttttaacgatggtaaccatagtagggttcatcgacagccagcgatggttctgaacacttgacatgtattaactcacctaatccccacattttacagacaatgcaaaggaggctctgggaggttgagtgacttgccccaaagtcgcacagctcctaagtgaaggattcggagtggactccaggcagcctggtctgactccctgcactgcgctgtgcttatctctggccccaatgccgccatgcagaagtgtctgggggcactttgtctctgtcagacagaattcggagatgtgtatgcttgccctggtatggcacttctctttttttgagacagaatctcactctgtcaccctggctggagtgcagtggcatgatctcagctcactgcaacctccgcctcccaggttcaagcaattcttgtgcctcagcctcccaagtagctgggattatagatgtgcaccatcgtgcctagctaaatttttgtacttttagtaaagatgttgttttgctgtgttggccaagctgatctcgaacttttggcctcaagtgatctgcctacctcagcctcccaaagtgctgggattacaggcatgagccaccatgcctggcagtgtggcacttcttacgtgtgttcagcggacactgtttatcttctgtccctccaagacggtgctgagctcaggtcgttcattactggcagacaactgctgatttccaacagaattgccatcctcttctcccctgcgactttcagagtgtgacctcagactcaaaaattagaagtgaaaacatcttaaaaactatcaccttttcttcctaatcctcctctcccctccctgtcttccttgttgtccccatctaatgaactatcatggcaaaaagagcccatttctggtcattttctgtggcctttcaaactcccacctaccccactgctcctgggtgcattacccgaaagctgagacttcagtgcagaaagtgccaggccctctgtccccccagatcgccttccttgtcttccctgtgcttgcctgtcacattgtgtgggttccagcgctggaaggaatgaggaacagattctctggttctccttttgaagtttaccttcgctccaccacttctgagaccttcccggaagttgccccttgtttctctcctctccagggctgccccagagctgcctctcacctcttcctgctgtcaccccaccaccatcagggcagaagttgggacaaagcctctcctactggctcctgcttttctcccttaggtccagcctcctcttctccatcttcaggagtctccttctccactcacacgtcatgacttcagcacctcgcatcagtccagaatatgactgcttgttcaagtgccacctttctcatgcatttttttctagtgacaatcacagccaccctgtggggcaggagtgtcatcatccccatgtttcaaatgaagaattgcagttcagagagggcaagtgactggcccagcctcaacagctagccagtggaccccaccagggcttctgactccagtccgggttccctttccacccaaatccatggagggagctgagccgagaacaggtgtccttcaggaagacgtgaagccaaagcctccacctccaaactcaggggcccagggagtccaggcacccatccactcacaaggctggatatggtgcattccaggagaggggttgggggcgagtggcctctctgtgtacccgtggggatagatgcgcaagtggcatcgccacatcgtgagtcctggcttcatgggtgagctccaggtccaacgagaagccaagcagggggcccttcaagctcagctttgggcccgggtcggggtacagggtagagcgggcctccccagcccctgccatgaggccaaggcagtgcatcgttcgcagcgtacattcagaaaccaaagcctaggagctggttatcattccggtttacagctgatggaagagcaggtgcttccgagaacccacagtgctctttggccagtgacccaagggtgcctctgagaggcctcgcagcacccggaggtgctgctgaggcaacgccctgactgtaagaaggaccattcatcctcagagagtggccgtgatgctgctgcgacagtcccaccatccctcccgactctcactcccaacagacttcccactgtaaagctgaactctccagcaaatcacctctcgccagactctctcctcactctctctgggtccactagaggttcctcagcctctctttgccttggttttcccagctgtaaaatggagcaaagagggcctatgtacccacaaaggtgtggttggagcgactcctcctacattagggcctcgagtggggcttcatgattggttggtggaggtctccaaacccacccagtgccaccgaaggctgagactgcagatgcaatgccacaggtgtccttcctcagcctgggcagctgaacatcatgtgtaaaacggggataataagataataacagccccttgcacctatgtggctgtgaggattaaacaagataaatgtgtaacagtgcctggctatagaaatatttactcttgttattaagggaagaatatgtgtggctaaaaagggatcgaagatgtaaaagccaatccctccccctctagcatatttaagggtaatgttgagttggtttgtggaccatttgctgcctgttagagctggaaggtagggaccccctctcaacagcgatgctacaaattatacccattggaggtcaaccaaaagacaaagcttattggctggacatggtggctcacacctgtaatcctagcactttgggaggccaaggcaggcggatcacttgagatcaggagttcgagaccagcctggccaacatggtgaaaccccatccctactaaaaatacaaaaattagctgggcgtggtggtgcacacctgtaatcccagctactcaggaggctgaggcaggagaatcactagaacccaggaggtgaaggttgcagtgagccgagatcgcaccactgtactcaaaccgaggcaacagagggagacgcaatctcaaaaaaaagaaaaaaagacaaagcttgttaataccagcatattgttaagggaataaagtaggctgcagaacaactggtgtaatatggtgccatgtagggaaaattacatgtgtgcataggagaggggtctgcaaggttgtgccctaagatgttagagtggttcctttgcttttctcttttataattttgtatttgacttttaaataaggaccataaatcacttttataaaatacattctctccagcccctactactcctttaaagaataagagtggtttgcccaagaaagacagttttttttgctctggttttcttgattctgacatcagaggaaactccttctcatccacttggggctctgggttcaggggattcatttcaggcagattaaagtggtgaccaggggcattcgtggacacagggagggacaggagcaccatcagtttgtctcacacaaccactgtcatcctcactgaaggctgttgcctgatcaaaaacagtattgggccaggcacggtggctcacacctgtaataccaccactttgggaggctgaggtgagtggatcacttgaggtcaggagttcgagatcaacctggccaacatggtgaaaccttgtctctactaaaagttcaaaaattagccaggcgtggtgggtgcctgtagtcccagctacttgggaggctgaggcaggagaattgcttgaacccgagaggtagaggttgcagtgagccgagatggcaccaccacactccagcctgggcgaccgagggggactctgtcttaaaaaaaaaaaaaaaaaaaaaaaatatatatatatatatatgtcaaaaatggggtagtttttagatctatagtagttctaaaaacaaaggccatccaagcatgacagatttacaagcactattggctattccagtagttacaatggaggagagaagcttttagttaaaacaaacaaacaacacaacaaacccagaaaccttaggtcaaaaccaaaattgtcctctcagacacaatctgggaattttctcatgacagtgggcattagccaactgacatcagcagcaaccatccgtgtgcacacagtggcaccacctcctcccaaaaagcagccttcatctatgccctcatacaatcgttgattattctctttggattgaggcccggaattatttaagtttcttcttgccagcatgagtctttcctttctgtatgctccttatcttctctctttaatttggcagttctgcttgaaatctgggtctttcattagtagtagttcaatttggttccagaacattctgtggtgtgatgcaatgtgaccagagctcacacttcagagctcttcaagggccagtcttactgagcacctcccagtggctgcctgtgtgctgggcgccacttgtggtgggcaggagagaggaggggacacaaaaggagacacagctccttcttagaagctcaaagttggggaccagctgccacagaagagtatgtttagcatctgagacaccaagatccagcgtcacaagggtgtttattaagcctcctcatctctttctttttctttttttttttttttttcctcaggcagtcttactctgtcacccaggctggagtgcagtggcatgatctcggctcactgcatgcaaccaccacctcccgggtttaagcaattctcctgcctcagcctccccagtagctgggattacaggtgcccaccaccacacccagctaatttttgtgtttttagtagagacagggtttcaccatgttggtcaggctggtctcgaactcctgacctcagatgattcacccacctcggcctcccagtgtgctgggattacaggtgtgagccaccgcgcctggccttgctgttgattcatctatagtatgtttgacttgatgacctccagttaccttagacagaggttctcatctaagctccaactttccatttcctttgtcctcgtctttccccttaacccctccacatttctctcaaaatcaccccacttctaaaaaatactgtttatttttcttttaaatttcaaattatctatactcattgaaataaatcaaaatagcatggaataagcgaaaaaaatggatcccacccttccccactcccattccctagggctaaccatagttaaccatttaatgactaggtttttttgttgttgttattttttatttatttattttgagacagagtcttactctgtcacccaggctggagtgcagtggtgtgatctcggctcactgcaacctctgcctcccaggttcaagcattctcctgcctctgcctcctgagtagctgggattacaggtgcctgccaccacacctggctaatttttgtacttttggtagagacagggtttctcaatgttagccaggctggtctcgaactcctggcctcaagtgatctgcccaccttggccttccaaaatactgggattaaggtatgagccaccgcacccagccctcctgggctcttttcctttagttgcactcgctccccgctcctggagtagagggatttccgagagactgtgggctccagccttcacctaggcccaggactaggatgcctgccctaacatttatctttataccttaaagcaaaacagctggaccataagcattcaagaacaaactgtgaataaggagaaagttctcccaggaaacaagagctttagttatgttgggccagcccttatattccttagctgttaccagtcactgcttgatttaatctcggctatcacttggcctgacaggtctgctgctggtgccaggatgtctgggttttgaagcctggctccattacatacttcctgtgtgaccttgggcaacttactcaacctgtctgttcctcagtttccccagctgtattatgtcagcataatagtttgttgtgtgaattaaatgaggtaataactggaaatgcttcaaacatggttcctatcatgagaaatcctgctttccgcctaaatgtgctggaaaattcctggtggtgcagaacaggagaccagagcaaaggaaagacagggtgcagaagccaaaaattaccttggagaacaaagcgcatgttaaggttatttttggattctaggtttatctctgcttggtcttcagttacctacaagagatccatttaggggatttttgtttgtttttaacgatagctttattgagatataattcatatgccataaaagtcactcttttaaaatgtttccggtatattcacaaggctgtgcagccttccctgtccttgattccagtctgagtttttaactgaagggataaggaggaccacgctttccccagaccagaaccgcgggccagggggcgattccgctgagtcaccgcgggcgcctggtgcgcggcggcggagcccgggaccttccttggctgccccctagcgagggccgcagcgcagcctgagacacccgccggggccgctccacggccgtcggatttagactggaagctcggtccaggtccccagcttgatgcgcccgcggtgtaggagaccagcccgactcgagcttcccctgagcccctggactcttgactccagcagggcctgggtaatgaacgtcagctcccctttcccaaaggggttgctctgttgggaaggcacccgtttgatacagtagcatagagatgggttttagcatcaaaatatcagaattcaagccttgctctctgcttactagctgtgtgaccctaaaaaggtttctgaacgtctctgagcttcagtttcctcatcattccttctcacggggtggttgtgagcattacagagatcctctctgtgaagcccctgtgagtggctcatcctgagggctgaaataaacatgttattaataatccaaaactggcaagggatgttgactggtccccctcccttgcccaaggagctttctagaacctgagttatcattaccaaactgtactgccttgagtaagaaagttagaaggaatgggaaggatggtggcaggtggaggaaggcggattggtcatcacctccttgcagcaagaaacagccccagatcgtgggaaacctacagacctgctagacagactaggagcaaaagctggggctttaagaatccccagggaggttctcctgagagagtagccagttggattttgtaagcagagatttgtttggggaggaggtgacaacgtagggagcagaggggcaaagctgtcgggaatcctgccttgagggcagggatgtgtgttggggggagttgggtcactggggctcggtggccttgggcaagtttctacctctcaggtcctttacccacctagggtcgccatcctgcccacctcacaggttacagtgagcctggatgcactgtcatgggcaggtgcccaggaaaatggcagacatgttccaaacagcacgcagcattccccagtgatgcccagggtcaccttggaggtgggcgagatgcctggggtttctcgtccaccccacaacacctcaggggacagccaaagctgtcccttcaggtaagctgcacagaagatgtgaactctgctgcaaagactctattctttgggagcaaaagggacccagggtctcacctgcacatccctgtccctgagggcctaggggttcttggaggccccagccttggcaaaatgaggaagaaggtgaaggttgtctgggcccctgccaggctccttcctcggccacgcactccccttcctgcacacacacccttctccctccaccccatctccattgttgtcagaaaagtcacaataaaaaggtccatattgtctagttcccatacttttaatttttaaaattttatttatttatttatttatgtattttttgagacagagtcttaacccaggctggagttcagtggcatgatctaggctcactgcaacctctccctcctgggttcaagtgattctcatgcctcagcctcccgagtagctgagattacagatatgtgccactatgcccagctaatttttgtatttttagtagagacggggtttcaccatgttggccaggctggtctcgaactcctggcctcaagtgatctgcctgcctgagcctccggaagtgctgggatttcaggtgtgagccaccgcactcggctccacacttttcacttattaaaagactgtggtgtccatcaatggatgaatgaataaaccaatgtggactatccctcccattacccaaggaatgaagcacggagccgtgccaagatctggattcacagtgaaagaagccagtcaccaaaagccacgtgctgtgtgacttcccttatacgaaatatccagaagagatacatccatggtgacagaaagtagatgagcagctggggactggcgaaggggagaagggggagcagctgtctatgaggtccagcctttcttctgggtttggtgagaatgttttggaactagatagaggtgatagttgtacaacattgtgaatgtactaaatgccactgaatcattcattttaaatcgttctttacgttgcatgaattttaagtcaatcaaaaacagttgtttgaaaagagaaaagcctatgggtagcggcagcagtgattggatttatgattcgattccatggctcatccctcccctgcctcaccccctcgccctccgacgtcttcttcttttactctgaactgttatctttgttctcatctctctctctctctctcaaccctgcagacacttttccctttctttgtctgcccccaccctccagatttccgtgtctccagtgtctccctacgaggcatgaattgagactgggagggtgtgattctgaagaaggcaccaacagtgactcagctagccccttcccccaccccgccccccgggcctcaatttagctaaaaaaccacagggacggactcaggaggcaatacctttccaagggtccctaaaaaatgtcccattttagtgtccaggtttcactcaactttagtgcctcccctaaaatgtgttccttacctcccaccccactgcatctaagtcactgcctgagaaaacaggattgaggaaaggagaaaggaagagagagagagaggaggagagagagagagagggaggaaggctgatggatttagaaaagaagaaaacaagtggtctgaggaaaacagccttggtgtgtttattttcctgtctgtgtatcgcttctcggccttttggctaagatcaagtgtattttcctgtctgtgtgtctcgcttagattacagggatctgtgggtgatgacacgtctggtccaggctgcgtagtcacctcaagggcatgcttattgatgtgtttttcaattcactatctttgcatgggagtcccaggccaagaggcacagctgcgccatttgtctgttggtttagatatcctttatccagttcttccagagaaatcatcctgcccttctggaggaggtgggcagcaggggtcagagatgggagggaaaggaaggagccaggtccttggctaggatgccagggtcccctgcctctcacctggcctgggctggaggcctcctgctgtcctgtcactgatcactaccccgccccagcctcctgagttagaagacacaggctaaagtagagtatttcttcattgaaaaacccatacaaaataaaggttcataaaaaataaaaatttagactgggtgctgtggctcacacctgtgatcccagcactttgggaggccaaggcaggtggatcgcttgagccctggggttcatgaccagcctgggcaacatagtgaaaccccatctctacaaaaaatacaaaaaattagccaggcatggtggtgcatacctgtggtcccagcttctcagcctatggacccacatagaatacaatgtcagcataagaagggagccctggggtcaccaaatggtttgggcggcaaagaacctgaaggttgagagaagtggcttggttacccagctgttggatgtgagacctggccactgcttcttccataccctagacctgcaccctgacatctcaagtaaaaagttgggggatgttttatggtccaggatgaaggaagggcagtgaggggcagcggagcatcactttgcatttctgtctgcctcttactggctgtgtgacctggggcaggtaacttcccagactcctgggaatcataacacctatgatgatgatgatgatgatgatgatgatgatgatgacacctacctcaaggattgccctgaagggtcacagagatgcctgcaaggcacctgcatggagcaagcgccccttctctggcaggtgctgggtgagcactacctgctgccaggccctggggctatggcactgcgtgaccctgcaagtcctacctggcgaagctgtcgttcttgtgctcagtcagtgttggttgtaagactgagaagagtcacttcattttgctctccagggacatctttctgggtcctattttctgcctatgtcaagtagcgcctcaaggatgctcctgaaaatgggcttgtctttcttaacatggcaggtaggtcccaaagcattagcatggggcagctgacctagcccagccaatgcagtgcagtgactcttgcaaccgagtctaatcagaaggtccatgaacctacgagcatttcctgtcccaggatcagggtggaggctgagcctccctgcttagagattcttcccatgcattccacttttttccccaaaagaaaatattgacccttgagaggcacacagtttatttattttgcatagtaaatagtagcctgtattttaaggatgagttgatttctgcatcagcccctgtaggtcatcagccttctattggtgcatctgactctctctagccctgcagggatggtggagggggaggggaaggagggatctttattggaaaccaggacagtgagactcattgccctgtcatctgctctgtggtgctgaatgaggcagcccaacagagaaataccctgagcgagcatccccagcctccaaaacagtggcgcattgccctgagtcctgggaatgacctttgattctcctgctcctgacttggaacccatggaaacctctagaagcagctgaggaaaacccaacatgaaaagcagaactccacactgagaatataggaggtgatcggaacatacaatgattcttgctaagaccgattcacagtttttcttttttttcgatcgaagaaatactggagaagcctaaagaaggagtctaaaaactctggcacgtgggccaaaactgtccttgagctaagaatgattttcacatttttaagtggttgaaaaatgaaataaaataagatgatgttttgtgacacatgaaagctatgggaaattcaaattctaatatctataaatagtgttttatcagaacacagtcatgctcatttatttatgctcgatggctgctttcccgctacaattacgttgagcagttacaacagagaccacgtggcccacaaagccttacaatatttactatctggccctttccagaaaaaaatgtgccgactcttgaccttaacctcagcaatttgggaggccgaggcaggcggatcgcttgagctctggagttcatgaccagcctgggcaacatagtaagactccatctctacaaaaaatacaaaacattagccaggcatggtggtgcacacctgtggtcctagccactcgggagactgaggtgggaggatcgcctgagcccaggaagtcgaggctgcagtgagctgtgatggcaccactgcacctcagcctgggcgacagagcaagaccttgtctccaaataaataaataatgcaaagtaaaataaataaaaccatataaaaaggaatcaatttaaaattataatgaaagctggccgggcatggtggctcacgcctgtaatcccagcactttgggaggctgaggtgggtggatcacgaggccaggagatcgagaccatcttggctaacacggtgaaaccccgtctctactaaaaatacaaaaaaaaaattagccgggcacagtggcgggcgcctgtagtcccagctactcgggaggctgaggcaggagaatgtcttgaacccgggaggtggagcttgcagtgagccgagatcgtgccacttgcagtccagcctgggcgaaagagcgagactccgtctcaaaaacaaaaacaaaaacaaaaacaaaaaaaaattataatgaaagccaaggggcatagtagaacaaattttctagagctcattaagtcaaatgagtcaccagttagtaaaacgcagtcacggggaagagagggcaggattctttgaagcagcggctctcctaaaaacaacccacccttgtccagctgccttccctcctgagggtgttccctttgactgtgtgacccccatcccctatttcccaaccgtccaagcccacctctagcataatacgagcttttaatccctctccctgaccccaacccgattttgaagcccagtctagtattttctcaaatacacttcttggctccattccttcctttccatcacctctgccttttcactgcatgcttggaccactgcagtcagctccctatgaacagttgctctctacccatccaatcggccccgcctgctgctgccaaattcaccgagggcacctctgtggtgctgcctgtggacaaagtccaagccagccacctcacccacctacaggtgagtggggagcagccagcgtgtccagtggtttaccccatcgccacagacttggtgatgtgtcgatgtgcagagaaggggtgttggcagccacaacacaagcaaccccgccccatgtgagatctaagatgggcgtgctgggagccacctctgagaatccaacagaaggcagaggggagaacggctcacacggcacaaacactccttccttttttttttttctttttcctttttgaaaggagtctcactctattgcccaggcaggagtgcagtggtgcaatctcagctcactgcaacctccgcctcctaggttcaagcgattctccagcctcagcttcccaagtagctgggattacaggtacactccaccatgcccggctaatttttgtgtttttagtagagacggggtttccctatgttggccaggctggtcttgagctcctgacctcaggtgatctgcctgccttggcctcccaaagtgctgggattacaggtgtgagccatggggcctagcctccttccatttaaatgtatgcctaatttgcccattgagaacggctgagacgcattttaagtggccagggtctacttagagttagtgctcatgaccaggcccaggtcaagcctggctggccagatggtgcctttgacctgctctgtctctgtgcaaaggaatgagctgaaggatgggggtgcagtgtgtgggcagtgggctggggctggcaggactcagtgactaagggaagagaactttcctcactaccagcctgtcttttcagggcaccgcggggggctttgggacttggtgatgaacacagcacagagagctgtccagcatgcgggtccctggcttctcacacttcccaggctccttcagaggctctctccaaagggagctgctctctctagaacccatgaatttggaatataggcaaccactgcattggggaccactgacctcaaacatagagaccagagcaaatggggctcatcacgtgaaactcatctggaactctagcaggttcttttatatatatatatatatatatatattttttattattatactttaagttctagggtacatgtgcacaacatgcaggtttgttacatatgtatacatgtgccatgttggtgtgctgcacccattaattcatcatttacattaggtatatctcctaatgctatccctccccactccccccaccccacaacaggccccagtgtgtgatgttccccttcctgtgtccaagtgttctcattgttcaattcccacctacgagtgagaacatgctgtgtttggtttttttgtccttgcgatagtttgctgagaatgatggtttccagcttcatccatgtccctacaaaggacatgaactcatcattttttatggctgcatagtattccatggtgtatatgtgccacattttcttaatccagtctatcattgttggacatttgggttggttccaagtctttgctattgtgaatagtgccgcaataaacatacgtgtgcatgtgtctttataacagcatgatttatattcctttggttatatacccagtaatgagatggctgggtcaaatggtatttctagttctagatccctgaggaatcgccacactgtcttccacaatggttgaactagtttacagtcctaccaacagtgtaaaagtgttcctatttctccacatcctctccagcagctgttgtttcctgactttttaatgatcgccattctaactggtgtgagatgttatctcatggtggttttgatttgcatttctctgatggccagtgatgatgagcattttttcacatgtctgttggcgaactctagcagcttcttttcacaagttcatggagagaggtttcccactgagggaatcacatctgtctgatcaaaagaggcttgggaaatggctctcctgttcattccctgaaaacctctgatggaaccactgccactgtggcagccccagcactggcaccccagccatgattggtgccccagccacatctctgctgtgagccccagagccctggttaattaatcatccacgtgttgatggggagaggcccattcacaaaagcgacataaagcccagggagacgtggccgtggcaagaagggtgtgggactacattccgcccccaactgagagattcagaaaccagaaaaaaatggaaaaacatactgtgctcttgggtgggaaaactaaatatcatgaagggagcaatttttatagttttggcctataatacaattccagccgaaatcccagtggaactttgagaatttgcaggaaaaaaaaaaatgtctaaagtacatctggaagacaaacttacaagaaggtcaaataattttgaaaaagaaaatgatatctaagcccacctagagaataagacttgagatccaaagctaaatcaggaggctctagcaaaattgacagataagcaggacagagtgcatggtgcattcacctggggaagagggcagattggtctacaaataggcctgggtccactgactttagctgttatatttggggagaaacttttcaacctcactccatcttaaacctaaaaatattccagatgaattaataaatataaaaaattagaccactaaaaatgtagaagaaaatggatgatctttctataccatagagcaatggaataaatcacaaaggaaaacagatttgactatataaaacttaaaccctgcccatcaaaaaccatcagaaaccaaaataaaaggcaaccaactggagaagatagttgccacaaatatgatcaagggttaatgttattcataaattaagagcccacacaagtcattagaataagcactgagacctgaacagacaagcaaaaagaatgagagtgggtcggcgcggcggctcatgcctgtaatcccagcactttggaaggctgaagcaggcggatcacttgatcccaggagttccaacaccagcctgagcaacatggtgaaaccctgcctctacaaaagtcataaatattagccgggtgtgatggcacacgcctgtagtcccagctactcaggaggctgaggtgggtggatcacttgagcccgggaggtagagtctgcagtgagccaagatcacaccgctgcactccagctggagcaacagagtgagaccctgacttaaaagaaaaaaaaaaaaaaagaggagaaaaatgctgatctcactagtaattaaaacatcaggccaggcgcagtggctcacacctttaatcccagcactctgggaggctgaggcaggcagatcacttgagatcaggagttctagaccagcttggccaacatggtgaaatcccgtctctacaaaaaatacaaaaattcgccaagcgtggtggcacatgcctgtgatcccagctactcgggaggctgagacaggagaattgcttgaacacgggaggcagaggttgcagtaagctgagatcgtaccattccagtccagcctgggctacagagcgagactctgtcccagaaaaaattaaaacatcacatatttaaacaactctaggatatcatttaaaaaaacattaatagactgttttttagagcacttttaggttcacagtgaaactgagtggaaggtacagagacttcccgtatgttccctgccctccacgtacagcctcccccactgccaacgtcctgcaccagagtggtacacttgttacaaccaatgaatcctcattaacatatcattatcacccaagttcatagtttacattagtaaaacatcatctttcatctataagcacaaaaattttttggcatttatttaggtgtatgattaactcagtgttgacaagactcacacttcatacccacttgcactgcatctgagaagcaattggtgtctacagccgctacaccctcaacaagcccgatcttgtttgaaaagcaattggtgatgcttctcaaaattctatggacaaagtcagccgggcatggtggctcatgcctgtaatccctaaactttgggaggccgaggcaggcagatcacctgaggtctggtgaaaccctgtctctactaaaaatgcaaaaattacccaggcatggtggctggggcctgtaatcccagctactcgggaggctgaggcaggagaatcgcttgaagcaaggaggcggaggtttcagtgagccaagattgcaccactgcactccagcctgggtgacaagagtgaaactccatctaaaaaaaaaaaattatggacaaagtttttcaaaaagatatttaatgcaactttatttgtaatattggaacatctgaggccatttcagtgctaactattaggggatggttaggaaaatatggtacatatgtggaaaggaacatttggtagttagtgcccctgatgtttacaaaggcttttagtgaccaacaaatgctcatgctataatcttatgtgaaaaaagcaagtagcataattgcaactatatttttaatgcatagaataaaaggctagaaggaaatatcacagatccttgacatacattcccaaacctttgtaaatccgcggattcatgaaaacagacacatttgcacaagtgcctgatcttttctgttatacattcattagaagtcaagccctggtgccacaaagtatctgccttttcaaatgtgatcagaatgttctcttttgcttcaaggccatttttcacgaagcagtggcatttttgcctcttcatcagagtcaccgtgtgccctggaggactgagaacagcagagccgttttaggatgggacagggcagccaggaggattgggctcactccctactgagtgcctcactcccgtacagcccccatagaggaagaggggttcaaatttattcctcagccagatggcatgtgccgcctgtcctggaatttcacatcacttatgatggaccaaaattccaaaagctgaatccatgattgtcaaagtctggtatggcaggatgtcaacagtaatcgtttctgggcagagggatgattttctcttcccatcttgctttgtataaatacattttctataataaggttgtattacttttctcatcaagaaatagcaaagtactgttttactcaaaatatgaatagagccaggcatggtggcagcttatgcctgtaatcccaacactttgagaggcggatatgggaggatcactttagcccaggagtttgagaccagcctgggcaacatagtgagacccccgtccccactcccccaaagaaaacccacaaagcatttatcctggattattcacaggggccaaaaaaaaaaaaaaattcaggcctcctatagccatgagctacgaatatgaaaatatgcaaatgtgtaagaaaagccagcacatccgatttttacttttactttcacacctctgtccaccatgttccaagagaagaaacttggtcattgaaaggaatagatcaaatccaaagaacaaaaccactgtgctcattaaacttcttagtgttcacaaagctttagctgcaggttgaatggggcaacccgaattggctggctcacctgggctgcagggagcagagatcgcgacactgcactccagcctgggcaacaaagcgagactctatctcaaaaaaaaaaaagttcataaattcaaagttatgaattatttttaaaataataataatttacaataaagatgaggacaaagtgtgagtaaatggtggtttctatccagctctgttgagctgaagtggcatctccctgctggggcttttggggaagaagggtgtgtgttgctcttcagatcccaagcctcatgcccctactgggccctgtggggtgcttctcagcccaccaggagagccaccgttggaacacacacgtgggggacctggtgggtgccggtgtggtgaatgggggccacagcctgactccaggaagccagcaaactcggagctggaggagtcaggacacccccgatgagtcaagagttggttttgctgccagttgacatctgattgaaccatctcttcacttctccgtgcctcactttccttaccagacaggctctgctgatgctgtccctctcctgttcagtcgtgccctcaccgttaaagagaaagagcaaactgctgggcagcagcattgatttttttaatgaagtggaaagagagctgggaataacaagtcgggcccacctcacctgcctcacctggtgggtttatttgttttgtttttttttttttgttttgagacagagtttcaccctgtcacccaggctggagtgcagtggtgtaatctcagctcactgcaacctccacctgccaggttcaattgattctcctgcctcagcctccccagtagctgggattacaggcacctgccacatgcctggctaattattgtatttttagtagagatggggttttaccatgttggccaggctggtctcgatctcctgacctcaggtgatccacccacctcggcctcccaaagtgctgagatcacaggcgtgagccaccatgcctggccgtcacctggtggtgttgaatatgaactgctgcggtgttggtaaattaagcaagcagatagatgtaaataacgcttgggcaggaatatggagcacgggatgaggatgggcggccaactgttagagagggtagcagggaggctgagatctgcctgccatgaactgggaggagaggctcctctctctcttcacccccactctgccccccaacactcctcagaacttatcctctcctcttctttccccaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaaggttagtggacagccatgcacagcaggcccagatcactgcaagccaaggggtggcgggaacagtttgcatccagaattgcaaagaaattttaaatacattattgtcttagactgtcagtaaagtaaagcctcattaatttgagtgggccaagataactcaagcagtgagataatggccagacacggtggctcacgcctgtaatcccagcactttggaaggcccaggcaggaggatcccttgaggccaggaatttgagaccggcctgggcaacatagcaagaccccgtctctaaaataatttaaaaattagccaggtgttgtggtgcatgtctatagtcctagctactcaggatgctgaggcagaaggatcacttgagcccaggagttcaaggttgcagtaagctgtgattataaaactgcactccagcctgagcaacagagcaagaccctgtcaaaaaaaaaagaaaagaaaaaagaaagaaagaaatttaccttgagttacccacatgagtgaatgtagggacagagattttagggccttaacaatctctcaaatacagggtactttttgaggcattagccacacctgttagcttataaatcagtggtattgattagcatgtaaaatatgtgactttaaacattgctttttatctcttacttagatcaggcctgagtggcctctctttagcaagagttggttagccctgggattcttactgtagccacattaataaacaacatcgacttctaaacattctataataccatcttttggccaaattgacttcgcctcttcctctctctttccaaatgaaatgtgtttcatttcactgtcagaccacatggttggggaccccacagagcacacagccctccctctgccttcccatgctggcccttcacccactgctggagtgccaggttggtccaagggttggaccaagttgtctgaggttgtctcaaggttggtcgaggctgtctccgcgctgggttgtgctacaaggagcccttctttccatgggtgtggctggcagtgagtgctcacagcaacagcccacagtgcagcccgagggcaggatggactcagtccctgcctccatacccatttctaaggaggcaaaatggcaaacactctacttttctcttttaatgctaaaaataagaaaacaccttgcagcccagggtatgggtagtgcatggaagccgtggagttgtgaggtgggaagtgacctctgctggatatgtctattcaggaagattgctggagtgggtggggtctctgggaggtcccctgagtgtgggaagctgggaccaccagctttctcgcacagggagtggccatcccagcttggagaggttccaggactggttgggaggcacgtttcagatttctatctgttgaatcagcgaagatattggattatgaggaatttgggaattaggaaagtgggtgcaggtgggttgggggtaggtgaaggaagacatgggcgtattgggggagcaggggctgctcagaggtgttccagaagctctgggtgaggaggtgagagggaccggggaatgcagctcggcccagcctccctgcctgaggtcagccatcacgtggtgatggcaagatggaaatgtgctttctgactgctccagccagtgctgccagattcagctccccagggagggcacctgagaggctccaagccaggagatctgttttctcctttgttttgtttttttttgttttgttttgttttattatactttaagttctagggtacatgtgcacaacgtgcaggtttgttacatatgtatacatgtgccatgttggtgtgctgcacccatcaacttgtcatttacattaggtatatctcctaatgctatccctcccccctccccccaccccctgttttctcctttgaatccttcttagaggccgggtgcggtggctcacgcctgtaatcccagcactttgggaggctgcggcaggaggattgcttgagcccaggagttccagaccagcctgggcaacatagtgagacctcgtctctacagataataattttaaaaattatccgggcatagtggcatgcacctatagtcccagctactcaagaggcagaggcaggaggatcacttgagcccaggaggcggaggttgccgtgagccaagatcccaccactgcactccagcctgggcgacagagacccccatgtcaaataataataataataaataaatccttctcagtcccttcctcactgtgtccccctccactgaatttttccacctcctctcccacttcccccactcccgctttccctctccttctctccccactccatctttttctttctctgctgtttctcgtccctccctcctctccatcccacaacactgcctaccctgtccctgccccaccctggtgctcaggatgtgtgaagtgaggggtggtagcccccaagacctcaaccccgaaggttagcctgttgaaaccactttctcccagctgcccccctggcagttggtgctgctgggggaaactgggattgggggccagattttgcctcttttcctgacaaagagagatgaagagttctctcaccaggtgcctgggactggggtgtgggtgtcccagcctatcccagcgcatctgttctgcatcatgattaatagtgctgctttcagccgggcgcggtggctcacacctgtaatcccagcactttgggaggctaaggtgggcagatcacaaggtcaggagttcgagaccagcctggccaacatggtgaaacctcgtctctactaaaaatacaaaaattaaccaggtgtggtggtgggtgcctgtagtcccagctacttgggaggctgaggcaggagaatcacttgaatctgggaagcagaggttgcagtgagccaagatcgtgccactgcactccagcctgggtgacagagcgagactccgtcctaaaaaaaaaggagttttgctctgtcgcccaggctggagtgtagtggcgccatctcggctcaccgcaacctgcgcctcccgggtgcaagcgattctcctgcctcagcctcccaagtagctaggattacaggcgcctaccaccacgcccggccagttcttgtatttttagaagagacggggtttcaccctgttggccaggctcgtctgggactcctgacctcaggtaatccgcccacctcagcctcccaaagtgctgggattgcaggcatgagccaccgtgcccagtcaactccttctcaaaaaaaaaaaaatagtgctgctttctctttcaagtgtcctgatttgggtgatagtaaatgccactctacttataagggatctacctcagaatgctaattgggacatttttgtagcactctactgttggcagcaggtgatgctcacaacagcccgtgagggtggatgacgtccgcttcacagatgacaaaggagcctcatgctcagaccgtgggctgccagagcaggtccatggctgcagccccacatggaccatatttcccccttgtcactctttccaccaagctcccttggaacttcagttattaagctctcttgggtggaatccaagttagaatcacaacatgtgcctcatatggattgtgccagtgaaaaatgacattctatttagaggcagggcagcctggcttagagtcagtttaaaatatgtattatgctgcaacaaatgtaccatgatcctgtaagatgttcacaacaagggaactggatgtggggtatactgtctgtactaacttcacaagttttctgtaaatctaaaactgttccaaaataacaagttcgtttaaaattaactccaggagaccaggtacggtagctaatgcctataatcccagcacttcggaaggctgaggcaggtggattgcttgagcccaggagtttgagacaagcctgggcaacatggtgaaatcctgtctctaaaaaaaatcacaaaaattagccaggtgtggtggcgcattcctgtagtcccagctacttgcggggctgaggtgggagaatcatctgagcccaggagtttgaggctgcagtgagctgtgattgtaccactgcactccaacctgggcaacagagcaagaccctgtctcaaaaaacaaaaatgaaataaagtccaggaaagaagtaggttttaccactcttattttctgaagagaaaactaaatttaatgtgtaaagtgaggacaagttcaccaagttagtgtttgagttgcctaaaatatgtttgctaaaactattcaaagctttcacataaaacatgatcagaagttctatgccaaaacatatgtgtgtgtatatatatatgcactatatatactgtatataaaaatgcaaaatctaaattgccaaccttttagaaattgctctgaaaggaaagcatttcaagataatttgcttacccaaagaatatactttccaagaaagcaagtaatacttaaggtgttcataatcctcatcaaattaattcttgctactgaaagcttacaaggagctgttttgatgtcgggtgtgacaggtttgacttggcagaaggtgtcactttactaacaacattttaaataagtgacagaagacaagaaactacacgttaaatgccagaacaaagagtgtctaagtggatgctaagagttgaaatatggctggatacctgcccaagagagctgaaaagtagatgaaagttggttacctataaactagtgcaccctaatgaattaaaaggtgttgatgagttaacttgttatgccttccagataagacatgcaaatggggcttcttcctccttcactacttccaagggatttaacaaggagaccaatgcaaatgataaggactgtagggctcaagctggggacagattggggaaagggggaccatcatgcccatatagatgtccctgtgccctggcagtcaaggctgctgaaaaataacaaaacccagaagtctgcgtgatgctgcctctccatttgtccaaagccttcttgcggcagtttgcaggcttttgcaaaagctccaggaccaaggagctatgttcatgctggaagcttgttcaggattagctgttctttgtgggatgggtgcagccagggccaggtgtccagggacagtgttttaacaaagggcatgaggtgtctgatctcacagtggaactccacttgcctttttttcatcttctcattctgcttcatgcacagaaccagccccatcctgaaactgactctaaattactcccgccccaggtggagtgcctttctcggagttcaacagagccttcctgtcgcccaagggacaactccactgaatgcccaagccacacccaaaacctaacaagtaaaaaccaaattctgtgctcccccatcctgggccattcctggtttctctactgctgttggtgataccaccatcagcttgtccatcatgaccctggccagttcctcccacaaccctccacagcacccagggacctcacctccattccatccgacacagatctcctcaccacaaaccttggttttgcaacagcagccatgagacctttacaccctccgcccttcatcctgtcccccactgaggccccagagccattccttaaagcagcgcgccacaaactataacccacaagccaattctggtacccagcctgttttgcacagccagtgaactgacaatgatcttttcatacagccagaaaaacaaaacaaaacaaaaaacaacaaaaaaaaaccccaccattctgagcatgtgacttccatgttcaagatgtctcatgttcagaaaggcccctggaaaaggaggaaggggagctgggcacaaagggagaccctctcagctgagctcctcccatccagacattttcctggacttcctatccaatgacttcccttagcttcttatcagccacccctgtctgcccaggaggctggaagatgtggccttttaactgggcacagctctgtcctctatcatatcagggctctgttcccaaggagggtagagagaatggacaccaggtggaccctcagcagtctgtgccacagagggagtgtttgcaatttccagactaaaagtccccatgtgcttgacggggtatgtgactacaacgtgatgcttgacttttcctcatatgaccagagccactttgtccatctggtacaatgtcagctatctgctaggggccctccaggattcccagtcaattccatatctgcatcaccaccattggcactaaataaaataaaatactcaagttcctgctggtgagcatgagcagtgctacactgggcccttcaaccaaggtgacatgataatgactgaaaataatcactgccacttattggggacgtctcatctgccaggcatggtacaaagtgctttaaataagcattcaacaatttcatgctgacagaagccctgtgagccagtggagctactactatgcccattatacaggggagaaaactgaggcagagagaggttaggtaattcgctcagcctcacacaaccaataggtggtggagccaggatttgggccccatctgcctgactctctagaggctctatcttccagtcttccagagttgagtctaagccatgaataggacaattagacagcagaggaaacccattcagccaccatgtgcatgaagagtaaggaatttctgtcatacagaggggagtgaattcactgagctgagagctgaggaaccattgatctgatggctgagacaccactgggaagactggagaggcttttctgggcatgcagtgccaggcacaggaggagctgagggaagatgactaagaggtactggcaaagaattcagaaattctgatggaagctttacatgttaccatcacatccatccatctatccacccatccatccacccatatcttcctccctccacccaatcatgcatacatccagtcatctatacaccacccacccacccatccatccatccatccatcccttcatccatcccatcatccatccaattatacatacatccaatcatatatctgtacataatccattcttccctcggttcatccatccatccattcatccatccatccacccatcccttccttcatccttcctatcatccatccaatcatatatctgtacataatccattcttccctcggttcatccatccatccattcatccatccatccacccatcccttccttcatccttcctatcatccatccaatcatacatatatccaatcatacatctgcacatcaccagctcatccatctatccatttatccatccatccttccttccatccatcattcatccatcatacatacatctaaccatacatctctacatcattcattcttccatcgattcatccaattatccatcattccttcctccatccatcccattatccatttgatcatacatatatcatctatacatcatccattcatccatccatccatccatccacccatatcttcatccaatcaatcatacatacatcgaatcatctacacatcacccatccatccatccatccattcatctatccacccatccatccatccatccatccattcatctatccacccatccatccatccatccatccatccatccatgtaaccatccagtcatatatccaattacacatccatccagttatacattcatacatgcatctaatcattcaattatacatacacacatccatataattctacatccaattatacctccatccaattacacattcatacacccacctaataaattattaattcatatatccatccatataattatacatcaattatacatccatctaatcattcagtaattcacccaccatccagtcatctatccaataatacattcatccaatcatccatccatccatccacccattcatccatccatccgtccgtccacccatcatggtatgagccatgatttaccacgatggtcccctgtggacagcccaggtggggcagaactgaagggaagcccagggctgcccccataaacatttgcctcctttacatggatgagaactagatccacatgtataaatcctcatgatttgaaggtgcttttaccaacattcactcatgggattctcccaggagctctaggaggaggcaggtagagttgaggtcatctcacgcattttacagatgaggaaacggaggccctgagaggcaggtccaaggccacctgaccagaaagaagtggaactgggacttgaacccagccatcttgccccttggtcccatgctctctagcctgtaactcctgcttcctggtggggcatctccaggaggaccctatcggctggccatgggcctgccctggagtcttttgctctgtgtggccatccttcctccctcaggagagtgtgtgctcccagagcacaggctgtatcttctgagcattttgtcccttcccagtacctagcactcagctctgtatacattgggctctcaagaattctcaaccttccagagtgtaaggccttgacctgctcagccctggatactgcatgatgcattgataagcccataaaataaccagggcagattgactcccagtggccaaagtgccacagggaagggacaattcagcccttctaggaggaggaggaggtagttttctcatttctattaaggcaacaaaagctgccttactaaggacattcttggtggagggcgtgactgtcaaccactgtgatcatttgggcctctcttgcccaggcttcccattctgaaaggacagttttattgtaggtacacatggctgccatttcaaatgtaactcacagcttgtccatcagtccttggaggtctttctatgaaaggagcttggtggcgtccaaacaccacccaatgtccacttagaagtaagcaccgtgtctgccctgagctgactccttttccaaggaaggggttggatcgctgagtgtttttccaggtgtctacttgttgttaattaatagcaatgacaaagcagaaggttcatgcgtagctcggctttctggtatttgctgcccgttgaccaatggaagataaacctttgcctcaggtggcaccactagctggttaagaggcactttgtcctttcacccaggagcaaacgcacatcacctgtgtcctcatctgatggccctggtgtggggcacagtcgtgttggcagggagggaggtggggttggtcccctttgtgggtttgttgcgaggccgtgttccagctgtttccacagggagcgattttcagctccacaggacactgctccccagttcctcctgagaacaaaagggggcgctggggagaggccaccgttctgagggctcactgtatgtgttccagaatctcccctgcagacccccactgaggacggatctgaggaaccgggctctgaaacctctgatgctaagagcactccaacagcggaaggtgggccccccttcagacgccccctccatgcctccagcctgtgcttagccgtgctttgagcctccctcctggctgcatctgctgctccccctggctgagagatgtgctcactccttcggtgctttgcaggacagcgtggtgggagctgagccttgcgtcgatgccttgcttgctggtgctgagtgtgggcaccttcatcccgtgtgtgctctggaggcagccacccttggacagtcccgcgcacagctccacaaagccccgctccatacgattgtcctcccacacccccttcaaaagccccctcctctctctttcttcaggggccagtaggtcccagagcagccatttggctgagggaaggggcaggtcagtggacatctgatcttggtttagtatccttcattttgggggctctgggtgtggcctgggcctctggactttggccacggtgtttgttccagcccttctcctaacctgtcctttccagacactcggcatctaggttattagcacctcgcatactttctgacatgctcctcagtcctgattttgaccatcttctcttgcttcccatctgtgtcagtcaagactgcatttggctgtaagaaacagaaaccccaactaactgtggcatttacatgaagaggtttacttttctcacataatcagatgtctagacttggccagcacctcaagggtcattgatgctctcctgtctttattttctgtcatctttagtggttggattgttgcctcatggttacaaagtggctgctgcacttccaggcatcacatctgcctttgaagcaggaacaagttgcaaagtaaagtggccaaaagggccctgaaactaaatgtgtccccttaggaaagcaggagttttcttgcaagtggcaatcttctgcttatgtctcattggccagagctgggtcttacggccaccccttgctgcgagcaaggctgggacattgagcattttgccgtccaacctctttagcagaataaaccaagggggaagaacgttaatagtggcttttgagtcactagttggcagtatctgcccctctatctttccatcctccccatggagtttcaaggttcctttctcagtacttcttcaggctctgcacgttcatttggatcttgtgtcttggggtgaaaaactggcccaagtgtctccccaagcatccacctttggattaatttggaaaatggctgtcaagtgcccgcctcttgcttggtataatgctacagctttagaggacgcagcaggcatgggccttgccgctgaggttcttagcctcatgagaatatccagatcagattctcttggctccttcttagagccagtgatgcaagacacttcctgctcatcttgtcgggacggttttacaagttgcctgccatcctgagaaagtctacaaaacgatgccagacctcatgccagcttcccaagccttgactctcagtgctccctcaacaggattctggaagaatctcccaaacaagtcgcaatcccctctggaccctgtgcaggcatgagactcaagagcattggctcccacccctggtggagggaacactgctggggctgggatcttgcctggttgctccgcctgcacccaagacaaccataattaaaatgtccttcattgaacttggaaagccttcaaagctgacaactccttatgtgtacccggaaaggcctgggagtgtgccagggcattgctcgggagggacgctgatttggaagcatttacctgatgagagactgacagcagctcctggtagccgagctttccctcctgcctctgctgtgaaggtggacccatccaacagtcaaatgcctgactctggacaggagcggacctatttattgccatgcaagggactctgcacttttgaattgtgggtcatgggcttggatttaggggttagagctgggagaagtcttggaagtcacctagagatgacactgccattttgcagatgaggaaaccgtccaatcaaaatggaccaaggacttgcccaaagcctcacagcaaaaccataggcccccgcactaaccccagagtccctgtgctgtcttaagaatcaaatagttgtaagcaatcatctggttttcagtatttcttcttttaaaatgcctggggccatgcccagcagtctgtttcactgcagcgtttacacagggctgccgggctttcctggtggatgagctgggcggttcatgagccagaaccactcagcagcatgtcagtgtgcttcctggggagctggtagcaggggctccgggccctacttcagggctgctttctggcatatggctgatcccctcctcactcctcctccctgcattgctcctgcgcaagaagcaaaggtgaggggctgggtatggctcgtcctggcccctctaaggtggatctcggtggtttctagatgtgacagcacccttagtggatgagggagctcccggcaagcaggctgccgcgcagccccacacggagatcccagaaggaaccacaggtgagggtaagccccagagacccccaggcagtcaaggccctgctgggtgccccagctgacctgtgacagaagtgagggagctttgcgtgtttatcctcctgtggggcaggaacatgggtggattctggctcctgggaatcttgggttgtgagtagctcgatgccttggtgctcagttacctccctggctgcctgccagcctctcagagcatttagggccttctggacttctagatgctcctcatcttgcctcagtcagcgcgtcagttccagagacttctctgcagggttttctggggcaggtggtggcagacccgtgccttcttgacacctgaggtcagtccaccctcctgctcagactgcccagcacagggtcacctcccaaggggtggaccccaagatcacctgagcgcacagagggtgcagatgactggaccacaccttttggtgatcttaatgaggtggtcccagaggagctcagacatgcaatctagcatccagttctgggactctgtctccttttcaaacgtattcatgtagaacaggcatgacgagaatgccttgtcaacatgggtgatggggaatcaatcagacagggcgccgggctcaaggctgcagtcacccaagagtggctcagcccaccaggccctaggaaacgcctgcacagcctggagctcctggagtcatttccttcatgtcttcttcactgcacttacgtaaagatgccagccattggtttggtgatttggagggtgcccagttgcccaacaagaaatgcagaagaggcctagccaggatttcaccagcagtggagagtagagaagatgtggccagaaaagagtttcctttccctcctaaagatggtactccctgcagctactggggaagcctgcagcattctctagggctctgtgtgttgagagcagccccaccctggccccttctgagtgcatttctgctttgtgacttgatccgtgaagtcccctgagatgggcagaggggatgtcctcgaagctggggcagagcctcatccttgaacgtgaaggacgtttgaagactgtggcatgatcacaggatgagatcacagggaacttgagtttctctcctcctctcccttcacagttatttcactgagggaaatccctcccctgcccagaatgaaaactctagccaactcttgacttttccatcactccaaagtagttgaaagtacattagtctccacagtggcaaaacagtgtgcaaaagctaaataattagaacagccagtcccatgtgacagtcaaagcttctaactccattcaaagttgcagccattcccctcgagggctggcagggaggggaggggtaagagaaacaggaaggttcttactgagttggtcctggtgtgagctgcgtcacactccctgcagaggtttcaaggagactctctctctctctgtctccatggggaccttatttgaattcttctactcttaccccagcctgccatctccagctatcctcccctgaagagcccttctgctgcgctggattctggtggccatgtcatctcctcggccccgtgggagtctgaagatctggctgcagcctcacctctgaggtcctgctagttgccacctcttaaacatgatctgaggctcccatgcactctgacctgtgcccacatggggcccacgggaaacacgctggcaagcaaactgtgggtgtgcagacggttctcagggctgcagcacctgtcctttgctctgcccccaaagcaaggccagcccatcttccatcctctagtgttccttggtggggccctgaccacagtccaccaggtccctaaccagaggggacacacaccaggtgtcctcaatgtattgccttgaaacagttgtgctgggactgtgatggggggtggccatgtagccacccccaccacccccaagccactctctccaaggaaatcctcctaaagatccctttacatcctccatgtggtggggaggttctagagttgggtgcatgtgtcttcagctactgacaatgcagaccttagttggcacctcgctctggcctatcctgtttgctgttcttggcgctccagtgaaactccccatgggccatccagttggggtgcagtgtggccacccccttgcaggttcctgccttgctggagagcacagggccctcctggctcttgtaaaacactccccatggtacagagaggccagcagtgatgtgaggcccaacctccctccatggtgttcccaagcagctccctttctggggtcaaggggtggcaaagacagtgcagcgtccaatttctgactcaagccgggcctggctatcgcagctctgcactgtgtgtgacagcaaggcaactcacccagtgccgtggcagtgaccgtgtccgaggaagcctcctcacaccctctgtctcaaggactctggcatttagctggacttgctgtagctctgagcctttctgccattgccatcaccttgtcagaaactcaggccgaatctgcactcagagttgtgcccaggcagttgagccaacacttgctcagcgatattgtcacatgacaaggcactgtcaccactgggcgtcgtgggtagcgcagtgtcggctggatggacccggagggtgtctgtgtcatgctagtgctagtgatgggagccccgtgagcccattgcccgccctcccatgccctcagcagctgcctggggacagccaatggcctgggtgtttctgaggctaccacatggcttccaggaaactcgagaacctttctctcccttgcctacactcttcacacaggcctgtgctggccagcggtggggatccggcattcctatcttaggtgcagaaagtgactgactcattgcaggcctgggagataagactgatggcccagccagcaagatgtatggatttctcagaggcagtggcctctgtcattgtcctcaggaaatgctggtgattctggtggcctgaggtcaatgcatgtcaacgtggccaacttgccttataaactttttttctggacaattgcgtgcactgtcctgtaacagtgtcctgttgtttatgatgcagaaataggtgtttttaaagcctattgattttggtactattaatgtggtcaggaactttctcagtctttcttgtttggggtgagctgtggcttcctaaacaggaacccaagacacccccaaaagctgctcaccagcactgccagcctccctcttaccaagtagcacccgttcaggacattctgcgaaaggcatttgcccagaagttgggaggaaggaaatgtaacattttggggcacctaccatatgccaggcaccaggctaaacgtgttcacacaaattctcttactaaccctcaccatccttctacaagacaaactagtatcttcatcttggggttcaagatgaggaaatggaggctcagagaggttgaatgaatgccggtgcctggatatgaaccccatctgcctgactccgcaacccaggcaaagtctttccttgaacttcccagcagccactgcttagacacagcctccacaaccatggctcagcagcaaattgcttctctgacctcactcagcctgtgtgtccttgttgagtgaggcattcaggaccctggtcccaaagtggagaaagtctttcctactaggtcatagctacacctgcatgtgggtgctgtgccttttgtttagtgaacttttatcaccagcatcctcagcaatgacatttgcagagaagccagagctgaggcaccttggtattcttgggatgtgactttcctgaatgtttaagggaaaatgcccgaaggtacagagagcttggtttctagtaaacaataactgtcttgcttttaccccccttcatttgctgacacatacaccagctgaagaagcaggcattggagacacccccagcctggaagacgaagctgctggtcacgtgacccaaggtcagtgaactggaattgcctgccatgacttgggggttggggggagggacatggggtgggctctgccctgaaaagatcatttggacctgagctctaattcacaagtccaggagattttagggagttggttcttatcaaaggttggctactcagatatagaaagagccctagtggtttttttctaataccatttctgggtaattcctaaggcatttagtgttctgaaagatgctagccttgtccagcctgggagttgagaatgaatgtctaacagaaactctaggccgggcgtggtggctcacgcctctaatcccagcactatgggagacccaggtgggcagatcacctgaggtcaggagtttgagaccagcctggccaacatgtgaaatcctgtctcactacaaataaaaaaattagccgggtgtggtggtaggtgcctataatcccagctactcaggaggctgaggcaggacaatcgctcgaacccaggaggtggacgttgcagtgagccgagatcgcatcattgcactccagcctgggcaacaaaagcaaaactccgtctcaaaaaaaaaaaagaaactcaaatatgtgtgacaggcgattctcactgcaggctgccctgtggctgatccaggagcaaggccttaaccatgtcatccccaagcgattgcttgtaaactttcttctgtgcagccttcaacccttattatgattttcttctcaggaaccaaactgctgtattcaagaaaggcagctttgtgtaatcatttatcataaatatcttaagaaaaatcctagagattcctaattttaggaaatgggagacctatggtactgatataatgtgggctgggcttgttttctgtcatttgctagataaatgaacttgagagcctactgtaaaatgtggaagcttctagattgcagaagggctggaaagacactgttcttttctcccgagtgatgggatctgtccagtatttagagctgcctctgaggccatctgattctaggagactctgcctcgttgaggatattttgaggcctaactacacattcctgcccccagagaggtcacagcctatagcaggctgatgtttctcatgtcacatggcacagaaaggcacattttcgttctcaggctaacaaagagcttcaaaaactattagaagggacagtggctataagagaagaacctcagtcaatgtgtgaaattaactaggaacctggctcctgtttcttttaggtcatgtttttcagcttaggtaaaactagaggctttgataaagcatgacctctagaaatcattgcttttcataaatggaagtgggtttgagttttttctactgattgttagtgcaggtgatgtctacatgcccccagaacatattccatgcaacaaaaaaagcccaggtcaccgtctttgctgggaacttgacttttgtgctcactgaattttaagctttctgacagcagcctggaatcatggagggataaagtacctattagtaagatggaaaaaggtgtttcaggttggagctgcagtctgttgagagtaagctatgggaaggcctgtatacgaggggtggacttttcttctgtaagtgtccagagaccaggcctcctgaagagggcatgggggcttaacttacctggactactgtgtttacaatactcatttatcttgaactcctcctaacccctgagaattgctacatttagtatttgctgagtacttcctagcatcctagggaatcaatagaacattctcccaaccaggctgggtgcggtggctcatgtctgtaatcccagcactttgggaggccaaggtaggcagatcccttgaggccaggagtgcaagactagcctggctgacatggtgaaaccccgtctttactaaaaatacaaaagttagccaggcatggtggtacacacctgtaatcccagctacatgggaggagtaggaggcaggagaattgcttgaacctgggaggtggaggttgctgtgagccgagatcatgccactgcactccagcctgggcgacagagtgagtgagactctgtttaaaaaaaaaaaaaaaaaagaacattctcctaacctggcttcttcctccaggggtgtaattaatcatgtcagtttcctcattgatacacacacacacacactacaatcctgtatccattacttttcaaggtacatttactatttacgtttggggtccttgtctcttttttaatagtgtttcttaaagtcttgtattatatcagagtacagtaacatcccagtcaagagcactctagtaagctctaggaggaaagcgacttccggaaggcagtggagacctgtcctgttggggcagcataggggcagcccctgcctctggtcagttctggcgctcaggctcagggttgcctctgggctgttcttcccagagactgacaaagggctcccataaggcacctgcagagcctgtgagaagctgaagtcaatgttttcctgacaccagttgatctgtgcaggatccattgatttaaccacctgctgtgtggcatgcactgtggtcgatgccaggaacaggaattggaggggcccatgagcatggccagtatcacaggctggaggtgctgctgcgctctgaccgggcctcttggggatgagcccatgtcaaccaccttgcctccgatggggtcgggcccacaggttacctttgtgtgtccatgaccacaccttcctccccgacctcatccaaatctctttcttttccaagcccctgaatccttcagggctgcaggttttgtttaaagcagagctggtgagttgcataggttgttgcgttgggactagatggggtgttcaaagagttgggagttaaaaaacataaagggtatttattaggagaaccaaggagtgtaattctcctgttcttaatatgcggccaggttaatgaatgtcacgtgaatgaaccagaaaaaaatgaagtgtgcccttgatcagctgggttggtgtgcagcaagctgtgtgaccaggggacagcagtggtcctgagggccgtcactgtctgccgtgcagagcccttcctcccacgggggcctacctcacctgtgccaagggcttgtctgtggtcagtgacctggatagatctgaatggggcttctttttcgaggagtcttatggcaggtctctcagtaaagactccattcttgatgatcacacattttggattttccaaatctgtcagagaatgggcttgaggcggggtttgtgggcactagtttcactggtttcatttaccaaaaaggggagcagaagtcaagtatggtggctcatccctgtaatcccagaggcaagagaattgcttgagcccaggagttcgagaccagcctgagcaacataaggagaccccgtctccacaaaaatgaaaaataacattttagtcagacgtggtggcatgcatctgtggtcccagctgcttgggagggtgagatgggagggttgtttgagccctggagttaaagttgcaatgagctgtgattgcaccactgcactctagcctgggtgacagaacgagaccctgtctcaaaaaaaaaaaaaaagaaagaaaaaaaggaaaaaaaaaactcatgcctgtaatcccagcactttggggaccggggtgggcagatcacgaggtcaggagatcaagactatcctagccaacatggtgaaaccccgtttctactaaaaatacaaaaattagccaggtgtggtggcacgtgcctgtaatcccagttactcgggaggctgaggcaggagaatcgcttgaaccagggagtcagaggttgcagtgagctgagatcgtgccactgtactccagcctgggcgacagagtgagactctgtctcaaaccaaaaaaaaggggtggggggcgggggcaggagaacagtgagaggtagggagaggaaaggggattctcgctacacccaaaccagataccatctagaggctagaatctttgggaggctcaaattccctagaaagcaggagaagcttctgtagccctcccgctttcccagtagattaagcccagggcggctccagatgtgtgacatgctctgtgcccaaccagagcccatcataggcagaggaataacacccacaccagaagggccctcggaggtcaccacgtccaagaaccctctttacagatgaggaaactgaggcccagagaggggagagccacctagcgagctggtggcggctagaccaggagagctgtcattccaagcaagcaaaggcaacgagacgagcccagagctgtgctcccatctctttgttagggggcctgggatgccctctcagtgtcattttgtccaggatgatgctccctctcttaagcgattaatgcgcccttgctaaccttttgctatcgctgcctcttcaaaccagaggagttgagagttccgggccggcagaggaaggcgcctgaaaggcccctggccaatgagattagcgcccacgtccagcctggaccctgcggagaggcctctggggtctctgggccgtgcctcggggagaaagagccagaagctcccgtcccgctgaccgcgagccttcctcagcaccgtcccgtttgcccagcgcctcctccaacaggaggccctcaggagccctccctggagtggggacaaaaaggcggggactgggccgagaagggtccggcctttccgaagcccgccaccactgcgtatctccacacagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaaggtctgtgtcttgagcttcttcgctccttccctggggacctcccaggcctcccaggctgcgggcactgccactgagcttccaggcctcccgactcctgctgcttctgacgttcctaggacgccactaaatcgacacctgggtgcagctgctccactccctcggcctcctcccgtgctcaggctgtggccgcacgcgcccctcacgcttgcccgccactctgcatgtcaccagcacccccgctccgtgctccccaccttgtttgactctctggccacttgatttgtccacaacggcccatcagcccacaggaggtttggtgggtgccttccaccgacaggatgacgggtgccctcatggtgtctagaactctccaaccctcccatgtaggcataagcagccccactttgcagatgaggaaacggaggctcagagaagtacagtaacttgccgaaggccaatgagtagtaagtgacagagccaggtttgggatccaggtaggttgtctctgaaagacacgcctgtcctgcatcccacaacgcctcccaggaggtgctggagtgtggacgcctaacacagagatgtgcagggcacacacagcaggtgacacacacagcatccagaggtggcccagagctcatgctgtgcctttggcccagtgccctgcccccacccactctgccttgtggcaggaagacaaggagcagacacaagatctccctggtccacatgccaccacctccctctgcagaggacaaggggatcctcatgctggcattggagggggttgagcagggcccaccttgagccctcaggagcacgaccacagcagccctgcagggagggattggtgggaggagagtcccaagtatcagggagaggagagttggtgtcccacaggagacctcagagccacaaggcgagcttgttcataaatttgggacccttagcatttcacagttatttgcagagcccagaaatggatgttactgaagctcacagttgcaagcatctgttaaatttttattagattttacttttagggaaaactttgaaatgctataaagaagcctgtgtttaaaagttaagacagaggctgggggcgatggctcacgcctgtaatctcagcactttgggaggccaaggcaggtggatcatttgaggttaggagttcgagaccagcctggccaacatggtgagaccctgtctctactaaaattacaaaaaattagctgggcgtggtggcgggcacctgtagtcccagctactggggaggctgaagcaggataagtgcttgaacccaggaggcggaggttacagtgagccaagatcacaccactgtaccctaagcctgggcgacagagtgagactctgtctcaaaaaataaaataaaataaagttaagagagaaaaaaatatatcctatatcctttgttaaattccaaaacagtaggggacaaataactgacttgacaggttactacaatatttcctgaaatgatgttttcttgaatactggcctactagaggttcataggtgtgtttggattaaaaaagagttccatggcccagtgactgggggaaaaaaataaaagactaaagtaagttaaacaggcttttctgctgcaggacttgtcagagcctttaatgtactaatggccattgtgaccctctgagaaggtcacagagtgggtttcccaaacttacttgattctacctgctaacatttcctggaggaagtttgggaaatgccgatttagcagattcttttgttgtgccgtggatggtgctggttgatgtgggcaaaacaaagaacacgtgagtcagatccgcctggggctcttactaaagtgcaggttcccaggtgccactttaggcttacagacccagttgtggggtaagcctgggagtcttttagcaggtgattctgccacatagtatagttggaaaacctctgggcatactcattgctggtccctctagaaatccaggtgacaatagccaatgagaagctccaagagacccagttgtccatggggtagagggaatgtgatattgaaaccaaagaagaaaatctatgatcagttttcagcagtgactgtcaagagaaggagaagggtgagttagcgctgatgctggctgacaggtcagcgggttggtttcaccaaggagtgtgatgaaggctgatgttgtctgtgggaatgtatgatggtaactggtttgtagctaatttggggaagcagtgagaattcgtgccctttgaagaccagtaagtggcaagaaacccaccaggcctggctcagggctgggctgggcttggctcgtctcagagcagctggggctggtggccaaagccaccattagtgaggggcaggccctgggggtacaaccagcaactaggggacaaagacaaccctgccagcctctcctattctggaggcgtgtgaccagaaatggagatgggttggtcagcataagatggccaggaaggtggaaatcaggactgctggcaatctagccacatgggcaggggagccgggtggttccaggcagtttccaaggccaagagggtgagcaggcacctcacagggaatcagggccaagcctggctgcagtgtggagacaatgcacccacccccatccttggatcttgcaggaggctgggtcctcactgagctaccaacatccatggccctgaggcttttaaaacacccatccatggagtggggctggtcccagtggggtgaggctgaccctggcagaaacagggcaggagcctgtgggttagggagactgcaccttccttagatagcctccatgccatcatgtccccgtgacagtttctgctgcgtcccctctgcatggtcccaccctcggccagcctgctgccccctcttgccaggttgcgctaatcagtgaccccagtgtgctgtgttgatactaacaatgcgaggcctagcagattcaagggaaaagagaaccaactgggtttccaccagacccaactaaacaaacatggacctatcccagagaaatccagcttcaccacagctggctttctgtgaacagtgaaaatggagtgtgacaagcattcttattttatattttatcagctcgcatggtcagtaaaagcaaagacgggactggaagcgatgacaaaaaagccaaggtaagctgacgatgccacggagctctgcagctggtcaagtttacagagaagctgtgctttatgtctgattcattctcatatataatgtggggagtatttgtcactaaagtacagctgtcatttaaagtgctttgtattttggggcaggcttttaaaaagtccagcatttattagttttgatacttaccccagggaagagcagttggcaggttcatgaagtcatgctcctaattccagctttcttagtgtactttcagtgagaccctgacagtaaatgaaggtgtgtttgaaaaccaaacccaggacagtaaatgaaggtgtgtttgaaaaccagccctaggacagtaaatgaagccatcttctcactgcataaactgcacccagatctttgcccatccttctcagtatttcacttcacccattgtttactgtctcaatgactggggaaatgtctggggaaatgctcccgtaattgcacagtggcgtttttcctggaaaatcccaccatggctctagataagacctatttttcttaaaggtatctaaaatttccagcataaattctgtctgaaacacctgaattttaatcagtactggagcccggagggcatctccagttgccacatagctctgagcattcagtggtgtgttgagggctgctcccggaagtgcctgcagagtcagggctccccagcctcatctagtgaggcagtggaagggcctgtggggatttggagagctggcctgggtctctgaagtgatagtgacagctgcttgtcaatcacggtgcacatttagtgccgggggcagggggcagggaataccagcctcatgcatgcatgcattcatttgttccttccttcattcattcattcagtacacatgggtacaacatccctgccctggagttgcccagagtctagggaggggaaagatctattaccctgggcctcggccagctggggagtgctgctggtggagaggggccgtgtgcagcgagggaaggaggagtcgtcaatacccccaccccagctttgctttcttgtcatcagccccagggccccagcctgtgtccctcctctcccattgctacttcatctcctgggtcctccttaccaagcctgaccacacagagggccttggccgcttccatggggaattggaaagcaataagatagcatcccctagaagcccagtgaagtctgggacaggacccttctctgagctctgacttgctcttggaaacacttcgaggcttagcctccccactttgtttcccaagagtgtgacctgttcccctccaaacacccccttctcctccagggccatgcccacccgtcaaaatcccccacgggcaggacgaactgtgggtgtcagtcaccatctatcctgcatcctggttccagggccccccccagccccgcctccatagggacaggcgtgcagacacccgtccctggctgcttcctcttgtggaatgggttcaaaagtaagcagtgttgtttacactgacaaactgaaaaaaaaagaaaaagagataacattggaggcttggcacagtggctcatgcctgtaatcccagcactttgggaggctaaggtgggaggatgtccccagcccaagagttctagaccagcctgggcaacatagcaagaccccatctcaaaaaaaaaatttaattggccaggcagaggtgggaggatcacttgaacccaaagggtggaggctgcagtgagccgtgatggcaccactgcactccagccagggcaacagagggagaccctgtctctaaaacaaacaaacaaacaaacaaacaaaagagttaacattggccagattaggattcaccagatagtgttaatattagtttgatttgagactttaatcagaaagcacatgtgtggtgggggtgggtgtaacctaagtcaggtagaatctttccaacttggggggggcacactcctgattgtagccatatgagtctgtcagtgtggtggaagagaccatgggttaatgggcaggtaaaaaagcaccttgcctggaattgagtagaaagtaaggcccttcagaccccgtgacacacttggggacattttcttgagtaacatcctaagattcatgtaccttgatgatctccatcaacttactcatgtgaagcacctttaaaccagtcgtctccaaattcaggggcacagtaacatccaacaggctggagaaagaacgtactagaacttccattcctttttcatgtcctcttctaaaagctttgtcagggccaggcgcggtggctcacgcctgtaatcccagcactttgggaggccgagacgggtggatcacgaggtcaggagatcgagaccatcctggctaacacagtgaaaccccatctctactaaaaatacaaaaaaacgagccgggcgtggtggtgggcgcctgtagtcccagctactcgggaggctgaggcaggagaatggcgtgaacccaggaggcagagcttgcagtgagccgagattgcaccactgcagtccagcctgggcgacagagcgagactccgtctcaaaaaagaaaaagaaaaagaaaaagaactgtgattggggaggacggtcactttcctgttcttactgatcagaagggatattaagggtacctgattcaaacagcctggagatcactgctttcaaccattacctgccttatttatttttagttactgtccttttttcagtttgtttccctcctccatgtgctgacttttattttgattttatttatgtttatgtttaagacatccacacgttcctctgctaaaaccttgaaaaataggccttgccttagccccaaacaccccactcctggtagctcagaccctctgatccaaccctccagccctgctgtgtgcccagagccaccttcctctcctaaacacgtctcttctgtcacttcccgaactggcagttctggagcaaaggagatgaaactcaaggtaaggaaaccacctttgaaaagaaccaggctgctctgctgtggtttgcaaatgtggggtttgtttatttgttttttagcctcaaagacctttcttcaaatgagttctggcatagaagcaccgtgtaaaatagttagaattctgggcaaaggggaaaagagagctgggggccatccctctcagcaccccacaggctctcatagcagcagctcctaagacacctggtgggaccttggtttcgaaatcgctactctaaggctgggcacggtggctcacacctgtaatcccagctctttaggaggccgaggagggtggatcacctgagatcaggagttcgagaccagcctggctaacatggcaaaaccctgtctctactaaaaatacaaaaattagccgggcgtggtgttatgcgtggtggtaatcgcagctactcgggaggctgaggcacaaggattgcttgaaccccagaggcagaggttgtagttagctccagcttgggcgacagagcaagaccctgtcgcaaaaattgtttaaaaaacaaacccaaaattgctactctcattgggttcctttgcccattcctgattttggcaagagaaatgcttccagattgccctgatctgggtaggacagcatcacgccatagcaacactgccccgtgagctcactgccccctcaactagcttgtggtccttggttaatgtcagtttcttttttgagtttgtgttatgtctaagggtcatctgctgggtaacggaacccagggactgccctagtccctagactgtgccatgcccgactctgccagctttgtcagtgatgctggtgctcgcctcctcgggtgctcgcctggtctgagcacacccaaggagttcttgaggccttagggttgtttgcgagagaatgaaagaacacgacctagctctctttagcatccttggtcaggttcaacactgcccccaggggcctctggtggagccaaccaccatcagccaaataaatccataattagagtcagaaaatggatgtctgcatatgtgtagtgcactaatgtcctgccgatgattgacatggagtggagagtgacctgatcattgctgtgagctctgctggccttggcacaactcatgctgataactaatgcacacagttcctctgggaggaaatgtcctcagggaacttggagtttgggtggggatgtgggtttgtgtgcccagcaagcccttgtggttgtagcagacactagtggcatctaggaggcaaagggtcaccccagtcttagccacgttttgagtcaaggtggcggagtggggctggtgttgactcttggtggcagtaacttttcccaatggtgaaaaacccctctatcatgtttcatttacagggggctgatggtaaaacgaagatcgccacaccgcggggagcagcccctccaggccagaagggccaggccaacgccaccaggattccagcaaaaaccccgcccgctccaaagacaccacccagctctggtaagaagaacgttctcttgaatcttagaggaagctgaagctctcagaggtacagccttcattttaggaggccttaggccactgagaatgaataacccctggcagctggtcagcagcttgcagtttactaagcactggagtcttcattgccttctcagtccttttgatttctgaggcaaatgttgaatccctacctttttttttttttttcttttgagacagagtttcgcttttgttatccaggccggagtgcagtggtgtgatctcagctcactgcatcctccacctcccaggttcaagcgattctcctacctcagcctccctagtagctgggattacaggcacctgccactatgcccggctaattttttgtatttttagtagagacagggtttcaccatgttggccaggctggtctcgaacgcctgacctcaggtgatccacctgcctcggcctcccaaagtgctgggattacaggcatgagccaccactcccagcctgaatcctcactttttatcaatgaagaaattgaggctgattctgcagcatgataaaaaaaaatacagaaaaaggaaaaaaaagaaagaaatcgagcctctgagagtttgcttgactgagtctaaccagctcattttaaacccgaggaaaatgcagtcacatgactactaagtggcagctctcggagcctctctggccccaagtccagggttccatagaggcagccccagcatggcatgttttcagtccccaaatgagactctggagacaaatgtctctggagacagagcagcagcctggataagtcacaatgggtgacgtcactcagggctcaacccctgggcagcttaacttgctagggacgttaggagtctgctgcaaaacctgagggtcttagctgagcagtcacaggctgggcccgttgccctgggctcctgtgagtaaaacccagtcaattttgagtacccagtaaggcatccattgagttattttgcagccaggagtgctattaagaacagtcgcggctgggcgtggtggctcatgcctgtaatcccagcactttgggaggccaaggtgggcggatcacctgaggtcaggagttcgagaccagcttggccaacatggcaaaaccccgtctctaataaaaatacaaaataattagctgggcgtggtggcgggcgcctgtaatcccagcttctcaggagggtgaggaaggagaatcacttgaacccaggaggcagaggttgcagtgagctgagatcgcaccattgcactccagcctggatgacaaaagtgagattccttctcaaaaaaaaaaaaaaaaaaacagtcgtcctctttggggattagggacagcctgcctgcctgcccgagcacttctctcttccattgccccagtgaagtattccaggcccctgggtttagactctgcaccatgtaggggtgtctgacctgcacttgctccttggtggcacgggcagcctatggcacttgctgcgggctgtgaccaaagcctggcctggatcttggatcttggtgactctgcttctccctggcctgagggagctgcccagagcctgcccaccacctgctgcgtgtctttgcggtggcatttctcgcacacatgccgtgcggtggcacccccaaggatggccattcactaaggcccattgtttttgtcttttcgcttcgtgttttctggcctggtgtttttctcatatacatgtgatccagggataattcccagaattttgacaggattttaagtagcgtttggatcctgctgtttttttttcacttaacatcgggccagttgactcacactctgttttttgttgttgtttttttgagacggagtctcactgtgtcacccaggctgaagtgcagtggcacaatcttggcatactgcaacctctgcttcccaaattcaagcagttttcctgcctcagcctcctgagtagctgggactacaggcacaggccaccacgccctgctaatttttgtatttttagtaaagacagggtttcaccattttggccagcctagtctcgaactcctgacctcaagtgatccgcccacctcggcctcccaaagtgctgggattacaggggactcacactttgtaacaacctgaaacaacgtgatgcatttccctttgggtcttacctgctcttcggtggctgcctgcaggtggagagaccctcccccttgggcccctcgaccttgtttcagaatggggcccctgctgggccagctgtgggtgcctgccacgtgaaggactcattaaggccctgtttaagcctgatgataataaggctttcgtggatttttctctttaagcgactaagcaagtccagagaagaccaccccctgcagggcccagatctgagagaggtactcgggagcctacttcgctgggagcagcctccctttgcgtgtgtggccattcactggcttgtgtttctagagccgggaggacccttttctgcaatgcagggttcacacagggttcgcagcctgaagatggagcagtccgaattctcttccctgtgcagtttgcgcagctgtgtttgtctgatgggctttctaatcctgtgtgctctccttgacttcagggacaatggcattacaggcatgagccaccatgcctggctgtctccctatgtttcagatgaagacataggcttaaggaggtcaggtgacttgcccacgaccactctgtaaataagaggcatgaaaagtatttggagccaccaccaccaagcccactggtcaccctgggtctctgaagtcagggaggcaggaggatgggaggtctgaggaggcagagaggctgagcctggaggccctggaggccgaggccccatctgttgtttccttatgtggaaaataagaggcttcatttgtcctattgccacagagcgtactacttcaggaacatccaagacatggaaatccgcagggcacggtggctcacgtctataatcccggcactttgggaggttgaggtgggagaatcgcttgaggccagaagttcaagaccagcctgagcaacatagtcagaccccgtctctataaaaaacattatttttaaaaaagacatggaagtcaaattctaaaaactggtgctggctgggtgcggtggctcatgcctataatcccagcactttgggaggccgaggcgggtggatcacctgaggtcaggagttcaagaccagcctggccaacatggtaaaacctctactaaagaaatctttactgaaaatacaaaaatccagtctctactaaaataagtctctactaaaaatacaaaaattagccaggcgtggtgctgcacacctgtaatatcagctactcgggaggctgaggcaggagactcgcttgatcccatgcagcggaggttgcagtgagccgagatcacgccattgcactccagcctgggcatcagaataagactccgtctcaaaaaaaaaaccacaaaaaaacaaaacaacaacaaaagaaaactagtgcttattcgtcactggccaagctgcccattggctacatgggtgcttcaaagagctgcccttctccaggtctggccagcaggtatgtgttacagcaaatgcctggggcagcggcaggggcattgctgcgggaagcttctggacttgcaggaaagctaagttctcagactgcaggggagctaagcacacctcggcacagggtgaggcctgcggttctcagacttcagtctttgtggagcttgagaaaaatgaggctttgcaggtcccacccctagagattctgctctatccactcttgaaggggatcgagaaatttgcattttgcaactcccactttcctccttgaaagctccggagattctgacgcagggttccgtgggccacactttggaaaatacagacccatgagatagaataccagactgttgaagtgtaacgggggcctgggaagtgcagtaacagaagcaagtttgagggtaaaggacacccagaggagggagggacagcatctgcatggagaggagaagagaccccccagcagcttccagggtgttggaagggtgcgctagtaactgctatgcatggcaggtggggaactgtacgtcagggcacagcagcatgaagcggtatggctcgtgtggacagctagggacaggcaggcgtggagcaggcatcctgttctgaaggccaaatcccacagaggagccagggtgctggcaggagccctgaactagccgaacagctgaacagctgaacattcaccctgtggggaaagggtcagaagcgtccaggcttgagggcacagctgggtctcgtcactgcatcacccttatttaggataaaggccctgaagaattgtattagaggttggcaaagcatatctaccacctcctggagccacgctggccgcagggattataattatttccattttcaaattaaggcctctgagctcagagaggggaagttacttgtctgaggccacacagcttgttggagcccatctcttgacccaaagactgtggagccgagttggccacctctctgggagcgggtattggatggtggttgatggttttccattgctttcctgggaaaggggtgtctctgtccctaagcaaaaaggcagggaggaagagatgcttccccagggcagccgtctgctgtagctgcgcttccaacctggcttccacctgcctaacccagtggtgagcctgggaatggacccacgggacaggcagcccccagggccttttctgaccccacccactcgagtcctggcttcactcccttccttccttcccaggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgagagtggctggctgcgcgtggaggtgtggggggctgcgcctggaggggtagggctgtgcctggaagggtagggctgcgcctggaggtgcgcggttgagcgtggagtcgtgggactgtgcatggaggtgtggggctccccgcacctgagcacccccgcataacaccccagtcccctctggaccctcttcaaggaagttcagttctttattgggctctccactacactgtgagtgccctcctcaggcgagagaacgttctggctcttctcttgccccttcagcccctgttaatcggacagagatggcagggctgtgtctccacggccggaggctctcatagtcagggcacccacagcggttccccacctgccttctgggcagaatacactgccacccataggtcagcatctccactcgtgggccatctgcttaggttgggttcctctggattctggggagattgggggttctgttttgatcagctgattcttctgggagcaagtgggtgctcgcgagctctccagcttcctaaaggtggagaagcacagacttcgggggcctggcctggatccctttccccattcctgtccctgtgcccctcgtctgggtgcgttagggctgacatacaaagcaccacagtgaaagaacagcagtatgcctcctcactagccaggtgtgggcgggtgggtttcttccaaggcctctctgtggccgtgggtagccacctctgtcctgcaccgctgcagtcttccctctgtgtgtgctcctggtagctctgcgcatgctcatcttcttataagaacaccatggcagctgggcgtagtggctcacgcctataatcccagcactttgggaggctgaggcaggcagatcacgaggtcaggagttcgagaccaacctgaccaacagggtgaaacctcgtctctactaaaaatacaaaaatacctgggcgtggtggtggtgcgcgcctataatcccagctactcaggaggctgaggcaggagaatcgcttgaacccaggaggcagaggttgcagtgagccgagatagtgccactgcactccagtttgagcaacagagcgagactctgtctcaaaacaaaataaaacaaaccaaaaaaacccaccatggcttagggcccagcctgatgacctcatttttcacttagtcacctctctaaaggccctgtctccaaatagagtcacattctaaggtacgggggtgttggggaggggggttagggcttcaacatgtgaatttgcggggaccacaattcagcccaggaccccgctcccgccacccagcactggggagctggggaagggtgaagaggaggctgggggtgagaaggaccacagctcactctgaggctgcagatgtgctgggccttctgggcactgggcctcggggagctagggggctttctggaaccctgggcctgcgtgtcagcttgcctcccccacgcaggcgctctccacaccattgaagttcttatcacttgggtctgagcctggggcatttggacggagggtggccaccagtgcacatgggcaccttgcctcaaaccctgccacctccccccacccaggatcccccctgcccccgaacaagcttgtgagtgcagtgtcacatcccatcgggatggaaatggacggtcgggttaaaagggacgcatgtgtagaccctgcctctgtgcatcaggcctcttttgagagtccctgcgtgccaggcggtgcacagaggtggagaagactcggctgtgccccagagcacctcctctcatcgaggaaaggacagacagtggctcccctgtggctgtggggacaagggcagagctccctggaacacaggagggagggaaggaagagaacatctcagaatctccctcctgatggcaaacgatccgggttaaattaaggtccggccttttcctgctcaggcatgtggagcttgtagtggaagaggctctctggaccctcatccaccacagtggcctggttagagaccttggggaaataactcacaggtgacccagggcctctgtcctgtaccgcagctgagggaaactgtcctgcgcttccactggggacaatgcgctccctcgtctccagactttccagtcctcattcggttctcgaaagtcgcctccagaagccccatcttgggaccaccgtgactttcattctccagggtgcctggccttggtgctgcccaagaccccagaggggccctcactggcctttcctgccttttctcccattgcccacccatgcacccccatcctgctccagcacccagactgccatccaggatctcctcaagtcacataacaagcagcacccacaaggtgctcccttccccctagcctgaatctgctgctccccgtctggggttccccgcccatgcacctctgggggcccctgggttctgccataccctgccctgtgtcccatggtggggaatgtccttctctccttatctcttcccttcccttaaatccaagttcagttgccatctcctccaggaagtcttcctggattcccctctctcttcttaaagcccctgtaaactctgaccacactgagcatgtgtctgctgctccctagtctgggccatgagtgagggtggaggccaagtctcatgcatttttgcagcccccacaagactgtgcaggtggccggccctcattgaatgcggggttaatttaactcagcctctgtgtgagtggatgattcaggttgccagagacagaaccctcagcttagcatgggaagtagcttccctgttgaccctgagttcatctgaggttggcttggaaggtgtgggcaccatttggcccagttcttacagctctgaagagagcagcaggaatggggctgagcagggaagacaactttccattgaaggcccctttcagggccagaactgtccctcccaccctgcagctgccctgcctctgcccatgaggggtgagagtcaggcgacctcatgccaagtgtagaaaggggcagacgggagccccaggttatgacgtcaccatgctgggtggaggcagcacgtccaaatctactaaagggttaaaggagaaagggtgacttgacttttcttgagatattttgggggacgaagtgtggaaaagtggcagaggacacagtcacagcctcccttaaatgccaggaaagcctagaaaaattgtctgaaactaaacctcagccataacaaagaccaacacatgaatctccaggaaaaaagaaaaagaaaaatgtcatacagggtccatgcacaagagcctttaaaatgacccgctgaagggtgtcaggcctcctcctcctggactggcctgaaggctccacgagcttttgctgagacctttgggtccctgtggcctcatgtagtacccagtatgcagtaagtgctcaataaatgtttggctacaaaagaggcaaagctggcggagtctgaagaatccctcaaccgtgccggaacagatgctaacaccaaagggaaaagagcaggagccaagtcacgtttgggaacctgcagaggctgaaaactgccgcagattgctgcaaatcattgggggaaaaacggaaaacgtctgttttcccctttgtgcttttctctgttttcttctttgtgcttttctctgttttcaggatttgctacagtgaacatagattgctttggggccccaaatggaattattttgaaaggaaaatgcagataatcaggtggccgcactggagcaccagctgggtaggggtagagattgcaggcaaggaggaggagctgggtggggtgccaggcaggaagagcccgtaggccccgccgatcttgtgggagtcgtgggtggcagtgttccctccagactgtaaaagggagcacctggcgggaagagggaattcttttaaacatcattccagtgcccgagcctcctggacctgttgtcatcttgaggtgggcctcccctgggtgactctagtgtgcagcctggctgagactcagtggccctgggttcttactgctgacacctaccctcaacctcaaccactgcggcctcctgtgcaccctgatccagtggctcattttccactttcagtcccagctctatccctatttgcagtttccaagtgcctggtcctcagtcagctcagacccagccaggccagcccctggttcccacatcccctttgccaagctcatccccgccctgtttggcctgcgggagtgggagtgtgtccagacacagagacaaaggaccagcttttaaaacattttgttggggccaggtgtggtggctcacacctaatcccaacacctggggaggccaaggcagaaggatcacttgagtccaggagttcaagaccagcctgggcaacatagggagaccctgtctctacaattttttttttaattagctgggcctgttggcactctcctgtagttccagctactctagaggctgaggtgggaggactgcttgagcctgggaggtcagggctgcaatgagccatgttcacaccactgaacgccagcctgggcgagaccctgtatcaaaaaagtaaagtaaaatgaatcctgtacgttatattaaggtgccccaaattgtacttagaaggatttcatagttttaaatacttttgttatttaaaaaattaaatgactgcagcatataaattaggttcttaatggaggggaaaaagagtacaagaaaagaaataagaatctagaaacaaagataagagcagaaataaaccagaaaacacaaccttgcactcctaacttaaaaaaaaaaatgaagaaaacacaaccagtaaaacaacatataacagcattaagagctggctcctggctgggcgcggtggcgcatgcctgtaatcccaacactttgggaggccgatgctggaggatcacttgagaccaggagttcaaggttgcagtgagctatgatcataccactacaccctagcctgggcaacacagtgagactgagactctattaaaaaaaaaatgctggttccttccttatttcattcctttattcattcattcagacaacatttatggggcacttctgagcaccaggctctgtgctaagagcttttgcccccagggtccaggccaggggacaggggcaggtgagcagagaaacagggccagtcacagcagcaggaggaatgtaggatggagagcttggccaggcaaggacatgcagggggagcagcctgcacaagtcagcaagccagagaagacaggcagacccttgtttgggacctgttcagtggcctttgaaaggacagcccccacccggagtgctgggtgcaggagctgaaggaggatagtggaacactgcaacgtggagctcttcagagcaaaagcaaaataaacaactggaggcagctggggcagcagagggtgtgtgttcagcactaaggggtgtgaagcttgagcgctaggagagttcacactggcagaagagaggttggggcagctgcaagcctctggacatcgcccgacaggacagagggtggtggacggtggccctgaagagaggctcagttcagctggcagtggccgtgggagtgctgaagcaggcaggctgtcggcatctgctggggacggttaagcaggggtgagggcccagcctcagcagcccttcttggggggtcgctgggaaacatagaggagaactgaagaagcagggagtcccagggtccatgcagggcgagagagaagttgctcatgtggggcccaggctgcaggatcaggagaactggggaccctgtgactgccagcggggagaagggggtgtgcaggatcatgcccagggaagggcccaggggcccaagcatgggggggcctggttggctctgagaagatggagctaaagtcactttctcggaggatgtccaggccaatagttgggatgtgaagacgtgaagcagcacagagcctggaagcccaggatggacagaaacctacctgagcagtggggctttgaaagccttggggcggggggtgcaatattcaagatggccacaagatggcaatagaatgctgtaactttcttggttctgggccgcagcctgggtggctgcttccttccctgtgtgtattgatttgtttctcttttttgagacagagtcttgctgggttgcccaggctggagtgcagtggtgcgatcatagctcactgcagccttgaagtcctgagctcaagagatccttccacctcagcctcctgagtagttgggaccacaggcttgcaccacagtgcccaactaatttcttatattttttgtagagatggggtttcactgtgtcgcccaggatggtcttgaactcctgggctcaagtgatcctcctgcctcagcctcgcaaattgctgggattacaggtgtgagccaccatgcccgaccttctctttttaagggcgtgtgtgtgtgtgtgtgtgtgtgggcgcactctcgtcttcaccttcccccagccttgctctgtctctacccagtcacctctgcccatctctccgatctgtttctctctccttttacccctctttcctccctcctcatacaccactgaccattatagagaactgagtattctaaaaatacattttatttatttattttgagacagagtctcactctgtcacccaggctggagtgcagtggtgcaatctcggctcactgcaacctccgcctcccaggttgaagcaactctcctgcctcagcctccctagtagctgggattacaagcacacaccaccatgcctagcaaatttttatatttttagtagaggaggagtgtcaccatgtttgccaagctggtctcaaactcctggcctcaggtgatctgcctaccttggtctcccaaagtgctgggattacaggtgtgagccaccacgcctgcccttaaaaatacattatatttaatagcaaagccccagttgtcactttaaaaagcatctatgtagaacatttatgtggaataaatacagtgaatttgtacgtggaatcgtttgcctctcctcaatcagggccagggatgcaggtgagcttgggctgagatgtcagaccccacagtaagtggggggcagagccaggctgggaccctcctctaggacagctctgtaactctgagaccctccaggcatcttttcctgtacctcagtgcttctgaaaaatctgtgtgaatcaaatcattttaaaggagcttgggttcatcactgtttaaaggacagtgtaaataattctgaaggtgactctaccctgttatttgatctcttctttggccagctgacttaacaggacatagacaggttttcctgtgtcagttcctaagctgatcaccttggacttgaagaggaggcttgtgtgggcatccagtgcccaccccgggttaaactcccagcagagtattgcactgggcttgctgagcctggtgaggcaaagcacagcacagcgagcaccaggcagtgctggagacaggccaagtctgggccagcctgggagccaactgtgaggcacggacggggctgtggggctgtggggctgcaggcttggggccagggagggagggctgggctctttggaacagccttgagagaactgaacccaaacaaaaccagatcaaggtctagtgagagcttagggctgctttgggtgctccaggaaattgattaaaccaagtggacacacacccccagccccacctcaccacagcctctccttcagggtcaaactctgaccacagacatttctcccctgactaggagttccctggatcaaaattgggagcttgcaacacatcgttctctcccttgatggtttttgtcagtgtctatccagagctgaagtgtaatatatatgttactgtagctgagaaattaaatttcaggattctgatttcataatgacaaccattcctcttttctctcccttctgtaaatctaagattctataaacggtgttgacttaatgtgacaattggcagtagttcaggtctgctttgtaaatacccttgtgtctattgtaaaatctcacaaaggcttgttgccttttttgtggggttagaacaagaaaaagccacatggaaaaaaaatttcttttttgtttttttgtttgcttgtttttttgagacagagtttcactctgtcgcccaggctggagtgcagtggtgcgatctccgcccactgcaagctccacctcccgggttcatgctattctcctgtctcagcctcccaagtagctgggactgcaggtgcccgccaccacacctggctaatttttttgtatttttagtagagacggggtttcaccgtgttagccaggatggtctcaatctcctgacctcgtcatctgcctgcctcggcctcccaaagtgctgagattacaggcgtgagccaccgtgcccggccagaaaaaaacatttctaagtatgtggcagatactgaattattgcttaatgtcctttgattcatttgtttaatttctttaatggattagtacagaaaacaaagttctcttccttgaaaaactggtaagttttctttgtcagataaggagagttaaataacccatgacatttccctttttgcctcggcttccaggaagctcaaagttaaatgtaatgatcactcttgtaattatcagtgttgatgcccttcccttcttctaatgttactctttacattttcctgctttattattgtgtgtgttttctaattctaagctgttcccactcctttctgaaagcaggcaaatcttctaagccttatccactgaaaagttatgaataaaaaatgatcgtcaagcctacaggtgctgaggctactccagaggctgaggccagaggaccacttgagcccaggaatttgagacctgggctgggcagcatagcaagactctatctccattaaaactatttttttttatttaaaaaataatccgcaaagaaggagtttatgtgggattccttaaaatcggagggtggcatgaattgattcaaagacttgtgcagagggcgacagtgactccttgagaagcagtgtgagaaagcctgtcccacctccttccgcagctccagcctgggctgaggcactgtcacagtgtctccttgctggcaggagagaatttcaacattcaccaaaaagtagtattgtttttattaggtttatgaggctgtagccttgaggacagcccaggacaactttgttgtcacatagatagcctgtggctacaaactctgagatctagattcttctgcggctgcttctgacctgagaaagttgcggaacctcagcgagcctcacatggcctccttgtccttaacgtggggacggtgggcaagaaaggtgatgtggcactagagatttatccatctctaaaggaggagtggattgtacattgaaacaccagagaaggaattacaaaggaagaatttgagtatctaaaaatgtaggtcaggcgctcctgtgttgattgcagggctattcacaatagccaagatttggaagcaacccaagtgtccatcaacagacaaatggataaagaaaatgtggtgcatatacacaatggaatactattcagccatgaaaaagaatgagaatctgtcatttgaaacaacatggatggaactggaggacattatgttaagtgaaataagccagacagaaggacagacttcacatgttctcacacatttgtgggagctaaaaattaaactcatggagatagagagtagaaggatggttaccagaggctgaggagggtggaggggagcagggagaaagtagggatggttaatgggtacaaaaacgtagttagcatgcatagatctagtattggatagcacagcagggtgacgacagccaacagtaatttatagtacatttaaaaacaactaaaagagtgtaactggactggctaacatggtgaaaccccgtctctactaaaaatacaaaaattagctgggcacggtggctcacgcctgtaatcccagcactttgggaggccgaggcgggccgatcacgaggtcaggagatcgagaccatcctagctaacatggtgaaaccccgtctctactacaaatacaaaaaaaagaaaaaattagccgggcatggtggtgggcgcctgtagtcccagctactcgggaggctgaggcaggagaatggcgtgaacccgggaggcggagcttgcagtgagccgagatcgcgccactgcactccagcctgggcgacaaggcaagattctatctcaaaaaaataaaaataaaataaaataaaataataaaataaaataaaataaaataaaataaaataaataaaataaaatgtataattggaatgtttataacacaagaaatgataaatgcttgaggtgatagataccccattcaccgtgatgtgattattgcacaatgtatgtctgtatctaaatatctcatgtaccccacaagtatatacacctactatgtacccatataaatttaaaattaaaaaattataaaacaaaaataaataagtaaattaaaatgtaggctggacaccgtggttcacgcctgtaatcccagtgctttgtgaggctgaggtgagagaatcacttgagcccaggagtttgagaccggcctgggtgacatagcgagaccccatcatcacaaagaatttttaaaaattagctgggcgtggtagcacataccggtagttccagctacttgggagaccgaggcaggaggattgcttgagcccaggagtttaaggctgcagtgagctacgatggcgccactgcattccagcctgggtgacagagtgagagcttgtctctattttaaaaataataaaaagaataaataaaaataaattaaaatgtaaatatgtgcatgttagaaaaaatacacccatcagcaaaaagggggtaaaggagcgatttcagtcataattggagagatgcagaataagccagcaatgcagtttcttttattttggtcaaaaaaaataagcaaaacaatgttgtaaacacccagtgctggcagcaatgtggtgaggctggctctctcaccagggctcacagggaaaactcatgcaacccttttagaaagccatgtggagagttgtaccgagaggttttagaatatttataactttgacccagaaattctattctaggactctgtgttatgaaaataacccatcatatggaaaaagctcctttcagaaagaggttcatgggaggctgtttgtattttttttttctttgcatcaaatccagctcctgcaggactgtttgtattattgaagtacaaagtggaatcaatacaaatgttggatagcaggggaacaatattcacaaaatggaatgggacatagtattaaacatagtgcttctgatgaccgtagaccatagacaatgcttaggatatgatatcacttcttttgttgttttttgtattttgagacgaagtctcattctgtcacccaggctggagttcagtggcgccatctcagctcactgcaacctccatctcccgggttcaagctattctccttcctcaacctcccgagtagctgggttgcgcaccaccatgcctggctaacttttgtatttttagtacagacggggtttcaccacgttggccaggctgctcttgaactcctgacgtcaggtgatccaccagccttgacctcccaaagtgctaggattacaggagccactgtacccagcctaggatatgatatcacttcttagagcaagatacaaaattgcatgtgcacaataattctaccaagtataggtatacaggggtagttatatataaatgagacttcaaggaaatacaacaaaatgcaatcgtgattgtgttagggtggtaagaaaacggtttttgctttgatgagctctgttttttaaaatcgttatattttctaataaaaatacatagtcttttgaaggaacataaaagattatgaagaaatgagttagatattgattcctattgaagattcagacaagtaaaattaaggggaaaaaaaacgggatgaaccagaagtcaggctggagttccaaccccagatccgacagcccaggctgatggggcctccagggcagtggtttccacccagcattctcaaaagagccactgaggtctcagtgccattttcaagatttcggaagcggcctgggcacggctggtccttcactgggatcaccacttggcaattatttacacctgagacgaatgaaaaccagagtgctgagattacaggcatggtggcttacgcttgtaatcggctttgggaagccgaggtgggctgattgcttgagcccaggagtttcaaactatcctggacaacatagcatgacctcgtctctacaaaaaatacaaaaaatttgccaggtgtggtggcatgtgcctgtggtcccagctacttgggaggctgaagtaggagaatcccctgagccctgggaagtcgaggctgcactgagccgtgatggtgtcactgcactccagcctgggtgacaaagtgagaccctatctcacaaagaaaaaaaacaaaacaaaaaacccaaagcacactgtttccactgtttccagagttcctgagaggaaaggtcaccgggtgaggaagacgttctcactgatctggcagagaaaatgtccagtttttccaactccctaaaccatggttttctatttcatagttcttaggcaaattggtaaaaatcatttctcatcaaaacgctgatattttcacacctccctggtgtctgcagaaagaaccttccagaaatgcagtcgtgggagacccatccaggccacccctgcttatggaagagctgagaaaaagccccacgggagcatttgctcagcttccgttacgcacctagtggcattgtgggtgggagagggctggtgggtggatggaaggagaaggcacagcccccccttgcagggacagagccctcgtacagaagggacaccccacatttgtcttccccacaaagcggcctgtgtcctgcctacggggtcagggcttctcaaacctggctgtgtgtcagaatcaccaggggaacttttcaaaactagagagactgaagccagactcctagattctaattctaggtcagggctaggggctgagattgtaaaaatccacaggtgattctgatgcccggcaggcttgagaacagccgcagggagttctctgggaatgtgccggtgggtctagccaggtgtgagtggagatgccggggaacttcctattactcactcgtcagtgtggccgaacacatttttcacttgacctcaggctggtgaacgctcccctctggggttcaggcctcacgatgccatccttttgtgaagtgaggacctgcaatcccagcttcgtaaagcccgctggaaatcactcacacttctgggatgccttcagagcagccctctatcccttcagctcccctgggatgtgactcgacctcccgtcactccccagactgcctctgccaagtccgaaagtggaggcatccttgcgagcaagtaggcgggtccagggtggcgcatgtcactcatcgaaagtggaggcgtccttgcgagcaagcaggcgggtccagggtggcgtgtcactcatccttttttctggctaccaaaggtgcagataattaataagaagctggatcttagcaacgtccagtccaagtgtggctcaaaggataatatcaaacacgtcccgggaggcggcagtgtgagtaccttcacacgtcccatgcgccgtgctgtggcttgaattattaggaagtggtgtgagtgcgtacacttgcgagacactgcatagaataaatccttcttgggctctcaggatctggctgcgacctctgggtgaatgtagcccggctccccacattcccccacacggtccactgttcccagaagccccttcctcatattctaggagggggtgtcccagcatttctgggtcccccagcctgcgcaggctgtgtggacagaatagggcagatgacggaccctctctccggaccctgcctgggaagctgagaatacccatcaaagtctccttccactcatgcccagccctgtccccaggagccccatagcccattggaagttgggctgaaggtggtggcacctgagactgggctgccgcctcctcccccgacacctgggcaggttgacgttgagtggctccactgtggacaggtgacccgtttgttctgatgagcggacaccaaggtcttactgtcctgctcagctgctgctcctacacgttcaaggcaggagccgattcctaagcctccagcttatgcttagcctgcgccaccctctggcagagactccagatgcaaagagccaaaccaaagtgcgacaggtccctctgcccagcgttgaggtgtggcagagaaatgctgcttttggcccttttagatttggctgcctcttgccaggagtggtggctcgtgcctgtaattccagcactttgggagactaaggcgggaggttcgcttgagcccaggagttcaagaccagcctgggcaacaatgagacccctgtgtctacaaaaagaattaaaattagccaggtgtggtggcacgcacctgtagtcccagctacttgggaggctgaggtgggaggattgcctgagtccgggaggcggaagttgcaaggagccatgatcgcgccactgcacttcaacctaggcaacagagtgagactttgtctcaaaaaacaatcatataataattttaaaataaatagatttggcttcctctaaatgtccccggggactccgtgcatcttctgtggagtgtctccgtgagattcgggactcagatcctcaagtgcaactgacccacccgataagctgaggcttcatcatcccctggccggtctatgtcgactgggcacccgaggctcctctcccaccagctctcttggtcagctgaaagcaaactgttaacaccctggggagctggacgtatgagacccttggggtgggaggcgttgatttttgagagcaatcacctggccctggctggcagtaccgggacactgctgtggctccggggtgggctgtctccagaaaatgcctggcctgaggcagccacccgcatccagcccagagggtttattcttgcaatgtgctgctgcttcctgccctgagcacctggatcccggcttctgccctgaggccccttgagtcccacaggtagcaagcgcttgccctgcggctgctgcatggggctaactaacgcttcctcaccagtgtctgctaagtgtctcctctgtctcccacgccctgctctcctgtccccccagtttgtctgctgtgaggggacagaagaggtgtgtgccgcccccacccctgcccgggcccttgttcctgggattgctgttttcagctgtttgagctttgatcctggttctctggcttcctcaaagtgagctcggccagaggaggaaggccatgtgctttctggttgaagtcaagtctggtgccctggtggaggctgtgctgctgaggcggagctggggagagagtgcacacgggctgcgtggccaacccctctgggtagctgatgcccaaagacgctgcagtgcccaggacatctgggacctccctggggcccgcccgtgtgtcccgcgctgtgttcatctgcgggctagcctgtgacccgcgctgtgctcgtctgcgggctagcctgtgtcccgcgctctgcttgtctgcggtctagcctgtgacctggcagagagccaccagatgtcccgggctgagcactgccctctgagcaccttcacaggaagcccttctcctggtgagaagagatgccagcccctggcatctgggggcactggatccctggcctgagccctagcctctccccagcctgggggccccttcccagcaggctggccctgctccttctctacctgggacccttctgcctcctggctggaccctggaagctctgcagggcctgctgtccccctccctgccctccaggtatcctgaccaccggccctggctcccactgccatccactcctctcctttctggccgttccctggtccctgtcccagcccccctccccctctcacgagttacctcacccaggccagagggaagagggaaggaggccctggtcataccagcacgtcctcccacctccctcggccctggtccaccccctcagtgctggcctcagagcacagctctctccaagccaggccgcgcgccatccatcctccctgtcccccaacgtccttgccacagatcatgtccgccctgacacacatgggtctcagccatctctgccccagttaactccccatccataaagagcacatgccagccgacaccaaaataattcgggatggttccagtttagacctaagtggaaggagaaaccaccacctgccctgcaccttgttttttggtgaccttgataaaccatcttcagccatgaagccagctgtctcccaggaagctccagggcggtgcttcctcgggagctgactgataggtgggaggtggctgcccccttgcaccctcaggtgaccccacacaaggccactgctggaggccctggggactccaggaatgtcaatcagtgacctgccccccaggccccacacagccatggctgcatagaggcctgcctccaagggacctgtctgtctgccactgtggagtccctacagcgtgccccccacaggggagctggttctttgactgagatcagctggcagctcagggtcatcattcccagagggagcggtgccctggaggccacaggcctcctcatgtgtgtctgcgtccgctcgagcttactgagacactaaatctgttggtttctgctgtgccacctacccaccctgttggtgttgctttgttcctattgctaaagacaggaatgtccaggacactgagtgtgcaggtgcctgctggttctcacgtccgagctgctgaactccgctgggtcctgcttactgatggtctttgctctagtgctttccagggtccgtggaagcttttcctggaataaagcccacgcatcgaccctcacagcgcctcccctctttgaggcccagcagataccccactcctgcctttccagcaagatttttcagatgctgtgcatactcatcatattgatcacttttttcttcatgcctgattgtgatctgtcaatttcatgtcaggaaagggagtgacatttttacacttaagcgtttgctgagcaaatgtctgggtcttgcacaatgacaatgggtccctgtttttcccagaggctcttttgttctgcagggattgaagacactccagtcccacagtccccagctcccctggggcagggttggcagaatttcgacaacacatttttccaccctgactaggatgtgctcctcatggcagctgggaaccactgtccaataagggcctgggcttacacagctgcttctcattgagttacacccttaataaaataatcccattttatcctttttgtctctctgtcttcctctctctctgcctttcctcttctctctcctcctctctcatctccaggtgcaaatagtctacaaaccagttgacctgagcaaggtgacctccaagtgtggctcattaggcaacatccatcataaaccaggtagccctgtggaaggtgagggttgggacgggagggtgcagggggtggaggagtcctggtgaggctggaactgctccagacttcagaaggggctggaaaggatattttaggtagacctacatcaaggaaagtgttgagtgtgaaacttgcgggagcccaggaggcgtggtggctccagctcgctcctgcccaggccatgctgcccaagacaaggtgaggcgggagtgaagtgaaataaggcaggcacagaaagaaagcacatattctcggccgggcgctgtggctcacgcctgtaattccagcactttgggaggccaaggtgggtggatcatgaggtcaggagattgagaccatcctggctaacacagtgaaaccccgtctctactaaaaatacaaaaaattagccgggcgtggtggtgggcgcctgtagtcccagctactccggaggctgaggcaggaaaatggcgtgaacccggaaggcggagcttgcagtgagcggagtgagcagagatcgcgccactgcactccagcctgggcgacagagcgagactccgtctcaaaaaaaaaaagcacatgttctcgcttctttgtgggatccaggagatagagaatagaaggatggttaccagaggctgggaagggtagtgaggggatggtggggggatggtcaatgggtacaaaaaaaatagaataagacctagtatttgatagtgcaacagggtgactatagtcaataataatttaattgtacatttaaaaataactaaaagatagccgggtgcagtggcttacgtctgtaatcccagtactttgggaggctgaggtgggcgtttgagaccagcctggccaacatggtgaaaccccatctctactaaaaatacaaaaattagccaggcatggtggcgggcgcctgtaatcccagctactcgggaggctgaggcaggagaatcacttgaacctgggaggcagaggttgcagtgagccgagatcttgccactgcactccagcctgggtgacagtgaaactccgtctcaaaaataaaaataaaaatacagctgggcacggtggctcacgcctgtaatcccagcactttgggaggccgaggcgagcggatcacaaggtcaggagatatagaccatcctggctaacacggtgaaacccggtctctactaaaaatacaaaaaattagccaggcgtggtggcaggtgcctatagtcccagctactcacaaggctgaggcaggagaatggcatgaacctgggaggcggagcttgcagtgagccgagattgtgccactgcactccagcctgggcgagagagtgagactccgtctcaaaacaaaaacaaaaacaaaaacaaaaacaaacacacaacaaaaacctaaaagaatataaatggattgtttgtaacacaaaggacaaatgtttgaggggatggataccccattttccatgatgtgattattatacattgtgtgtctgtatcaaaacatctcatgagccccataaatatatacacctaactatgtacccacaaaaattaaaaaaatatattttttaaggtgaagagggaggcgagatgctggccttaacccctaacccgttgttctccctgcaagctgtccacagggcctctcagactcgaggttcagctatatggatgcatgagcttggtccccagccaacatgggagacacttcaccatcggcagcagctacagcacaggaaccctgggtcactgccatgtcccctctgtgactttgtttaaacagaaaatgatgctctgggccggctgtggtggcccacacctataatcccagcaccttgggaggcgggggtgggcagattgcctgaggtcaggagttggagatcagcctggccgacatggcgaaaccccatgtctactaaaaatacaaaaactagccaggcatggtggcacatgcctgtaatcccagctacttgggaggctgaagcaggagaatcacttgaacccaggaggcagaggctgagtgagccaagatcgtgccaatgcactccagcttgggtgagggagtgagactccgtctcaaaaaaaaaaaaaaagaaagaaaaagaaaagaaagtgatcctactggaaccatgcttactcccctccccacctcacactgtgtagaaattagtgctgtcggccaggcgcggtggctcatgcctgtaatcgcagcactttgggaggccaaggcaggcggatcacgaggtcaggagatcaagaccatcctggctaacacagtgaaaccctgtctctactaaaaatacaaaaaattagccgggcatggtggcaggcacctgtagtcccaactacttgggaggctgaggcaggagaatggcatgaacctgggaggcggagcttgcagtgagccaagatcgcgccactgcataccagcctaggtgacagagtgagactcagcaaaaaaagaaagaaagaaagaaagaaatcagtgctgtctatacttctttctgcagtgatggaaatattctgtatctgtgctgtccagtatagtagccactagctacatgtggcacttgaaacatggctggtacagttgaggaagagtggctgccatatcggacgacacagctatagattctgtcaccccaccccgagagtccagagcggggacttctgccttaggccctattcagggctgatttttacttgaacccttactgtgggaagagaaggccatgagaagttcagtctagaatgtgactccttattttctggctcccttggacactttgtgggatttagtctccctgtggaaagtattccacaagtggtgccactaccccagctgtgagagcagctgggagctgcttttgtcatctttccctggaaagtcctgtgggctgtctcttcctcatgccttgtcccatgcttgggcatggtgtcaagcgtcaggagggagaaagggtccttatttatttatttagagagggacccttcttctgttcccaggctggagtgcagtggtgcgatctcggctcactgcaacctccgcctcctgggttcaagtgattctcctgcctcagcctcctgagtagctgagattacaggcacatgccaacatgcccggctaattttttttttttttttttttttttttttttttttttttttgagatggagttgtactctcattgcccaggctggaatgtaatggcacaatctcggctcactgcaacctccacctcctggattcaagcaattctcctgtctcagcttcccaagtagctgggattacaggtgcccgccaccatgctcaactaatttttgtattttttttttagtagagacgaggtttcaccatgttggtcagactggtctcaaactcctgacctcaggtgatccacctgcctcggcctcccaaagtgctaggattacaggcatgagccaccacgcccggcctgaaagggttcttatttagtgtgcattttgacattcaatttaattccaaggtcttgtggggtcatggtttacaggatgttgatatagaaaagacttcacttaatgggccgggcgcagtggctcatgcctgtaatcccagcactttgggaggccgaggcaggcagatcaggaggtcaggagattgagaccatcctggctaacacagtgaaaccccatctctactgaaaatacaaaaaattagctgggcgtggtggcaggcacctgtagtcccagccactcggttggctgaggcaggagaatggcatgaacccgggaggcggagcttgcagtgagcagagaccatgccactgcactccagcctgggcgacagagcaagactctgtctcaagaaaaaaaaaaaaaaacagactttacttactggaagccaaccaatgtatatttagagtaatttttcctgggctgagctgtcatttacttttgcagtatctcaagaagaagagtttacagtgtaaatatttgatgcacactttgattatatagatgaagcaaactattttcaagagctttgcaaggacttacttgtatccaaacaccattctaaaaggagtcttacctacttctaaaggctggtctctacttggaaccacttgcttggccctggttcaagtcctgctgcaaacctggaagtcctgtcattgtcttcttccctccagagcagtggcacccaatctaatttttgctgtgccccagcagcccctggcactttgccctgtagactgcagacctcatgtaatgtatgttaagtccacagaaccacagaagatgatggcaagatgctcttgtgtgtgttgtgttctaggaggtggccaggtggaagtaaaatctgagaagcttgacttcaaggacagagtccagtcgaagattgggtccctggacaatatcacccacgtccctggcggaggaaataaaaaggtaaagggggtagggtgggttggatgctgcccttgggtatatgggcattaatcaagttgagtggacaaaggctggtccagttcccagaggaggaaaacagaggcttctgtgttgactggctggatgtgggccctcagcagcatccagtgggtctccactgcctgtctcaatcacctggagctttagcacgtttcacacctgggccccaacctggagaggctgaccaatgggtctcaggggcagctcggttgctggagtttttgtttttatttatttttatgtatttaaggcagggtctctgtattagtccattctcacactgctaataaagacatacccaagactgggtaatttataaaggaaagaggtttaatggactcacagttccacatggctggggaggcctcaaaatcatggcggaaggcaaaggagaagcaaaggcatttcttacatggcgacaggcaagagagcgtgtgcaggggaactcccatttataaaaccatcagacctcatgagatttattcactatcatgagaacagcatgggaaagacccgcccccatgattcagttacctcccactgggtccctcccatgacacatggaattatgggagctacaattcaagatgagatttgggtggggacacagccaaaccatatcagtctccctctgtcatccaggctggagtgcactggcatgatctcggctcactgcagcctctacctccctgggtcaggtgatcttcccacctcagcctcccaggtagctggaactacaggtacctgccactatgcctggctaaatattttgtatttcctgtggagacgaggttttgccacgttgcccaggctggtcttgaactcctgaggtcaagcaatatgcccacctcggcctcccaaggtgctgggattacaggtgtgagccacagtgctcggcctaagtcactgcagtttttaaagctcccaggtgattcttcagtgcagtcaaaagtgagaactggctgggtgcggtggctcatgcctgtaatcccagcaccttgggaggcgaaggtgggcagatggcttgaggtcaggagttcaagaccagcctggccaacatggtaaaaccccatctctactaaaaatacaaaagttagctgggtgtggtggtgcgtgcctgtaatcccagctacttgggaggctgaggcatgagaattgcttgaacccaggggacagaggttgtagtgagccgagatcgtgccactgcactccagcctgggcaacagagtgagattccatctcacaaaaaaaaaaaaaagcgagaaccactgtcctaggccctgatgtttgcaggcaactaaaaaaggaagtggacatccccagtcagctgtggcgcaccaagaacaagtcatgggaacataacctaattttctaaatgggttactaggcacttagagcaaaacaatgatgccgaaatcctgatttcagcaaagcctctgcctgcctgtcttggaagtatccacatgaggctgctggggccttggtgtccccagcagtttctagtctctaggtcttgctgtgggtgtctgtgcagtgagggtgtgtgtggcgctgggtgagctctgtctaggcctggcacaggatgcggtctggtagctgctgcttctcttctgcagaagcgcagccaagcaccctctggggtttcaggcccacacccagcctgaagttctgggagtggctcactttccaaccttcagggtctcccagcagctgactggggagtggtggagggaaaagggattgtattagtccgttttcacgccgctgatgaagacatacccgatactgggcagtctaaaagatagaggtctgatggactcacagttccacgtgactggggaggcctgacaatcatggtggaaggtgaaaggcttgtctcacacggtggcagacaagagaaaagagcttgtgcaggggaactcccctttataaaaccatcagatctcgggagacttattcactatcatgagaacagcacgggaaagaccctcctctatgattcaattacctcccaccaggtccctcccacaacatgtaggaattgtgggaactacaattcaagatgacatttgggtggggacacagccaaaccatatcagggcgtcccagaaagggtatagggtctgagacccaagtcagcatgagaaagtatgcttctcatggtggcccagttgggtggaagtggcagccgggccgtctttccaccaggccactcaagtagcagctgagagacccctgccctggccagtccccgccctcccctcttgccactgcctctggttctgaacagatgggcaccctcatcttgtatttgtgattaatgtctaacaatgtagttttgtgagaagggtttgctgatacagccttgctgcagatgctgcgaactgtggcctggggcagaccttacctccagacacgccctgaggcaggggagggcactggcccgtagctggccgagagctctcgggttgcgcgacagggatacttttcagcggctgggtcgctatccaaagtgagaaaacgaggagggaccaggaggctgtccgcctcaagagatgtgggggccaggtccagttatctggggaagcagtaagcttctctgctgtttctaaccccaggcctcccctggtctaaggcagggcctcccagcctcggggcactttaaagatatctgggcctggccccatccccacagtctgactgagtgggtctggatagggcctgagcattggtgatttcctgggtgaaaggaggcccctcacagtctctggaagcttctctgtgttaggaaaagctctgggcttgactctgctttgaaagtcaagatccgcaaatcctctcagcctcagtttctccttcagcaagatgaaatggaaatgctgtacctacgtcccggggtggttgtgagacccaaaaaagacaatgttctggaaggttcctggtgcgttgcagtcctctaagaacctgagttagagccacgctgagtctcagcttcttggctccttctgtttcaaactcgtccatgtgatagctcaggaagggtaggcagggccctgccccctactcagaaaacaccatcctggtcctggggatccccgcagcattagtcccctgttttcccagtgtattgagaaaaattgctaacaagcagtggggcacaccaccagcctcctgggttcctttcagtttggggatttttggacattcccaggaatgtcttaaaaaacacttcaaaaaacattaacataaatatttttatcaaagcctgtattaaatggtctttcaagaaaatacagtaacaggtcaggcatggtggctcatgcctgtaaccccagcactttgggaggccaaggcaggcagatcacctgaaatcaggagttcaagaccaacctggccaacacagccaaatcccatctctacaaaaaatacaaaaattagctgggtgtggtggcacacacctgtagtcccagctacttgggaggccgaggcaggagaattgcttgatcccggaggcggaggttgcagtgagccgagatcgtgccactgcactccagcgtgggtgacaaggtgaatctttgtctcaaaaaaaaaaaaaaaaaaaagataaaatacagtatacagtaatagagaacaatccttttttcaaagtagtgaccccaaatgaacaaaatatgcatctagcttaaatgcgaacctggttttctctacgcccattcaagcccctgcaataggggcccttcaccccgcatccatggactcctaaaattatatggaaaatggctgtgtgtgagtgtggatggacatgtgcacacatatttttggctttaccagatgctcaaagagcctaggacccaaaaagggctgagaatgaccgtgtcggccacttcagggtcatcaggaattgctgtgcactgctcacttctccagtgaacactttctgcttctgtgtttcctggtatcctttgggactcctggctaggtcatgtgtttctctactttcaaaagggcttcagccaggcacgatggcatgagcctgtagtcccagttgctctggaggttaaggtgggaagattgcttgagcccaggaatttgaggccagcctgggcaagtagataggtagatgattgatagatagatagatagataaatagatggatagataagtcgctagacagtcatccatccacccatccacacataaaaaggcctttgtcatgtcatgttttgtggcccacctgccagtgttgcccacagttgctgcccctccaaactcatcagtcactggcaaacaggaggaatgtgtggctcatgtctgggcatcagtggctgtgggagacatccttgatcttctccagcttctccttccacattttcctttgcaatctggcaatatctattaaaataaaatgtgcatgccttttgacctaagagcttcacttctaggacccacttacacgtgtgtgacatgatgttcatacgggtttatttatctgaggttgttcatacacaccattgcctgtaatcactaaaggcgggagcagcctacacatccatccacagaggagtagatgccttttggtacatccgtggcgacggaatactaagcagcctgtgtatctatacactcacacgtgtttgtttatgtgtggaatatctctggagggtacacaagaaacttaaaatgatcactgtctctggggagggtacctgggtgcctgggaggcaggtcagggaaggagtgggcacaggtattaccaattggaagacaataaaaacaacagctcctggccaggcgcagtggctcacgcctgtaatggcagcactctgagaggctgaggcgggcagattgcttgcgtccaggagttcaagaccagcctgggcaacatagcaaaaccccgtttctattaaaaatacaaaaaattagccaggtgtggtggcatgcacctgtaatcccagctactcgggaggctgaggtgggagaatcacctgagcctgggaggtcaaggctgcagtgaggtgagattgtgccaccgcactctagcctgggcgatagagcaagaccctgtctcaaaaacaaacaaaaaacagtccctggcactctgggccaggcctggcagggcagttggcagggctggtctttctctggcacttcatctcaccctccctcccttcctcttcttgcagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcacccctgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtgggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcggcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaataaggtctccattcatggattccaaggacaagaaagtcatatagaatgtctattttttaagttctttcccacgcacccttagataatttagctcagaacaggaaatgatagtattaataaaagctggacatcaggattaacagctctctctggggccctgaaggtgagagttctcagacttgctcatttgcagttgcttctttgtgatgctggcaaaccatcctagtcccattcaaagggcaatacaaagccttgtggctgacctcacgatgcagcactcagtttgcaagaccggcaccagtgtatgcaaacctgagaaggttggggatgaggatatgggatctttcatccctggaaatttagtccagaggcctggggctggagcagaacaccaagccaatcagcttaatgaatggcttagattcctgctaggtttgcagagctgccttctttcctttggtaccttattatagattgaggagtatttctgctaaaccaagatagggataaccagatagcatcttcatagcaatgccacaaaggaaaacaaaaacaaaacagtaatccatcatattattccttagtaactatgccaaggtcatgatactgaatccttagattgtttcaaaatactacttttctttgctcttcctgatgtgtttgccaccgcaggcagatgtttaagtaaaacagattttaactgcagctacaaaagcagcaacaggccagcaaaagagaagtgctatctcagagagcatggctttcagagccacaagagacagcctcactggctgtttcagcttgactgccatgcaaagaagagagcagagggagaaccagccccacccacttattcatcttgtacaaaaaaaaagcacctaccagcctaggctacatagtgagacactatctccacaaaaaacccacgaaaactagctgggtatggtggcacatgcctacagtcccagctactggtaaggctgtggtgggaggatctcttgaggccaggaaggagatccaggctgcagtgagccaagattgcaccactgcactccagtctggacaatcgagcaagatcccatctcaaacaataaaaaaaaaaagcgtgtaacctcctcagaagaaagatgttataatctcaggcagcaggcaagaaccaatccaggctctaagc

SEQ ID NO: 2 - DNA, 6794 bp, Homo sapiens
aagggccctgaaactaaatgtgtccccttaggaaagcaggagttttcttgcaagtggcaatcttctgcttatgtctcattggccagagctgggtcttacggccaccccttgctgcgagcaaggctgggacattgagcattttgccgtccaacctctttagcagaataaaccaagggggaagaacgttaatagtggcttttgagtcactagttggcagtatctgcccctctatctttccatcctccccatggagtttcaaggttcctttctcagtacttcttcaggctctgcacgttcatttggatcttgtgtcttggggtgaaaaactggcccaagtgtctccccaagcatccacctttggattaatttggaaaatggctgtcaagtgcccgcctcttgcttggtataatgctacagctttagaggacgcagcaggcatgggccttgccgctgaggttcttagcctcatgagaatatccagatcagattctcttggctccttcttagagccagtgatgcaagacacttcctgctcatcttgtcgggacggttttacaagttgcctgccatcctgagaaagtctacaaaacgatgccagacctcatgccagcttcccaagccttgactctcagtgctccctcaacaggattctggaagaatctcccaaacaagtcgcaatgccctctggaccctgtgcaggcatgagactcaagagcattggctcccacccctggtggagggaacactgctggggctgggatcttgcctggttgctccgcctgcacccaagacaaccataattaaaatgtccttcattgaacttggaaagccttcaaagctgacaactccttatgtgtacccggaaaggcctgggagtgtgccagggcattgctcgggagggacgctgatttggaagcatttacctgatgagagactgacagcagctcctggtagccgagctttccctcctgcctctgctgtgaaggtggacccatccaacagtcaaatgcctgactctggacaggagcggacctatttattgccatgcaagggactctgcacttttgaattgtgggtcatgggcttggatttaggggttagagctgggagaagtcttggaagtcacctagagatgacactgccattttgcagatgaggaaaccgtccaatcaaaatggaccaaggacttgcccaaagcctcacagcaaaaccataggcccccgcactaaccccagagtccctgtgctgtcttaaggatcaaatagttgtaagcaatcatctggttttcagtatttcttcttttaaaatgcctggggccatgcccagcagtctgtttcactgcagcgtttacacagggctgccgggctttcctggtggatgagctgggcggttcatgagccagaaccactcagcagcatgtcagtgtgcttcctggggagctggtagcaggggctccgggccctacttcagggctgctttctggcatatggctgatcccctcctcactcctcctccctgcattgctcctgcgcaagaagcaaaggtgaggggctgggtatggctcgtcctggcccctctaaggtggatctcggtggtttctagatgtgacagcacccttagtggatgagggagctcccggcaagcaggctgccgcgcagccccacacggagatcccagaaggaaccacaggtgagggtaagccccagagacccccaggcagtcaaggccctgctgggtgccccagctgacctgtgacagaagtgagggagctttgcgtgtttatcctcctgtggggcaggaacatgggtggattctggctcctgggaatcttgggttgtgagtagctcgatgccttggtgctcagttacctccctggctgcctgccagcctctcagagcatttagggccttctggacttctagatgctcctcatcttgcctcagtcagcgcgtcagttccagagacttctctgcagggttttctggggcaggtggtggcagacccgtgccttcttgacacctgaggtcagtccaccctcctgctcagactgcccagcacagggtcacctcccaaggggtggaccccaagatcacctgagcgcacagagggtgcagatgactggaccacaccttttggtgatcttaatgaggtggtcccagaggagctcagacatgcaatctagcatccagttctgggactctgtctccttttcaaacgtattcatgtagaacaggcatgacgagaatgccttgtcaacatgggtgatggggaatcaatcagacagggcgccgggctcaaggctgcagtcacccaagagtggctcagcccaccaggccctaggaaacgcctgcacagcctggagctcctggagtcatttccttcatgtcttcttcactgcacttacgtaaagatgccagccattggtttggtgatttggagggtgcccagttgcccaacaagaaatgcagaagaggcctagccaggatttcaccagcagtggagagtagagaagatgtggccagaaaagagtttcctttccctcctaaagatggtactccctgcagctactggggaagcctgcagcattctctagggctctgtgtgttgagagcagccccaccctggccccttctgagtgcatttctgctttgtgacttgatccgtgaagtcccctgagatgggcagaggggatgtcctcgaagctggggcagagcctcatccttgaacgtgaaggacgtttgaagactgtggcatgatcacaggatgagatcacagggaacttgagtttctctcctcctctcccttcacagttatttcactgagggaaatccctcccctgcccagaatgaaaactctagccaactcttgacttttccatcactccaaagtagttgaaagtacattagtctccacagtggcaaaacagtgtgcaaaagctaaataattagaacagccagtcccatgtgacagtcaaagcttctaactccattcaaagttgcagccattcccctcgagggctggcagggaggggaggggtaagagaaacaggaaggttcttactgagttggtcctggtgtgagctgcgtcacactccctgcagaggtttcaaggagactctctctctctctgtctccatggggaccttatttgaattcttctactcttaccccagcctgccatctccagctatcctcccctgaagagcccttctgctgcgctggattctggtggccatgtcatctcctcggccccgtgggagtctgaagatctggctgcagcctcacctctgaggtcctgctagttgccacctcttaaacatgatctgaggctcccatgcactctgacctgtgcccacatggggcccacgggaaacacgctggcaagcaaactgtgggtgtgcagacggttctcagggctgcagcacctgtcctttgctctgcccccaaagcaaggccagcccatcttccatcctctagtgttccttggtggggccctgaccacagtccaccaggtccctaaccagaggggacacacaccaggtgtcctcaatgtattgccttgaaacagttgtgctgggactgtgatggggggtggccatgtagccacccccaccacccccaagccactctctccaaggaaatcctcctaaagatccctttacatcctccatgtggtggggaggttctagagttgggtgcatgtgtcttcagctactgacaatgcagaccttagttggcacctcgctctggcctatcctgtttgctgttcttggcgctccagtgaaactccccatgggccatccagttggggtgcagtgtggccacccccttgcaggttcctgccttgctggagagcacagggccctcctggctcttgtaaaacactccccatggtacagagaggccagcagtgatgtgaggcccaacctccctccatggtgttcccaagcagctccctttctggggtcaaggggtggcaaagacagtgcagcgtccaatttctgactcaagccgggcctggctatcgcagctctgcactgtgtgtgacagcaaggcaactcacccagtgccgtggcagtgaccgtgtccgaggaagcctcctcacaccctctgtctcaaggactctggcatttagctggacttgctgtagctctgagcctttctgccattgccatcaccttgtcagaaactcaggccgaatctgcactcagagttgtgcccaggcagttgagccaacacttgctcagcgatattgtcacatgacaaggcactgtcaccactgggcgtcgtgggtagcgcagtgtcggctggatggacccggagggtgtctgtgtcatgctagtgctagtgatgggagccccgtgagcccattgcccgccctcccatgccctcagcagctgcctggggacagccaatggcctgggtgtttctgaggctaccacatggcttccaggaaactcgagaacctttctctcccttgcctacactcttcacacaggcctgtgctggccagcggtggggatccggcattcctatcttaggtgcagaaagtgactgactcattgcaggcctgggagataagactgatggcccagccagcaagatgtatggatttctcagaggcagtggcctctgtcattgtcctcaggaaatgctggtgattctggtggcctgaggtcaatgcatgtcaacgtggccaacttgccttataaactttttttctggacaattgcgtacactgtcctgtaacagtgtcctgttgtttatgatgcagaaataggtgtttttaaagcctattgattttggtactattaatgtggtcaggaactttctcagtctttcttgtttggggtgagctgtggcttcctaaacaggaacccaagacacccccaaaagctgctcaccagcactgccagcctccctcttaccaagtagcacccgttcaggacattctgcgaaaggcatttgcccagaagttgggaggaaggaaatgtaacattttggggcacctaccatatgccaggcaccaggctaaacgtgttcacacaaattctcttactaaccctcaccatccttctacaagacaaactagtatcttcatcttggggttcaagatgaggaaatggaggctcagagaggttgaatgaatgccggtgcctggatatgaaccccatctgcctgactccgcaacccaggcaaagtctttccttgaacttcccagcagccactgcttagacacagcctccacaaccatggctcagcagcaaattgcttctctgacctcactcagcctgtgtgtccttgttgagtgaggcattcaggaccctggtcccaaagtggagaaagtctttcctactaggtcatagctacacctgcatgtgggtgctgtgccttttgtttagtgaacttttatcaccagcatcctcagcaatgacatttgcagagaagccagagctgaggcaccttggtattcttgggatgtgactttcctgaatgtttaagggaaaatgcccgaaggtacagagagcttggtttctagtaaacaataactgtcttgcttttaccccccttcatttgctgacacatacaccagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaaaaaaaaaaaaaaaagg

SEQ ID NO: 3 - DNA, 6816 bp, Homo sapiens
ggacggccgagcggcagggcgctcgcgcgcgcccactagtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctgcgactaagcaagtccagagaagaccaccccctgcagggcccagatctgagagaggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 4 - DNA, 5724 bp, Homo sapiens
ggacggccgagcggcagggcgctcgcgcgcgcccactagtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 5 - DNA, 5631 bp, Homo sapiens
ggacggccgagcggcagggcgctcgcgcgcgcccactagtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 6 - DNA, 5718 bp, Homo sapiens
ggacggccgagcggcagggcgctcgcgcgcgcccactagtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 7 - DNA, 5811 bp, Homo sapiens
ggacggccgagcggcagggcgctcgcgcgcgcccactagtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 8 - DNA, 5637 bp, Homo sapiens
ggacggccgagcggcagggcgctcgcgcgcgcccactagtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 9 - DNA, 6762 bp, Homo sapiens
ggacggccgagcggcagggcgctcgcgcgcgcccactagtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 10 - DNA, 5544 bp, Homo sapiens
ggacggccgagcggcagggcgctcgcgcgcgcccactagtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 11 - DNA, 20 bp, Artificial sequence
  Synthetic oligonucleotide
ccttccctgaaggttcctcc

SEQ ID NO: 12 - DNA, 103001 bp, Mus musculus
aactaatgaccaatttcaaccggaagcttgacatccagcaggtggccatctccagcaggcaccttaaaaattcagctctcacgctgtacaaaggtgtttcaataaacgagataaaaataactctgtgaaaatgccttgcagtgtgtgtggtaagcatcattagaacttacctatttaaagatttttcttcgctagggtcacagtcaagatacctaatgggtttgaatgctccttgatttaaaaacaattgtttttccacgaaaacgctaaagtgtattacggtcaagttaatctgtttcgagaagatttgtcaaggaaatcattcactttggccattaacaaaaacaggtggaaagaaagcaagcgaacagctggccctggaaatagaaagtgcgctctatctggagggccacattttaggagactcggggagcctgtagcacttggggttcacggtgtaccgcctggagagccagcgagggtgaactgaaaagcggacgatgaccgacacaccgctctgcctggaccccgcggggaccgagggaggggacccctggactagacaaaggctgcagccccgggaggccgggtgcgcgtgcgccacggaatgcgcgtgcgcgcggcggtcagcaccgcggcccgaggcgtggcgggacccggggagggaagcgcggagaggggcgagccgagcggcgcggagagaggagggttgaggggcgctcgcgtgcgccccctagcggtcggaaggggaggtgcccgcccaggcctcgctgcccgcccctcccctagggaggttcgcgctcccgtggctcgcgcctgcgctgcccgccggcctccagaacgcgctttctcggccgcgcgcgctctcagtctccgccacccaccagctccagcaccagcagcagcgccgccgccaccgcccaccttctgccgccgccgccacaaccaccttctcctccgctgtcctcttctgtcctcgccttctgtcgattatcaggtaagcgccgcagctctgaaatccagtcccctctggccctgtgcgccgtcctcctgccacccctcgccccagagatggatttagggagctgggcatcaatcaggcgcaacggcgggggcctctctgcgccctaggcgctggggagctgggaaggaacgcgtgcaagagatgcacgcacgcgagcgaatgcgtgtatctatgagtgctggggtccccactatcggctccgcgcaaatgaggggggctgtgcccgcagggtggggcgcggatgacggtgacctagggaaggggttgttctgtaatctggatctcagccttttgccgcaatgggcgtgggtctgtgagtgtgcatgggggtccaacgacgacccccgaaacagaacctgaaatcggatttctctgctcctgattgccatcagccctaaggaaggtgtggatcgggttctagaaaagatgactgccttcatgtctctccacctccctgacagcgattctgacacggccttcactggccctgatttaaggcggtggcccgccccctccccttttcctccttcagaagccggcaggggacatttgggggctgggaggaatccggaggatggggaggggtccaggcgctgtcacgttagtcgcccttccccctgcgcgcgcttggtacagtgacaggggcagcgccgtgcccgagagaaggtagtgcgcggatcccaatgtgggagctcgaagagtcaggggtcacctggcctcgcggtgtcagcgggctggctggcagcggctccccgctcgagggcgcgcagcgttgcagccaggcacagacccacgcgagggctgcaggtgcatttcggggcgcgtttctttctctgcgttctgcgccgggctcccggcatgtgcgctccctgccggaggcgcgggctggcgcgcagggctcgcccctcactgcggcagtgggtgtggacctgggtgggcgaggaggggggagtacgatgtgtctcctcccgctgccacccctctccgttttttcccccctctctgaacgcgaggtgccatctttttgcggcgagtcacgtctctatcgtagcatgccaagagctgtggccaggctttggaggacccggcttggacccatgttaaagaacagggggtgatgaacacggaagccttggcgttttgcctctgcagcggtgcgcaccagagcgttcacctgaggtcgtctaggggagctcgctgctttgttcgtcgtgtgatttaggatctgcgctcccagccctggttttttgggggagtgtgcggggggaggcgggaagctttcattcgacgaggatatttgcctacgcactcggagcagctccatccttttttcgccactcttttcagaccctttgttggatctacagacttgcggcagcccactccccagttcgacccagagccacatgggtgggattcgagatagactctcgtgtgcaggagccctcagaggaagcacatccctccccacacccaggccatcctgaggaccacaccccacttgtgtttcactgctaggggagccagataaccctcgggaatggggagattcggcaagctcaactccgcatccccaccaccaccaccaccgttgaaccctgttgcctctcccggtgggagagtgtgaggagagggtatgagatcccctgggatactgcggccacacccatccctcccaacccaaggagagagggttaagggttgccataggaaccacaccagccagaggaggaacctctgggcgttacctcaccccctacctcccaccccctacctcccacccccccccttactccgcggagatttagaatcctggggtagtctgatagtgcctggaagacacgaggaaggggcccctcatccctgatgcgagcatcattctcttggcactggcaaccatagccatccagagagagagagagagagatgcctctaatgtattttattataagcgtgctgactgatattattttattaatatcatttaaatgagggactttcatttttaaaaaaatccacatctacaacagcccttgaaaccagagagggatagaaagctaagaggtcccagcctccccaccgtacagtaagctcggtcttcgaggttttctggttaattttaccgctgaatactggtgcgggtaagcagttttgagaccgatggatgagcttcttaagggccgaattgcccacgtcgactgctcactcagattgatgtaggctgggggtggaacgccctctcccgatcaaaccgcggcctgaggctgtggcatcgtggccgggcccgcctggcagaaatttcgcgagcatctgaaagataggttcaaatactacccatccctcgccacacgacgggctgctgggaacaggattcttaagctagaagaagccaggctcttgggctggatgcggaaaggggaaggctgggagggggggctgactatagcgggcccgttgggcaaaggtgtcacagcggaaaaatcagcgggagggggaggagacagaacgttttacccagtgcagaaacaaagctcagatcacagcgtcttcgaactcccattttgcactgtgttgaggggtcgagtgaggtactgtgctggccaaggcccgtgttgttaattaaagatcgtgggggttgctgctgagtttgagagactccattcctggctctgccctttaacaagccagtttcattcacctcagaagctagaagtttgtgctgaagccagagctgtggagcgctgagctaggctcatccgaaagggttcaagagccaagatttcacccaccaacctccgcgggtgtgtgctcccttttatgtccgggaggccctatttatcctctagtgttcaagaaattgtaatcccaagtctttttttccggctggttagcattgggtgtatgacagggtcagagacaaactaaccaggccattttcggctcagtccccgggctccacaagactgcccaaaattttcacggatcctacgccacccattcaactgtcaggtctcatagcagaaaaagaagtatgaagccatggtaggttaattcggtggttacaattctgagatgtcatctggggttcaatgatactgcttgtgtttgccacataagaattgcttgcccactcgccatttgcaaagcttctctaaacaaatggctgtccagataatttgtgtgcccattcctcccccagctgccaggccgggtgtaatgactgctgatctgttctggacagtttgcctcaccagcagatgtgtatgatatagtaccctagcttggctttgcggttcaccttcggatggtttgaatatttcttagcttggctgggtggtttgtttgttttttttaaagatgaatgacagcccacggtgtgcctctttgccctctgcacagaccctgtctgtctacacaattgtgtgttggtggaagggtgtggcttttaagtggcttctagtgtcaggctccgttccccagagaggaacaaaggaagcagagagagtgcccaggaattgcaagaagaaaacttggttggattgagttaaggtcaggcttgctcgttgagaacatgagcccttgtacagagcaagctctctggcccactcgtccttggagggctgggtcagcctggggatgcgatggggctggagtggaatttctgcagactgacttctcctcccctttaaacatcaacgtgtgcagttcatttgttggaaatgttaaggacttgtgttgcattccgtctcccctgagcatactgtatatggctcttgtgagttcctggctacgtgtatgaacatacattgctttttttttttcagttgaatatatgaacctttcccactatgccttttcttatatttatttagttaagttcttccttggtcccctcttgaaaatgaatcacttcaaaagaatgtttttagatatctaatataggggctagatatagagtgggtgtctggcccaccattgtttgacagatccgtgggtaaaatggagagatacaggtcaggagggcatccaagagaacatagagaggggcaaatattcaagctcacagcatccgggaagctcggggacaaatccagtctattttgtctatgtgtccagttggtgcccacaccgctgtctgtgatctgctggacacctttgctctgcctggggccccataatgtgcacaggtggctctcctgccccttgttgttgaaggggaattaagagcctagagggggctacctgtgggggtggccatgttccggctgcaggtgactaggatggagatggggagcctcactctctcatcagccactgtagtgacactcattggattcctctgcaagcaggagttaggggtacctggccaagtgagaaggagaaaagtccctggctcctaagcagggcctcataaggctcctgagacttgggtcttttcctggaaaaccaaggtcctcattggggttgctgaagtatctggaaagtggggtgtgttgggggcatcttgtgggtagaggcgggaaatgctgctaaacattataggacagctgccaaatgtcactcactggctctggcatagagaagccctgctgtagaaacagtgttttgctggcactgttatctcaattgtatatagtgcctgtcttctaggagtgctgcatgctgctggagatgatctcatatagttctcactgtgaccctctgacacatagggaagccgaagcccagagaggtttcataaccaactaaggatgcacagcaaagaacacacagctaagccccaggagatttaatggaactttgtgggctagcctgggaagctaatcgggctttccaaccacccagttcttttgtaagagtctgattggctcacaggctggaataagatacggaagtgtggatttgagtccttggtgtctgctttggacatctgggacaaaggatttccctccaccttggtactctgcttatcaaacagagagcaagtctttcccggaaaccttaacccgacctgactgagcgattctccactcatctgtttcgtgaagggccagccaggtttgactggcttaaacataactgttaattaagataggggcatcaagggcagggaaaatggctgtcccctcgcatgagggccttcctgagttcagatctctagcgcttacataaaagctgagtcctgctgtatgcatttattaaaccattgctgggcagggggtgaaaacaggtgagtccctgaaactcactatccagaaagcttagccaaattcacaacctctgggtctctgaaaagcagggggggggggaggcacagagggggaggggggagagtgagtgagagagggaggaagatatcagttggataattggattcagctgtggccagggtgaaatcactgaggtttggggggtgtcagattcctgtcttagattctgtccctcccatagcttgaacattggcacccatggctgaggcccagccctgaaactgtggatatatgtggagctgtgagcacaagaagctctcgttgcaagcatcccaagtctccgtgggcatcgtgggccctgcaggctatgcttgctgcctggccagactccaaagtacatttgcattatttctctgtgccacagtgccactgagaccgcttcagccatgggctcactgctgctgaccgtgagaggtgaggcagccgaggtgagcaggcccctcctagcccgccagtgccagcaatggaacacatactgtcacacttgcttcactactgtcaccttgccaaagcagttgctggcaaataattccttccccgcctccacttgctagacttctgcctgtagggctcccagggaacatggggggcagtgttgaggttacccccagctagctgtggcattctagattgttctctaggcatgtggatagaatgggcatctgcaaaatgaaacctcaagggacttccaggcgggtatcttaacctttcctcttcgctcatgtgccagcaggctcttcactgagcccaatcagcttctcaatagaaaatcgcaggaagtgtatctcttacctgagcaattcagagaagtgttgtaagcgcctggagagcaggtgcccagcacagagggcatttagaggtcatttggtttagaacccttggatgccagtgccagcttcccaacaatccaacctatggtggagcttagctacttagtaactggctttttttatttttaatatattcatatgtggcctggggtattatagggcctgtccagaagattgcctaggagtttaaatgaggaaatgtgatgcttgccatggcctggcatgtgactaaatggcattcttaaccatctgtatcctgggccactggtgttggaaaacatgcagcagataaatcgcaccaagaaagaaccaagcccagatcctctttcggaaccaggcattgttgtttgggcggaagtggatctccaagatcacacacacccatgtttaagtgtacacacagaactcttgtgtacagggacttatctgctgtcctgcttctgtctgcttccttcctgaatcctgctccatgccaggattagactgcaaactccccacaccagccttccctatacacccactggcgctctctccctctgcccccccctccctccctccctcatggaaagggtgacctggctcagtgtgttaggcccgaggtctcctggtttactcattgaagcaaagtgactactggtgactgagagcgtaatgacagtatcaaaaggcagtgcgggggggggggggggggtgagattgcaaacgaagtggctcccctcctacagaagtcagtagcccggtgagtggccttcatggctgacagtgtcaagagagacaaatgttaagtaatccatagtcctgagaaatgaagcctgggaattccaggcacccaataaaccgagtagagtgtgagccaatagaacaagccacagaagcctcctcaatgtggaatcaaggccaagggcgtaatagagcagttttggatcatttttaggaatcatttttccgcttcatttaattatttatttgtgtgtgcgtgtgtgcatgtgtgcatagatgagtatgtaagggtgccctcagaggtcaaaacagcatcaggtctgacctcctggagctggagttacaggtggttataagccatggatgtgaggaccaaacctgagtcttctacaagagcaatacatggtcttaaccacttaccatctctctatccccttgaacctttaaaatacagtgtgtgtgtgtgtgtgtgtgtgtgtgtgtgtgtgtgtgtgtgtgtgttgtgtgtttatttactgttgtattaattatctaatctcactcaggggtttctacctgtattagtcagggttctctagagtcacagaacttatggataatttctaaatagtaaaggaatttattgatgacttacagttggcagcccaattcccaacaatggttcagtcgcagctgtgaatggaagtccaaggatctagcagttactcagtctcacgcagcaagcaggcgaaggagcaagagcaagagctagactcccttcttccaatgtccttatattgtctccagcagaaggtggagcccagattaaaggtgtgttccaccacacctttaatcccagatgaaaggcgtagcccagattaaaggtgtgttccttaaactcggagattcaatcttctggaatccatagccactatggctcaagatctccataccaagatccagataaggatctccaagcctccagataagggtcactggtgagccttccaattccgaattgtagttcattccaaatattgtcaagttgacaaccaggaatagccactacaatccaccccttgtcaacttgacacaaataatatctcatgttcacatgaaacaataacaaggttgtaaatacgcctaacatgatataactatccctcgtacaatcgcaaacgcattagtaaatttacaatgggcattcatattactttataatcctcgtttctgcaactggttacgtggccttaattggtatttataactaccttcctctactacccattctgtatttccttcaccttcagccagcacctcagcaggtcttggctcttttcctggaggattgacccataccttcattcctgatgggtctgcgtcctttgtcatcctgcttggattaggctgttgtagtttcccattgactttaatcacaggacatggtagtactaagagacgccctaagggatctcctgcactccagacataatcttgcttaccaccattgtgaagaggtaatccaatttccccatggtaatctggatctatcacccctcctaacactgttattccttttttagcctgttggtttaagggcattagaagcccaaaatgaccagggggaagtctgagcttccagttcaatgaaatgtttgttgtagctcctggtaggagcactcccctctctggagccaaaacttctaagccagcagaacctagagttatggggacaggaagcaaaaattttcctagagggtcactaggagtgatagtaagtggaactattccgttttccaccccttgattcctggacccatgaatcctggctatgggtgaaactgtaccatatattgagcgctgattcaaagcatatactgccttctgaagaactctgccccagcctttcaagctgttaccacctaattggcgctgtaactgcgtcttcaaaaggccattccatctttctatcagcccagctgcttcaggatgatggggaatgtggtaagaccagtgaattccatgatcgtgagcccactgtcgtacttctctggctgtgaaatgagttccttggtcagaagcaatactgtgtggaataccatgacgatagatgaggcattctgtcagtccgtgaatggtggttttagcagaggcattacgtgcaggaaaggcaaatccataaccagaataagtatctactccagtaagaacaaaacgctgtcctttccacgaaggaagtggtccaatgtagtcaacctgccaccaggttgctggctggtcacctcgaggaatggtgccatatctggggctcagtgttggtttctgctgttggcagatctggcaatcagcagcagctgtagccaggtcagccttggtgagtggaagcccatgttgctgagcccaagcataacctccatctcgaccaccatggccactttgttcatgtgcccattgagcaatgacagggatggctggggagagaggctgattgtccacagaacgggtcatcttatccacttgattattgaactcctcctcagctgaagtcaccttttggtgagcatttacatgggacacaaatatcttcacatcctttgcccatttggagagatctatccacatacttcttccccagatgtctttctcaccaattttccaattgtgatctttccaagtccctgaccatccagccaatccattggctacagcccatgagtcagtgtataatcgtacatctggccatttcttcttgcaaacaaactgtaataccatgtgtactgcccgaagttctgcccactgtgaagatttcccttcacctgtgtctttcaaggttgtcccagaaaggggttgtaatgctgcagctgtccacttctgggtggtgcctgcataacgtgcagagccatcagtaaaccaggctctagtcttctcctcttcggtcagttgatcatagggaacaccccatgaggctataggtgcatgcttggcagcagatggcattgtaacaggagtagaaaccataggcatttgagcaacttcttcatgtaacttgcttgtgccttcaggacctgctctggcccgatcacgtatataccacttccatttgataatagactgctgctgtgcacgtcccactttatgacttgcagggtctgatagtacccagctcatgatgggtagttcaggtcgcatagtgacttggtgtcctattgtcagacgttcagtttccactaaggcccaatagcaggccaagagctgtttttcaaagggagaatagttgtctgcagatgatggtagagctttgctccaaaatcccaaaggccttttctgtgattcacctacaggggcctgccagaggctccaaacagcatctctatcagccacagacacctcaagtaccatcggatctgctgggtcatatggtccaagtggtagagcagccagccagtaaaatcatttttcgattttttccccatcttgtagaaagctttgtgcactgaatcacctaattcattaagaaaatcaagggcattagcttctttaagttcggaatatagtttcaaccatgggtcttcaaaattctctgagctcccaggagggagagaatctggagaggtttcaatatttgaaagtgctggtggatcaacaagccaattccagtattttaaaagattcatccttgtacttctgttactctagaaccactcctggtaccaacttctgtattagtcagggttctctagagtcacagaacttatggataatttctaaatagtaaaggaatttattgatgacttacagttggcagcccaattcccaacaatggttcagtcgcagctgtgaatggaagtccaaggatctagcagttactcagtctcacgcagcaagcaggcgaaggagcaagagcaagagctagactcccttcttccaatgtccttatattgtctccagcagaaggtggagcccagattaaaggtgtgttccaccacacctttaatcccagatgaaaggcgtagcccagattaaaggtgtgttccttaaactcggagattcaatcttctggaatccatagccactatggctcaagatctccataccaagatccagataaggatctccaagcctccagataagggtcactggtgagccttccaattccgaattgtagttcattccaaatattgtcaagttgacaaccaggaatagccactacactaccctaccttagatcattcagttcccaggtaaacaacccaaagcttttatatttataataagccttaatcagtactaaagctgggcagatatcttctctcgactattcctatctacttcgctagccataaccctgaattctaacttgccatgttccgtctgagctgctgctcttaatttcaattggccagccctcatggccatgttttcatgactcacctaccctatggcatctcctcttctctccaccttctttcctcaaggtccctgcctcagaccccagcctgggaaccaaaactccacctatctctctcttctgccgagcagtaggctgtagggataatttgtggggcggggctacatagcatcacctgggtctgctcgaggtcctcctccttcctgggggcaaccagggccagtatttagcattacaatagcaacagaccaaacctcaacactttgtgtgtgtgtgtgtgtgtccacatgtgggctcacagaacaatttgtgagggtcagttctctccttccaacgtctgagtcccggacacaaactcgggtcatctagtttaggaccaagcacttgtttggccatcccactggacctatttataccttttgggggagaaaagtcttatttgtgtgtgcatgtatgtgcgtgcttctatagtgtatgtgtatgcatggtatgtgcatgtgtacgtgagatggaagccaggagacaaaacatcagattctccatacattgccatccaccttatttgtttatttatttattaaaatttttactttattattttattttatatgtatgtgtgtttggtttgcctatgtgtgtgcaccatgtatatgcccggtgccccccacagaggccagaagagggtgctggatcctctgggactggacttatagatgtttgtgaactgccatgttgctactggaaatcaaacccaggtcttcagaagagcagccagtgctcttaaccactgagccatctctccagccccctattagtgtttgaggcaacatctcttgttgaatccagagctaagtaattgactagaatggctggttcatgacatctgggatcccctgtccttgcttgagttcaccatgctggtgttacagatgagcatcattggtgttgcgaatctaaaataagagtttctagggcttggtgggaggtggcttgggaggtgaagtgtttgccacataaacataaagacctgaattcaaatccccagcgtacacatacaagccagatgaggcagtgtggttcctcgtgctttcaagactctccccagcctctagcttgtacttatagaagtaatgcaatcgttatagcaaagatataatagccacgtgatggttccagttacctgattagtatagcaaataggatagacagagaaggcttcagaacaagcccagatgaccctgagcaggagcttggttgtggcaggaggttggcctggctgcagacaggctctgtggatcccagccttaagaaggtctttcatttctcctacaaaaggccagaaaagcacctaccagggagacatttgtgttaagttaaaggcaggtatactcctctctgcccactcttgcctgtctttaaggcccccagaatacacatcagacacaactttaaactcctcttaggaaaaagttttattcaaggtccaacaatagaaagaaagtttgaaatccacaagaaatgaatccgtagtcactgataatggtggcacttcctctgtggcccttccactccctgccagcgaggaggagagggacatctgtgatctctggtccctggtcttccttctccttccagcctgtggctcagtgttgttttcccagaaagagtgggtgatgctgtcctggtaccctcgccctagcggtgacacagaagatccaggcatgtactttgatcttgatgtcttcctccctgggacacaaaggatgtcagaaaacctctgtgtccataactgcctgtcaattatgtcaattagtctgcagccagcataaagtgataagcagggagcgctgggaagactcacagagggtgccttctccaggacagctcttccaccagaaggcttcaaactatgtgtaaacaaaattcaagggggcttaccaagcaaaaacaaatgaagcaaaacaaactaacaaaaattcttaaaattgttctctgtcaaatctagccctgccattcatttacactgcaattcaggaggtgtgtgtgtctgtgtctgtgtctgtgtgtgtgtctgtgtgtctgtatgtatgtctctgtgtgtctgtgtgtatgtctgtgtgtatgtatgtctctttgtgtatgtctgtatgtctgtgtatgcatgtgtgtgtatctgtctgtgtgtgtgtctgtgtgtgtctgtctctatgtctgtgtctctatgtgtgtgtatgtctgtgtgtgtgtgcgtgtatgtgtctgtgttgtatggcatatgctttcacatatgtgagtgtgtggaggccactggctgaggtgtggtgtcacccactatggccccacaccttatttttaatttttattttattattttatatgtagaagtgtttgcctgcatgtatgtttgtatgccatgtgtgtccctggtgcctgtagatgtcaaaagagacatcagattacctgagagttacagatggttttgagctgccctgtgggctctgggagttgaaccaggtcctatgaagagcaggcagagctctttacctaccaagtttcctcaccagcccctccacaccttactgagctcaacattttcattagaggagtgactagccaacaggatccatctgtctgtctgtctgtctgtctgtctgtctgtctgtctctctctctctctctcacacacacacacacacacacacacacacacacacacacacacacaccgacccatccagtgtttcagacatatgctaccacagctggtttttatgtgggcacagggattgaactttgctcttcatgtatgagaggcaggcactttagccactgagccacccctcccccactgagcctgaggaacattttttttttttttggtttttcgagacagggtttctctgtgtagccctgactgtcctggaactcactctgtagaccaggctggcctcgaactcagaaatccacctgcctctgcctcccgattgtgagtgctgggattaaaggcgtacgccaccacacctggcctgaggaacacttatatgtcccccaaattacttgtcttaccttatccttccccggtggccttaccccccaaaagaggtttgccgatgagctaataatctgagaccagtcctaagagaaggttggacttaaagactgactttggttacacagccataaatgtgaatattgactattgtatgtctggggtttttatgttgagcttgctgaaaacctggtcactttttaactttgttattggcagtgcatcagaatttaagtaaatttatgttaattgtcagaggaaaagttggcactgtgagtaagacagagtaaaccacaggatgcctctagggcacaatgaaaacccacagcgtggacaaataggaacaggtctctttcaaagctgcccattgcctggaagactgagacagcatttggaggtctcaccagcctacttcctgcctagtctttttccagaagaacacaggaaatactacatggcttgaatctgctgtgactactgtggggtcaggctctgccccgcccctatgcagccagctcaggaggatagactttgaaaaagaaaataaaaatccctaaatccgcttaaacgcttaaacgtggaagatcttcacactccccaggaccttctttaagctggttgaatattctcctccaataaacctcttgggaatgatatgcctcccaggtggctcagtgctggggtgagcccctcctcccggctccaaagtccagcccttcctcctcctccctcctccttcctccctcctccctgtgcctgagaaaaggctagtcatggatgcactgttccctgcagagtgctaataagggctgagtgcattaaaatatatgccttgctttttttttttggtatcataaaatattattaacaatcacaaggcagcccttaatggctgttggagaatttcagaaaaacatttctttggataagcaggatttgtgccataagttgacagtcgggaccaaggcttagaataagatttaagacttgatccatcatttgttgctgacaaaactgaactacagaagccagtgagctgctcacggtgtggtcttggtcatcacccactgctgcatcctgaatttaactgccagtttcccgttggccaataagagatttttgacaaagatggagaattcctagacctctgtccttcgtctgcaaaatgtgaatgatacagtctccccttggaatggagtggatggtaaggcctcagccttgatgtctactgagtagtcacagaactggaatgaaacagtccatgagaaatccttggggtagctcagaagtcagtgtcccactggaactgctacagctccctgatcagaggtcccaagtgggctgagtgtagcctgaagtggcacagctgctccctgccaccttgctcctgtttccagccagccggaggcagctggtcctctgtagtgaacactaggtcagcacgggtccatgtccagaaggataaacaggcggccacagcccagtctgtccccagtgtagagaggaggagaccccaagtcagggtgcaggacagtgggaggggtgacctcaagtgatttcgagacctctccggaaaggagctgggacttcctcttggtctccagttgtgacccttgagtggaatttcctgggagtgtgcccatttttttatggaactttcagtgtcttcctaattggtacagacttgacacagaacctcacattctaaattttcccagggtggattctccctgcctgtccatggtagcatatgacagaaatccaccaaggtgagagcaaattcgagccagaccttgactttgatggcatgatacttttgtggctcattaactgaatggctttaattgagccgcagctgggctggagttcattgacttagccctgtgaacggaaggtttctttggcctcgtcctccaacccccagcactggggattgaactcagggcttcatgagttctatcacagagctacatctgctgcctgaatggaaggttcctgaagaatgaggctggtgggaaatccagtgtgcaaccccatcctgtcaggggcattggctgcacctcagtgagctccagtgtctactgaccccacgtcactgctccttagaccctcaccatctgatcttcccgcttgggctgttgaaaccttccatcttcccatcccccgagtggtgaaatgcagtcttcagctctccctccatatcctggagtggacctgtcggtttcccgcagacattgcccacccagctttgtttctccagctctgcttttactgactccaaaatgagttcttacagggctggagagatggctcagtggttaagagcactgattgctcttccagaggccctgagttcaaatcccagcaaccacatggtggctcacaaccatctgtaatgggatctgataccctcttctaatgtatctgaagacagctacaatgtactcatatacataaaataaaatcttaaaaaaaaaaagagttcttaccctggcatcgttatagaaataattataattcctagagtgaattctaaagttatactcgatgcagtttgtatcccattgccaaacacttcccagagcaaaattctgtctgtcatgttcatgtccacccacaaatcccttgtctgtcacctgctaagccgatcctgccctcacgccccgcccagaccctggatttcttctcactgccagccagctgccatgtcttcttcatagatggcaccttcttaattccctcatcttccatggcattgtgcaggtggtaccctcctcacctgtacagactccgcagtctctgtcccctctgagttgtccccacctgtgtgcactagatccaggagccccacacacttcctctcccatgcattcccactggagtagcttatgtctagctgtcatcctctttcttgtcttgctttgtgtgcatttcctgcctcctccaggttatctgtcctctcagagcagacactgtgcatcaccaggcactcctggacctctggggcataccacagtcacccaacccgcactcagagccccgaacgttctggttttgtttgggtttttgttttgttgttttgttttaagacagggtctcactctggagcctaggctgacctgaatctcactggatagtctaaggaactttaaacttgtggcaaacctcctgcctcagcctcctgggtgctgggattacaggatgtgcctgctagcacactctgggtgaatatggctatgcaggctgatgctgggtgagcctgacctgtttaaagaagacgagcctttccctgctgatccacagtcccactgctctgagcacctaaaaataaaaatcacattttcagtgagatacttgattgcagtctgttctactttgaaccagccaatcaaccagtctcccccgtgatccagatccgttatctgtatgaagacgcacagaacacagcaactaccggttgcatttattttactaaaaactgtgccttggtccccgtttccccacgagactgggtcattttgcaaaggagcaaagaaatttgggggcttaagaaagagccaaatggtgtcttaaacccaggtctactccatgtcaggcccttgggtctcctccccaaggctccatgctctctgccagcgccgcgaggagccagaggacctgtctgtaagaatctgggaagcatgcccacgtgaacgggtcacttgtaacgtgagcccggagaaattattttaacctgaaagcaagcctctaaaccaggctgctgcttcatgtggcccatgacggcaaaaccaaatttagcatcacagacaggtccaccctgaacaaactccactctcttaaaaaagtcttccagctcacagaaagcatgggcaaacattcacacccaccacagtgccaggaggttcccagtgatcagaggctgggggatgagcgcgtaaatgaactcagggaagagacagacagtgtttccaggaggagagtcagttaaaggggacaggttccctggatgcgacaaaggcatctttgctgctgggggaaaaggtgggttgatgggacagagggagaccccgtggtccttctcttttaaaggtcctgagttcgattcccagcaaccacatggtgatacataaccaactgtaatggggtctggtgcactcttctggcgcatacagatagagcactcatatatatattaaataaataaaaataaaatttaaaaaatgtaccataaccaaaaggcacagttcctggagaccacagtcccacttagtgggtcctgtggcgctgcagaatgggtgtctgtgccagcccttatgtgtgcggcagcaggtacaagggtgtttgttacaatggtccactagataacccacagaaggctacgcacattcgtgtttggggacctcattgccgaaggtcttcacggtgactccaacaatgaggtcccacttgctagtaggacaaaccctgcccccatagggggccccaaactttcaaagaacacttgagaaaaacctttctggatttactaaaatttggtgtgatttgggatgaatttagagaagacgagtcattttaagttgtgggtgtttgagatcagtgactgacatgggtcacctaaagtaggcccaggagtagggcagtagtggtgcacacctttaattccagcactcaggaggcagaggcaagcagatttctctgagttcagagccagccagagcaacagagtgagaacctgtctcaaaaacagaagggaggcttcggaggagtgggcggagcctgcgggactcaacagaagacaccaaaagaagaaagagccaggtctttctctgtgtagccctggctgtcctggaactcactctgtagaccaggctggccttgaactcagaaattcacctgcctctgcctttcaagtgctggaattaaaggcgtgtgccaccaccgcccggcctaagagccacatcttttttttttttttttttttttactgtatataattatttatttttaatttaagtcaaataaattatttgagggattttgattcctttgctgtccatccaggtcacctgaaagagaagacaatgagagatgtgttaatgagttaagtgattcagggaaatgtatgttatcctctcagaaaaacacagcactatcaaatcagtgcaggattttctctgtgaagaacatcaatctttggtcacagcgttaagagctgtgggttttatagcccaaaaagccctctttgaaatgacctaagagccacgtcttacgtagcacagttttaggttttactttaaagaagaaagcaatctgtgtctttacttatggtacaaactctcagatctctgaccaatccagccggccaggcagccaggcaggaaccatttcataacataagttgtgcttgggtgtgggtgtaatttgtgggtgcagtgttcccatagtgttgcagtggaggataggtgagaaaaatcccactgtccaggcagccccgaggagctgaagaggagcaggcggagactgcgaggctcatcaggctgccctgggaagcaagtgactgctggggctggggtggtccaaggaagctgaggtcaaggcccacactcacccatatacatctccaaagatccagaagacatggaaggggaaagagccacgtcatcaggttaacgaagccagttaagtaactcgtcttcagacgcaagttatttttcccctgaaactaaagtctaaaaataggtaaattaatgagtgtctgtctatttcaaaccaagttgatttctcccctttccctctgctatacccattgtgtgtaattgcagtgatagcatctcataccacggttttaaaccaccctggggaacaggctccttagctaacccagtatgttgaagggactttcacccatgtgtccccagcctctggcattctgcagggcacagaacaagagctaggcaccatggaatggatgaataaatagataacggatccatgaatgaacgtagtatgagttaaagcggcacacaggggaaggacaatgtactttggtctgtgtgttcgtctctctaccttctggagttctgcatttggatttggcatgctcacagtagaaacatgtgcacagtgtgtgtgtgtgtgtgtgtgtgtgtgtgtgtgtgtgttacaactgcctttggggatttcttgtttgtttttcaagacagggtctctctgtgtagctgtggctgtcctggaatttgctctgtacaccgagcttggctatagttgtctgtttaaaagttcagtcaggtgcctttaagttcccctgaatcctggaatggcatcatgagccataagtcccacatcagtggctgtgaacagggtcgggtctgtggcctgggttactggcattccagctgactcctagagtttcagttctgcatagaggtgttagattgtcagatttgggttggagagatggctcagcagttaagagcactggatgagcttctaaagggcctcggtttcattcccagcactcacacaatggcttaccactgtctgtgacttagtcccagaggatacagtgccttctgctcacctccaaaggcatggggcatacacatggtacacacaggcacatgtaagcaagacacccatatcttaaataaataaataaataaataaataaataaataaataatttttaagcagctggaattgccaaaatagaacacagggtgtccaagagctggagagatggctcattggttaaatgatccagaggatccaagttcaatgcccagctcccatatcctgtaacttcaactccaggggaattctcttctggcctctatggccctgcactcctgtgcacataccctactccctcacatacatacccataactaaaaatataatctatgaaaaaacacacagggtgtccagttgaatttggataccaaaataataataataataataataataataataaagaggctacaaaatgtcccctttagatttccagaggagccttcaagggaagtggatcaaaatgcttctatcatttcctgtcacaccacacacacactgaaaagccctggactggaggccactgttaaagactgacagccattttctcatgtgaagaggccttccaagccaaggtgagcacaggccagctcagtaaaggtttctttctgtcattgtctttcacacccgatctgttccccacactcaaaggatccactgttctggacacataagggctctgggatcatccagaataggccatggtgctaagaccccaggattactctccagataccaagggcttatagaagagaaaatgtacctggtggaagaaagagcaggaaaggagaatgaacacgaaaagctatttggtctcacctataataggtggctggtccacaaggtgagatgtcctcactgttcaccagacagcagaaatccagaatggtgtgggcggtgactgcagcttattaaccaggctgcagcctttgacagagcctcagagtcggagaggaactgcctaagagagtggtccttaaagtagtctttattctccaccccaccccaccccgtgtatgacctgttaaaaagataaatatgtacaccaatgcaccagctcacctcagaggatatatctgttcgaagcatctagctggattgattgcttggtgggtcacatctgtaattatagacttgaggtgctaagtaaggattataaattcagaatcaacccgggaaacacaaaaagaccctgtcccagataacactcttaactccccacaaaagctcaaggccttctacaaagactgttcttttaaacacacacccctccccgccagtggcatgcctgggaagaggttctgatctgccctaaccatcttatgtcacacagaaacagggaagcggttgcaccaggcccctgccagctatcctcaacacttcactgccttacacagaaccttccagttaagccacaggatgagactagccgaggagcagccaggcatgcttgtacacacctttaatcccagccacttgggaggcagaggcaggcggatctctgtgaatccgaggccagcctggtctatagagtgaattccaagacagctagggctaccaagtagagagagtctgtctcaaaacaacaacaaaatagcttagaagcatggcatacatgggtatgtacatgtgtctgtgtaaaacatgtttataacataagacatagttgaacccgggaaccaaatgttcttgtagacccacagatggacgggataggtgagcaaggagaaatgggttagtgtgtttaagtccctaccttaacatagatgggaacgaacgatctacactgcccagtggggaactgcatgtcatgtgacaacatagatggaactatctgatttataatcccaaatcctttttatgtcaaaaatagccccactgctattttttcctcttggtgctttcacatagatgacttcattgcgtgcccctccaggccgttccagagccagtggcacttcctgtttggaaggggaggaaatgggtgctgcctgtctccatctccctaccttcctgggtgcagatgccacaccctgcccctgcacagtcctcccccaggcagctcatcatggtgagcgggactccttcagaatccctgcctgctcacatcacccctgcttcctctgaaggactcagagaacatactggagaccctgcggagttaaaggagccgaggcgatgtggtacctcagtgttctccaagtctttagcaagtacacgcggttaccagccttggcaggctagctgagcacctggcagcctcagccgggagaaccttccagatgggctgtaggcttccagcacaggtgccgagaatgatgctcttcccaggctcagacccttgggctggtcacccatgtggtccaggtcccaggtcttgggcccatttttggaatttctatttggagatgtgggggtggccactgagcatgggacagatggtgcattcctggttcagctcattgcctcagcacaatgagatcgtgccagcatgtttgcagatgctttctggttttctccttaaggaagtcgagctccaaagtggcttgatagctttcccatgggcccttagctcatagcagcctcactgggcagtgcaggggctgcttggcctttttcctgtattgcttcctgagagctctcctcccatccttgctctggatgctctggtcccccaacccagcacccatttaccagaggcctagcattgccctttgacccttcccagtcctccaaggaatccagactgtagccttccagtaggaatagggtggctgtgtggatgccttcctgtgcattggaatcccacaaacacaccacacaaacccatcatctgcaggctgcagaggacacgtatggagtccacaagacacggaggcacatctgcagagccctgctgtgtggcccaggtgagagaagcaggagatggaagagttctagacactgactggtgtgcagctgaggcctgcctggccctcagcgctgtccagcaccacctaacaacatgcatggatgtaaacttcacacaggattgccatatagcgggacagagacacagctcaacatgaatccaaggaaatctgaatgaggacatttcttactctgtttccctccataggcttttaggtgaaacttagctggtaatatccctttatttgtcctaatagttgggttaggaattgaaaatattggatcaagactcttaagaatttctccattgtttgggtttttttttaatcttaaaaaagcaaaaacagaaacaaaccactttagatttatttattttaggtatatgagtgttttgcacacatggatgtctgtgcaccacgctcatgcctatgcctgaggtggtcagaagaagacatagaaaaccttggaactggacttagggattgttgtgaccccatgtgggtgctgagaatcaagccctgttctctgcaaacataacaagtgttcttttgttttgttttgtttgtttgttttgttttttgtttgtttggatttttgttttccgagacagggtttctctgtatagccctggctgtcctggaactcactttgtagaccaggctggcctcgaactcagaaatccccctgcctctgcctcccaagtgctgggattaaaggtgtgtgccaccacgcctggctataacaagtgttcttaagcaatgagccatctctttgttgttttggtttgagactggatctcatgtagacagggctagccttgaacttctgcctcctgccccatctctcaagtgctgggattatagacatgtgacgctacatctggatttccatcaggagagagtattcctttaggataccccttacctgtggaaagcctggtacagagccagtggtagagacatcccagaagcaattgtccttgtccatgaattccatacctggcctgaggcccagacatccagtttgagcatgtcctcaaacaaacccaacttagccagagtccagtggctgcttagcgttctctccctgcacacaggctttgagagtcaagaggcccagctcccaacctacagggcaggactgctttccctggagaggctgagcagcctggaagggaggctaagggtaccacaaagtgtttacaatagtaggtggcaacacacttcttcctcctcttcctcttcctcttcctccccctcctcctcttgtttttcaagacagggtttttctgtgtagctctggccatcttagaacttcctctgtagaccaggctgtcttcgaactcagagatccacctgcctctgcctcccaagtgctgggattaaagggacagcacctttcatattaaccaagagacagggtataaaaagctcaatggagcatcatgtcacctagtgccccttgcgattgctgctgtaggtccctctcccccactgccaccaatgatctgaagacaattaagcccagagtggccagatcatcctaatttctcaagatcagttggaaactatattcccattagggctggcaagatggctcagagggtaatgtcacctgctgccaagtcagacagcctcagttcatagtctacacagtagaaaaaaggagcaagcaactcccaaaacttaccctctcctccacttgtgcaccctgacatgtatacacacacacacacacacacacacacacacacacattaaataaaatgtaatacaaaaattttaaagaaatttcgctgccatcaaaaatccttgtcatacatctttaatcccagaatttgggaggcagaggcaggaggatctctgtgagtgaaaggagaggcaggaggatctctgtgagtgaaagggtgctacattgtgagtttcaggctagccagagctgccaagtgaaatcatctggagatagagagagagagagagagagagagagagagagagagagagagaacgagaaccttcttgtcaagcattggcaactaagctggggaaattaatatgttcctggggtgtttgtctgatttggacttattgtagaagaagcacttgaggcagacagcaggtagaacttcctgagataagtaggactgtaaggaaggggacaggaaggtaagtcagagatccatggatcaccagcagctgggagctgtgccgtagtgatgaagccccttgagcaactcctgaatgtccctctctgcctctcattcaccagacaaccttaagccacaccctaagagtgcctcttagaacttgattccctatggagaggcgtaatgggctctccctctgcagggtgctgagaggaccaagcacagggactctgtctgggaacaggaatataatgaatgccctgtctctttccctccttctcatggctgctactggggcttgtgaattctggagcgacagagacatcccctgccctctggaagcctctaggaaccagagagctagaccacatggaggaaaacaagggaagtggggtctagaggaggtttggggagctcacagaacaggtgccctgccacccgcaaaaccttggtatggtcatggccgggtccacagtgtgtcttccatagtgagtttactctcaatcttagccagagtaagatgtccgggggatgtttagccctcaggctcctgtgagggtgacccatgacctccacagtcatgtctcctgttttgttttttggtttttggggttttttttttgttattttttgtttggttgggtttttttgtttgtttgtttgttttgttttttttctttttggtttttcgagacagggtttttctgtgtagccctggctgtcctgtaactcactctgtagaccagactggcctcgaactcagaaatccccctgcctctgcctcccaagtgctgggattaaaggcgtgcaccaccacgcccggctcagtcatgtctctcctgagctcaccagcagatctcttacccaagtactctccactgcttaattttcctgagaaatacatactagtgaagaaaaactccacccctaccccaatgagagttccgtgccctaaggtcatctgccctgtggtttcattgacaaggcagtgagatgttaaatgcacacatgggggctaggggttcaaaccctaattctgctaaagtcctgactgtgtggccctgggtgtgtcattcatgcctctgtgcttcagctgcttcctctgtgagactaagagaacgggttacagaaacagcggactgagcggagaatgccataagtccttggtgtagagctcgtgggagctgtgtaagagatgggacggaaagaaaagatccttaagtgtcttctacatgcatttcccaagccctccatacacagttcccaccgtgccttgggtatggggcaggctgcaacagatctctgcaggaagaagaggctgcctcggccccaggtggtgacctctgacacagctccgccattccctggtggtgtctacaatctgcagcctcgaaaggtcatttgcagcccatgcagtgacagtcaccaatgaggcccctggactcaggctgcggccctgcgcagaggtcgtgagaatggggcatgcttacgcacaacccccagacacaggggagccctgggttggttttcctgcctccaggcctgaagccttgttaacctgttcattcctcacccagcctctgaaggtaaacaagactcccatcagtgccagagctggggaagaaaggccagagctcacagacccggggaataactgtgacatcctgtcatgtgacccaggacatcagctcctgggctccctcgggaaccattcatttctattacacaggcaccacggcttcctgtccccagagccatccttaggccaactcacccatctcaaccaggagagctgctcccagccacctggtgtcctggtgtgctttagtcagggtttctagtcctgcacaaaacatcaagttagggaggaaagggcttattcagcttatacttccacattgctgtccatcaccaaaggaagacaggacaggaactcaagcaggtcaggaagcaggagctgatgcagaggctatggagggatcttacttactggcttgctcaacctgcttttttttttagaaccaagaactaccagcccagggatggcaccacccacaatgggccctcccacccttgatcactaattgagaaaatgccttacagctgaaactcatggaggcatttcctcaagggaaactcctttctctgtgataactccagcttgtgttctgttgactcaaaaagccagccagtacattagaccccctctcaacttgccacacaaacacaccactattaagcctcaacccttactttcttattcatcctcaagatcaaaataactttaaaagtcccgcagtctttacaaattcttacatattaaattttcaatacctttaaaatattcaatctcttttaaaattcaaagtctttttacaattcaaagtctcttaattgtgggctccactaaaatactttcttccttcaagaaagaaaaatatcagggcacagtcacaatcaaaagcaaaagcaaactctagctgtccaatgtctgtggttcactcacaatcttcttggctcctccaagggcttgggtcacttctccagctctgccctttgtagcacaccttgtcttctaggctccagatgcctgtactccactgctgctgctgttcttggtggtcatctcatggtactggcatctccaaaacactgctgtcttccactgtaactaggctaaaacaatagcttctcataggcgctcttcatgaagccaagcctcaattccttcgcatgaccccttcagtcctgggacatcaattgctactgaggctgcacctttaccaatggtcttccatggcctcacagtgctgagcctcagctgctcttcatgaccccttccttcatgccttcaaaaccagtagcacctgagtgactcttacacattaccaagtccagccataacacaaggtacaaccttggctatctctgacacacagcttctttgtgctctaagaaaccacttcccagaagatttcacctcaatgatgctggtctcttcttttttttttttttttaagatttatttatttattatatgtaagtacactgtagctgtcttcagacaccccagcagagggcatcagatcttattacgggtggttgtgagccaacatgtggttgctgggatttgaactcaggaccttcagaagatcagtcagtgctcttaactgctgagccatctcatcagccccctggtctcttcttaatcaccactaatttcttagctccagctaaccagcatcaatagtcccagtaatgcaaaggtttcactttagtagttctggtgtcttgttaatcacagctgattcttcagccccagctaaccaaaaccacagaatcttcacaataaaaatagcaacagtcctgataagagtacctctgaaattccatcttctgcactgttttcaacattctcttccaagctcctgcagaacatcccacagagctcttaacatcccaatggctcttctagctcaaagttctgaagtccttccacagcctccccaaaacatggtcaggttgtcacagggataccccactatgctggtaccaatttctgtcttagttagggttttactgctgtgaacagaaaccatgaccaaggcaactcttataagacaacatttaattggggctggtttacaggttcagaggttcagtccagtatcatcaagatgggaacatggcaacatccaggcaggcctggtacaggaggacctgagagagttctacatcttcatttgaaggctgctagcaggatactggcttccaggcagctagtatgagggtcttaaagctcacactcacagtgacacacctactccaaaagggccacaccttctaatagtgccgctccttgggctgagcatagacaaaccataacatggtgatgccaggacaccatcctcctggagtggtagtgcttcaccctgcaaaccacgagcacaggacactgctgctcctctctagctggagtcggctcctggcagaggagcagtgctgcctgacatcagaagcacagcatttcactcccatggcctctctgcagctgtattgagagctaactggggctgagacatgtcttgcaaggaagagcccttgatgcctgagccatgggtggaggcaggcttgtttcctgcgctctccccaactccacggtctgtttcaccttgcctccttcctcttgcctccttgtgtcagacagaatgatggaaatatgcagagtcatgctggaagcacttcttgagtgtttggaagccagacagaatggggtcagtggtccctgaacctcttcccccgggtctccataagaagagaccttgccatgggttctaccagccatgaccacacctttcctccttcagctcagactgtctctgcttcaaaggcagcgtgtgttcaggcagacctatacagtgcgggaaagagggcgttggtaaggaaggcatttgggatgcacgatgcacagagaacctttaaaaaggctgatgctccctgatggagaataccacttccagggatctgtcctaaggaaataattagtgaggggaaaaaagggttactattacatgcctgccatcccagctctgtgtgggctcaagcaggaaggtcctgagttagaagccagcctgggctacatagaaaaaccctgtctcagcaaatgggggacaatggtttacagatgaagatgtttatcgagctgttgttaacagtagaaaatctcagacaagctacaatttcaccttctagcacagtgttctcaacctcccatatgctgcgaccctttaatacagaccctcatgttatggcgaacccccaccataaaatattttttgctacttcatagctgtaactttgctactgtcatgaatcataatgtaatacctgatgagcaggatatctgatgtgtggcctccaacccctgctctaggagataaatatgtagtgtagtgcatagcacttagaaaattatgtctgcggaatttttagaattatttatttgtttgtttgtttactgtatgtgagtacactgtcactgtcttcagacctctgtagttgctgggaattgaactcaggaccttaggaacagcagccagtgctcttaaccactgagccatctctccagcccccagccctggaatttttaaaaatatgtatttagttttattttacatgtgtacctagtacccacagaagagggcaacagatcccttggaactggattgcagatggttattagccaccatatggatactggaaaccaaacccagatcctctgcaagacccaaaagatctcttaatgctgagccatctctccagtcctccagaacaaaatacttcaaaatgtataggaaaggctgagtgtggtggtgtacgtctgtaatcccagcacatgggagactaaggcaggaaggtcagtgcaggttcaagtccatcttaggctacatgtaagaccctgtctcaacaaacaaacaaacaaataagcaagcaagcaagcaaacaggggaaaaaggaaatgggtagaaagattaattggaatatctccattagtcgcgagcagttctatctctatacttcataatatagacacatagccaggcgtggtggcacacgcctttaatcccagcacttgggaggcagaggcaggaggatttctgagtttgaggccagccgggtttgcaaagtgagttccaggacagccagggctacacagaaaaaccctgtctcgaaaaacaaacaaacaaacaaacaaacaaacaccataatataggaacatgttacttcctaaatatgaagtttaaatctagagttaagactgtcccatggtgtatatctgtagggccagctgttccataagctataagctaaggcaagaggatcacttgaccccaagagtttgggtcagccagcataagagtaagatcatgtctcctagtattaaataggttgaagaggtcttttagagaataaaacacctgccagtcattgtgtctgtaaccccaaagtatagtctagctaaggcaagaggatttacattcaaagccaacgagagctacatagtgagaccccatctcaaaacaaacaaaataccaaccaatccatgaaggatatggttggacgatggacctacccagcttgagcccacccccaaaggtcacctgaaatgcttgaaactaggagtgttttagattgggggggggggcatgggaggtttgggagcatttgtttagatatactagttaaatattcctgatcccaaagccctaaatccaagtttcagagactgtggagagatggctcagcagataagaacacttgctgcataatcaccaagactggaggtcggatcctagcattcaagtaacaaactgggcatgcatcccaagcatgccatcgccccagctcttgggagaatgggggtaggatgatctatggggcttgctagcttccagcctagcctagaaaatgtgggtgccaggctcaaggagagaccctgcctcaaaggaatcggctgagagtgatagagaatgttgccttcttctggcctgtacacatgtgtgcatacacagacacatacatacatacatacatacatacatacatacatagacaaaaagtatttggggtttttgttggtggttggttttgagacagggtctctccgtgtagccttggctatctggaactcactttgtagatcagactggcctcagacccacagagatccacctgtctctgcctcccatgtgctggcattaaaggcgtgcgccaccatgccgggcctgggacattcaccctgtggtagtatcatgtgattgtcacaaaggctctgtaggaagtagactcattctttgaagggaaacttgtctacatcttctctatgccttgcccccttctcagactgaacatggcttccccctcccccaggaagtttcttagggacgtagaagcagctgaacctcacatacttcagggaaagatgtgaatatgaatattcatgcaggcacatatgcactttataatataaatatatacctaaatagagacagctgaagtatggcccttttagcataggtaaggctgaatcttgggcctctggtgtttattgtattacttaagcaactcttctataggtttgaaactttccaaaatggatcctcggaggaaggagcaccagtcagccctctgccctttgctacaatgggaatatactttgagcccacaataataatttgacagtttaattcgttgagtgtctggtgagccttccgtgttgggagtgggaactcttgggctgtagctgtccaatgtccacacttccagggtacagatgtttcgctccactgacctgtagggtgagaggtcctggtttgatgtgctgactcagcaaagtgcactggctgaagttcatgccttggcagcctgctcctccccgccctgcagtggcccagcctcccacccccagcctcagattccacccattgccttgcaccgcctcttccccacctcacaacccttgcttacggttcttttccccttttcttttttttctttttaaagatttatttatttcatgtatgtgggtatactatcactgtctccagatgcaccaaaaggtggcatccgatctcattacagatggttgtgagccaccatgtggttgctgggaattgaactcaggacttctggaagagcagtcagtgttcttaaccgctgagccatctctccagccccttcttccccttttctatttctatgttctcccccatggcctcccctgcctgtatatattaaactaaataactctccttgggctggtgagatggtccagtgttattaccacataaatgcaaacccaaacataatgacctgagtttcatcctcagaacccatgatggactaagagaactgactcctgaaaggtattctgtgacctccacacatgtgccatgggacacgtgtgtgcgtgtgcacacatacacatggttaaaatcccaaaggtattaatagttaaagaataaattaagtaagtcagcaaccttaatccttatgacaacgtaaagccagggtctctctcattcatccttcgcccgcttctgttcccagctgggtctgatttgggagtttttgcggttgttttgttttgttttgttttgtttttgctttctgtactggtcttgaaccagggccttatacatcttaggcagacactctaccattgagccatatctctagtcctttttattactttaattttaaaacaaagtctcagtaaattccccaagctgcccttggcctctttatatagcccacagaggccttgaacttttgatcctcctgcctcagcctctgaagtagctgggatcacaggcccagctccagatgattgtttgctgaggtgtccttgacagttccaacatgttcaccctcttcacaagaatccatagggacctgggctttgcctttcccccaaagcacctgtagttatcagcagagccagcaagaccctgtagtttcctgttccagcccctctgcctggattgtcccccaccccatccctacccccgcctcactctccttgagaactcgtccacatcctttgggtcacaacatgaatttcacttccccagggaacttttctctcaacctgtccaggcttatgacctacttaggaaaatacagaaagagctcaatgaatgttacccacgccccaaagcaaaagcaaaacctcattgtgttttaatcctacttaaacctcccctctttccttgtgagtctccccacctgcaatccagtcccctaattctcaggtatagattgcctgctcattgttcgatgttaatagatccctgaagcacaggctcccgggtagctgaccatgatgtgcccgcctctgccaagctaagtcctctctggatggggtctggtgctccaaggagggctttatgcacctgtagaatttgtcttgttgctaaggactatgggaggagatggtgagatggctcagtggacaaagacacttgctaccaagctgacaacttgagttcaaaccccagatcccttgtggtggagggacagaagtgactccaactagctgtcctctcaccctcacatgcttttgccatgacatgtgcatacacacatacatgatcagataaaccagataggtgggtaggtagataaacagagatagatgataggcagacagatagacagatagacagacagacagacagacagacagatatatacatacatatatacaaacatacacatacatacaaacatatacacatacatatatacatatgtacatacatacatacatacatacacgtttcttagacttggtttttttccagcatgtgtcacaaagcaggaaattgaggctcagactggttatgtcactcacccaagatcacatggccagcacatcaaaaactagaatttgaattcagcttcatggtgtttttatttgtgagttcattacaataaaattatattttttaaagagcagaatttgaattcagctttatgatttcttaaaaattttgagttcattacaataaaattatatttattaaaaaacaacaacaacaaaaccgaataagctggctatgatatctcaggcctttagtcccagcacttgggaggcaaaggcaggcaggtctacaacatgagttccagagttccaggatagtcagggctacacagagaaaattggtctcaaaaaacaaaatagaaaaaaaaaaaaaaactcagaatttaaacctagatctgtcagccacagcctgtattttaataccttccacccccaataaccgatttccaagattagatgttctatctatgactttcatccccagaacacatgataactattgtttaggaacgagcccacggcaattccaaatacacctatttctgctttgtcacagctgtctggtccctgccaacagaccagggaagtaggtcacggctgcagggaccacgcgctggctgctgcttctgtttccgggaccaggggtggtcaaagcctgccaggagtatcaccctccactggccctgcttgggtttcatatcctgagcttggggagccgtgaccaggtctcacagtgctgactgttgccttctagaattctctgtccgtggctgctatgtgaggcagtgtctagaggctcgggcactggcaacaagcgggtattgatgagatgagctaggagtgtgtctcttcattctcctggggaccgttgcaaactgagagctgttgagtgctgaaggagggttacctcttaactcacctacagaacagtcacatggagctaaccttcgctttaatagcccacctctctctattattttcaaagtaaccatggtgtgagctgtttgtaacaatggccagagaagaaactgttactttaatgaactcactgtgccaacaggttttgggggttgtttgtttgtttgtctgtttgttttttgggtttttttttttttaagtcaaaataaatggctgggcatggtggtgcatccctttactctcggactcatgaggcaaagacaggtggatcaggcggatctctgtgagttcaaagccagccctgagcttcagagtgagaccccgtctccaaacaaaattaaataaaattcataaaaaatttcagtaaatgttagtcaagatgtagtgttccttgcctttaattccagtgcatgggaggtagaggcagaaggctcaacaaaagtacgaggccagcctgatctacataatgagttccatgtcagccctgtctcaacagctcacctccctgcaaaataatgcagacaaagagatgagagttcgccttggtgacatgcttacacatacatgcctgtcattccagtacctgggaccctgaaacaagaggattgccatgagtttaagacttgcctggtctgcgtagcaagattttgtcataaaggggtggggcctggggatgtaactctgttagagcacttgcttagcatatctgaggccccgagttcaatccccaccactacaaagaaaaaacaaaagtgtctatccatgtatgcctaagttttgatcttaccattgcagcctgatagtgtgaagctttgttttgttttgacttttgtttgtttgttttttcaagacagggtttctctgtatagtcccagctgccctggaactcactctgtaaaccaggctgctaacctggaactcacagagatctgcctgcctctgcctcctggatgctaggaataaaggcataccatgcccagcttgtttctattttagatatagggtctcacttgatagccccggcctggaacttgtggccaatccccctgcctcagcttcctgtgttggggggttgacttcagatttgctatgaagtcagtgatgacctcaaacttgatacccctgcctctccaaactctaggatacaggtctatgtcatcagttccagtttgtgcagttctggggtccagcccaggaatctgtacctcttataataggcactgtacacactgagctatatcctcagtcttggagaaatggtgtgtgtgtgtgtgtgtgtgtgtgtgtgtgtgtgtgtgtgtgtgtgaaggagagagggcctagcagttagaagtattgtctgctcttctagaggactcgggttcagatcccagcatccctttcaggtagctcacaaccttctgtcttcagctccaggcagccccctggcaaccgtgggcctacagaacacacgttcctcacaaacatggaaactgctggcccagactggcacagcccagaggaaaagccaactttttccctttgtcactcacagactgggccacttcccatatcttgtgtagcctcatcatagtgacatccactctccttcccaccgagaaggtgaaccctggacctgaggccacctcgagcttttctcctttgagtttaacccttgtgggttaacgactgggaaaagcacgggcaagcacttggctctgtctccccgagctagctgcctgcatcccagcctcatggggtgacctccggtggaaggtcagcagctgacgttgtgcttggccttgtccctcctcagagaccttcccctcctcccagcagtggggcagactcccttgggtagcctgcctctgctctgatcttccctccaccattcccctccccggcaggggcctcttcaaggcttcattatgcaacctgatatgttcttggcccaagacctgcctggagccgtggtcaggagtgtccttgtaggcagctctggtgctcaccactcctgtcctttggaacccagaggtggcaacggcatctggattgtacaggactgctggttccccaacagtctcaggacccaaacttgggagcaatgctctttgttaatgtccagctcaaaggcccagcagtctctggctcctgtggcccttagggagctatcccctgaagcgggtgcagactttcaacattctttgggcctcatgcctctcccatctgggtccctccaatatattctctttccagtcactctttcccacccctctgcctctctctcccatctcaggaaagcttgtgaggtagcacatgcaggcctggaagtgagacccgggaggggagtgtggagagcactctacgtagccactattccactctcccggccattcaagaggtgcctgccccagacacaagtattgacaggtgtccagtagctcaaaatagtccattcaggagtctctaccttcaagatgcttactatcagtgggaaaccagaggaaggcgtgacactgataacctgcctgcccagtgtcaagtggaagacaagaaactcagcgattcctgccggtgtctccagattgttcaattctagctcaatcctccagggaaggagacaggaccagggacaccaactatggaattcttccaaagagattgtgatgatagatctgtttctggtgtccctaagctttctctgctctcccagccttaaagggcagggggagtcctctgaaacccaccctttgccccagcaccttccctggtagccatctggtctccttgtccatgatctcccgccctttcctgttcatgaacgctctactttaatgtgttaacccagcatgtggcctgtccctctccatgcagggacggttgatctgcaaatcctgtggcccaccagccctagaaggtgtcctctggcagggcctttgccctgtgccctctgttccctggtgggaagtagcctgaattccaccactgccctatcttttgggatccccacctcacctccaatatcaatcctgagagtctacaatgggcaagccacctagacagtttaatgcagacagaatttccctagtgacatggcttaagccaaagccagattgacagcagggaactctgtgtacaagccattcctcgggatgacttgtttatcctatggatcctgaggtccctgtggggagactggagacagggaagctgagaccagccaacagcagactctgcgggccagctgggagtcctagcttgtgcccaggttcaaagggcatctctatttttagacacggacaaagaaaagaaatttaatttctcacctcttatgcaactttttatttggggtgttttgttgttaaattgatgttttgcttttaggcagagtcaaactatgttaccctggctggcccgaaattcactatgtaaaacaggaaccaggctggccttgaactcacagacttgcctctgtgtctgtcccacctcattgctgggattagtcatgaactgccacatccagcccttatgtaacttttgaaaacactctcctgttcttcttctttttccctcttctttgttttttgaaataggatcttgctgtgtagcccaggctagccctgaacatgtaataattctgcctcagtttccccagtgccaatagtataagtatggacccgcgacacccacctgtgtatgattttgctggtgactcctgggacactcaaaaacccagaggaggcacgtagcatgattatcattctgtttttccctcttgatgtgtacatgtgcacacagtacaaccacacatacacatggacacatgtgtgcacacatcctcaaaccaacatacacatgcttgtgtgcatatgcacacatatacacatgcatatgcacacctacacatgtgcttgcacatacacaaacacatatgtgtgcatgcatgtgcatgcactaatgtgctctccctccctctcttccacgatgttttgtcctttttaattttccgagtagaatttcatttcctaagttctttgggcttctttctcattcccttcttccccccatctttccctgaatcccccttcccccactcccaccagcctccctgtttctggacataattcccatgcaggccttctttgggtctttctgctgctcagtggacacttggcaaccagcaaggccgcggcttcagcagagtgttcttgaagctcagaacctaaatgtgtgcccgggcctcctttctcctgcctctccctgacctagatttctgctgtgtccactctaagtggtccaggtctacagtgccagtctgtcctgggcgggctggatctggtgcaagcatagtttgttttgcccaaatgagagcatgtttattgtcctttatcagctgtcgacatggcagatgttgggaaaatgctacagtgaggaaagcaagcattcacaggggtctcctgagggggcgttcacaaggcggtgtctcccaaggggacagctccctgccatacttttaaagaaatgcctattcttcctgcagcctgagggtttttgtctccattcacaaacacgttctctttaaaatgatattcatgaataatttctacagttgctcttcgctcttgaaggggcctttgtaggcccctcacccctcccacaaagcggttggcttcacgaagggtgtgccaggacagggttttggcatagtccccggggtgtcacgggaccaggggatgatctcactgggtccaccatctgccctgcctggccagaggctgcatcgggagggccaaggggcaccacgtgccctgggtattttcaaacaagaacctttagagagagctgggaattctttggctaatgggtgtttctccccttgtaggctcccaattctataaaactccagaggcttctgcaagggcagcctcaggcagagtattattataacacaacctgaagcaaacctgtaccatatgaggatctggggtgagctgagacatttgccctgagaggctgtttggtctgaaaatacagctatcccataaaggtttagatgcccccataaggctttaatgataattaccagagaccgcccttatgtagctcttaaaacacatgtattaattgtttaaccctcacatcttctagatatctgaggctcaaagaggttgagtgactgaaccagggtcacacagctagtaatgaaggatccaggcagccaagcctgactttgccctgtcctgttgtgaattctgttcctgctgccaccacttagaaggaagggggtgcgggcaaccagatatggtggcacgcatctgtactctgagcacttgagaggctcaggaagggttcagagccaacctgggctacatagtgatgccctgctcccacccctccgctgaagagagaactcaaacctcagtgcccttgacccagggggtcatttcttcctttccgtgtgtttgtgatggacactgtttattttctgtctgtccaaggctacaccaagttcagggcattcattacagagggacaactgctccttagccacagctcctgacactgccctgcctccacacccttcatgacatgtcctcagactcaaaaaataggaatgaagacatctaaaaaaattgctaccttccctcccccaattctcctcttccttccccagcttccttgttatccccatctaatgaaatgtcttccccaaaaagagccaatttcggcctgtttcctgtggcctttcaagcccctgcctcccccacggcttgtgggcaaatccccagcaagctgagactccggtggaacaaagttccagactcagtttgtccccccagatctgcttcctctttcctgtgctcacccaaggacagcactaggagtgaggggccagtaccctaatcttccccggggttgccctttaccctacctctgctgaaaccaccccagaggtcccccacttcccctcccctccaactgacccacccggcccttggatgccatctctcacctctatctaagcagagttcaggttagccctgaatgagtctcttctggataatccttatcgtctctattttctttattatttctttattttttatttatattagtacactgtagatgtcttcagacacacaccagaagaaggcatcggaccccattacagatggtggtgagccaccatgtggttgctgggaattgaactcaggacctctggaagagcagtcagtgctcttaaccgctgagccatctctccagccccttctctatttttttttttttttttgaagtctccttattgggtgtccatgtgtcatggtgacaggtctcttcaggctactgcagttgcagccactaatgtgccacctctcacaagcagtattttagtgacactcacagccatcccgcatgactgcagtagttctgccctgttacacaaaaaaaaaaaaaagaccctgcagtttggatggggaaggtggcctcaacatctcccctctggtgagtttcccagctgacttaaacatacctggcttttgacttcacactagtattctgtctagacaagtctagggagagatgtgaaccaagaatctccctcagggtgatgattcagatccccaagctagaaagttggagagtgggccacccattcacctagtaaactgagcccgatgcattctgggaggaggtgggaggaggttggggtcgccccattccggttcagagaggcttgatgcaaagaggccatctctgatcctgaggtctgacttcaggggaacctggctcaaaaggagcaaagggggtccccctgggctctcctgtgagcctccctaagcccctgccatgaagatgagggtgtgtgcttccaggtgctgtctagaagacagatgagggctgaggggaaggttcagtgggtaaattagcacttgaggtcaagcattagaacctgaactggaatccctgtttaaagccagacactgtcacactgagtgtctgtaatccccgtgagtccagaggcagagccaggagactctctataagctggcagaccagctaacctgataacccacaaggcgagaggtgaggactgacacctgaggttgtcctctgacctccccatgcacaatgtggctgtgtaggtctgcagttagacaagacctcacccagcgtcagactgaaggcagggtgatttaaggatagaattgaagaaacgcacaagtagtgcctggctatagaattatttgctcttattaaaggaaaataagcagggtttacaaagagggaggctgcaaaccaggtatggtagctcttgcctgtaatcccagcccttgggagattgagacaagaggatcgggagttcaaggccaagctgacctacatataagaccttgtctcacaagacaggggagaaatggaatggagggtttaaaagcttcccttatccatccaggcctctgcattcagaggactcgggtagacttaaagtggtgacagggacactggtgtctcttgaaccagtcaggctcagcaacccacctctacccattaagatatgtcacatgatagctcctgcccccaaattacttgggtctggggtgtgtttcagtgacaaagcaattacactaaagacacacacaaacacacatacacacatacacacatacacacacacacatctcagcaagggaaagtagagggaaaccaaagccacacagacaatgcagactttatttttggggaagggagagttgagacagggtctcactatgtagatcaagtttgtcttgaactcctagagatccgtctgcctctgcctcccgagtgctgggattactggcgtgccccacctttaatgctcaactatttggccatatcagtggttacaaggcattttagttcaaacaagcaaacaaaatccccagaaactcttacttaaaaccagaactgtagccaagcatagtaatacacacctccactcctagcaaggggcgggggtggctgggagggagggtcaagcaggaagatcatgagttcaaagctctcctgggctatttagcaagatatttcaaaaataccagaactgtcctctcgggcacaatgtatggcttttctaccctggtggacagcagccagctgacatcagccatggcatctgtgtgcacacagcagcaccacctcctctctggagcgacttcccttcatctttttgcacaagtgcccgattgttctcccttgatggaggctcagaatcatttcatctcctcaccaacgtgagtctcctctgtgtatgctcaatttcctcacatttaattcggccattttgcttgatacttgggtccgttattgctctgttctagaatgttccgtggtaaaccgcaatgtgatatgacgatgagctcagaggtcagccttgcaagagcacctgtcctcacctgcccttgttagagagacgagtggtgacatccaaaaggatatgtaacttctgagcctccagaacctgccctcaagagggcccttaagccttggaggacgatcttgtctttcagtgcagcctgatcccctggctgttctgtgtccatacgggagggctttcctcgatgccttggctccagtggtggcaagactgcctgctgcttcagccgcctttatatggaaaagtgcaggagctggaccacaggcatttgcatctagagtacgagaagggcctcagaggaatccaggtttttattttcaactttggttgaaccagtgttctcatactccttagctgacatcggctattgctgataactcacggagcctcaaacttgacatgtagctgaggagagcctgtgtgttagtcagggtttttattcctgcacaaacatcatgaccaagaagcaagttgggagccaggcgtggtagtgcacgcctttaatcccagcactccggaggcagaggcaggcgggtttctgagttctaggccagcctggtctacaaagtgagttccaggatagccagggctatacagagaaaccctgtctcgaaaaacaaaaacaaaaaaacaaaaaaaaaaaaaagaagaagaagaagcaagttagggaggaaagggtttattcagcttacactttcacattgctgttcatcaccaaaggaagtcaggactggaactcaagcaggtcaggaagcaggagctgatgcagaggccatggagggatgttactggcttgcttcccctgccttgctcagcttgctttcttatagaatccagcccagggatggcaccacccacaatgggcctcccttgatcactaattgagaacatgccttacatcaggatctcatggaagcattcttcaagggaggctcctttctctgtgataactccagcttgtgtcaagttgacccataaaaccagccagtacagctggaattccctccgtctcccaagtgctaggatttgaaccctggctctttgaatactttcttcatggcttcgacaaattaatctttctaacctatggaagcaaggacagcaaccagctgttgtagccattaaataaaccaataccaggaaccacttgcaacatggtttctgcccttaggagaatcctgcttcctgcctacagacactgacagagtccaggggaacagaacagaagcagaagagagatgcagtgaacacggcaaaaaaaaaaaaaagcctcttttaaaagatttcctactttatatgggagggtgttttacctgcatatatattttgtgccacatgcatgtctgtgcccacagagataagaaaacagcattaggcatggttgtgagccaccagatgggtgctgggaaccaaacctccggtcctctggaagagcaacaagtgttcttgacctctgagccgtctggtaagcccttgattttttttttttttccagacaaggtttctctgtgtaaagccctgattgtcctgaaactctctgtagacctggctggcctcaaactcaagagacccacctggctctgcctcccaagtgctgggactaaaacgtgtgtgccaccacagcccagctcagcccttgatatttttttgaacccaggtctttctgtgttgtcccagttggccttgctagtggtgtggtcaaaactaacccttgagagcaatgtccattgggaggtaatttggaaactctatctctacttgcttttcaccaaatctgcctgctatctattctagatttagtgggtttctttggggcgatggggttgtttgttgaaacacatatgtttctatgtatatatgtaggaaggtaggtagcctcagctggcctcgaactcccagagatatgcctgcttatgcctctcaagtgctgctggggttaaaggtgtgtgcaacaactcccagttagaatttgttgttgtcgttaaaagaaaaacagacaaacaacactttatggacataattaacatgatgtaaaagtgatgtttcaatacattcacaaagcaatgcagtcatcccttttaacccgaggccagtttgggtttttaaccggaggttgctgaggacgatccatccttcctgagaagcagaacctgactgtggcagatccgctgagtcacagcgtcggtgtccgagctgaacccgggtctggcgtctggcgccccctagcgaggcctgagtggcagaaaaccccactcggcgctgcccggagcaccgcgggccccccaccccacccctcgcacccaggaagctgctgggtccaggtctcacaggcgtctgaatcacaaacctcagaccagcctcactccgcccttttacgggggaatgaaggcttgtgggaaatgaagcgagcgatgtgggacagtcgcttagaaatgggttttagtggcaaaagatctgagttcgaactctgcttgccctagctacacgggttactcaacgtctccaagcctgtttcctcacatatgagaaggtactaattattccttctggcgtggttgtacgtcagacatgatgctgtaaactgcaggggctggagtttagaaccactgtatttaattatgcaactacctaagggtgttgaggcccctctcttacccaaggagctctccagcagctcaagggctggttataagccgttttgccaggagcaggacccaggcaatgaagccattgtagctacatctattcaagaaacaaaccccagactagggcagtctgcagtccagggaggttcccgtctcccttgggaagaagactggaagatctgggagctaggaaagaccagggcgtccttctggagcctgaagggaactgctaattggtatttgtagacttctccagcaagttgaagctccagaagggaaatcactccccagaccccagcaccactgggaaccttgctcccagggtagcagggttggaagccagaggatatcactcatgtcctcagtagactgtcaagtccctgtggctgcttgcccatcagggattctcatccggcccaccgtaaaggtttacagtgagctcagaggctctgccatggaaagaagccgaggaaaaaatagcagacaaattcaagagagcaagcagggccccaggagtgtttcagagagtcagcaaggtggccaattactgagggttcccttgcccacctcctgacacttgagagggccacccaagatggcaaatgggctgtacagaagacagaaggaccgatgtctttgagcctgagtagccgctgttcttcaggaacagaaggacctgggacttcatctgcacatccctgggcctccaaaggtcggggaagtgcatctgttaaggtggcaatagtcatgtcaatggagatattgatctaaaaggatagcctgcaagggctcccgaggggcctatgaggagcactctgtcaacaactgccttctttggcaggtgtctggtaaatacttcctggtgccaggtcttgaggctctgaggcaggtgtcccacgtgcctcttccactcctgtgtcatcagtgtgactcataaggctgggaaagggtcatcccctcttgctctccagggatctgttctttctggggtttggtccagtgaaacctcaaggatgttccagaacattctttttttttttttctcatcaccctccctcccttcctcttctctcttccttctgccttccctacactgcagatggaactcagggccgtgtacatgctaggctagtgttctaccatggagccccatccctggcttggattggctcctaggtcctgaaatcccagccgaccaagcccagccatggagggtataaagaaggaaccagatcctaagggcacctgccctcccaggactggggacctacctacttacacagtttttcccatcccccacaaaggaaaatcgtgacccttgagatgtgcacggtttatttattttacatagtaaatagtagcctgtgtttttaaggatgatttaatttctgcccagaccctgcaattcaccctgcttccagggggcgtgtctggacctctagctctgaccggaaggtgggatgtgtgcaggggcagagggatcattaggagctgagtcatggcctggatggtggtgtatgcgtgggtttaatgagacaactgagcagtcggacacaccatggaccaccatccccaatctacaggtaacctgcagagcatcgcccagatgcaggagtgattcttggctctccagctcccacgatgggatatatggaaacttctagaaacagctgaagacaatccaacatggaaagcaagcaaaactccacattgagaatacaggatgtccaagaacataccaccctgaatgtgggtgatctcatctgatctcttagactctacacagcgtcagacctgactggtgcttagatgggagaatataaagaggagtcgctgtgcagggattcttgctgataactcatttatttacagatttttatttattttttaaataattatttatttattttatgtatatgagcacactgtagctgtcttcaggcacaccagaagagtgtgtgggatcccatgacaggtggttgtgagccatcatgtggttgctgggaattaaactcaggaccgctggtagagcagtcagtgctcttaacctctgagctatctctccagcctcctagttacagacttttaaattattattattatttggagagtataaaaaatatactagagaagggacagcagttaagaacactggctgctcttccagaagacttgggttcaactcccattaccccatggcagctcacaagtatctgtcattccagtcccagagaatctgaccctttcacacagtcataccggcaggcagaacaccaatgcacataaaataacttttaaaagaaatgaaaatatactggagaagagacaggtctggccatccgctggtcctagcactcaggaggctacgacaggaggattaagagtttgaggctcacccaggtctcaaaagtacaatgaagggctgaggagacggttcagtgcataaagtgcttgtagcacaagtgtcaggacctgagttcaaatccccagaaaccacatgaagctagacacaagcagtgatcttcagcaaacagaatggccccgttgtcctgtccccatggtgtccctttcccttacccctctttgcctagccatctaagcccctgcctttagtgtacttgtggattttaatcacctgcctaactgcactctggctttgaaaatcaatctaccagtttctaagccacacatcctgtgcctatgtcttccttttcatctcctttacccacatctagttctgacctttgaatctctccctggacatggttgtcacccccatgccagcaggtactcttatactcccattagccctgcacattgctgccaagttcaccttagggtacctctttggtgctgtccctgctggtgagcagagtccaaaccagccacttcatcctcctccagacactgctatgccctgcaattgactcgattcataactgactgggtggacatgcaggggctggaggagggtgctggcagatcttcccgtctagctgcaaggcagggcgctacctccgtaaagatgcaagcgcagcctgcacaggggcctgggttccacagtcctggccatagtcagctggcattcacagaacaaagcagccactctcttcctcccagcctcctttcctgaggagagaggccctgcaatcaccttccctccataactacttacaagtcaacagcacttctcagtgatatggaaccatcgggaatggctgtaagtcactgcctcatcttgcctcatcttcttatggcttcagatatgagctaatgaaatggcagatgacaggagacagaaatgtcacctgctttagtggggatgctgaacactcattgagactgggcatcagttttgccggatgggaaactgaggctctcctactgtgacctgcggggaggagtggctctctccttgtccccaactccataccctaagaagtcagatcactagactcagcatcccacctgtaaccaattccattgtctttttttcctccaggctttgaaccagtatggctgaccctcgccaggagtttgacacaatggaagaccatgctggagattacactctgctccaagaccaagaaggagacatggaccatggcttaaaaggtcagtggggtggacatacacaactggccagtcacagtggagcacagtctgtgttctcctcagcaggagctgacaagaggggttcaaatccaagattttaaaggagttgttcataccttacaacactaggacatcaggagaaggatgtcttgttaattcgaatggactaagaaaagtaaaaattcgagggggttgaatattcaccttcccagatagcatgacaaaaggggggggggcggtcattctcagttctacagcagtccttagctacacaaacctggccatctggactctgtcaggcaagagtatgacggcaccaggttgggtgtgacatgttgccactatccagtaattccagtatccaagtggcagaggcaggaggattgctgccagttcaaagccaacctggtgtgtagagtgagttcgagttcagccaggactacatagagagatcctgtctcagactgtcttagggctagagagatggctcagtggctaagagcactcgctgccctgggcatggtggcggcatggtggcgcatgcctttaatcctagcacttgtgaggcagaggcaggcggatttctgagttcaaggctagcctggactacagagtgagttccaggacagccacagccagggctatacagagaaaccctgtctcaaaaaaacaaaaacaaaaacaaaaacaaaaaaaagcacttgctgctctgctaggggacctgaatttggttcctagcacccagtcaatcaggccacacaatctgttaaccccactactgggagacttacaccctgcacctacctaccttgcattcgcagatttacatacatatgcataaacaaaaataatgggggggctggtgagatggctcagtgggtaagagcacccgactgctcttccgaaggtcaggagttcaaatcccagcaaccacatggtggctcacaaccatctgtaacaagatctgactccctcttctggggtgtctgaggacagctacagtgtacttataataaataaataaataaataaataaataataatggatgtcaattattttggatttttttgttttgttttgtttgttttttcgagacagatttctctgtatagccctggctgtcctggaactcactctgtagaccaggctggcctcgaactcagaaatccgcctgcctctgcctcccgagtgctgggattaaaggcgtgcgccactaccgcctggctggatgtaatttttttaaaaaaattttattaggtattttcctcatttacatttccaatgctatcccaaaagtcccccatacccacccaccccccacttccctacccacccactccccctttttggccctggagtttccctgtactggggcatataaagttggatgtaaatttttaaagcacttctcagatttagtttaattaattaaactgcattggcctctgcagagtctataatgtagtcttctggctctgttgacttggcctcgaactctttcaaactccttcaaactcgatggttgtccatttcactctgtgtgtgtgtgtgtgtgtgtgtgtgtgtgtccctgagcgcccccacagctcctgcacacacacacacacacacacacacacacacgagagagagagagagagagagagagagagagagagagagagagagggagggagagagagagagagagagagagagagggagagagagagttgctgatccttggaaggtgctgtgaaacaagagtgtgctgtactacttagggctgttctctcaccaaagaagcctaagacacccaagagatcttgctatgccaggtttaggatgagctgtttgtaggtgtagcctggcagctttgacttggcaggaggtatcacagtaccaacaacagctcagataaatgagtgacacgagagactgttttgaatgatgactagagacgacatgctgaatacacagtgtcttagtggatgctaaacattgggttatgagtagatgcagccggaaaggctgtggggtgggggaggtggtcacacaggctcctgaattaaaagacgctgatgtgtaacactttccaggccttggaaaggggcttcttctcccttttctctccctccaaggataatgtaaataagggggatgggagtccacagtgagtctgagggtgagtcttgaggtcagcctaggatacatgagacacggtgagaaaataggagaagggcccttctttgcctgggctctgaaggaggctgggaggtgtctcatcactcagtacccgaatgcctttggcctttctgctttgtgttctgctttgtacatagacccagctgcccctccaccaccccagaagcccagaatctcactgttgcccaagggacagccttcatctccactgaatgccccagccacactgccacctagcaagtaccatccatgctcagtgctccttgcccattgattcctcagggccctggccaaactttatatgactttctgtagctcccggaggagcttacaccatctcatctaatctagacctccttgacacaatccccagcagctttgtgacttgaactcgatccctgctttgtccagtcatcaccaaggacggagaggtgcttgtctgtctgtctgtctgtctgtctgtctgtctgtctgtctgtctgtctgtttgaaagacagtctctctacctagccctaactatcctggaactcactgtgtagccctagctggcctcaaactcgtggatctgcctgcctctgcctcctgagtgctggcattaaaagcatcaccaccatacccatcccaagagccatttcttgtgttacagcattgccctggcaagcatggccctcaggccaatagcccctccctacatgtgtccagcctcacacacactgtccctgagaatggttctcgctcttcacatggttgagaaaaccagccaccatgagtgtatggcttctatcttcaaggcgtctcaggagtaagaaagatgtggcctcctaggttggcacagccctgtcctgaattctatcagaattctgttttcaaggacagcagatggaatgggccccagctgggactcctctttgccaggacagtagatgttcagcctgaacctgaggggtgggtgtgttagttagagttttatttctgtgaagagacaccatgaccacagcaactcttataaaagaaagcatttaactaggattgacagttcagaggtttttagtccattgttatcatggcaggaaacaaggtggcacgcaggtagacgtggtgatggagaggtagctgagatttctacatctggatcagaaagtagaaaaaaatgacagtgacacactggccaggccacaaagcacaccccacaatgacacacttcctccaacaaagccacacttactccaacaaggccacacctcctaattatgccactcccaatggggccatttttattcaaaccaccacaaggggtattaaagtgttatggagcatggggtgatactggacttaaccccatggccagagcatttgttcctctgttacaatgggcagctggctcttagagcctctggagagtcccagccaacatctgcaccataacaaagaataaagaaaaaagaaccacatgtattcctattgatacacagagctaccctaaaaactcttcaaccatgatgtcttatttagggtttccttagttgtgaagagataccatgaccaaggcaactcttaggacagcatttaattggggctggcttacaggttctgaggttaattggggctgatttacaggttcagaggttcagtccattatcatcatggtgaatagcattgcagcgtccagacagtcacggctctggagtagctcagagttctacatcttgttccaaaggcagctaggagtaagatctctaagctcttccccacagtggcatacttcctccaacaaggccacacctcctaacagccactccctggaccaagcaaattcaaagcaccacacaaggtgaagtaaacaccaccagaatggccacctcccatgatgagtcttacgtgctggacaaagagcattatccccgcttcgagcacagccatgctaacgaaagcctaggggagtcagcaaagaaaatccttcagccattaatgcgttccctgagctgaacaatgaggagctgtgacctgaaggtggagcggccactgggaagaccagagagggctttctgctcaagcaggagcaggctttccagatacattgtcaggttgtcaatataagggctttgcttccgttgaacctgggtgattaaaaggtatatcttcacacaccaggaggtggtgttatacacctttaatccctgcactcaggaggcagaggcaagccaatctctgtgagttcggggccagtatggtctaaagagtgagttccaggatagccaggtctgcatagagaaaccctgtctcacccacattctcatccaagaaccccaagagtggaacatggttgtgtaaggggcactggtcccttcctggtttgatcatcgggctacagtgtccacactaaagagattacagctcaataggacacagttctcaggttcctcttgaggtctgagggctcactgtatatgttccagagtctcccccacagccccccgccgatgatggagcggaggaaccagggtcggagacctccgatgctaagagcactccaactgctgaaggtggggcctccctactcccaccactccattcattcagcttaccttgctttgagcttcccctcccaactccacgctacgcactagggttgcggctgcgcactgtgtgccaccctcccggctgcactcgctgcttcctatcctgatggctgagatgcgctagcccggtgctttgcagggtagtgggagcggggacctgcttctatgcctggctcaacagtgtgcgccttgagcatcattatcgtgcgtgttgccctgaatgttccatgcttgaagaagctggtctgtgtagcacagcgaggctcacacacagctcaacagggttcccatcctgaatctgctaccttccatgaccccctcagaagcctccaccttacttttctttcagggattcaaagaccagaatgctcatttggctgaggagggggcaggtcagaagtctcccgctcatctgttaggagcgggttttggtgtggcatcttcgtttcttgggctctggattttgggcatggagtacgtttcatctcctgtaatctgttcactccagatatgtggtgtctaggttgtcgcaaagctcattgtcttttttcctgaaatgctccctggtcggctgtcttgcttcccaagtgggttagttatagtgccactgttggctgctggaacccaaaagctctaccaactgaagaggctttttgccttgcaatcataagatacccggacatgggcatagctccctaacgccatgtatgcgcttctgtctttgtcttctgccatcctacgtggttggactgttgcttcattgttgaagaatagctgctgcgctcccaaacactgtcatctgccttggaacaagaaccatggctggtgagacagctcagaggatgaaggcgtttaccatgcaagcctggggacccgagttcaattccccagaacctttgtaaaggaggaaggacagaaccgactctacaaagttgtcctttaagttcccacacactgtgatggcatgtgcccacagagatgtattacacacgtgttccataataatgaatgaaagacaggcagaggtggtgcacgcctttaatcccagcacttgggaggcagaggcaggcagatttctgagtttgaggccagcctggtccacagagagggctccaggacagccagtgctacacagagaaacctgtctcaaaaaaaaaaaaaaaaaaagcaaataataaaaataagaatgaatgaaattgaaattaaaaacaaaaacaaaacaaacaaaaacagtcaccaaaaggaccccgaagaagccaatgtgttgccaaaactaaatcaagagttttctattataagctcctcctggatgttttcattttcattcactggccagaactggctctctggccaccgtgggctgcatggctgtccccaccttgcttttggatccattgagtagaagaaatcaaggaaaagggacatggagaatggcttttaactaatcgtccatagctcctccagccttccaaggaggttggagcttccactctgagccctttctgcatccctgaacatggcttgggcgttctgttacagaaagagaatttggccaggccctctcccgcagcatctgcctttggataaatccagaacatagctgtcaagctcctgccaattgttcggcatgctggcccatccccggaggatgcaggtggcatggcccttagcctggtgacaacctcctggctagatttcctttgcccttctgaaaaccagcgatgcaagacacttcctgctcatcctgttagaacaggtctgcaagctgtctgccaccctgagaaagccttggagccaaagctggggcaggcttcccaaacgtggctttcactgctctgtcaccattgtccaggaagaaacaacccactcatgcagtctggacctgtacatgcgtgggcccccacatgcctcactcaccctcagggagggaggcagctggggctgagctcagcgagctgttttatctgcaccagacaggatcataaggaaaatagcctttgtggactagctgtgtggaattctgggaatcccttgaaaccggccactccttgggtgtgcctcgcatacctagtagagaggctgggggttcgggaggagctaaagtgcttgctttgaaaagatgcccccaggtcctggtctgggatctcttatctcttgcatcatcgaggaaactggatctggcctggttattgccacataaggaactctggtgccttttaatgagatgtgaggaggttgggggcagggaattagaagtggtagaagttttaaaggtcacctacagatggcactcttgttctacacttgtggaaacaaaggctagggtgacaaaaggcttactcaaagccccacaggaaagcggctcagtctatttctcagcttcctcatattgcctgatgagtcccagctgcggggcccctgcattgcagtgaggatcacacagctgacccccaggagtcaggctgtgtccctcgtcatccttacatgacccctgtaggctttccaggcagggggtcctcctgttagtgtgcttcctggggagatgggcaggaaggaggactgtggccctgctttgagctggcttttcttgcatgtggctggcaggccccactccttgcactgcccctgcataagcagaggggtgactgttggtggtttccagacgtgactgcgcccctagtggatgagagagctcccgacaagcaggccgctgcccagccccacacggagatcccagaaggaattacaggtgagggtgagccccggagctgccttggacaggtcaccctcatagtcgggtgcagcagatgagtaagccgctctggcttctggggctcctggctatttagcagtggcggccttggagtctgactgcctcccacaaggctacagagtcctctgattcctgctggcccccacctcagtcagtgtgcctattagaaaggttctcaacagagttctctggggaaggtttcctgccttcttcacattttctccaggcccagcaatagtaagtagcaggtcacctttaggggacagtttactcccaggagcaccaagcaggctacatcgactacataactgaatccttctgatggtctaatgggatggtgctgttgttcggaaatgtgacctcacgtccatctttcgggttcggtctatgtagtctgctctaccaatgtgggtgacaaggaattaggcagcgaagcagcaaaaagaatcctgggctcggacgctcagtccttcgtaagtcatcctgcagggacggagggctaggcaagaaatcatcatagcccagagcctggtaccttccctttgtgctgtcctccatggcctcagggagaactccagcagttagcaggatgaacccaagttgcccagttagctaccgagacatgcctgatctcaccgtggcagagatgggaagaccccttgctgaggagtgcctctctgcaactatgccagtcactcctcctccttacagtgatccaatgtctgactaaggcaactctagggtctgttagggctcgtcgtggttttaggggatagactccatgtgacaggtggcggcacggtggcggtcacactgcatctgcagttaggaagcagacagcagcgaatgctggtgcttagctggctttctcctttttccccaggaccccagcccatgggatggtgacacccatattcagggtaggttttccctgctcagttaaacctttctgtgaatacccccggaagatgcgcccaggggtgtgtctcctaggtggttccaagtctagcaagttgacaatgaagagtgagcaccctaggagcctatagccagccttctctcgcacacccatggagaatgcccactacagagccaccccggctttcaaaatgtgtttctgctttgcaaagtgattgactggcagtagtggcttctgagacagaaggggttagatgttcttaaatcctggccaggcctcagccttgctcatgaaggatgcttgaaggtcacagacaccttacgggggtgaagtcatgggtcatatgatactttttgtctctggctattctttcaaaggttttttgtttttgtttttttttttagattcatttattttatgtatgtgagtacactgtcaccgacttcagacacaccagaaaagagaatcagatcccattacagatggtcatgagccaccatgtgggggctgggaattgaactcaggacctctgcatgagcagtctgtgctcttagccactaagccatatctctacatcaaagttttgtcattgaagaagtttgtcctccgtttaggatgaaagctgtagactttcccaccactcctgggtaatcagaaatgctttatagtctttctggtctcaaaacaggccacaaaggccaggacacagctcacttagttggtggactgcctggtttagcatacgtgattcccagggcatcccccaccatgctatagattgagtgtgatgatggagaaggaagcctcacagcttataaacactcatctgaatggccagtcagatgtgacagtcaggcccacagccttgttctaaaccagccgctgctctgagctagtgcggggcaggtgggtaggaggggacagtggcatctctgcaggagcttagaggccaccttttcccatggaacatctcctgagtgcccagacacctgacagagatgactcagtgaggaaagctttgttttggctcatgggctcctgtgtcctccttggacagagcattgcctgtggtggaggaagctcttcttggtagatagagagcccaaaggagtgtagctttgggtgtgatttctccttccttcctctgcgttccgtactgccttaacccccagcccatgggatggtaccacccacattcaaggcaggtcttcccttcagctaatcctatctgtatgtatcttcaaacacagccacaaatggaccacaacagcccctgggtggttctagatctaggcaagttgaccatgaagattagctaccacagagaccattcttgaatccttccatgctctccccacttcagctcctagctcctacttccgtaaagatggattcctctgtgtcagattctggcggatgcgtcctccttcttctcagggacccagctactgcatcacctctgtgaaagacaactcgttctcactagtggcccgttaagcaggatctaagatgctcagtgcccacaattccgtggtgaagatgtcagcatccttggctgcacagcctgctggcagacttacaatgcacctttgctcagtcaccctagaagggccagcccttcccatatcctgttgtgtcccctgactgccattcagcagatcccaagccggagagaacagaagccaagtgtctttctctgggtggtgcattcaagaccacggggtgaagctacgcatgcctgttatcctagcatttggtaggctaagctggaaagatcaagagttcaaggcctgctgagttacacagtgagtgcaaagccagtatgggctgcatagcaagactctagggtgccactccagccccaggccgctcacctctgggtgggtttagagttgataaggttcccagctgttgatggagttagggcctcaccttgtgctttcttgagcacatgtttgctgcttcttgggtgctcagtaaagctgcccaaggatgctgtgtgactctctacctttgggaggcctgtctccctagagagcagggaaccttgtgcctccctgggaacattcaaagccagtagagataccaactcctgcagtgccttctgccctgccccttgagatttatgatttcctcatgccccttgctgagctcagacatgataccatcccccatccccccccacatcaggggcaggatatcacactactgcactagcatgggaagatggatagactctcttagcaccagggcggtcactggtggagtccgggaatgcctctatctctactaaggactccgtgactgcttcgttgggtttgctgtccctctgagtctgtcttccatttccttaatcttactccgaccagatccattgttggactttgccttccaggcagttgacacagtgtgtggccagtactgtgttatctgacaaggaactgtcacatacacccataaaaagcagcacaaaagacctagaagtgtctgagctgataacccgtcgggtggagagcgtctaaattcacttcctgtggccacaccctcagacggccatctggctacccttgacaagtagtgactcgggctctttccttgtgccggccttgggaagggtgctggtttgtagagactgaccacctcggtctagagacaaggcaggtggcccagctggcactgtgtgaaatgcttctgaaggcagcagcctttggtgtgcctcaaggccttgaggtcaacttgggtcgctgacctcatctagactagtgtggtgtgtcatcccattgttgggtcctgtttgccccttgttcttttttgtttgtttgtttttgtttttattttttgtttgttttttgagacagggtttctctgtatagccctggctgtcctggaactcactttgtagaccaggctggacttgaactcagaaatccacctgcctctgcctcccgagtgctgggattaaaggtgtgctccatcacgccttgttcttgagattagtgcccatatggtccggaccttcctcagtcttctttgtttgaggtgactgttggcttcttaaataaaacccaaaggccctgaaaagctatccattggccctgttggcctccctccagccaggtagtgctggggtttttttttttgttgttgttttgtttcgttttttttaaatgtatttattatatgtatgtgaatacactgtagctgtcttcagacacatcagaagaaagcatcagatctcattacagatggttgtgagccgccatgtggttgctgggaattgaactcgagacctttggaagggcagtcagtatttttaaccgcttagccatgagccatctctccagtcccaggtagtgctgtttttaggagtgatgtgtggcaaggtttaaagagaaggagcatagcagcttggagcatctccttcctgtcttcaggccttcaccatcctcctacaagacaaaccctagtccctccttaaggtttccaataatagaggctcagagaagctggacaggggatggcttctgggtacacaccatcaaaaataatctctccctgaaccccagtcctcccaaagtgctggccagagcaggcagtgccactgtgactcatctgtgagcccctaagttctggcatctttgtggagccaggcaaggcatcaggaccctggacccctatggaaaagctctttcctcctcggtcgcagtcacctggaaggcacgtgagtgctatggattttgtttaggcacatgtcatcaccactgtccctagccgtggccattgcagaggagcaaaacactgcagctggccctgcagggtgcccatgtgcacacacacgaggaacacaggtacagagagcatgggttgcaacaagaaataacagtctcccttctgtcctctccctttgctgcctcggaccagccgaagaagcaggcatcggagacaccccgaaccaggaggaccaagccgctgggcatgtgactcaaggtcagtggcccaaatcgactttgggactcagattgggaggagggagatgggagcactcagtgggctctaccttgaaaagacatcttagcgccaggtcctagggcacaagttcaggagacccgaaggagttgccaatatcaaagttggcctctgtagacacagacagcgttttcttataccatcctggttcaggtcatgcctagcccggggatgaggccagtgggtgacgctcccacagggtgccctgtggcctgtcaatcacatcaccaatgcacttgtcattcgaacttccttctctgcagtcttcacaccttattaggattttcttctcaggaactaaattgttgtgttcaggagaagtagctttctgcagctgtgggtcacagatacgctttaaaaaaaaaaaaaaattttttttaaccttagagagtcttaggaactggagatctctgacatagagcaagagctggctttgttttcttccatttttctggataaatgaacttgagagtgtcctgtgtagcagccagacatacagatagatggcagaggagctggggggtggtgtcctttgctcgataaggtgacaagggatttgtcttttgtttagcggtgctggccgcctgtaaagggctctgcctcactgacctcaagagttaatcatcctttaggtaacttggacactagactgctgaggaggacttgggggagacttttggtcatggtcctgtgtttaccctgacatttgccttactgcccctccaccactcccagagagtctgcacttaaaaaacagttttgttttgagataagatcctcccagccccagctggcctccatttgtattcaaggatgaccttgatctttgaccctcctacttccaccttcttgggaatgctgtggttagaggcatgtgctgccacaccccgtttacaagattctgtgaccaaactcagagcttcttgtggactgggtaagcgttaaccgagctgcatcctggcccctgcttcatatttactgagtactttacagtccccggggagccgcgtgggtggtcctcccagcccagctcctcctcctatgagctttaattatgcttaggttcctcaccagtatgcacacacagcaaagtatgcttatggtgtatgcttactgtatacatttggggccttgtctcaacttaaatggagtctttgaagatattacagtacatcagactccaggacaccccagtcaaaaataatttctaggaagtcttttctttctttctttctttctttctttctttctttgcttttgttttttctttctttctttctttgcttttgttctttctttctttcttttctttttttttttttttttttttttttggtttttccagacagtgtttctctgtgtagccctggctgtcctggaactcactttgtagaccagcctggcctcgaactcagaaatctgcctgcctctgcctcccaagtgctgggattaaaggcgtgcgccaccatgcctggccttttttttttttttttttttttttgaggggtgaaggttatgtttgtttgtttgttttcaagacagggtttttctgtgcattcttggctgtcctggaactcactctctgtaaaccagggctagcctccagctcacctacctctgcctcccaagtgctcaccactgcccggcctagggaacattttcttgaagtcagtagaggaccaaatctgttgggtggcatcatagtccatggctgtcttggcctctggcctgtgagccagccggtgctcagtagcccagacccatgctggtttcctacttcttgaaacagaccaaaggcctcaataagtttcctgagaaggaggagaaattggagggtttttttttccagacagtagctggtgtatgcggaatgcattcattgatcagtggccatgtgatagccttaatactgggagcataattgaatgtaactgtgaactcatcctgccataaagaaggagggccagggcagaattgggtcctttgttggtgggtcccactgagttagagacagaggtatagtgtgcataaataatacttatgtgtgtccagagttacacacatgtgtatacacaggtcctcccctctccttcctgccccttatccagtttctatgtccccaaggcctttatggttactggttttattaaagaaaattgggcagatgattgggatattttatggggaccaggtaggctgttcagaaagttcagttatgttaagtgtggtggcacatgcctgtaacctcagctcctggaagatcaggagttcaaggtcatccttggccacatagtgagttgggccagcctcaggtccaggagatgctgtcacaagcaagcaatcaaacagaaccaggggatgcttgttacggacctcacagtggactccctactccactcccagcattctcagggcacagtcaaataccctagagagggttccacatgactatcacatgactgatcccaggtttgtttgttttgggttttttttggtgggggaggacacaacaatatgcttagattcgatgagatgttggatagggtgcggggtgagcagtcagagcccagtcagagccagagccccagtgctcctgcttcccctgaggcaaaaggcagcccatgaaaaatgatttcagagctataaatgtgctgctcttccatgcacaggagtaatgaggtggcctcttaaccaaggctccatccctaccctcatagcttgggcatttttcaaatctgtcagagcagggctagaggctgggtgatttatggcacccgctggtttcacctcttcagagagaagagagatagacagagatagacagaggcagagaggcaggccactgtcataactcagccaagagcatctggagcccagaatcttctaagggcaatggtaggtgagcccgcaatcacatgctgaacagccaaagaggtgccagcaggctctgggccagccaaaaacaccagagggacctcagagctcaccaagtccaagtctcccttttacggacaagaaaactgaagcccagagagggtattaagcaagctagaggcagacagaccaagagagcatttattccaagtaaacaaaagcaatgagctgggtctgagagaggtctgttaggggagtcccagtctttttttatggcaagtgctatggtatcctttaaaacagtatctttactcttgataaccatttgccatcactgcctcttcaaaccctgaggaggtgagagttcccagccagcagagggagatgcccaagaggcttctaaggaggagcggggtggtgttcaagtccagcttggactgagcacagaagtttctgggggtctcactgccactctaggcgagaaccagtcagatgctctggccttggcagtcacccgctgtcccctgaagcctcctcccacccgcccagcccctcctccactagctggtcctcaggagcccatccaggatcagggaccagaacgggcccaggagtgcctgcccattctgaaacttccctctgctgggtatctccacacagaaccggagaaggtcgagattttctcacaaagtttgcttgtcgaaccagggcgtcgtgaaggccaggccccagatttggggacctcagactggacccgccaacaggtgtctagcatgtctggggctcccctcctgccacaaggcctcagagaggctacatgtcaaccttcgggaacaagacctgaggacatagagaaaagccacccggcctctgagctgctgcggcgagggccacctcagaaggaaggctggggccaagacaggctggggagtgaggaagaggtggatgaagatctcaccgtggatgaatcatcccaggattcccctccctcccaggcctccctgacccctggcagggcagcccctcaggccggctccggctctgtctgcggggagaccgccagcgtgcctgggctcccaactgagggttccgtcccacttcctgctgatttcttctccaaagtctccgcagagacccaggcctcacagcccgaagggcctggcacggggcccatggaggaagggcacgaggctgcccctgagttcacgttccacgtggaaatcaaagccagcacgccgaaggaacaggatttggaaggggctacagtggtgggggtccccggagaggagcaaaaggcccagacccagggcccctctgtgggaaagggcaccaaagaggctagccttcaggaacctcctggcaagcaacctgcagccggcctgcccgggaggccagtcagtcgggttcctcaacttaaaggtctgtggtttacgctccctcactcctcccctggtaccctccaggctgtggacactgtcacaagcttctggccttgactcctgcaccctgagattccaggataaccactaaacccacgcctgggccgacgccccctccccagcccctgccctcagcctctggtagcacgtgttcctttcacgcttgccctcgcagttgtgcatgtcaccaacacccacctgtgctttctgccctgattggctctctgccacttgatgtatccaccgtggcccaccaggccacgggagattggcaggcatacggcatagagtgggtagcagcatcactcattttgaagaggcttatgattgatattgacattcacagtctagaacattcccagctctctgaaataggcttaattagccccactttgcagatgagaaaactgagcctcagagcagtaaatcaaccttgtgatagacctccagattaaacaatggatggagtcaagttcagaatccagttaggtctgctctgaaaggcacactcttgctgtggttcccacattgtcctccggaagaggcgaggctggagtgtgtcctcctggtgcagagacactaagtccacctgcctgatcaacagacagcttgtcacatttgaaaggtgacagctacctctgtagaggtagctaatggcccaataacctctcccctgtttctctgtatagccctggctgtcctggaacccactttgtagactagactggcctcgaactcagaaatctgcctgcctctgcctcccgagtgctgggattaaaggcgtgcgccaccacgcccggctctctcccctgttttgactgtagcagtaaagcatgtcttccagttccctagaggccctcctctcagtcagggtcaggcagagtccatgtgtggaactcctctggagccctttggagcccgcagaatggcagggacattcatgactttaagattctacaatcttaaaggcatttggacttttgagatggctcagtgggtaaagctacttaccaacaagcttaacgatgtaaattccatcctgaagacccacaaggtgccaggacgtaccatatgcttatagagattcgagggcagctgtgggagccagttctcggttgttctctgatttccatactcgtgggaagacatgtatgtctcacacacacacactcatgtctacttatacaataaataaacaaatgtaagagtagagactacgtgacattttagaattatttccagaacctagaaactgatgactcttaagctcatagtcaaaatcatcttttaactatgggttagacactacttatagagaacaccccaatatatcatacgtgaaagcctgtcccggtggtggcacagcctgccaccctagcactccagaggctaagactaaaggagtactgcaagtttcaggccaacctgggctacatagtgagtttaaggctagcctgggctacaaagcaagatcatattttttaaaagaatctgggcttggtgccccatgcctttaatcagcactgtactcaggatgtagaagacagggatcaggggttcaagaccagactccgccacataagaccctgtcttaaaacaaaagcagaggcagaggaaggggaagcctgtgtcttgtttcaaaatccaagacaaaggagttgacgttctattgaattcttgtcctccagatgtctgggacaaggggcagggaaggcacagttccatggcacagtgaatttagaaaggaaaaaataaataaacagacctagcaaagctcaacaacttccactcactgttgtaggctcatcagggatcttgctgtgctgtgcataggaaccctctaaggaggaggcgtcctctggattccacaaatatatttgatgcctcctgctgacatcttccagaacagggcttgttgagtcctcctggaactcaggaaatgctgactagcgggctgattggcagtgatgatggcaataagcaggttactccagtgaaagagttcggcaacacaaaaaaaaagtggcagagtttattagctacatcgtggcacagacacaaaggccaacgcagcagaaactacacagaagcatgccacggggactgcttgcaccctaggcttgggaattattcagacagtcaggtgttccattctcactgagcctgggaacagtcgcagttgcagcttccttggttagtgacagtctagccttgaggaaatggtgatctttccccgagtcagaagtggactggcacagccgtgtgtcttaaacagacacacagggttgacagaagaaggaacgcacaagagctcctattgctttgcagagtcttgggtgcctgttggttgcacacacagccttggtgcgtctcggagtctccatgctatgtcccaggtgattcctccacagtaaaacggggagacctctgggttggagagtcagcgctggtcactcttcattcacttgcagggagcctcaaggttaacagagctgggcttctgtgagcagcatggcctggaatggggtttggcatggtcagcgtaagatggtcgagaaggtggatctaaggacccttcctagcatggggcaggaaaatagaggtggctccaactgggccttgaggccctagagggttaagtgcgggtctcacaggaacgcaaggccaagcctggccacagtaagagacatccacaaccctgatccaatgatcaagctatagcctgagctaggctgggctgtccccatccatggtcttggggcatttaaatggcccagcattcggtggggttaccgatgaggagagtgagcaaccatagcccacctgcgcctctaacagcacctgctgccttcctccgcatgcccccaccctcggccagcctgcttcccctctcaccaggctgctctgagtgacggccctgtgtgctgtgtgatactaatgatgtgagacctcgaacatttgaggactaagaaaatcagccggtttcaaccaaaggacgctcattccaccacccctgaactcctgatgtccctgtgaacttaagcacggggcatcacgtgcactctctattttatattttaccagctcgtgtggccagcaaagacaggacaggaaatgacgagaagaaagccaaggtaagctactgacccgccggtcctagaaggctctgcttcctgcctgtgtttgttcttgccgtaacgtgtggagagtgtgtgtcactagagtacaactgtcatttaaaggccattgctttgttacaaaggcagtgtttctgtcttgatatttgacacagggaagggtggtcagctgtactcagttcctccccaacacctacctcctgccattccctcacagtttaaatgaaaactgtgtgtagttcggagttagcactagagatctaggctcagtaaaccgagagtgtttttaatatacacagcgtatccagggagccagggagctatgctcgaggagtgaagtgatcgccagataaaggcagggaggattctgggtgagcactcaaagagaaaagcagggaagcaaacaggacaggaagcgtcagtgcatccaagcagcgaacagcaagcggggcaggaggaacagctggaactgcgcctggggagagtaggcaggtggcctcggttgctgagttgtatgtggggctgccttgtgttctgaattagcagaacctcctttaaatgaaatctggaaaagtgtggggtagaaagcgtggttcagagcgataaggaatctttaaagaaagagaaagaaggaaggaaggaaggaaggaaggaaggaaggaaggaaggaaggaaggaaggaaagaaacaaagaaagaaagaaagaaagaaagaaagaaagaaagaaagaaagaaagaaagaaagaaagaaagaaagaaagaaagaaagaaagaaagaaagaaagaaagaaagagtggttgagtggttcagggtaagcctttgccagtcttcagcaaccaaccccagtagataaagagcctgctcgccacagaccagcctcccaaactccgcctgtcttccgttttgttcatctgtcatcctctccctccacgcctttttgtcttagaaaaccctgaagctgtatgtgcggtgttattttcctggaaaatcccaccacggcccagattgaatcaactggacagtccctaggaatgctgcccagcatgtctggcctgtaattagtgccagagctcccagtgagtctccagatgtggatggtgctagctaaagtctgggagagagtccactcccagaagggcctgttctgtactagttagggctccctgtcacaatgctcaggagacaggcagccaagcctcgtgcaggacttagccctgccccacttcttaaagcgtgagtgacagttgcttgtcagtcaagatgctcattgagttgcagggtaagtgatggggaatcctggccccatgcttgcacaagcatgattgtactcatcattcaagtatacactcaggacacagtccctgccctggactgactcacaggagagcatggagccagacttattacaaatctaggctgggtagaggaagcttccagtggagagagagggatcttgcccagctagggaacaagggactccagtacccatcctagctttcctcctgtgaaagcagccccagagagtctgtcccctggactctcctcctggtactggtgacagagattcggccctgaccccttcactgggaattttgatagaatgaggaaacactgtagagtctctggtcaggactccagaacagcctgcacttggtctctctgttctgttttctccccgcccacctttgtttcttttgagacaaggttgctctctgtgtagccctggatgtcctggaacatgttctgtagaccaggctggcctcaaactcagaaatctgcctgcctctgccttccaagtgctgggattaaaggcgtgcctctcagtttcttatttccaacagactggtctcaacagtccctgcagcccaccagggtcatacttcccagtcagagtgcgcagaggcagccatgatgtcagctgtcagccttctggctagatccttctcggaagacatccagccacatagacaccaatttccttaggtagaatgttctgaggttaattttgaccagaccaagattccatcaatggcgttaattagatttgagtctttgtttaatcagaaagtatgtgtgtctatgcatgtgcatatgtgtgtgcattcatgcatacgtgtgtgtgtgtgtgtgtgtgtgtgtgtgcatgcgcgcgtgtgcatgtgagcacatgtgtatgtatgtgtgtgtctgtctgtctgtctgtctgtcagtccttggtgacatctgacagaagcttttggttgactctggcacaatcctccttgaagtattagggtttggctttttggtgttttttgggcatagtagagcgagagggtagggtgttagtgagcctgcataagtgcttctcctgaggttgagcctctgacccacgtcacagtaacattctttcttgagaaaatcttgctgcctcttttctggcggtctccacctgctttactaagtgggaaacacctttaggccagcactctccaagtcggacctacaacaaggtccaattgctaaggaaagaacgtagccgcacgtctgcctgtgcttcaacctccctcttacaagtcctacgccatgtcactttcagtcatgcccatcccacatcagctgtgtagcatgcgcgtagctgatgggtgcccaagagggcagagttgccgtcctttcttactggaagggcggtggagagggctgctttgagctgtgactcgccttctttgtgttgagattttgtttatccttttctccatttgtttccattccccgtgtgctgactttcattttgattttatttatgtttatgtttaagacatccacaccttcctgtgctaaagccccgagccataggccctgccttagccccacacgccccactcttggtagctcagaccctttgatcaaaccctccagccctgccgtgtgccctgaaccagccacctctcctaaacacgtctcttctgtcacaccccgaaatggcagtcctggaacaaagcagatgaaactcaaggtaaggaaaccaactttggcgagacccaggctgctgtaccgtggtgttcaaatgctttcttcagctaaactcacaggcagtgctacatggttgaaggaccagggaaggcactggggactcggagccctgctttgtgcctccctcttccctggagatagaagtccctgataaccaggggatccgagtttgtaaattagggctctgagctgagcatggtaactgatgcctataaccaatcccggcactgagcatgatttccgtaagtcagaagctagcttgagctatacagtgagctccaggctagcctgggctacagtgtgagtgaggttctagcttgagaaataaagaaagaaaccaggtgtggtagtgctcataatcccagcaccctggagtaagaggcaggaggctcaccgcaattgcctagatggccagtaatatgtagaaagtctcttcctcttaaaaatgaatagatgaataagtacatgataaagaaagtctcagctccacctagaattcttacggtaactggccacgcctcgtggtgggtggcgtgtcctccagttgcacccattcatcccgacagtcccttctctcctcccttcctcccgagtctgtggtttctttatgtagctcatgccatgtctgatggtcatctactgaccatgagacccatagccactcacagtctgctggctggctgtgtttgctctgcctcaagctcccagctttcagccattgttcatggccgagtccccaggggctgtggagagagccgaaaaagacatggtttggccgtctgtgagttcttctgaggcctgaagcacatgtgggtcttcagtggcctctgttgaacccagtctccgtcagtcagttacatctatacttaagagtctgaaaaatgggtgtccatccaccaggtgtggtcttccagggtcagcctactgatctaccctgaccctgatcttgcttcaagcagtcctgtcattttataacacaggctagctcaccatgcgtctaagaagatcctggtacttgacatgcaggtgaactgtggggctgtgtgctccacaagccctcaagacttgtacaaggccttctcagcatctgtctccaccatcaccctcttcagtcaaggtgggaggatggggctgacttttccttggtggcggtcacctttcccagtggtgaaaaccttccaccctgattcggtttacagggcgctgatggcaaaaccggggcgaagatcgccacacctcggggagcagcctctccggcccagaagggcacgtccaacgccaccaggatcccggccaagaccacgcccagccctaagactcctccagggtcaggtgagactactctctggagttttaatccagaagcttccagaaggtacaacccttgcctcaggaggccactgacaattcgtaacccctggcacctactaacccttgtggtttgtcttctttttaatcctttgacaattgtctgaggtagctagtgagtccttgccttttaattggtgaagagatcttgctcatttttcaagcatgaggaaaatgcagtcacgtgattaataaattggagaaaggagcatggcctctaatccatggctctggggaggcagccccagcataaagtgttatcaggtcccctacctctttggaggatcagtagtctgggcgggaaaccgtgggggaatctccaacatctcaatgtcacgcagctgggttgttaggaaccttctagtaaagctcaagggtcatagctgagtgatggcgtgctacggtcattactccagtctcctgtgaataaaagccagttggcattaagaacctggccaggctacagtggggctgtttcacgtctaatagagataccaggaatagaagccctgaggacctgctcccagaagggtttttggatgccttgtgactctgttgcctggcctgcattgctccctggtaatacccaggaggcctgtgggcccctgtgtctagcctggctcttagtgtccctgttctcttggcagaggcagcccgtattgcctgctgcatgcttctggtattgtaaagggtgttgcctgagaattggctgtgttttccctaagcagctgtggttctggtgtgtgtgtgtgtgtgtgtgtgtgtgtgtgtgtgtgtgtttctcatacactgatggtctaggagcattaatgatgtgctaagtggggttcagttgtggcacttcacccttaacgccttgctggtctcactcccacaatagcctggagcaggggctcttccaggctcacctgtgcttaggtggctccgtgctgattggctacatcttccttactggtcccagcatctgtccctgcttgtctcttatgctaggcagaccagccagctgctgtcacctgaagtactcagagagactcctgtgaacctgattataatgaggctgtctgtggatttttttttctttttaagcttccaagcaaccgcagagaaaactaccccctgcaggggcaaaatctgagagaggtaactctgggtccattgccctgaatgggtgggcagaccctcttggtgtgtattctgccctcgtgcttgtccgcaaggctgggagggagtgctgcttcagcccaggctaccccagagaggcccagtctcgaacttacttacttctcatgtgcggtttgcactggggcatctatggactacatcctgtgtgttcagatttacttcgggaactaggactgtttgtgggatggggaacatgtgtctggagacatctgggagtgttggaggtttggaccgtctctaacctcagtcccagtgggtaaatatcactgataggcagagggttttgctgggccgagtttttgtttgtcaagacacggcgtcacatcaagcagtggtggtgcacgcctttaatcccagcacttggattaaaacaatggggggctggagagatggctcagcggttaagggttaagagcactgactgctcttctgaaggtcctgagttcaaatcccagaaaccacatggtagttcacaaccacgcataatgagatctgacaccctcttctggtgtgtctgaaaacagatacagtgtacttacatataataattaataaatctttaaaaacaacaacaatggatctttgagtttgacaccagcctggtctacagaatgagttccaagtcaaccagggctacacagagaaaccctgtctcaaatagtaataataataataataataataataataataataataataaaatagttgttaatcccagtactcgggaggcagaagcaggaggattggaggatttctgagtttgaggccagcctggtctacagagtttgttccaggacagccagggctatacagagaaacgctgtctcaaccaccaccccccccaaaaaagaaaggcatctctatttgccaaggctggtctcttggctcaaataatcctcctgcttcagcctccaaaacaagggacactgtggaagcacatcacccccacctaactttttctgcatttagcgaatgctcaaacagcacaacagatgctgtgctggacccttcatgacctccctgcctcacctgtcctaaagatttatttttattttatacgtatggatgtttgcctggtccccacagaagccagaaaggacttgcagacagttctgagccaccatgtgggtgctgggaatggatcctgcatcctctggatggtcagtcagtgctctcaactgctgagtgatctctctaacccccagatacccttcccgtagcaaaatcctgagggctctgatccctgcatctttctgacgaggacacaggcatagatagagagggtcactggctcaactgaggctacattgcaagtgttggggaagagacacaagacacagctagctaccctgtatccccggggacatggaggctgagctaagcagcaggtccacatctccccgagcccgatggctttagggggtcctgagaagtgggagactggagctgaacgctgacgctttgagggggatcagctgcagtgagcttatttgctatgggagcttcttgctgtgggctgggaagcttgagaaagaccagttttgcaagcccagtgcctctgtcatatctggttctatcagatctggatggggggcccccgaaatttgcataccataagtcccacctctagctaagaacatcagaggtcttcaaacacaaaacaaaacaacccttggggtgtggtggctcagtgaactctaggtgtgtgggacatgcagagggtaccatcctggaagcaaacggaggtgaaggatgcagggtgggagaagtccccacagctttcctgggtgtttagaaaggtacaagggggagggcattctggggttcagcaatgcggggtgtggcttgtgcagaaagctctggtcaaggagattcatgctgttctgtggccccagagttggaggagctctggatgtgtgtgagaagggtcaggaggaccccagcttgaaggctacagtgggggccccagtcactgtaccacccctcagttgagggaaaggacacttcagggcttagggagtgtggatcacccctgggaagaggtatgtcatatattggggcactgatgagaattcagttgcttcctaagaaatagctgtcattgtatcaaagtgacagagcaggagtgatgcttctctgcaaatagtcttcatggtcttctcccagagccccctctggatcctggcagtgggctcccagtccagtggcctcagggactttgcagacgtcgcccccaccccgcgcgccccccacgtggcctcagtcccttcctctctcccgactttccaggtgaaccaccaaaatccggagaacgaagcggctacagcagccccggctctcccggaacgcctggcagtcgctcgcgcaccccatccctaccaacaccgcccacccgggagcccaagaaggtggcagtggtccgcactccccctaagtcaccatcagctagtaagagccgcctgcagactgcccctgtgcccatgccagacctaaagaatgtcaggtcgaagattggctctactgagaacctgaagcaccagccaggaggtggcaaggtaagggtgtggctggtcttctgggaagccagactcccacctagccagacttgctcccccccaccctgcccccacacccctccaaccccggaagcacacgggccaccaaggcactggctcctcccttcccctctttagccttgctaattgggcaggtgacagccacttgtctacagggacccacctatggtgacctatggattttgctaagtaccgcccttgttgggcttggctttcacccttggttctggggaaattcatggttttaatatgagcagctggttctacggcagtcatgaaatttgtagacctcttgtccccaggctcgtcgaggctcctccttcctcgctttctcctgagagttgctcaggctacatatactcagggttctggaatctgcaacagagaagagattatgtgggtgtcagcaggcttaactccccacccctgagtttctcccgagtcctcctctgtgcacccctggatgtcccagtctcatcttagaaggcacaagcccgtgggattggggtcaccctactgacttcatttcaacacaaccataaaggccttatctctagacagtcctgttgtgaagttgaagtatcaacatgtaaatgagaatacacacagaatgccacttccccacccagtgctggactgaggggaggcatatgtggggcgtggggctgtgaaccattcctcagtctaccatctttgtctttgggctgggtgcttctcggacaacacacctctgcaggcaaacggagctcctcagagtgtgggccaatgtttaccctccatctgagcagtggacactcttcctactgtcgaattaggttggggcttgtcactagagggaaattgactagagctctggtgggcaccttccctgaacactgtgctttgttgggggagccctaggccttcctattggctctgcacagtggggtttgtgcagcgtctaggttaaagtgtatagctcacagggacttggtagccatcagcacacagtacctgcaagtcagagaccctgaggcagacacagccacctcctctctttccaccatctcagatgccaccctagactctcagccactgtcaggctcctgtcccttctgtcccttccttcttagagcatcacccgttcccattcattctggtcctggacagtggctccccaagcactgtatccagagacctccatgactctgccgaccccaatttgggcttctcaatgtcacctctgctgcccactccttgggaacctctggttcttgccttgctttttctgacctgcagggggcgccatctgcctagaacgcccttccctcagctctcccctgggaacttccccctaggactccttgatttgcatcccatgatgccccaagggtgctgagccttcatggaactgggcttctggatcttatcaaggactcaccagaactgcaagaccctacagcgtgggcggctgccctgtcagcccctgcccatggcggggtggcttaccaggctgggcagttctttcaaattagtaccaggaactggcccgaacagggagtgcaacattctgcaatgttccatagaaaaaaagggcttggctagcctcatctcaggagctgtgctgggctatgacgacttgccattgtctacagaggagacaaagtgacttgccttgggacttcaggggtgacagcatgggatgtgacaggaccatggaacagagggcaggagagttgtcttaacaagccctcagcccttcaagcaaagagcagcgttgccgcctccagaagctatgataagccttttattccaggctgatggaccagacctgtaacacccattgatgggagcttctgagagctgagctgacatcttggatccctgaaatgtctgtttaatgagaccctacctcaaaagaatgattggccatgaaaggcaaaattctgagagtctgaagaatagggccagagatgtggctccaggaatagtgtgtgcctagcatgcttggagccttgggttcagtccccagcaccagataaacacttcctggtcttcctggtagagcacacctgtagtcccagaactgcagaggtggaagaagttcaaggtcgatctccaccacaggcaaatttcaaggatagccaggtcatcaccattgtcatcatcatcctgagattttttttttttaagatttatttatttattatatgtaagtacactgtagctgtcttcagacactccagaagagggcgtcagatcttgttacagatggttgtgagccaccatgtggttgctgggatttgaacttcagaccttcggaagagcagtcgggtgctcttacccactgagccatctcaccagccctcatcctgagatttttaaaaagggcttgggtctggtgagatggctcagtgagctaaaggtgcttgctgccaagtctggcgacggagttcaattcccagggcccacgttgttgtcgcgagccgtcctctgacctccaggcgctcacctccccacaataagtgaataaagcagggctggagagatgactgagaagtcaagggcccttgctcttagcaagagttctgtccccagcacccactgcaagctgctcacacctgcttgttcagctccatgtcaccttaccctttctcctgaccttctcaggcatccatacacaaccacacagagagagagaaagagagagagagagagagagcgcctgagacggctcaacagttaaaggcactggccaccaaacctgaacacctgagtttggtcctcagaacctacaaggtggagggcgagaactgactccaacagggtttctcctctgatgaccttcacaccgtgtcacaagcatacacccacatccgcacaaatgaattaatgtaatttaatttttttaaaaaaggtactgaagactacagacccctctggagtggtgtttaccttgaagaaatggactgcaggggactctgcagacattgagtgtcctgaggttcatttagattttccatcaagctaagcaagagtgacacatgttatcctagcaacctcaggttggaccaaggagtctgtttagcctggactccacatgaaggaagatcttgattaggagatatgcgagaggcatctactcttctgccttagactttcccctcggcttccatggttgcttcggtgaacgtgtttcaccctaagatttcacagtgaggctgtgttaaagggaaatgcagccggtcagacagccttaccagagaatacaggaaggagtgtggaaccaggagacaacagctacttggaagttcttttaaatgtctaatgctctctaccccagcctcactgtgacccccagggacagagcctaccccagcctcaccgtgacccccagggacagagcctactccagcctcaccgtgacccccagggacagagcctaccccagcctcaccgtgaccccaggaatggaccggagtgtttccatggtacagtccaggctgaggctcatgctcagtgctttcttatcatgctggcttttggtcctccccaaaccgtggggtcctgagcacctgggtcttcctttggtctctgccatgaccccatatacaggttccgcaggtaccttctcagctcagacacagctgggccggccctaggccttccatgtccatccagagtcggcccctccctgtgcagcatctgagaggagagagtggggagttcacagtgagaccagggtttaaaccgtgttttttaaaaattactcactggagcagggcatgatagagcatgcctgtaatcccaggaggacttgctgtgagttcaagagtagcaagttctatgggtagcaaattccaggccaggcagggaggcttgtatcaagatattctcttaaaaaaaaaaaaagaaaaacaaaaacaaaaattttaaaaataggaaaggaaaagaagactctactgtccagtgatctgttgtgcgatctcacgttctgcctctggagcaatgggtctcaattctcaaccttcctaacgctgcaccctttagtacacttcctcatgctgtagtgacccctaccacatatcataaaattgcttgcaatgctactacaaaactgtaactttgctactgttaagaatcataatgtaaagccgggagtggtggcgcacgcctttaatcccagtactcgggaggcagaccctgtctcgaaaaaccaaaaaaaaaaaaaaaaaaaaaaaaaatcgtaatgtaaatatccatttgacaaaaatcttaggtgacccctatggaaaaaaaagaaaaaggtcatttgacccctccccccaaaagggtttcaagtcacaggttgagaacctctgttctagagactagaggccctggaaccttagatccttgtgtgtcagggcttctcatggagaagtgtgttcttggaatggaggaaaccaggcgtactagtcatctaggactgtagcatgtaggaagctgaaggcagaattgttagttcaagaccagcctaggccaccctcaaattaaaaaaacattaaattatagaaattcacctgtaaccgaaagaaagcattaaataacaggagatgatagtaaagtaagagatggaaagacaagccaggggtgtggctcgggggcccgagacccatcctgacactgtcaccaaagctgtagcccgggatacttgagattctgtttagatagatagatggatggatagatagatagatagatagatagatagatagatagatagatagatagatagatagataaaggtggcttccaccagtaatcccagcactcaggaggtagaggtaggagagtcaggcattgaaggtggtgcagacctttaatgccagaactcaggaggcagaggcaggcagatttctgagttcaagtccaggctggtctacaaagcaaattccagagcagccagggctaagtagtgagaccctgtctcataaaagaataaaaataaaaaagaaagaaatgttgccgggtgtggtggcacacgcctttgatcccatcactcaggaggcagaggcaggcggatttctgagttcgaggccagcctggtctacagagtgagttccagaacagccagggctatacagagaaaccctgtctcaaaaaaccaaaaaaaaaaaaaaaagaaagaaagaaagaaagaaaggaatttcacttctttcctaaccagttttcagctactgtccccctggttgactaagcagatgaagagctcctctgaagttttccaggtccacgcaggacataacctgtgagtcccccagtcaaagcttgtagaacctggaagagcccttgggagtgccggggttgtagtaccacaggtgaccacaagatggcagaggagcgctgtagacagcagcctgtgttcttggcccactcacagttgttgctttcttctctgcaagtgtattgcgattttctgagtcgtcttttccgctaactggtggtcttttttctctcatctctgcccatttctttttgtttctgtctgcctgtccttctgcttcaccactgattatacagagattgaaatagtttttaaaatatgttaacatttgtatgtgggcggggggggggagttgcatgtattttaaatgcaacatattcactgacacaggaaaaaaaagccttttgtttgtttgtattgttttggttttttgtttttgttttgtttttttgtttttttttttcaagacagggtttctctgtatagccctggctgtcctgaaactcactttgtagaccaggctggcctcaaactcagaaatccacctgcctctgcctccaagtcctgggattaaaggcgtgcgccaccaccacgcccggcttgttttgttttttaaaagacaggagttcatttattccaaactggtctcaaactcacgtggttgacaatggccttgaagtttctgatattcctgcgtcctctcaccaagcactaaaactacaggtttgcatcaccacacccagttttatacactgtgagggcttaaatccaggaccttttgcgtcctgggaaagtcctctactatctgagctccgtccccagctactgccactgttgaaagggtagctgtaatggcacctgacctccggcaccttattgccttttgcaggtcggctagactggatacaggcaggctttcttggactctaaggaaaggctttctggaaccccagctggccacgtcaggttcatgttccaaatgaggtgatgcccggcctagcccagttagtaccagcagtctgggagctccttgggctagaggcttggtgccagcaagggattccctgagagttctgagcccaaacagaggtagaacaaagtccacctagtgtttgacatggctctgggaagtgtaaactaaagtgggtccagccagcttctcaggaccactccctgcccacaaacacttcctcctgaatcgaaggccctcagctgaaaacatcctccattcgctccaggatgtttccagtgtccggtcagagccaaagcccagcgccgtggaccgggccagctgcttttgctgggaacggcattgcaggctccagtttcacaacgccagccaccaccctctccccctctgaacctcagtgttcccgagcgctttggcttaacatgaccactagcagtaattcaagtctgctttgtaaatatctatctagtgccaaacaccacaaaagcttgtttcctttcctgtggggttagaataagaaacacacacacacagggcggggtctctatgtatgtttcagaccgtgtgcgattgcttaatgtcctctgattaatttgtgtctagcagtcatcttcctccaaaaaccattttctttgtcaaatcatgagttatgtaatcagtcacatttcctttttcctcattggcttccgggaagctcggtgctgaatataatagtgccctcttcctcttattttattgttattattgttgttgttattattgttgttattattttgggttttcaagacaggatttctgtaaattcaaggctagcctggtctactactgaatttcagcatagctagggctacatagagagaccccgcctcaaaatagcattaaaaaaaaaagttaaaattaacagaggatagtctaaagtaactaagtattgtatgtgcacgccactgtgttcagaaggcagaagtggggttttatgacaagttctttaccctccctgagccatcccaccagctcagctcaagacaatctggtttgaagcctttagccttgccactccccagccttggacgctctttgtgataaggtgccttgcactggtgtgtctgtttggattgttgttgatctttcttctcatcacttgacccaggggactccttgtttacagttgtcactccacccaacctagtgacctcccagctctgtcctcctgttcccagccatgttctcttccagattccttttgtgacttccagggtgccatcccaggcagcttctgagccccctgaggagtcagggctcagacctggctctccccccacagcccaacaggtcagggggcgatgctccggtgtggttgtctctcactcttttttttggctatcaaaggtgcagataattaataagaagctggatcttagcaacgtccagtccaagtgtggctcgaaggataatatcaaacacgtcccgggtggaggcagtgtgagtactgtcgcagtctccatgaggtgtgctgcagcctttgctgtaacaagtgtcatgagtgtgtccttgtgagacattgcatagaataaatcatctagggctcaggacctcctgtgtccccacagtgtctgctgtccccagactcccctccctgttccatggatggcccagcaaagtagggagagcaacccgttccagctggcccttctctgagtagctgggtccccatggtaccatgtatgtgttcctagaagccccgtagatattggaaatggggttgaagacagtgcatccatgatgtgggctgacacctcttccctcatctctggggcagtctctgttggttggggagtggggagtccatatgatcccactagacactaggacttcctgtttcatgtcaaggtccaaaaggcccctagccacttctccatttttcatctgggtctccttcatctttgaagagaggaaaagattcccagttcaatttagcactcccctccccccaggacagggactctggttgcacagagcctaacaccgaggtgacaggtgtcagttgcctgcattgtttgccagagaaggggttttaaatgtctcctcctttaaacaccactggggacccccccccccccccccccgcacatcctcagcgagtccctgcaaagagatgttcttaggttccctcaatgggcttagggattccctttgttctcttggctggccggtgcccaacagggcaggatttgtaaagtggccccaggcttctccctgccagctccccctcccctgttctgggtcagtcccagaacggacctgggctctctgattgagctgcaggctccacctctcgagcgtgcgtgctggaatgcaggggtgggctctctttcctgaaagtgccgagcccacagggtttattcttgcaatgtgctcctgcctccggccccgacacctgccctgccttctgcctcttggaccgatggccccagagcagcaagtgctggccctgtggctactgccttctcacgcttactcaccgtgtctgccttttcctctgtcctccactccccgctggccgatcctgccacctcccttacttgttttgcttcctaaggaaaggcggggctgttggctacactgcggttactgccctgcctgccggttgccttctcaacgtatagaaagatatgggttgcgggaaagggtgatctttaagatcgccgtccttgtccctgggctctgtaatgttgtttatgatacgttagcttcccagcccaggctgctgtggttagggagggaagccagaagtgtctggttttgctcacgtctggtctgttgctgggtctgagcgctccagctcccaagggctgcagagggcagatgctcctgtaggggaagttgggaaaaggcagctctcagggcagctcggatcacatgctggttgtctgatgaggctgcagtgcctagggtatcagggaactttataactaaggtgtactgcagtttgccagcctttgagctaacctgtgaccttatatagaatcacaagatagcctggcctgtggcctgtggccactgttgactacagaatggacctgtggagaaagctttcttagggagggggactggtggcatttgggttgtaggattccaggcttcccatctaacttgctcctggctctctctagggtggtgggaactgcagcagggaagctgcttctccatctccacctcctggctgggctgtggcctcctctctcgggctgctctgtgggatctagtaccctagtcagctgctcccccattcctttcttctctggctatcccaagcttccaacatctcctgtcccccaacataccccctggtactgactctctaatgactctacctgggctctcccatcatcacctgccccagaactcctactggcctctgggtctgtttgcaggaggtccttgggtttatctgctccttacccaccctcctctgcaggggcaggctgacacccttatgacttatgaggccatggccctcctcttccatcaggtctgttagcagaacacacacacacacacacacacaccccataacacaaaacaccatcagccgtgaaaacgagggatttatccattgagacctccgtagaaaccactttgctttattgattcttgaacatgtggcacccctctcccaggacagatgagcctcctcacccctggaatgaccatggtctgtatgggagacccatgggactctgcaagtggcagctagtgaccagctaagctgtaaaaccacagatcagagagacaggcttccaggagccccatctgcctgggtggtctccttcagagtgtcctgtcaggaaagcaactgtcattttccagagcacacattactgctgggccacatgaagggcccttgggctggcgggtcccccccaccccccttccccagtgtgtcctgtctgctcactcatgcttgctgagacacttactgcagctgtggtttctgccgtgcgcctgccttctgttggtgttgcttcgctcttctcagtagaggctgaagtgttcaggagactatgtgtccagacaccaactggctctccttctgggtcatagagctggtctgggtcctgtttgccgactggtaatccaggaccatgggagcttcctgggtgaagcagactcaccctcaccagcttctcctgagtctttttctgaggagaagatgcagggctgtgaaggcttcttcctccactgggtgtcattggcctttcatcaataaaacgatgtatgcttttatatttaagtgtctgctacagagatggtcacgtacaaggaccaggcacctgcttttccatgagagtgtcttgtgtttctgggatgtttaggtccctaagcttcctggtgaggacccagctccctggggggtggggggtggctggatttggggagcctattttccatcctgagtgggggtgtatgcagcagctgatgggagccactgacaaatggtccatcagcctttgtgctgtgttacacccctagtaacataaccccctttattctctctctcgcccatcctctttgacctgcaggtgcaaatagtctacaagccggtggacctgagcaaagtgacctccaagtgtggctcgttagggaacatccatcacaagccaggtagcacttgaggaagacaggctttgggacaggagtgctggctggagtggggggtgcttcctagaataggccaacctgggcagtgaatgttaaatgtgaaagtactgtgtggccttggacctgggtctgtctctgttgtgagcctagacaaggggtggaggaaatggttttatatcccatcctgtggctctccagaaaagtctgtataaaaggaccagccatgggtttgattgggtccctcccacccccaaaggccggctctgtgaacatctcagctttgtcacagccaacatgggggacacttcaattgcatcagctataacatgagaacttttgggctgttgtctcatcttcgctgtaacattgttcaaagaaagcattcttcccaggaaaacaggccaggtcactccctctcctgctataccctagtgtggtgacctgagccacaggggaggttagggtgtctctcctcatggtgcatttgacaggcggtttgattccaaggtcttgaggggctgtggcttacagaatgttcctaagtaaatctccacagctggaaggcatccggcttaaaatttagtgtgccttctcttggccatgcttcctgtaccactgaacgatctcaggaggaagattctgcagggtaaatatcccatgtacacggtctcgatcagagaagaggcaggtagtttgcaagccctaaaaagggcttcctgtggctaaacctctttctagataaaggagagccctggcatctcctgaggatgggttgttctttggatccctggcccagcctgtgtggagccagtggggatgtcttatacctgatggtcccctcctgaatggtgggtgatacctgacctactttttttttttttaatcaagtctccagcagcccggagcacttcgctctgcctgctgtgagctcacttaacatatgctaagtcctccagaccacagactagggcaagaggctcatgtgggttgtgttccaggaggtggccaggtggaagtaaaatcagagaagctggacttcaaggacagagtccagtcgaagattggctccttggataatatcacccacgtccctggaggagggaataagaaggtaagggggtcaaatgggaggtcatacggggataagagggctagcagagtggatgaagacttcaacctctgggtcctgtccccttttggaggtagcatatcatcatatcctgcatatcagatatttgcatcatgattcataacagtagcaagattacagttatgaagtagcaaggacataattttatggtgtgtgtgtggggggcatcaccacggcatgaggaactatattaaagggctgcagcattaggagggttatcctagagggtcctctatggactctctggagatagagtttcaccctcaccttggtgtaccaagaacaaggcagaagatcaaaggcagatctttcccactagttaccagagggctagagcaaagtctgagctgactgaggccgtggtcagctgtcagggaggttgtctggcatccccagaaggtcatgcctcaggttgccatctcatgctttctactacttcctcttggactcctggttatggcattgttccctcgtcatcgtgtgtggtgtgtgtgtgtgtgtgtgtgtgtgtgtgtgtgtgtgttacatgcacatgagtataccggtgagcacacccatgcacctgtgtacagaggccagagcagaacatcaggtgtcttcccctttccctctggagacagtgtcctgaaccagaagcttgctgtttttgctaagctcagtgagctcctgggatggacctggttgcatccctcaatgctgaggttaaagtctacaggctcagtgagatggctccgtgggtaagagcacccgactgctcttccgaaggtccagagttcaaatcccagcaaccacatggtggctcacaaccatctgtaacgagatctgacaccctcttctggtgtgtctgaagatagctacagtgtacttacatgtaataaataaataaatcttaaaaaaaaaaaaaaggctacaggcatgcacagccaagctctgcttttttctttgggtgccaaggaattgaactcaggcttttgtgtttgcagagcaagcactcctacctatctccccagtccgatgacatcatttaagaaggactttttggtaaacccagaggcaggtggatttctgagagagagaaagagagaaagagagagagagagtcacaaataaaagtaaaaaaataaaccttaaaaagtaccctggaacggggtgtggtggcgcatgcctttaatcccagcactcaggaggcagaggcaggcagatttctgagtttgaggccagcctggtctacaaagtgagttccaggacagcctgggatacacagagaaaccctgtctcgaaaaaccaaaaaaaaaaaaaaaaaaaaaaaaaaaaaagaaaccctgtctcaaaaaacaaaacaaaacaaaaagacgttttggtatcataccttctattgtacatgccagtagctgccacctgaaattaccttagtaatctctggtaaagggtgtgaggtggcagtcatcagtagctctacagcaagcctgcgtctttgtggcttctttttatgcttttagcagcaagatggcaataatttagtaaatcagagtccagagctggtggtgtgaggcggcggtagagtgttgcaggatatttgatcacactgtgaacccccaaattgtgttctttactgaaaaacctgtttctagttatggtgtggtccaaccatttagcacacacctttaatccctctggctgggatacagacacgcccttggtacacacctttaatttctttttttttaaagatttatttattattatacataagtacactgtagctgacttcagacacaccagaagagggcgtcagatctcattacgggtggttgtgagccaccatgtggtagctgggatttgaactcaggacctttggaagaagcagtcagtgcccttacctgctgagccatctctctagcccacacctttaatttctttttttttttttttttttaaagatttatttatttattatatgtaagtacactgtagctgtcttcagacactccagaagagggcgtcagatcttgttacagatggttgtgagccaccatgtggttgctgggatttgaactctggaccttcggaagagcagtcgggtgctcttacccactgagccatctcaccagccctttaatttctaacagtaaagataaagttagtttgtagaaggaaacgctcatgtttgaaagtgacttctgaggggcagagaaagtaacaaatcagagaaagatccgccagaatgaatcagagataggctatgtccaatgaggagaaaggaaagggaagtgcagagagaggaaggaggcagcttggctgggacagctgtacagagacaggttgcagagagaacaagccagacacaggtgaagacagaatgagccagagaatgagaaggagccagaagattagaacagattgctagagtttgttcgaggccaagcagagcaattcaggagaggccaagagagaagtcagattgaatcaatcagttcgaagaggagctttagccagaacaactgagttggatcagccagagttcagaaagaactagaaagggtgagtttattcagtggtaaatctcagaggctgaaaacactctaggcctagattagattggatggaggctagaagcttttaggtctgggcctaggttagcagactgaggcagtaagcctcaagagacaattacatcaatgaaaaatatatttacagtagagcatgtgcttagcctgcttgaggctatgggctccatccacagcactgattgatagatgatagatagatagatagataggcattacatttgtaggatacatttacaacatgtaaaatgatgtgtgtgtgaggtcactcagggaagtgcctccacgtgcatcaacagggagtagataaattatgatgcagatgctgcaacggtatagcaactggcctgaagaagcactctgtggaatagcaaaataattaagtctgaagcttgccagcacacacctgtagtcagcacacacctgtagccagcacacacctgtagtcagcacacacctgtagccagcacacacctgtagccagcacacacctgtagtcagcacacacctgtagccagcacacacctgtagccagcacacacctgtagccagcacacacctgtagtcccagcaattgagagactgagggttcttggccagcctgggattaatagtaatatcttgcttcaaaaaacaagccactcgggcggtggtggcgcatgcctttaattccagcactcgggaggcagaggcaggcagatttctgagttggaggccagcctggtctacagagtgagttccaggacagccagggctacacagagaaaccctgtctcaaaaaaccaaaaaccaaaaaacaaaaaccaaaaaacccaaaaaaacaaaaaatccaagccactccagtgaaattgctcagtgggtaaaggtgcttgcagtcaagcttgatgccctgagtttggttcccaggatccagatgttaccaaggagaggaccacatgcccacaagaggatgcacgatgcatgcacacacgcccacacaaatgtaaaaagattagacatacagccaggcgtggtaggacgttactttaatcccatcactcggttgaaaaagtaactttattctcctgtagtaattgttacctcagaggcttactgcctcactctgctaacctaggcctagctccttaagcttctagcttccatacaatctaatctaggcctagagtgttttcagcctctgagacttactgctgaataagctcaccctctcttgttctttctgaactctggttaactgattcaactcagctgttctggctgaaaactactctccatgctgactgattcaaactgacatctttttaccattcactgaattgccctgcttggcctcgaaataactctggcaatctgttttaatcttctagctccttcttatttctcaggctcgttttgtcttcacctgtgtctagtttgttctcccttcaacctgtttctgtaaaattcccccagtaaaactgccccctccttctctcttcctcttcctctgctctctctctctctctttgttttgttttgtttttttttgttttgttttgttttgtttttcgagacagggtttctctgtatagccctggctgtcctggaactcactttgtagaccaggctggcctcgaactcagaaatctgcctgcctctgcctcccgagtgctgggattaaaggcatgggccaaccacacccggctttcctcttctctcttaaattgtctttctttcctctctccttgtgagagttgggtgtatcctatctctgactcattctgtcaaatcttcctctgattcatcactttgtctgcccctcaattagacatcactttcaaacatgtgtgtttctttctacaaactaactttaccttcattgtttgggattaaaggtgtgtactaagggcatgtctgtattccagccagatcacacagtcctagaaggtctttggatatgatcagagtagccatgttgctggattaaaattcctctatggtcagggagcagatcttctggagttctaagccagactggtctacatagtgatttccaagcagaccagagctaaatactggtatccttcttcatagtagataaatcgatatactaattaataaacacttagtgagcaataataaagcaagcatccagtatgaaaagcaaagtgcaaagccctgtgtacattgttcccatatgggttaagcatacacatgcatatctgttcctatgtgtgtagaatatttaaaggacacagaagcaatggtcacggtcattgggcaggcaggtgtgggatagaccatgcacagctattgctagttggaaaacaatacaagtagggtggtagctcacaaggatgtaagcaagttagatgggtcaaatgctttcctagcatgcaggaagccctgggtctgaccctatcattacataaactaggtgtggtggcatacccttgtgactccagccttggggggacaaggaggcaggaaggtctaaagtccaaggttatcctcagatggatagagaattccaggccagcctgtgctacctgagagcctgtttcaagaataagtaaatatggggtgtggtggtgcataccttacagcacttgcaagtcagaatcagacagatatctgagttcaaggtcatcctggtctataaaacaagatccaggacatccagagctacacagagaaaccctgtcttaaaaacacaaaaaatgaatgaacgaatgaacgaacgaacgaacgaacgaatgaatgaatgaatgaatgttttcctcccaccacagactagctctggtgtatagaaccttatcattaatctcactctctacatcttgcagattgaaacccacaagctgaccttcagggagaatgccaaagccaagacagaccatggagcagaaattgtgtataagtcacccgtggtgtctggggacacatctccacggcacctcagcaatgtgtcttccacgggcagcatcgacatggtggactcaccacagcttgccacactagccgatgaagtgtctgcttccttggccaagcagggtttgtgatcaggctcccagggcagtcaataatcatggagagaagagagagtgagagtgtggaaaaaaaaaaaaaaaaagaatgatctggccccttgccctctgccctccccgctgctcctcatagacaggctgacctgcttgtcacctaacctgcttttgtggctcggatttggctcgggacttcaaaatcagtgatgggaaaagtacatttcatctttccaaattgatttgtgggctaaaaataaaacatatttaagggaaaaaaaaacatgtaaaaacatggccaaaaaatttccttgggcaattgctaattgatttcccccccctgaccccgccctccctctctgagtattagagggtgaagaaggctctggaggctgcttctggggagtggctgagggactagggcagctaattgcccatagccccatcctaggggcttcagggacagtggcagcaatgagagatttgagacttggtgtgttcgtggggccgtaggcaggtgctgttaacttgtgtgggtgtgagtggggactgaaacagcgacagcgaaggctgagagatggatgggtggactgagttagaggacagaggtgaggaaggcaggttgggagaggggacactggctccttgccaagtagcttggggaggacagggtgctgcagctgcctgcagcagtcctagctagctcagatgcctgcttgataaagcactgtgggggtaacgtgggtgtgtgtgccccttctgcagggcagcctgtgggagaaggggtattgggcagaaggaaggtaagccagcaggtggtaccttgtagattggttctcttgaaggctgctcttgacatcccagggcactggcttcttcctccctccccgcaaggtgggaggtcctgagcgaggtgtttcccttcgctcccacaggaaaagctgctttactgagttctcaagtttggaactacagccatgatttggccaccattacagacctgggactttagggctaaccagatctttgtaaggacttgtgcctcttgggggacctctgcctgttctcatgcttggccctctggcacttctgtagtgggagggatggggggtggtattctgggatgtgggtcccaggcctcccatccctcacacagccactgtatcccctctctctgtcctatcatgcccacgtctgccacgagagctagtcactgccgtccgtacatcacgtctcactgtcctgagtgccatgcctctcccagcccccatccctggcccctgggtagatatgggcaatatctgctctacactaggggttggagtccagggaaggcaaagatttgggcctcagtctctagtcctacgttccacgaatccaaccagtgtgcctcccacaaggaaccttacgaccttgtttggttcactccattacttcctatcctggatgggaactggtgtgtgcctgcctggggatgaccttggacctctgccttttcttttatctaagtggatgcctcctaggcctgactccttgtgttgagctggaggcagccaagtcaggtgccaatgtcttggcatcagtaagaacagtcaagagtcccagggcagggccacacttctcccatctttcgcttccaccccagcttgtgatcgctagcctcccagagctcagctgccattaagtccccatgcacgtaatcagtctccacaccccagtttggggaacatacccccttgattgaagtgtttttttcctccggtcccatggaaaccatgctgcctgccctgctggagcagacggccacctccatagatgcagccctttctttcccgtcttcgccctgttacgttgtagttggatttgtctgtttgtctgggttcaccagagtgactatgatagtgaaaagaaaagaaagaaaaagaaaaaaaaaaaaaaaaagaaaaagaaaaaggaaaaaaaaaaggacgcatgtatcttgaaatatttgtcaaaaggttctagcccaccacgtgatggagagtctggatatctccttcctgacgtggctccaggccagtgcagtgctaacctgctgggacatcccatgttttgaagggtttcttctgcatctgggacctcacagacactggattgtgacattggaggtctgtgacattggaggtcaatggcattggccaaggcctgaagcacaggaccagctagaggcagcaggctccgagtgccagggagagcttgtggctggcctgttttgtatgaagatggtcctttctgatcacgacttcaaatcccacagtagccctgaaagacatctaagaactcctgcatcacaagagaaaaggacaccagtaccagcagggagagctgtgaccctagaaattccatgacgacccagtagatatccttgggccctctccaagcctgggccttttcaccatagagtttgggatggactgtcccactgatgaaggggacatcttaggagactcccttggtttccaagctgtcagccccctgaacttgcacgacctcctacagcttcagggactaggcctttgaagattaggaacctcaggcccacatcagccacttctgatgtacagttaaggacaatgtggagactaggaggaagcagccagcctttcccattaaagaactcttgagtgcccagggctacctattgtgagcttccccactgataagactttagctgtccatagaagtgagtccgagggaggaaaagtgtggtttcttcatcatggttacctgtcgtggttctctctcttacacccatttacccatcccgcagttcctgtccttgaatggggggtggggtgctctgcctatctcttgtggggtgatcagcccaaaaatcatgatttggagtgatctgatcagtgctgataggcagtttacaaagggattctggcttgtgacttcagtgaggacaatcccccagggccctttctttccatgcctctccaactcagagccaatgtctttgggtgggctagatagatagggcatacaattggcctggttcctccaagctcttaattcactttatcaatagttccatttaaattgacttcaatgataagagtgtatcccatttgagattgcttgcgttgtgggggagggggaggaggaacacattaagataattcacatgggcaaagggaggtcttggagtgtagccgttaagccatcttgtaaccccattcatgattttgaccacctgctagagagaagaggtgccaagagactagaacttggaggcttggctgtcccactaataggctttcgcaaggcagaggtagccagctaggtccctgccttcccagccaggtacagctctcaggtttgtggaggtaatctgtgaacttctcttcctgctgccttcttgtgatgtccagagcccacagtcaaatacctcctaagaaccctggcttccttccctctaatccactggcacatgactatcacctctggattgacctcagatccatagcctacacactgctagcagtggccaagatcacttcctttatctccatctgttctgttctccaggaaagtaagtggggatgagggtggaggtggtaatcaactgtagatctgtggctttatgagccttcagacttctctctggcttcttctggaagggttactattggcagtattgcaatctcaccctcctgatgaactgtagcctgtgccgttactgtgctgggcatgatctccagtgcttgcaagtcccatgatttctttggtgattttgagggtggggggagggacacaaatcagcttagcttagcttcctgtctgtgaatgtccatatagtgtattgtgttttaacaaatgatctacactgactgttgctgtaaaagtgaatttggaaataaagttattactctgaataaacaaaaatctctgttcatgagttccaagtacaagaaaacaatggtttacaagttctttcccccacttagaaaattcagaacaggaaatgaaggtaaatgccagacactggagtgagagctctcttggttcattctcacattcctctgctggcatactaccctccgacaaacagtactctgtgccttggcttacaaggcagcattcaagagctcctaaagcctgcgccagttcatgcaagctgaagaagtttggcgtgggcttcatttctggaacttaagtgtcgaggactggggctgaaacaaactcaagccaatcagctacaaacccagcacgcatacacaattgcactgctgcctttgtgccttggaatcttaccacatagaaatcaaggaatatttctgctggagataggacagcttcttgctcacagtattacaaaggaaaaaaaaaaaaaaaagacaacccatagtctcttgagacagggtctcttcatgcagcccaggctggtcttgatcctcctgactgctgggattatagaggtgtaccatgctgttaatgaagtgcaaggtcctcatgcatgcttaggcaacccaccctcaggcccaatttcttgttgaatgccatggcatgtagacagagtaccactgctgtgtgagcctatccactctatgtttgagacattattttcctacacttgtgccatgttatctggcctcttagctgcttagggattcttgggcctcagtttcccatctcacataggaacactgggatcacggatgcctggctttatgttggttctagattcaaattcagggtctgatcctgcacttcactgagctgcctcccttactccccagttctttggtaaatagatcaaggtcacgatctttagattcccaattttaaaacatttgtcttcctcagttcccaccaatgcctggcctctgcagacagttaatgcatataggaatttcctgcctaaaggcaggaaaatgatctaggaaagaacagctctgagccttggagaaggtgcacctgctcaccagccctgcatgtcagaactggacccttacactctttataagtagcggtagtctctcccagatgatgtgcttacttacttccattgattgcaacacctcaaagaagaaaaatccctattggctagaatttgaaattttaccctattgacctctcagaagaaagagtcaatgcatcaaccattttcactcaagaacagatagtatttttcaccttagtgaactgaacgactgatcacatcacacacccagtgttcccacaactgagagagaccaccagggcttagcaatgcagccccacagaatgctggggctgccatctctgtcagacacacagtccagtgtatcaagaaaagcctgacaaaactctctgtatctttttttattattatttttttcatttttcctttcttttttttttaagcactagtctgtgctttgcaaacagaatcatgacattaacaaagatcagcttctc

SEQ ID NO: 13 - DNA, 19 bp, Artificial sequence
  Primer
cggcacctcagcaatgtgt

SEQ ID NO: 14 - DNA, 20 bp, Artificial sequence
  Primer
tgtggcaagctgtggtgagt

SEQ ID NO: 15 - DNA, 22 bp, Artificial sequence
  Probe
ttccacgggcagcatcgacatg

SEQ ID NO: 16 - DNA, 20 bp, Artificial sequence
  Synthetic oligonucleotide
atcactgattttgaagtccc

SEQ ID NO: 17 - DNA, 18 bp, Artificial sequence
  Synthetic oligonucleotide
ttgtggcggcggcggcag

SEQ ID NO: 18 - DNA, 18 bp, Artificial sequence
  Synthetic oligonucleotide
ggtggttgtggcggcggc

SEQ ID NO: 19 - DNA, 18 bp, Artificial sequence
  Synthetic oligonucleotide
gagaaggtggttgtggcg

SEQ ID NO: 20 - DNA, 18 bp, Artificial sequence
  Synthetic oligonucleotide
cggaggagaaggtggttg

SEQ ID NO: 21 - DNA, 18 bp, Artificial sequence
  Synthetic oligonucleotide
gacagcggaggagaaggt

SEQ ID NO: 22 - DNA, 18 bp, Artificial sequence
  Synthetic oligonucleotide
aagaggacagcggaggag

SEQ ID NO: 23 - DNA, 18 bp, Artificial sequence
  Synthetic oligonucleotide
gacagaagaggacagcgg

SEQ ID NO: 24 - DNA, 18 bp, Artificial sequence
  Synthetic oligonucleotide
gcgaggacagaagaggac

SEQ ID NO: 25 - DNA, 18 bp, Artificial sequence
  Synthetic oligonucleotide
agaaggcgaggacagaag

SEQ ID NO: 26 - DNA, 18 bp, Artificial sequence
  Synthetic oligonucleotide
tcgacagaaggcgaggac

SEQ ID NO: 27 - DNA, 18 bp, Artificial sequence
  Synthetic oligonucleotide
gataatcgacagaaggcg

SEQ ID NO: 28 - DNA, 18 bp, Artificial sequence
  Synthetic oligonucleotide
tacctgataatcgacaga

SEQ ID NO: 29 - DNA, 18 bp, Artificial sequence
  Synthetic oligonucleotide
gcgcttacctgataatcg

SEQ ID NO: 30 - DNA, 18 bp, Artificial sequence
  Synthetic oligonucleotide
ctgcggcgcttacctgat

SEQ ID NO: 31 - DNA, 18 bp, Artificial sequence
  Synthetic oligonucleotide
cagagctgcggcgcttac

SEQ ID NO: 32 - DNA, 18 bp, Artificial sequence
  Synthetic oligonucleotide
gatttcagagctgcggcg

SEQ ID NO: 33 - DNA, 18 bp, Artificial sequence
  Synthetic oligonucleotide
gactggatttcagagctg

SEQ ID NO: 34 - DNA, 18 bp, Artificial sequence
  Synthetic oligonucleotide
aattggttacaggtggga

SEQ ID NO: 35 - DNA, 18 bp, Artificial sequence
  Synthetic oligonucleotide
aatggaattggttacagg

SEQ ID NO: 36 - DNA, 18 bp, Artificial sequence
  Synthetic oligonucleotide
aagacaatggaattggtt

SEQ ID NO: 37 - DNA, 18 bp, Artificial sequence
  Synthetic oligonucleotide
aaaaaaagacaatggaat

SEQ ID NO: 38 - DNA, 18 bp, Artificial sequence
  Synthetic oligonucleotide
ggaggaaaaaaagacaat

SEQ ID NO: 39 - DNA, 18 bp, Artificial sequence
  Synthetic oligonucleotide
agcctggaggaaaaaaag

SEQ ID NO: 40 - DNA, 18 bp, Artificial sequence
  Synthetic oligonucleotide
ttcaaagcctggaggaaa

SEQ ID NO: 41 - DNA, 18 bp, Artificial sequence
  Synthetic oligonucleotide
actggttcaaagcctgga

SEQ ID NO: 42 - DNA, 18 bp, Artificial sequence
  Synthetic oligonucleotide
gccatactggttcaaagc

SEQ ID NO: 43 - DNA, 18 bp, Artificial sequence
  Synthetic oligonucleotide
ggtcagccatactggttc

SEQ ID NO: 44 - DNA, 18 bp, Artificial sequence
  Synthetic oligonucleotide
gcgagggtcagccatact

SEQ ID NO: 45 - DNA, 18 bp, Artificial sequence
  Synthetic oligonucleotide
tcctggcgagggtcagcc

SEQ ID NO: 46 - DNA, 18 bp, Artificial sequence
  Synthetic oligonucleotide
caaactcctggcgagggt

SEQ ID NO: 47 - DNA, 18 bp, Artificial sequence
  Synthetic oligonucleotide
tgtgtcaaactcctggcg

SEQ ID NO: 48 - DNA, 18 bp, Artificial sequence
  Synthetic oligonucleotide
tccattgtgtcaaactcc

SEQ ID NO: 49 - DNA, 18 bp, Artificial sequence
  Synthetic oligonucleotide
ggtcttccattgtgtcaa

SEQ ID NO: 50 - DNA, 18 bp, Artificial sequence
  Synthetic oligonucleotide
agcatggtcttccattgt

SEQ ID NO: 51 - DNA, 18 bp, Artificial sequence
  Synthetic oligonucleotide
tctccagcatggtcttcc

SEQ ID NO: 52 - DNA, 18 bp, Artificial sequence
  Synthetic oligonucleotide
tgtaatctccagcatggt

SEQ ID NO: 53 - DNA, 18 bp, Artificial sequence
  Synthetic oligonucleotide
cagagtgtaatctccagc

SEQ ID NO: 54 - DNA, 18 bp, Artificial sequence
  Synthetic oligonucleotide
tggagcagagtgtaatct

SEQ ID NO: 55 - DNA, 18 bp, Artificial sequence
  Synthetic oligonucleotide
ggtcttggagcagagtgt

SEQ ID NO: 56 - DNA, 18 bp, Artificial sequence
  Synthetic oligonucleotide
ttcttggtcttggagcag

SEQ ID NO: 57 - DNA, 18 bp, Artificial sequence
  Synthetic oligonucleotide
tctccttcttggtcttgg

SEQ ID NO: 58 - DNA, 18 bp, Artificial sequence
  Synthetic oligonucleotide
ccatgtctccttcttggt

SEQ ID NO: 59 - DNA, 18 bp, Artificial sequence
  Synthetic oligonucleotide
atggtccatgtctccttc

SEQ ID NO: 60 - DNA, 18 bp, Artificial sequence
  Synthetic oligonucleotide
aagccatggtccatgtct

SEQ ID NO: 61 - DNA, 18 bp, Artificial sequence
  Synthetic oligonucleotide
cttttaagccatggtcca

SEQ ID NO: 62 - DNA, 18 bp, Artificial sequence
  Synthetic oligonucleotide
ctgaccttttaagccatg

SEQ ID NO: 63 - DNA, 18 bp, Artificial sequence
  Synthetic oligonucleotide
ccccactgaccttttaag

SEQ ID NO: 64 - DNA, 18 bp, Artificial sequence
  Synthetic oligonucleotide
gtccaccccactgacctt

SEQ ID NO: 65 - DNA, 18 bp, Artificial sequence
  Synthetic oligonucleotide
tgtatgtccaccccactg

SEQ ID NO: 66 - DNA, 18 bp, Artificial sequence
  Synthetic oligonucleotide
agttgtgtatgtccaccc

SEQ ID NO: 67 - DNA, 18 bp, Artificial sequence
  Synthetic oligonucleotide
tggccagttgtgtatgtc

SEQ ID NO: 68 - DNA, 18 bp, Artificial sequence
  Synthetic oligonucleotide
gtgactggccagttgtgt

SEQ ID NO: 69 - DNA, 18 bp, Artificial sequence
  Synthetic oligonucleotide
ccactgtgactggccagt

SEQ ID NO: 70 - DNA, 18 bp, Artificial sequence
  Synthetic oligonucleotide
tcagacctcaagaggaac

SEQ ID NO: 71 - DNA, 18 bp, Artificial sequence
  Synthetic oligonucleotide
agccctcagacctcaaga

SEQ ID NO: 72 - DNA, 18 bp, Artificial sequence
  Synthetic oligonucleotide
cagtgagccctcagacct

SEQ ID NO: 73 - DNA, 18 bp, Artificial sequence
  Synthetic oligonucleotide
atatacagtgagccctca

SEQ ID NO: 74 - DNA, 18 bp, Artificial sequence
  Synthetic oligonucleotide
ggaacatatacagtgagc

SEQ ID NO: 75 - DNA, 18 bp, Artificial sequence
  Synthetic oligonucleotide
actctggaacatatacag

SEQ ID NO: 76 - DNA, 18 bp, Artificial sequence
  Synthetic oligonucleotide
gggagactctggaacata

SEQ ID NO: 77 - DNA, 18 bp, Artificial sequence
  Synthetic oligonucleotide
ctccgctccatcatcggc

SEQ ID NO: 78 - DNA, 18 bp, Artificial sequence
  Synthetic oligonucleotide
ggttcctccgctccatca

SEQ ID NO: 79 - DNA, 18 bp, Artificial sequence
  Synthetic oligonucleotide
accctggttcctccgctc

SEQ ID NO: 80 - DNA, 18 bp, Artificial sequence
  Synthetic oligonucleotide
ctccgaccctggttcctc

SEQ ID NO: 81 - DNA, 18 bp, Artificial sequence
  Synthetic oligonucleotide
gaggtctccgaccctggt

SEQ ID NO: 82 - DNA, 18 bp, Artificial sequence
  Synthetic oligonucleotide
catcggaggtctccgacc

SEQ ID NO: 83 - DNA, 18 bp, Artificial sequence
  Synthetic oligonucleotide
cttagcatcggaggtctc

SEQ ID NO: 84 - DNA, 18 bp, Artificial sequence
  Synthetic oligonucleotide
gtgctcttagcatcggag

SEQ ID NO: 85 - DNA, 18 bp, Artificial sequence
  Synthetic oligonucleotide
ttggagtgctcttagcat

SEQ ID NO: 86 - DNA, 18 bp, Artificial sequence
  Synthetic oligonucleotide
agcagttggagtgctctt

SEQ ID NO: 87 - DNA, 18 bp, Artificial sequence
  Synthetic oligonucleotide
ccttcagcagttggagtg

SEQ ID NO: 88 - DNA, 18 bp, Artificial sequence
  Synthetic oligonucleotide
ccccaccttcagcagttg

SEQ ID NO: 89 - DNA, 18 bp, Artificial sequence
  Synthetic oligonucleotide
ggaggccccaccttcagc

SEQ ID NO: 90 - DNA, 18 bp, Artificial sequence
  Synthetic oligonucleotide
agtagggaggccccacct

SEQ ID NO: 91 - DNA, 18 bp, Artificial sequence
  Synthetic oligonucleotide
gtgggagtagggaggccc

SEQ ID NO: 92 - DNA, 18 bp, Artificial sequence
  Synthetic oligonucleotide
gagtggtgggagtaggga

SEQ ID NO: 93 - DNA, 18 bp, Artificial sequence
  Synthetic oligonucleotide
gaatggagtggtgggagt

SEQ ID NO: 94 - DNA, 18 bp, Artificial sequence
  Synthetic oligonucleotide
tgaatgaatggagtggtg

SEQ ID NO: 95 - DNA, 18 bp, Artificial sequence
  Synthetic oligonucleotide
taagctgaatgaatggag

SEQ ID NO: 96 - DNA, 18 bp, Artificial sequence
  Synthetic oligonucleotide
gaggacagaagggagact

SEQ ID NO: 97 - DNA, 18 bp, Artificial sequence
  Synthetic oligonucleotide
agggagaggacagaaggg

SEQ ID NO: 98 - DNA, 18 bp, Artificial sequence
  Synthetic oligonucleotide
agcaaagggagaggacag

SEQ ID NO: 99 - DNA, 18 bp, Artificial sequence
  Synthetic oligonucleotide
gaggcagcaaagggagag

SEQ ID NO: 100 - DNA, 18 bp, Artificial sequence
  Synthetic oligonucleotide
ggtccgaggcagcaaagg

SEQ ID NO: 101 - DNA, 18 bp, Artificial sequence
  Synthetic oligonucleotide
cggctggtccgaggcagc

SEQ ID NO: 102 - DNA, 18 bp, Artificial sequence
  Synthetic oligonucleotide
ttcttcggctggtccgag

SEQ ID NO: 103 - DNA, 18 bp, Artificial sequence
  Synthetic oligonucleotide
cctgcttcttcggctggt

SEQ ID NO: 104 - DNA, 18 bp, Artificial sequence
  Synthetic oligonucleotide
cgatgcctgcttcttcgg

SEQ ID NO: 105 - DNA, 18 bp, Artificial sequence
  Synthetic oligonucleotide
gtctccgatgcctgcttc

SEQ ID NO: 106 - DNA, 18 bp, Artificial sequence
  Synthetic oligonucleotide
tggtcctcctggttcggg

SEQ ID NO: 107 - DNA, 18 bp, Artificial sequence
  Synthetic oligonucleotide
cggcttggtcctcctggt

SEQ ID NO: 108 - DNA, 18 bp, Artificial sequence
  Synthetic oligonucleotide
cccagcggcttggtcctc

SEQ ID NO: 109 - DNA, 18 bp, Artificial sequence
  Synthetic oligonucleotide
acatgcccagcggcttgg

SEQ ID NO: 110 - DNA, 18 bp, Artificial sequence
  Synthetic oligonucleotide
gagtcacatgcccagcgg

SEQ ID NO: 111 - DNA, 18 bp, Artificial sequence
  Synthetic oligonucleotide
accttgagtcacatgccc

SEQ ID NO: 112 - DNA, 18 bp, Artificial sequence
  Synthetic oligonucleotide
cactgaccttgagtcaca

SEQ ID NO: 113 - DNA, 18 bp, Artificial sequence
  Synthetic oligonucleotide
tgggccactgaccttgag

SEQ ID NO: 114 - DNA, 18 bp, Artificial sequence
  Synthetic oligonucleotide
cgatttgggccactgacc

SEQ ID NO: 115 - DNA, 18 bp, Artificial sequence
  Synthetic oligonucleotide
aaagtcgatttgggccac

SEQ ID NO: 116 - DNA, 18 bp, Artificial sequence
  Synthetic oligonucleotide
gtcccaaagtcgatttgg

SEQ ID NO: 117 - DNA, 18 bp, Artificial sequence
  Synthetic oligonucleotide
tctgagtcccaaagtcga

SEQ ID NO: 118 - DNA, 18 bp, Artificial sequence
  Synthetic oligonucleotide
cccaatctgagtcccaaa

SEQ ID NO: 119 - DNA, 18 bp, Artificial sequence
  Synthetic oligonucleotide
ctcctcccaatctgagtc

SEQ ID NO: 120 - DNA, 18 bp, Artificial sequence
  Synthetic oligonucleotide
gtgcacgtgatgccccgt

SEQ ID NO: 121 - DNA, 18 bp, Artificial sequence
  Synthetic oligonucleotide
agagagtgcacgtgatgc

SEQ ID NO: 122 - DNA, 18 bp, Artificial sequence
  Synthetic oligonucleotide
aaaatagagagtgcacgt

SEQ ID NO: 123 - DNA, 18 bp, Artificial sequence
  Synthetic oligonucleotide
aatataaaatagagagtg

SEQ ID NO: 124 - DNA, 18 bp, Artificial sequence
  Synthetic oligonucleotide
ggtaaaatataaaataga

SEQ ID NO: 125 - DNA, 18 bp, Artificial sequence
  Synthetic oligonucleotide
gagctggtaaaatataaa

SEQ ID NO: 126 - DNA, 18 bp, Artificial sequence
  Synthetic oligonucleotide
cacacgagctggtaaaat

SEQ ID NO: 127 - DNA, 18 bp, Artificial sequence
  Synthetic oligonucleotide
ctggccacacgagctggt

SEQ ID NO: 128 - DNA, 18 bp, Artificial sequence
  Synthetic oligonucleotide
ctttgctggccacacgag

SEQ ID NO: 129 - DNA, 18 bp, Artificial sequence
  Synthetic oligonucleotide
cctgtctttgctggccac

SEQ ID NO: 130 - DNA, 18 bp, Artificial sequence
  Synthetic oligonucleotide
cctgtcctgtctttgctg

SEQ ID NO: 131 - DNA, 18 bp, Artificial sequence
  Synthetic oligonucleotide
catttcctgtcctgtctt

SEQ ID NO: 132 - DNA, 18 bp, Artificial sequence
  Synthetic oligonucleotide
ctcgtcatttcctgtcct

SEQ ID NO: 133 - DNA, 18 bp, Artificial sequence
  Synthetic oligonucleotide
ttcttctcgtcatttcct

SEQ ID NO: 134 - DNA, 18 bp, Artificial sequence
  Synthetic oligonucleotide
tggctttcttctcgtcat

SEQ ID NO: 135 - DNA, 18 bp, Artificial sequence
  Synthetic oligonucleotide
taccttggctttcttctc

SEQ ID NO: 136 - DNA, 18 bp, Artificial sequence
  Synthetic oligonucleotide
tagcttaccttggctttc

SEQ ID NO: 137 - DNA, 18 bp, Artificial sequence
  Synthetic oligonucleotide
gtcagtagcttaccttgg

SEQ ID NO: 138 - DNA, 18 bp, Artificial sequence
  Synthetic oligonucleotide
ggcgggtcagtagcttac

SEQ ID NO: 139 - DNA, 18 bp, Artificial sequence
  Synthetic oligonucleotide
ggaccggcgggtcagtag

SEQ ID NO: 140 - DNA, 18 bp, Artificial sequence
  Synthetic oligonucleotide
ttctaggaccggcgggtc

SEQ ID NO: 141 - DNA, 18 bp, Artificial sequence
  Synthetic oligonucleotide
gagccttctaggaccggc

SEQ ID NO: 142 - DNA, 18 bp, Artificial sequence
  Synthetic oligonucleotide
aagcagagccttctagga

SEQ ID NO: 143 - DNA, 18 bp, Artificial sequence
  Synthetic oligonucleotide
gcaggaagcagagccttc

SEQ ID NO: 144 - DNA, 18 bp, Artificial sequence
  Synthetic oligonucleotide
aggttttcaccactggga

SEQ ID NO: 145 - DNA, 18 bp, Artificial sequence
  Synthetic oligonucleotide
gtggaaggttttcaccac

SEQ ID NO: 146 - DNA, 18 bp, Artificial sequence
  Synthetic oligonucleotide
tcagggtggaaggttttc

SEQ ID NO: 147 - DNA, 18 bp, Artificial sequence
  Synthetic oligonucleotide
ccgaatcagggtggaagg

SEQ ID NO: 148 - DNA, 18 bp, Artificial sequence
  Synthetic oligonucleotide
gtaaaccgaatcagggtg

SEQ ID NO: 149 - DNA, 18 bp, Artificial sequence
  Synthetic oligonucleotide
gccctgtaaaccgaatca

SEQ ID NO: 150 - DNA, 18 bp, Artificial sequence
  Synthetic oligonucleotide
tcagcgccctgtaaaccg

SEQ ID NO: 151 - DNA, 18 bp, Artificial sequence
  Synthetic oligonucleotide
tgccatcagcgccctgta

SEQ ID NO: 152 - DNA, 18 bp, Artificial sequence
  Synthetic oligonucleotide
ggttttgccatcagcgcc

SEQ ID NO: 153 - DNA, 18 bp, Artificial sequence
  Synthetic oligonucleotide
gccccggttttgccatca

SEQ ID NO: 154 - DNA, 18 bp, Artificial sequence
  Synthetic oligonucleotide
tcttcgccccggttttgc

SEQ ID NO: 155 - DNA, 18 bp, Artificial sequence
  Synthetic oligonucleotide
ggcgatcttcgccccggt

SEQ ID NO: 156 - DNA, 18 bp, Artificial sequence
  Synthetic oligonucleotide
ggtgtggcgatcttcgcc

SEQ ID NO: 157 - DNA, 18 bp, Artificial sequence
  Synthetic oligonucleotide
cccgaggtgtggcgatct

SEQ ID NO: 158 - DNA, 18 bp, Artificial sequence
  Synthetic oligonucleotide
tgctccccgaggtgtggc

SEQ ID NO: 159 - DNA, 18 bp, Artificial sequence
  Synthetic oligonucleotide
gaggctgctccccgaggt

SEQ ID NO: 160 - DNA, 18 bp, Artificial sequence
  Synthetic oligonucleotide
ccggagaggctgctcccc

SEQ ID NO: 161 - DNA, 18 bp, Artificial sequence
  Synthetic oligonucleotide
ctgggccggagaggctgc

SEQ ID NO: 162 - DNA, 18 bp, Artificial sequence
  Synthetic oligonucleotide
cccttctgggccggagag

SEQ ID NO: 163 - DNA, 18 bp, Artificial sequence
  Synthetic oligonucleotide
acgtgcccttctgggccg

SEQ ID NO: 164 - DNA, 18 bp, Artificial sequence
  Synthetic oligonucleotide
gttggacgtgcccttctg

SEQ ID NO: 165 - DNA, 18 bp, Artificial sequence
  Synthetic oligonucleotide
gtggcgttggacgtgccc

SEQ ID NO: 166 - DNA, 18 bp, Artificial sequence
  Synthetic oligonucleotide
tcctggtggcgttggacg

SEQ ID NO: 167 - DNA, 18 bp, Artificial sequence
  Synthetic oligonucleotide
cgggatcctggtggcgtt

SEQ ID NO: 168 - DNA, 18 bp, Artificial sequence
  Synthetic oligonucleotide
ttggccgggatcctggtg

SEQ ID NO: 169 - DNA, 18 bp, Artificial sequence
  Synthetic oligonucleotide
tggtcttggccgggatcc

SEQ ID NO: 170 - DNA, 18 bp, Artificial sequence
  Synthetic oligonucleotide
gggcgtggtcttggccgg

SEQ ID NO: 171 - DNA, 18 bp, Artificial sequence
  Synthetic oligonucleotide
gggctgggcgtggtcttg

SEQ ID NO: 172 - DNA, 18 bp, Artificial sequence
  Synthetic oligonucleotide
tcttagggctgggcgtgg

SEQ ID NO: 173 - DNA, 18 bp, Artificial sequence
  Synthetic oligonucleotide
aggagtcttagggctggg

SEQ ID NO: 174 - DNA, 18 bp, Artificial sequence
  Synthetic oligonucleotide
cctggaggagtcttaggg

SEQ ID NO: 175 - DNA, 18 bp, Artificial sequence
  Synthetic oligonucleotide
ctgaccctggaggagtct

SEQ ID NO: 176 - DNA, 18 bp, Artificial sequence
  Synthetic oligonucleotide
ctcacctgaccctggagg

SEQ ID NO: 177 - DNA, 18 bp, Artificial sequence
  Synthetic oligonucleotide
gtagtctcacctgaccct

SEQ ID NO: 178 - DNA, 18 bp, Artificial sequence
  Synthetic oligonucleotide
agagagtagtctcacctg

SEQ ID NO: 179 - DNA, 18 bp, Artificial sequence
  Synthetic oligonucleotide
actccagagagtagtctc

SEQ ID NO: 180 - DNA, 18 bp, Artificial sequence
  Synthetic oligonucleotide
ttaaaactccagagagta

SEQ ID NO: 181 - DNA, 18 bp, Artificial sequence
  Synthetic oligonucleotide
ctggattaaaactccaga

SEQ ID NO: 182 - DNA, 18 bp, Artificial sequence
  Synthetic oligonucleotide
agcttctggattaaaact

SEQ ID NO: 183 - DNA, 18 bp, Artificial sequence
  Synthetic oligonucleotide
ctggaagcttctggatta

SEQ ID NO: 184 - DNA, 18 bp, Artificial sequence
  Synthetic oligonucleotide
gggactgaggccacgtgg

SEQ ID NO: 185 - DNA, 18 bp, Artificial sequence
  Synthetic oligonucleotide
aggaagggactgaggcca

SEQ ID NO: 186 - DNA, 18 bp, Artificial sequence
  Synthetic oligonucleotide
gagagaggaagggactga

SEQ ID NO: 187 - DNA, 18 bp, Artificial sequence
  Synthetic oligonucleotide
gtcgggagagaggaaggg

SEQ ID NO: 188 - DNA, 18 bp, Artificial sequence
  Synthetic oligonucleotide
ggaaagtcgggagagagg

SEQ ID NO: 189 - DNA, 18 bp, Artificial sequence
  Synthetic oligonucleotide
cacctggaaagtcgggag

SEQ ID NO: 190 - DNA, 18 bp, Artificial sequence
  Synthetic oligonucleotide
tggttcacctggaaagtc

SEQ ID NO: 191 - DNA, 18 bp, Artificial sequence
  Synthetic oligonucleotide
tttggtggttcacctgga

SEQ ID NO: 192 - DNA, 18 bp, Artificial sequence
  Synthetic oligonucleotide
cggattttggtggttcac

SEQ ID NO: 193 - DNA, 18 bp, Artificial sequence
  Synthetic oligonucleotide
ttctccggattttggtgg

SEQ ID NO: 194 - DNA, 18 bp, Artificial sequence
  Synthetic oligonucleotide
cttcgttctccggatttt

SEQ ID NO: 195 - DNA, 18 bp, Artificial sequence
  Synthetic oligonucleotide
agccgcttcgttctccgg

SEQ ID NO: 196 - DNA, 18 bp, Artificial sequence
  Synthetic oligonucleotide
gctgtagccgcttcgttc

SEQ ID NO: 197 - DNA, 18 bp, Artificial sequence
  Synthetic oligonucleotide
gggctgctgtagccgctt

SEQ ID NO: 198 - DNA, 18 bp, Artificial sequence
  Synthetic oligonucleotide
caggcgttccgggagagc

SEQ ID NO: 199 - DNA, 18 bp, Artificial sequence
  Synthetic oligonucleotide
actgccaggcgttccggg

SEQ ID NO: 200 - DNA, 18 bp, Artificial sequence
  Synthetic oligonucleotide
gagcgactgccaggcgtt

SEQ ID NO: 201 - DNA, 18 bp, Artificial sequence
  Synthetic oligonucleotide
tgcgcgagcgactgccag

SEQ ID NO: 202 - DNA, 18 bp, Artificial sequence
  Synthetic oligonucleotide
cggtgttggtagggatgg

SEQ ID NO: 203 - DNA, 18 bp, Artificial sequence
  Synthetic oligonucleotide
gtgggcggtgttggtagg

SEQ ID NO: 204 - DNA, 18 bp, Artificial sequence
  Synthetic oligonucleotide
cccgggtgggcggtgttg

SEQ ID NO: 205 - DNA, 18 bp, Artificial sequence
  Synthetic oligonucleotide
gggctcccgggtgggcgg

SEQ ID NO: 206 - DNA, 18 bp, Artificial sequence
  Synthetic oligonucleotide
ttcttgggctcccgggtg

SEQ ID NO: 207 - DNA, 18 bp, Artificial sequence
  Synthetic oligonucleotide
ccaccttcttgggctccc

SEQ ID NO: 208 - DNA, 18 bp, Artificial sequence
  Synthetic oligonucleotide
cactgccaccttcttggg

SEQ ID NO: 209 - DNA, 18 bp, Artificial sequence
  Synthetic oligonucleotide
cggaccactgccaccttc

SEQ ID NO: 210 - DNA, 18 bp, Artificial sequence
  Synthetic oligonucleotide
gagtgcggaccactgcca

SEQ ID NO: 211 - DNA, 18 bp, Artificial sequence
  Synthetic oligonucleotide
atggtgacttagggggag

SEQ ID NO: 212 - DNA, 18 bp, Artificial sequence
  Synthetic oligonucleotide
agctgatggtgacttagg

SEQ ID NO: 213 - DNA, 18 bp, Artificial sequence
  Synthetic oligonucleotide
ttactagctgatggtgac

SEQ ID NO: 214 - DNA, 18 bp, Artificial sequence
  Synthetic oligonucleotide
ggctcttactagctgatg

SEQ ID NO: 215 - DNA, 18 bp, Artificial sequence
  Synthetic oligonucleotide
caggcggctcttactagc

SEQ ID NO: 216 - DNA, 18 bp, Artificial sequence
  Synthetic oligonucleotide
gtctgcaggcggctctta

SEQ ID NO: 217 - DNA, 18 bp, Artificial sequence
  Synthetic oligonucleotide
gggcagtctgcaggcggc

SEQ ID NO: 218 - DNA, 18 bp, Artificial sequence
  Synthetic oligonucleotide
taggtctggcatgggcac

SEQ ID NO: 219 - DNA, 18 bp, Artificial sequence
  Synthetic oligonucleotide
ttctttaggtctggcatg

SEQ ID NO: 220 - DNA, 18 bp, Artificial sequence
  Synthetic oligonucleotide
tgacattctttaggtctg

SEQ ID NO: 221 - DNA, 18 bp, Artificial sequence
  Synthetic oligonucleotide
cgacctgacattctttag

SEQ ID NO: 222 - DNA, 18 bp, Artificial sequence
  Synthetic oligonucleotide
atcttcgacctgacattc

SEQ ID NO: 223 - DNA, 18 bp, Artificial sequence
  Synthetic oligonucleotide
agccaatcttcgacctga

SEQ ID NO: 224 - DNA, 18 bp, Artificial sequence
  Synthetic oligonucleotide
agtagagccaatcttcga

SEQ ID NO: 225 - DNA, 18 bp, Artificial sequence
  Synthetic oligonucleotide
ttctcagtagagccaatc

SEQ ID NO: 226 - DNA, 18 bp, Artificial sequence
  Synthetic oligonucleotide
tcaggttctcagtagagc

SEQ ID NO: 227 - DNA, 18 bp, Artificial sequence
  Synthetic oligonucleotide
gtgcttcaggttctcagt

SEQ ID NO: 228 - DNA, 18 bp, Artificial sequence
  Synthetic oligonucleotide
ggctggtgcttcaggttc

SEQ ID NO: 229 - DNA, 18 bp, Artificial sequence
  Synthetic oligonucleotide
ctcctggctggtgcttca

SEQ ID NO: 230 - DNA, 18 bp, Artificial sequence
  Synthetic oligonucleotide
gccacctcctggctggtg

SEQ ID NO: 231 - DNA, 18 bp, Artificial sequence
  Synthetic oligonucleotide
accttgccacctcctggc

SEQ ID NO: 232 - DNA, 18 bp, Artificial sequence
  Synthetic oligonucleotide
cccttaccttgccacctc

SEQ ID NO: 233 - DNA, 18 bp, Artificial sequence
  Synthetic oligonucleotide
ccacacccttaccttgcc

SEQ ID NO: 234 - DNA, 18 bp, Artificial sequence
  Synthetic oligonucleotide
accagccacacccttacc

SEQ ID NO: 235 - DNA, 18 bp, Artificial sequence
  Synthetic oligonucleotide
agaagaccagccacaccc

SEQ ID NO: 236 - DNA, 18 bp, Artificial sequence
  Synthetic oligonucleotide
ttcccagaagaccagcca

SEQ ID NO: 237 - DNA, 18 bp, Artificial sequence
  Synthetic oligonucleotide
ctggcttcccagaagacc

SEQ ID NO: 238 - DNA, 18 bp, Artificial sequence
  Synthetic oligonucleotide
ggagtctggcttcccaga

SEQ ID NO: 239 - DNA, 18 bp, Artificial sequence
  Synthetic oligonucleotide
aggtgggagtctggcttc

SEQ ID NO: 240 - DNA, 18 bp, Artificial sequence
  Synthetic oligonucleotide
gagagagagaataaaggg

SEQ ID NO: 241 - DNA, 18 bp, Artificial sequence
  Synthetic oligonucleotide
tgggcgagagagagaata

SEQ ID NO: 242 - DNA, 18 bp, Artificial sequence
  Synthetic oligonucleotide
gaggatgggcgagagaga

SEQ ID NO: 243 - DNA, 18 bp, Artificial sequence
  Synthetic oligonucleotide
tcaaagaggatgggcgag

SEQ ID NO: 244 - DNA, 18 bp, Artificial sequence
  Synthetic oligonucleotide
gcaggtcaaagaggatgg

SEQ ID NO: 245 - DNA, 18 bp, Artificial sequence
  Synthetic oligonucleotide
cacctgcaggtcaaagag

SEQ ID NO: 246 - DNA, 18 bp, Artificial sequence
  Synthetic oligonucleotide
atttgcacctgcaggtca

SEQ ID NO: 247 - DNA, 18 bp, Artificial sequence
  Synthetic oligonucleotide
agactatttgcacctgca

SEQ ID NO: 248 - DNA, 18 bp, Artificial sequence
  Synthetic oligonucleotide
cttgtagactatttgcac

SEQ ID NO: 249 - DNA, 18 bp, Artificial sequence
  Synthetic oligonucleotide
accggcttgtagactatt

SEQ ID NO: 250 - DNA, 18 bp, Artificial sequence
  Synthetic oligonucleotide
ggtccaccggcttgtaga

SEQ ID NO: 251 - DNA, 18 bp, Artificial sequence
  Synthetic oligonucleotide
gctcaggtccaccggctt

SEQ ID NO: 252 - DNA, 18 bp, Artificial sequence
  Synthetic oligonucleotide
actttgctcaggtccacc

SEQ ID NO: 253 - DNA, 18 bp, Artificial sequence
  Synthetic oligonucleotide
aggtcactttgctcaggt

SEQ ID NO: 254 - DNA, 18 bp, Artificial sequence
  Synthetic oligonucleotide
cttggaggtcactttgct

SEQ ID NO: 255 - DNA, 18 bp, Artificial sequence
  Synthetic oligonucleotide
ccacacttggaggtcact

SEQ ID NO: 256 - DNA, 18 bp, Artificial sequence
  Synthetic oligonucleotide
acgagccacacttggagg

SEQ ID NO: 257 - DNA, 18 bp, Artificial sequence
  Synthetic oligonucleotide
ccctaacgagccacactt

SEQ ID NO: 258 - DNA, 18 bp, Artificial sequence
  Synthetic oligonucleotide
atgttccctaacgagcca

SEQ ID NO: 259 - DNA, 18 bp, Artificial sequence
  Synthetic oligonucleotide
gatggatgttccctaacg

SEQ ID NO: 260 - DNA, 18 bp, Artificial sequence
  Synthetic oligonucleotide
cttgtgatggatgttccc

SEQ ID NO: 261 - DNA, 18 bp, Artificial sequence
  Synthetic oligonucleotide
cctggcttgtgatggatg

SEQ ID NO: 262 - DNA, 18 bp, Artificial sequence
  Synthetic oligonucleotide
tgctacctggcttgtgat

SEQ ID NO: 263 - DNA, 18 bp, Artificial sequence
  Synthetic oligonucleotide
tcaagtgctacctggctt

SEQ ID NO: 264 - DNA, 18 bp, Artificial sequence
  Synthetic oligonucleotide
cttcctcaagtgctacct

SEQ ID NO: 265 - DNA, 18 bp, Artificial sequence
  Synthetic oligonucleotide
cctgtcttcctcaagtgc

SEQ ID NO: 266 - DNA, 18 bp, Artificial sequence
  Synthetic oligonucleotide
caaagcctgtcttcctca

SEQ ID NO: 267 - DNA, 18 bp, Artificial sequence
  Synthetic oligonucleotide
tgtcccaaagcctgtctt

SEQ ID NO: 268 - DNA, 18 bp, Artificial sequence
  Synthetic oligonucleotide
actcctgtcccaaagcct

SEQ ID NO: 269 - DNA, 18 bp, Artificial sequence
  Synthetic oligonucleotide
ccagcactcctgtcccaa

SEQ ID NO: 270 - DNA, 18 bp, Artificial sequence
  Synthetic oligonucleotide
ctcttgccctagtctgtg

SEQ ID NO: 271 - DNA, 18 bp, Artificial sequence
  Synthetic oligonucleotide
tgagcctcttgccctagt

SEQ ID NO: 272 - DNA, 18 bp, Artificial sequence
  Synthetic oligonucleotide
ccacatgagcctcttgcc

SEQ ID NO: 273 - DNA, 18 bp, Artificial sequence
  Synthetic oligonucleotide
acaacccacatgagcctc

SEQ ID NO: 274 - DNA, 18 bp, Artificial sequence
  Synthetic oligonucleotide
ggaacacaacccacatga

SEQ ID NO: 275 - DNA, 18 bp, Artificial sequence
  Synthetic oligonucleotide
ctcctggaacacaaccca

SEQ ID NO: 276 - DNA, 18 bp, Artificial sequence
  Synthetic oligonucleotide
gccacctcctggaacaca

SEQ ID NO: 277 - DNA, 18 bp, Artificial sequence
  Synthetic oligonucleotide
acctggccacctcctgga

SEQ ID NO: 278 - DNA, 18 bp, Artificial sequence
  Synthetic oligonucleotide
cttccacctggccacctc

SEQ ID NO: 279 - DNA, 18 bp, Artificial sequence
  Synthetic oligonucleotide
ttttacttccacctggcc

SEQ ID NO: 280 - DNA, 18 bp, Artificial sequence
  Synthetic oligonucleotide
tctgattttacttccacc

SEQ ID NO: 281 - DNA, 18 bp, Artificial sequence
  Synthetic oligonucleotide
gcttctctgattttactt

SEQ ID NO: 282 - DNA, 18 bp, Artificial sequence
  Synthetic oligonucleotide
gtccagcttctctgattt

SEQ ID NO: 283 - DNA, 18 bp, Artificial sequence
  Synthetic oligonucleotide
ttgaagtccagcttctct

SEQ ID NO: 284 - DNA, 18 bp, Artificial sequence
  Synthetic oligonucleotide
tgtccttgaagtccagct

SEQ ID NO: 285 - DNA, 18 bp, Artificial sequence
  Synthetic oligonucleotide
gactctgtccttgaagtc

SEQ ID NO: 286 - DNA, 18 bp, Artificial sequence
  Synthetic oligonucleotide
gactggactctgtccttg

SEQ ID NO: 287 - DNA, 18 bp, Artificial sequence
  Synthetic oligonucleotide
tcttcgactggactctgt

SEQ ID NO: 288 - DNA, 18 bp, Artificial sequence
  Synthetic oligonucleotide
gccaatcttcgactggac

SEQ ID NO: 289 - DNA, 18 bp, Artificial sequence
  Synthetic oligonucleotide
aaggagccaatcttcgac

SEQ ID NO: 290 - DNA, 18 bp, Artificial sequence
  Synthetic oligonucleotide
tatccaaggagccaatct

SEQ ID NO: 291 - DNA, 18 bp, Artificial sequence
  Synthetic oligonucleotide
gatattatccaaggagcc

SEQ ID NO: 292 - DNA, 18 bp, Artificial sequence
  Synthetic oligonucleotide
tgggtgatattatccaag

SEQ ID NO: 293 - DNA, 18 bp, Artificial sequence
  Synthetic oligonucleotide
ggacgtgggtgatattat

SEQ ID NO: 294 - DNA, 18 bp, Artificial sequence
  Synthetic oligonucleotide
tccagggacgtgggtgat

SEQ ID NO: 295 - DNA, 18 bp, Artificial sequence
  Synthetic oligonucleotide
cctcctccagggacgtgg

SEQ ID NO: 296 - DNA, 18 bp, Artificial sequence
  Synthetic oligonucleotide
tattccctcctccaggga

SEQ ID NO: 297 - DNA, 18 bp, Artificial sequence
  Synthetic oligonucleotide
cttcttattccctcctcc

SEQ ID NO: 298 - DNA, 18 bp, Artificial sequence
  Synthetic oligonucleotide
cttaccttcttattccct

SEQ ID NO: 299 - DNA, 18 bp, Artificial sequence
  Synthetic oligonucleotide
acccccttaccttcttat

SEQ ID NO: 300 - DNA, 18 bp, Artificial sequence
  Synthetic oligonucleotide
atttgacccccttacctt

SEQ ID NO: 301 - DNA, 18 bp, Artificial sequence
  Synthetic oligonucleotide
ctcccatttgaccccctt

SEQ ID NO: 302 - DNA, 18 bp, Artificial sequence
  Synthetic oligonucleotide
atgacctcccatttgacc

SEQ ID NO: 303 - DNA, 18 bp, Artificial sequence
  Synthetic oligonucleotide
cccgtatgacctcccatt

SEQ ID NO: 304 - DNA, 18 bp, Artificial sequence
  Synthetic oligonucleotide
ttatccccgtatgacctc

SEQ ID NO: 305 - DNA, 18 bp, Artificial sequence
  Synthetic oligonucleotide
ccctcttatccccgtatg

SEQ ID NO: 306 - DNA, 18 bp, Artificial sequence
  Synthetic oligonucleotide
ttaatgataaggttctat

SEQ ID NO: 307 - DNA, 18 bp, Artificial sequence
  Synthetic oligonucleotide
tgagattaatgataaggt

SEQ ID NO: 308 - DNA, 18 bp, Artificial sequence
  Synthetic oligonucleotide
gagagtgagattaatgat

SEQ ID NO: 309 - DNA, 18 bp, Artificial sequence
  Synthetic oligonucleotide
atgtagagagtgagatta

SEQ ID NO: 310 - DNA, 18 bp, Artificial sequence
  Synthetic oligonucleotide
gcaagatgtagagagtga

SEQ ID NO: 311 - DNA, 18 bp, Artificial sequence
  Synthetic oligonucleotide
aatctgcaagatgtagag

SEQ ID NO: 312 - DNA, 18 bp, Artificial sequence
  Synthetic oligonucleotide
gtttcaatctgcaagatg

SEQ ID NO: 313 - DNA, 18 bp, Artificial sequence
  Synthetic oligonucleotide
tgtgggtttcaatctgca

SEQ ID NO: 314 - DNA, 18 bp, Artificial sequence
  Synthetic oligonucleotide
cagcttgtgggtttcaat

SEQ ID NO: 315 - DNA, 18 bp, Artificial sequence
  Synthetic oligonucleotide
aaggtcagcttgtgggtt

SEQ ID NO: 316 - DNA, 18 bp, Artificial sequence
  Synthetic oligonucleotide
ccctgaaggtcagcttgt

SEQ ID NO: 317 - DNA, 18 bp, Artificial sequence
  Synthetic oligonucleotide
attctccctgaaggtcag

SEQ ID NO: 318 - DNA, 18 bp, Artificial sequence
  Synthetic oligonucleotide
ttggcattctccctgaag

SEQ ID NO: 319 - DNA, 18 bp, Artificial sequence
  Synthetic oligonucleotide
tggctttggcattctccc

SEQ ID NO: 320 - DNA, 18 bp, Artificial sequence
  Synthetic oligonucleotide
tgtcttggctttggcatt

SEQ ID NO: 321 - DNA, 18 bp, Artificial sequence
  Synthetic oligonucleotide
tggtctgtcttggctttg

SEQ ID NO: 322 - DNA, 18 bp, Artificial sequence
  Synthetic oligonucleotide
ctccatggtctgtcttgg

SEQ ID NO: 323 - DNA, 18 bp, Artificial sequence
  Synthetic oligonucleotide
ttctgctccatggtctgt

SEQ ID NO: 324 - DNA, 18 bp, Artificial sequence
  Synthetic oligonucleotide
acaatttctgctccatgg

SEQ ID NO: 325 - DNA, 18 bp, Artificial sequence
  Synthetic oligonucleotide
atcgacagaaggcgagga

SEQ ID NO: 326 - DNA, 18 bp, Artificial sequence
  Synthetic oligonucleotide
aatcgacagaaggcgagg

SEQ ID NO: 327 - DNA, 18 bp, Artificial sequence
  Synthetic oligonucleotide
taatcgacagaaggcgag

SEQ ID NO: 328 - DNA, 18 bp, Artificial sequence
  Synthetic oligonucleotide
ataatcgacagaaggcga

SEQ ID NO: 329 - DNA, 18 bp, Artificial sequence
  Synthetic oligonucleotide
tgataatcgacagaaggc

SEQ ID NO: 330 - DNA, 18 bp, Artificial sequence
  Synthetic oligonucleotide
ctgataatcgacagaagg

SEQ ID NO: 331 - DNA, 18 bp, Artificial sequence
  Synthetic oligonucleotide
cctgataatcgacagaag

SEQ ID NO: 332 - DNA, 18 bp, Artificial sequence
  Synthetic oligonucleotide
acctgataatcgacagaa

SEQ ID NO: 333 - DNA, 18 bp, Artificial sequence
  Synthetic oligonucleotide
ttacctgataatcgacag

SEQ ID NO: 334 - DNA, 18 bp, Artificial sequence
  Synthetic oligonucleotide
cttacctgataatcgaca

SEQ ID NO: 335 - DNA, 18 bp, Artificial sequence
  Synthetic oligonucleotide
gcttacctgataatcgac

SEQ ID NO: 336 - DNA, 18 bp, Artificial sequence
  Synthetic oligonucleotide
cgcttacctgataatcga

SEQ ID NO: 337 - DNA, 18 bp, Artificial sequence
  Synthetic oligonucleotide
ggcgcttacctgataatc

SEQ ID NO: 338 - DNA, 18 bp, Artificial sequence
  Synthetic oligonucleotide
cggcgcttacctgataat

SEQ ID NO: 339 - DNA, 18 bp, Artificial sequence
  Synthetic oligonucleotide
gcggcgcttacctgataa

SEQ ID NO: 340 - DNA, 18 bp, Artificial sequence
  Synthetic oligonucleotide
tgcggcgcttacctgata

SEQ ID NO: 341 - DNA, 18 bp, Artificial sequence
  Synthetic oligonucleotide
gctgcggcgcttacctga

SEQ ID NO: 342 - DNA, 18 bp, Artificial sequence
  Synthetic oligonucleotide
agctgcggcgcttacctg

SEQ ID NO: 343 - DNA, 18 bp, Artificial sequence
  Synthetic oligonucleotide
gagctgcggcgcttacct

SEQ ID NO: 344 - DNA, 18 bp, Artificial sequence
  Synthetic oligonucleotide
agagctgcggcgcttacc

(19) United States
(12) Patent Application Publication
(10) Pub. No.: US 2017/0211064 A1
(43) Pub. Date: Jul. 27, 2017

(54) COMPOSITIONS AND METHODS FOR MODULATING TAU EXPRESSION

(71) Applicant: Ionis Pharmaceuticals, Inc., Carlsbad, CA (US)

(72) Inventor: Frank Rigo, Carlsbad, CA (US)

(73) Assignee: Ionis Pharmaceuticals, Inc., Carlsbad, CA (US)

(21) Appl. No.: 15/329,694

(22) PCT Filed: Jul. 29, 2015
(86) PCT No.: PCT/US15/42740

§ 371 Date: Jan. 27, 2017

Related U.S. Application Data
(60) Provisional application No. 62/030,596, filed on Jul. 29, 2014.

(51) Int. Cl. C12N 15/113

(52) U.S. Cl. CPC C12N 15/113; C12N 2310/11; C12N 2310/322; C12N 2310/3233; C12N 2310/315; C12N 2320/33

(57) ABSTRACT

Disclosed herein are methods for reducing expression of Tau mRNA and protein in an animal with Tau antisense compounds. Also disclosed are methods for modulating splicing of Tau mRNA in an animal with Tau antisense compounds. Such methods are useful to treat, prevent, or ameliorate neurodegenerative diseases in an individual in need thereof. Examples of neurodegenerative diseases that can be treated, prevented, and ameliorated with the administration Tau antisense oligonucleotides include Alzheimer's Disease, Fronto-temporal Dementia (FTD), FTDP-17, Progressive Supranuclear Palsy, Chronic Traumatic Encephalopathy, Epilepsy, and Dravet's Syndrome.

### SEQUENCE LISTING

The present application is being filed along with a Sequence Listing in electronic format. The Sequence Listing is provided as a file entitled BIOL0257WOSEQ_ST25.txt, created Jul. 20, 2015, which is 440 Kb in size. The information in the electronic format of the sequence listing is incorporated herein by reference in its entirety.

### FIELD

Provided are methods for treating, preventing, or ameliorating neurodegenerative diseases, including tauopathies, Alzheimer's Disease, Fronto-temporal Dementia (FTD), FTDP-17, Progressive Supranuclear Palsy (PSP), Chronic Traumatic Encephalopathy (CTE), Corticobasal Ganglionic Degeneration (CBD), Epilepsy, and Dravet's Syndrome by inhibiting expression of Tau or modulating the splicing of Tau in an animal. Certain embodiments are directed to methods, compounds and compositions for treating, preventing or ameliorating a seizure disorder by inhibiting expression of Tau or modulating the splicing of Tau in an animal.

### BACKGROUND

The primary function of Tau is to bind to and stabilize microtubules, which are important structural components of the cytoskeleton involved in mitosis, cytokinesis, and vesicular transport. Tau is found in multiple tissues, but is particularly abundant in axons of neurons.

Serine-threonine directed phosphorylation regulates the microtubule binding ability of Tau. Hyperphosphorylation promotes detachment of Tau from microtubules. Other post translational modifications of Tau have been described; however the significance of these is unclear. Phosphorylation of Tau is also developmentally regulated with higher phosphorylation in fetal tissues and much lower phosphorylation in the adult. One characteristic of neurodegenerative disorders is aberrantly increased Tau phosphorylation.

The microtubule network is involved in many important processes within the cell including structural integrity needed for maintaining morphology of cells and operating transport machinery. Since binding of Tau to microtubules stabilizes microtubules, Tau is likely to be a key mediator of some of these processes and disruption of normal Tau in neurodegenerative diseases may disrupt some of these key cellular processes.

One of the early indicators that Tau may be important in neurodegenerative syndromes was the recognition that Tau is a key component of neurofibrillary inclusions in Alzheimer's disease. In fact, neurofibrillary inclusions are aggregates of hyperphosphorylated Tau protein. Along with amyloid beta containing plaques, neurofibrillary inclusions are a hallmark of Alzheimer's disease and correlate significantly with cognitive impairment. 95% of Tau accumulations in AD are found in neuronal processes and is termed neuritic dystrophy. The process(es) whereby this microtubule associated protein becomes disengaged from microtubules and forms accumulations of proteins and how this relates to neuronal toxicity is not well understood.

Neuronal Tau inclusions are a pathological characteristic of not only Alzheimer's disease, but also a subset of Frontotemporal dementia (FTD), PSP, and CBD. The link between Tau and neurodegeneration was solidified by the discovery that mutations in the Tau gene cause a subset of FTD. These genetic data have also highlighted the importance of the 3R:4R ratio of Tau. Many of the Tau mutations that cause FTD lead to a change in Tau splicing which leads to preferential inclusion of exon 10, and thus to increased 4R Tau. The overall Tau levels are normal. Whether the Tau isoform change or the amino acid change or both cause neurodegeneration remains unknown. Recent data suggest that PSP may also be associated with an increased 4R:3R Tau ratio and thus may be amenable to a similar splicing strategy.

To help understand the influence of Tau ratios on neurodegeneration, a mouse model based on one of the splicing Tau mutations (N279K) has been generated using a minigene that includes the Tau promoter and the flanking intronic sequences of exon 10. As in humans, these mice demonstrate increased levels of 4R Tau compared with transgenics expressing WT Tau and develop behavioral and motor abnormalities as well as accumulations of aggregated Tau in the brain and spinal cord.

The protein “Tau” has been associated with multiple diseases of the brain including Alzheimer's disease, frontotemporal dementia, progressive supranuclear palsy, corticobasal ganglionic degeneration, dementia pugilistica, parkinsonism linked to chromosome, Lytico-Bodig disease, tangle-predominant dementia, ganglioglioma, gangliocytoma, meningioangiomatosis, subacute sclerosing panencephalitis, lead encephalopathy, tuberous sclerosis, Hallervorden-Spatz disease, Pick's disease, argyrophilic grain disease, corticobasal degeneration or frontotemporal lobar degeneration and others. Tau-associated disorders such as AD are the most common cause of dementia in the elderly. AD affects an estimated 15 million people worldwide and 40% of the population above 85 years of age. AD is characterized by two pathological hallmarks: Tau neurofibrillary inclusions (NFT) and amyloid-β (Aβ) plaques.

In seizure disorders, the brain's electrical activity is periodically disturbed, resulting in some degree of temporary brain dysfunction. Normal brain function requires an orderly, organized, coordinated discharge of electrical impulses. Electrical impulses enable the brain to communicate with the spinal cord, nerves, and muscles as well as within itself. Seizures may result when the brain's electrical activity is disrupted. There are two basic types of seizures; epileptic and nonepileptic. Epileptic seizures have no apparent cause or trigger and occur repeatedly. Nonepileptic seizures are triggered or provoked by a disorder or another condition that irritates the brain. Certain mental disorders can cause seizure symptoms referred to as psychogenic nonepileptic seizures.

Alzheimer's Disease (AD) is known to be a clinical risk factor for late onset seizures. Multiple AD mouse models recapitulate this increased seizure susceptibility. Within the last 5 years, many of these AD models have been studied in the setting of mouse tau knockout (tau−/−). Increased seizure susceptibility was ameliorated in these amyloid-depositing tau knockout lines. Further, tau−/− alone interestingly appeared to be protective against chemically induced seizures.

Anticonvulsants represent the common treatment regime for seizures. However, anticonvulsants are ineffective in a significant percent of people with a seizure disorder and for these individuals, surgery is the only option. Amidst the lack of available treatments for seizure disorders and neurodegenerative diseases, certain methods of the present embodiments provide methods for treating, preventing or ameliorating a seizure disorder and neurodegenerative diseases by inhibiting expression of Tau or modulating the splicing of Tau in an animal.

### SUMMARY

Provided herein are methods for modulating levels of Tau transcript (pre-mRNA and/or mRNA) and protein in cells, tissues, and animals. Also provided herein are methods for modulating splicing of Tau mRNA in cells, tissues, and animals. Also provided herein are methods for modulating the expression product of a Tau mRNA in cells, tissues, and animals.

In certain embodiments, modulation can occur in a cell or tissue. In certain embodiments, the cell or tissue is in an animal. In certain embodiments, the animal is human. In certain embodiments, Tau mRNA levels are reduced. In certain embodiments, Tau protein levels are reduced. In certain embodiments, splicing of Tau mRNA is modulated. In certain embodiments, the expression product of a Tau mRNA is modulated. In certain embodiments, hyperphosphorylated Tau is reduced. Such reduction and modulation can occur in a time-dependent manner or in a dose-dependent manner.

Several embodiments are drawn to methods of reducing or decreasing seizures in a subject. In certain embodiments, methods are provided for reducing the risk for seizure in a subject. In certain embodiments, the seizures are related to neurodegenerative disorders. In certain embodiments, the neurodegenerative disorder is a tau-associated disorder. In certain embodiments, the tau-associated disorder or neurodegenerative disorder is Alzheimer's disease, frontotemporal dementia, progressive supranuclear palsy, corticobasal ganglionic degeneration, dementia pugilistica, parkinsonism linked to chromosome, Lytico [symbol] Bodig disease, tangle-predominant dementia, ganglioglioma, gangliocytoma, meningioangiomatosis, subacute sclerosing panencephalitis, lead encephalopathy, tuberous sclerosis, Hallervorden-Spatz disease, Pick's disease, argyrophilic grain disease, corticobasal degeneration or frontotemporal lobar degeneration. Certain embodiments are drawn to a method of decreasing seizures in a subject.

Also provided are methods useful for preventing, treating, and ameliorating diseases, disorders, and conditions associated with Tau. In certain embodiments, such diseases, disorders, and conditions associated with Tau are neurodegenerative diseases. In certain embodiments, the neurodegenerative disease is any of Alzheimer's Disease, Fronto-temporal Dementia (FTD), FTDP-17, Progressive Supranuclear Palsy, Chronic Traumatic Encephalopathy, Epilepsy, or Dravet's Syndrome. In certain embodiments, one or more symptoms of a neurodegenerative disease is ameliorated, prevented, or delayed (progression slowed). In certain embodiments, the symptom is memory loss, anxiety, or loss of motor function. In certain embodiments, neurodegenerative function is improved. In certain embodiments, neurofibrillary inclusions are reduced.

Such diseases, disorders, and conditions can have one or more risk factors, causes, or outcomes in common. Certain risk factors and causes for development of a neurodegenerative disease include genetic predisposition and older age.

In certain embodiments, methods of treatment include administering a Tau antisense compound to an individual in need thereof. The antisense compound may inhibit expression of Tau or modulate splicing of Tau. In certain embodiments, the antisense compound is an antisense oligonucleotide. In certain embodiments, the single-stranded antisense oligonucleotide is complementary to a Tau nucleic acid.

The present disclosure provides the following non-limiting numbered embodiments:

### Embodiment 1

An antisense compound comprising a modified oligonucleotide consisting of 10-30 linked nucleosides and having a nucleobase sequence complementary to an intron/exon junction or an exon/intron junction or a splice modulation site of a Tau transcript.

### Embodiment 2

The antisense compound of embodiment 1, wherein the antisense compound is single-stranded.

### Embodiment 3

The antisense compound of embodiment 1, wherein the antisense compound is double-stranded.

### Embodiment 4

The antisense compound of any of embodiments 1-3, wherein the antisense compound comprises at least one conjugate.

### Embodiment 5

The antisense compound of any of embodiments 1-4, wherein the modified oligonucleotide comprises at least one modified nucleoside.

### Embodiment 6

The antisense compound of embodiment 5, wherein the modified oligonucleotide comprises at least one modified nucleoside comprising a modified sugar.

### Embodiment 7

The antisense compound of embodiment 6, wherein the at least one modified sugar is selected from among a bicyclic sugar, a non-bicyclic 2′-modified sugar, and a sugar surrogate.

### Embodiment 8

The antisense compound of embodiment 7, wherein at least one modified nucleoside is a 2′-MOE modified nucleoside.

### Embodiment 9

The antisense compound of embodiment 7, wherein at least one modified nucleoside is a morpholino nucleoside.

### Embodiment 10

The antisense compound of any of embodiments 5-9, wherein essentially each nucleoside of the modified oligonucleotide is modified.

### Embodiment 11

The antisense compound of any of embodiments 5-9, wherein each nucleoside of the modified oligonucleotide is modified.

### Embodiment 12

The antisense compound of any of embodiments 5-11, wherein each modified nucleoside has the same modification.

### Embodiment 13

The antisense compound of any of embodiments 5-11, wherein at least two modified nucleoside have different modifications from one another.

### Embodiment 14

The antisense compound of any of embodiments 1-13, wherein the modified oligonucleotide comprises at least one modified internucleoside linkage.

### Embodiment 15

The antisense compound of embodiment 14, wherein each internucleoside linkage of the modified oligonucleotide is a modified internucleoside linkage.

### Embodiment 16

The antisense compound of embodiment 14 or 15, wherein the modified internucleoside linkage is a phosphorothioate internucleoside linkage.

### Embodiment 17

The antisense compound of any of embodiments 1-16, wherein the modified oligonucleotide consists of 15, 16, 17, 18, 19, 20, 21, 22, 23, 24, or 25 linked nucleosides.

### Embodiment 18

The antisense compound of any of embodiments 1-17, wherein the modified oligonucleotide is at least 75%, at least 80%, at least 85%, at least 90%, or at least 95% complementary to an equal length portion of a human Tau nucleic acid.

### Embodiment 19

The antisense compound of any of embodiments 1-17, wherein the modified oligonucleotide is 100% complementary to an equal length portion of a human Tau nucleic acid.

### Embodiment 20

The antisense compound of any of embodiments 1-19, wherein the modified oligonucleotide is complementary to the junction of exon −1/intron −1 of the Tau transcript.

### Embodiment 21

The antisense compound of any of embodiments 1-19, wherein the modified oligonucleotide is complementary to the junction of intron −1/exon 1 of the Tau transcript.

### Embodiment 22

The antisense compound of any of embodiments 1-19, wherein the modified oligonucleotide is complementary to the junction of exon 1/intron 1 of the Tau transcript.

### Embodiment 23

The antisense compound of any of embodiments 1-19, wherein the modified oligonucleotide is complementary to the junction of intron 1/exon 2 of the Tau transcript.

### Embodiment 24

The antisense compound of any of embodiments 1-19, wherein the modified oligonucleotide is complementary to the junction of exon 2/intron 2 of the Tau transcript.

### Embodiment 25

The antisense compound of any of embodiments 1-19, wherein the modified oligonucleotide is complementary to the junction of intron 2/exon 3 of the Tau transcript.

### Embodiment 26

The antisense compound of any of embodiments 1-19, wherein the modified oligonucleotide is complementary to the junction of exon 3/intron 3 of the Tau transcript.

### Embodiment 27

The antisense compound of any of embodiments 1-19, wherein the modified oligonucleotide is complementary to the junction of intron 3/exon 4 of the Tau transcript.

### Embodiment 28

The antisense compound of any of embodiments 1-19, wherein the modified oligonucleotide is complementary to the junction of exon 4/intron 4 of the Tau transcript.

### Embodiment 29

The antisense compound of any of embodiments 1-19, wherein the modified oligonucleotide is complementary to the junction of intron 4/exon 5 of the Tau transcript.

### Embodiment 30

The antisense compound of any of embodiments 1-19, wherein the modified oligonucleotide is complementary to the junction of exon 5/intron 5 of the Tau transcript.

### Embodiment 31

The antisense compound of any of embodiments 1-19, wherein the modified oligonucleotide is complementary to the junction of intron 5/exon 6 of the Tau transcript.

### Embodiment 32

The antisense compound of any of embodiments 1-19, wherein the modified oligonucleotide is complementary to the junction of exon 6/intron 6 of the Tau transcript.

### Embodiment 33

The antisense compound of any of embodiments 1-19, wherein the modified oligonucleotide is complementary to the junction of intron 6/exon 7 of the Tau transcript.

### Embodiment 34

The antisense compound of any of embodiments 1-19, wherein the modified oligonucleotide is complementary to the junction of exon 7/intron 7 of the Tau transcript.

### Embodiment 35

The antisense compound of any of embodiments 1-19, wherein the modified oligonucleotide is complementary to the junction of intron 7/exon 8 of the Tau transcript.

### Embodiment 36

The antisense compound of any of embodiments 1-19, wherein the modified oligonucleotide is complementary to the junction of exon 8/intron 8 of the Tau transcript.

### Embodiment 37

The antisense compound of any of embodiments 1-19, wherein the modified oligonucleotide is complementary to the junction of intron 8/exon 9 of the Tau transcript.

### Embodiment 38

The antisense compound of any of embodiments 1-19, wherein the modified oligonucleotide is complementary to the junction of exon 9/intron 9 of the Tau transcript.

### Embodiment 39

The antisense compound of any of embodiments 1-19, wherein the modified oligonucleotide is complementary to the junction of intron 9/exon 10 of the Tau transcript.

### Embodiment 40

The antisense compound of any of embodiments 1-19, wherein the modified oligonucleotide is complementary to the junction of exon 10/intron 10 of the Tau transcript.

### Embodiment 41

The antisense compound of any of embodiments 1-19, wherein the modified oligonucleotide is complementary to the junction of intron 10/exon 11 of the Tau transcript.

### Embodiment 42

The antisense compound of any of embodiments 1-19, wherein the modified oligonucleotide is complementary to the junction of exon 11/intron 11 of the Tau transcript.

### Embodiment 43

The antisense compound of any of embodiments 1-19, wherein the modified oligonucleotide is complementary to the junction of intron 11/exon 12 of the Tau transcript.

### Embodiment 44

The antisense compound of any of embodiments 1-19, wherein the modified oligonucleotide is complementary to the junction of exon 12/intron 12 of the Tau transcript.

### Embodiment 45

The antisense compound of any of embodiments 1-19, wherein the modified oligonucleotide is complementary to the junction of intron 12/exon 13 of the Tau transcript.

### Embodiment 46

The antisense compound of any of embodiments 1-19, wherein the modified oligonucleotide is complementary to the junction of exon 13/intron 13 of the Tau transcript.

### Embodiment 47

The antisense compound of any of embodiments 1-19, wherein the modified oligonucleotide is complementary to a splice modulation site within intron −1, 1, 2, 3, 4, 5, 6, 7, 8, 9, 10, 11, 12, or 13 of the Tau transcript.

### Embodiment 48

The antisense compound of any of embodiments 1-19, wherein the modified oligonucleotide is complementary to a splice modulation site within exon −1, 1, 2, 3, 4, 5, 6, 7, 8, 9, 10, 11, 12, or 13 of the Tau transcript.

### Embodiment 49

The antisense compound of any of embodiments 1-48, wherein the Tau transcript is a mouse Tau transcript.

### Embodiment 50

The antisense compound of any of embodiments 1-48, wherein the Tau transcript is a human Tau transcript.

### Embodiment 51

The antisense compound of any of embodiments 1-49, wherein the modified oligonucleotide has a nucleobase sequence comprising at least 8, 10, 12, 14, 16, 18, 20, or 22 contiguous nucleobases of a nucleobase sequence selected from among of any of the oligonucleotides described in the present disclosure.

### Embodiment 52

A method comprising contacting a cell with an antisense compound according to any of embodiments 1-51.

### Embodiment 53

The method of embodiment 52, wherein the cell is in vitro.

### Embodiment 54

The method of embodiment 52, wherein the cell is in an animal.

### Embodiment 55

A method for reducing the amount or activity of a Tau transcript in a cell comprising contacting the cell with an antisense compound of any of embodiments 1-51 and thereby reducing the amount or activity of the Tau transcript in the cell.

### Embodiment 56

The method of embodiment 56, wherein the amount of Tau transcript is reduced.

### Embodiment 57

A method of treating a Tau disorder in an animal comprising administering to the animal an antisense compound according to any of embodiments 1-51.

### Embodiment 58

The method of embodiment 57, wherein the Tau disorder is selected from among: Tauopathy, Alzheimer's Disease, Fronto-temporal Dementia (FTD), FTDP-17, Progressive Supranuclear Palsy (PSP), Chronic Traumatic Encephalopathy (CTE), Corticobasal Ganglionic Degeneration (CBD), Epilepsy, or Dravet's Syndrome.

### Embodiment 59

The method of embodiment 57 or 58, wherein the animal is a mouse.

### Embodiment 60

The method of embodiment 57 or 58, wherein the animal is a human.

### DETAILED DESCRIPTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Additionally, as used herein, the use of “and” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. Where permitted, all documents, or portions of documents, cited in this disclosure, including, but not limited to, patents, patent applications, published patent applications, articles, books, treatises, and GENBANK Accession Numbers and associated sequence information obtainable through databases such as National Center for Biotechnology Information (NCBI) and other data referred to throughout in the disclosure herein are hereby expressly incorporated by reference for the portions of the document discussed herein, as well as in their entirety.

### Treatment of Neurodegenerative Syndrome and Seizures

A method of modifying neurodegenerative disease has been developed. Using the methods of the invention, it is now possible to alter the ratio of tau isoforms associated with multiple diseases of the brain. Advantageously, the invention provides a method of bypassing the blood brain barrier to specifically target the generation of certain tau isoforms in the central nervous system, may be administered for an extended period of time using proven technology, and has been demonstrated to provide widespread distribution of therapy throughout the brain and spinal cord where it is most efficient.

I. Method

The present invention provides a method of modifying a neurodegenerative syndrome in a subject by administering an antisense compound to the central nervous system. In certain embodiments, the antisense compound alters splicing of the nucleic acid encoding tau and decreases the amount of tau mRNA, pre-mRNA, and/or tau protein the central nervous system of the subject.

(a) Subject

According to the invention, the subject may be any subject that expresses tau. In some embodiments, a subject is a rodent, a human, a livestock animal, a companion animal, or a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In still another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In an exemplary embodiment, the subject may be a human.

The subject may be suffering from a neurodegenerative syndrome or may be at risk of developing a neurodegenerative syndrome. In some embodiments, the subject may be suffering from a neurodegenerative syndrome. In other embodiments, the subject may be at risk of developing a neurodegenerative syndrome. Neurodegenerative syndromes are as described further below.

(b) Neurodegenerative Syndrome

The method of the invention comprises modifying a neurodegenerative syndrome. In some embodiments, a neurodegenerative syndrome may be any neurodegenerative syndrome associated with tau. Non limiting examples of a neurodegenerative disorder associated with tau may include Alzheimer's disease, progressive supranuclear palsy, dementia pugilistica, frontotemporal dementia, parkinsonism linked to chromosome, Lytico-Bodig disease, tangle-predominant dementia, ganglioglioma, gangliocytoma, meningioangiomatosis, subacute sclerosing panencephalitis, lead encephalopathy, tuberous sclerosis, Hallervorden-Spatz disease, Pick's disease, corticobasal ganglionic degeneration, argyrophilic grain disease, supranuclear palsy, corticobasal degeneration, frontotemporal dementia, or frontotemporal lobar degeneration. In some embodiments, the method of the invention comprises modifying frontotemporal dementia (FTD). In other embodiments, the method of the invention comprises modifying Alzheimer's disease (AD). In yet other embodiments, the method of the invention comprises modifying progressive supranuclear palsy. In other embodiments, the method of the invention comprises modifying corticobasalganglionic degeneration.

As used herein, the term “modifying a neurodegenerative syndrome” may refer to curing the neurodegenerative syndrome, slowing the course of development of the syndrome, reversing the course of the syndrome, or improving the behavioral phenotype of a subject having a neurodegenerative syndrome. In some embodiments, the method of the invention modifies a neurodegenerative syndrome by curing the neurodegenerative syndrome. In other embodiments, the method of the invention modifies a neurodegenerative syndrome by slowing the progression of the syndrome.

In yet other embodiments, the method of the invention modifies a neurodegenerative syndrome by improving the behavioral phenotype of a subject having a neurodegenerative syndrome. For instance, the symptoms for subjects suffering from Alzheimer's disease may be the mild early symptoms associated with the neurodegenerative syndrome such as mild forgetfulness of recent events, activities, the names of familiar people or things, and the inability to solve simple math problems. The symptoms may also be the moderate symptoms associated with the neurodegenerative syndrome such as forgetting how to do simple tasks such as grooming, speaking, understanding, reading, or writing. Alternatively, the symptoms may be the severe symptoms associated with the neurodegenerative syndrome such as becoming anxious or aggressive, and wandering away from home. Subjects with AD may also have an increased risk of seizures. The symptoms for subjects suffering from progressive supranuclear palsy may include loss of balance, lunging forward when mobilizing, fast walking, bumping into objects or people, falls, changes in personality, general slowing of movement, visual symptoms, dementia (typically including loss of inhibition and ability to organize information), slurring of speech, difficulty swallowing, and difficulty moving the eyes, particularly in the vertical direction, poor eyelid function, contracture of the facial muscles, a backward tilt of the head with stiffening of the neck muscles, sleep disruption, urinary incontinence and constipation. The symptoms for subjects suffering from FTD may include personality changes, cognitive impairment, and motor symptoms. The symptoms for subjects suffering from corticobasalganglionic degeneration are similar to symptoms in patients suffering from FTD and Parkinson's disease and may include shaking, rigidity, slowness of movement and difficulty with walking and gait, cognitive and behavioural problems, dementia, sensory, sleep and emotional problems. In preferred embodiments, the method of the invention modifies a neurodegenerative syndrome by decreasing the risk of seizures.

### Definitions

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis.

Unless otherwise indicated, the following terms have the following meanings:

“Nonsense mediated decay” or “NMD” means any number of cellular mechanisms independent of RNase H or RISC that degrade mRNA or pre-mRNA. In certain embodiments, nonsense mediated decay eliminates and/or degrades mRNA transcripts that contain premature stop codons. In certain embodiments, nonsense mediated decay eliminates and/or degrades any form of aberrant mRNA and/or pre-mRNA transcripts.

“2′-O-methoxyethyl” (also 2′-MOE and 2′-O(CH₂)₂—OCH₃ and MOE) refers to an O-methoxy-ethyl modification at the 2′ position of a furanosyl ring. A 2′-O-methoxyethyl modified sugar is a modified sugar.

“2′-MOE nucleoside” (also 2′-O-methoxyethyl nucleoside) means a nucleoside comprising a 2′-MOE modified sugar moiety.

“2′-OMethyl” means a 2′-OCH₃ modification at the 2′ position of a furanosyl ring.

“2′-substituted nucleoside” means a nucleoside comprising a substituent at the 2′-position of the furanosyl ring other than H or OH. In certain embodiments, 2′ substituted nucleosides include nucleosides with bicyclic sugar modifications.

“3′ target site” refers to the nucleotide of a target nucleic acid which is complementary to the 3′-most nucleotide of a particular antisense compound.

“5′ target site” refers to the nucleotide of a target nucleic acid which is complementary to the 5′-most nucleotide of a particular antisense compound.

“5-methylcytosine” means a cytosine modified with a methyl group attached to the 5 position. A 5-methylcytosine is a modified nucleobase.

“About” means within ±7% of a value. For example, if it is stated, “the compounds affected at least about 70% inhibition of Tau”, it is implied that the Tau levels are inhibited within a range of 63% and 77%.

“Acceptable safety profile” means a pattern of side effects that is within clinically acceptable limits.

“Active pharmaceutical agent” means the substance or substances in a pharmaceutical composition that provide a therapeutic benefit when administered to an individual. For example, in certain embodiments an antisense oligonucleotide targeted to Tau is an active pharmaceutical agent.

“Active target region” means a target region to which one or more active antisense compounds is targeted. “Active antisense compounds” means antisense compounds that reduce target nucleic acid levels or protein levels.

“Administered concomitantly” refers to the co-administration of two agents in any manner in which the pharmacological effects of both are manifest in the patient at the same time. Concomitant administration does not require that both agents be administered in a single pharmaceutical composition, in the same dosage form, or by the same route of administration. The effects of both agents need not manifest themselves at the same time. The effects need only be overlapping for a period of time and need not be coextensive.

“Administering” means providing a pharmaceutical agent to an individual, and includes, but is not limited to administering by a medical professional and self-administering.

“Agent” means an active substance that can provide a therapeutic benefit when administered to an animal. “First Agent” means a therapeutic compound described herein. For example, a first agent can be an antisense oligonucleotide targeting Tau. “Second agent” means a second therapeutic compound described herein (e.g. a second antisense oligonucleotide targeting Tau) and/or a non-Tau therapeutic compound.

“Amelioration” or “ameliorate” or “ameliorating” refers to a lessening of at least one indicator, sign, or symptom of a disease, disorder, or condition. The severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antibody” refers to a molecule characterized by reacting specifically with an antigen in some way, where the antibody and the antigen are each defined in terms of the other. Antibody may refer to a complete antibody molecule or any fragment or region thereof, such as the heavy chain, the light chain, F_{ab} region, and F_{c} region.

“Antisense activity” means any detectable or measurable activity attributable to the hybridization of an antisense compound to its target nucleic acid. In certain embodiments, antisense activity is a decrease in the amount or expression of a target nucleic acid or protein encoded by such target nucleic acid.

“Antisense compound” means an oligomeric compound that is capable of undergoing hybridization to a target nucleic acid through hydrogen bonding. Examples of antisense compounds include single-stranded and double-stranded compounds, such as, antisense oligonucleotides, siRNAs, shRNAs, ssRNAs, and occupancy-based compounds.

“Antisense inhibition” means reduction of target nucleic acid levels or target protein levels in the presence of an antisense compound complementary to a target nucleic acid compared to target nucleic acid levels or target protein levels in the absence of the antisense compound.

“Antisense mechanisms” are all those mechanisms involving hybridization of a compound with target nucleic acid, wherein the outcome or effect of the hybridization is either target degradation or target occupancy with concomitant stalling of the cellular machinery involving, for example, transcription or splicing. Antisense mechanisms include, without limitation, RNase H mediated antisense; RNAi mechanisms, which utilize the RISC pathway and include, without limitation, siRNA, ssRNA, and microRNA mechanisms; and occupancy based mechanisms, including, without limitation uniform modified oligonucleotides. Certain antisense compounds may act through more than one such mechanism and/or through additional mechanisms.

“Antisense oligonucleotide” (also “oligo”) means a single-stranded oligonucleotide having a nucleobase sequence that permits hybridization to a corresponding region or segment of a target nucleic acid resulting in at least one antisense activity.

“Base complementarity” refers to the capacity for the precise base pairing of nucleobases of an antisense oligonucleotide with corresponding nucleobases in a target nucleic acid (i.e., hybridization), and is mediated by Watson-Crick, Hoogsteen or reversed Hoogsteen hydrogen binding between corresponding nucleobases.

“Bicyclic sugar” means a furanosyl ring modified by the bridging of two atoms. A bicyclic sugar is a modified sugar.

“Bicyclic nucleoside” (also BNA) means a nucleoside having a sugar moiety comprising a bridge connecting two carbon atoms of the sugar ring, thereby forming a bicyclic ring system. In certain embodiments, the bridge connects the 4′-carbon and the 2′-carbon on the sugar ring.

“Cap structure” or “terminal cap moiety” means chemical modifications, which have been incorporated at either terminus of an antisense compound.

“cEt” or “constrained ethyl” means a bicyclic nucleoside having a sugar moiety comprising a bridge connecting the 4′-carbon and the 2′-carbon, wherein the bridge has the formula: 4′-CH(CH₃)—O-2′.

“Constrained ethyl nucleoside” (also cEt nucleoside) means a nucleoside comprising a bicyclic sugar moiety comprising a 4′-CH(CH₃)—O-2′ bridge.

“Chemically distinct region” refers to a region of an antisense compound that is in some way chemically different than another region of the same antisense compound. For example, a region having 2′-O-methoxyethyl nucleotides is chemically distinct from a region having nucleotides without 2′-O-methoxyethyl modifications.

“Chimeric antisense compounds” means antisense compounds that have at least 2 chemically distinct regions, each position having a plurality of subunits.

“Co-administration” means administration of two or more pharmaceutical agents to an individual. The two or more pharmaceutical agents may be in a single pharmaceutical composition, or may be in separate pharmaceutical compositions. Each of the two or more pharmaceutical agents may be administered through the same or different routes of administration. Co-administration encompasses administration in parallel or sequentially.

“Complementarity” means the capacity for pairing between nucleobases of a first nucleic acid and a second nucleic acid.

“Comply” means the adherence with a recommended therapy by an individual.

“Comprise,” “comprises” and “comprising” will be understood to imply the inclusion of a stated step or element or group of steps or elements but not the exclusion of any other step or element or group of steps or elements.

“Contiguous nucleobases” means nucleobases immediately adjacent to each other.

“Cure” means a method or course that restores health or a prescribed treatment for an illness.

“Deoxyribonucleotide” means a nucleotide having a hydrogen at the 2′ position of the sugar portion of the nucleotide. Deoxyribonucleotides may be modified with any of a variety of substituents.

“Designing” or “Designed to” refer to the process of designing an oligomeric compound that specifically hybridizes with a selected nucleic acid molecule.

“Diluent” means an ingredient in a composition that lacks pharmacological activity, but is pharmaceutically necessary or desirable. For example, in drugs that are injected, the diluent may be a liquid, e.g. saline solution.

“Dosage unit” means a form in which a pharmaceutical agent is provided, e.g. pill, tablet, or other dosage unit known in the art.

“Dose” means a specified quantity of a pharmaceutical agent provided in a single administration, or in a specified time period. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual. In other embodiments, the pharmaceutical agent is administered by infusion over an extended period of time or continuously. Doses may be stated as the amount of pharmaceutical agent per hour, day, week or month.

“Dosing regimen” is a combination of doses designed to achieve one or more desired effects.

“Duration” means the period of time during which an activity or event continues. In certain embodiments, the duration of treatment is the period of time during which doses of a pharmaceutical agent are administered.

“Effective amount” in the context of modulating an activity or of treating or preventing a condition means the administration of that amount of active ingredient to a subject in need of such modulation, treatment or prophylaxis, either in a single dose or as part of a series, that is effective for modulation of that effect, or for treatment or prophylaxis or improvement of that condition. The effective amount will vary depending upon the health and physical condition of the subject to be treated, the taxonomic group of subjects to be treated, the formulation of the composition, the assessment of the medical situation, and other relevant factors.

“Efficacy” means the ability to produce a desired effect.

“Excitotoxicity” the pathological process by which nerve cells are damaged and killed by excessive stimulation by neurotransmitters.

“Expression” includes all the functions by which a gene's coded information is converted into structures present and operating in a cell. Such structures include, but are not limited to the products of transcription and translation.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.

“Fully modified motif” refers to an antisense compound comprising a contiguous sequence of nucleosides wherein essentially each nucleoside is a sugar modified nucleoside having uniform modification.

“Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

“Tau nucleic acid” or “Tau DNA” means any nucleic acid encoding Tau. For example, in certain embodiments, a Tau nucleic acid includes, without limitation, any viral DNA sequence encoding a Tau genome or portion thereof, any RNA sequence transcribed from a DNA sequence including any mRNA sequence encoding a Tau protein.

“Hybridization” means the annealing of complementary nucleic acid molecules. In certain embodiments, complementary nucleic acid molecules include, but are not limited to, an antisense compound and a nucleic acid target. In certain embodiments, complementary nucleic acid molecules include, but are not limited to, an antisense oligonucleotide and a nucleic acid target.

“Identifying an animal having a Tau-related disease or disorder” means identifying an animal having been diagnosed with a Tau-related disease or disorder; or, identifying an animal having any symptom of Tau-related disease or disorder including, but not limited to a neurodegenerative disorder associated with Tau.

“Immediately adjacent” means there are no intervening elements between the immediately adjacent elements.

“Individual” means a human or non-human animal selected for treatment or therapy.

“Individual compliance” means adherence to a recommended or prescribed therapy by an individual.

“Induce”, “inhibit”, “potentiate”, “elevate”, “increase”, “decrease” or the like, e.g., which denote quantitative differences between two states, refer to at least statistically significant differences between the two states. For example, “an amount effective to inhibit the activity or expression of Tau” means that the level of activity or expression of Tau in a treated sample will differ statistically significantly from the level of Tau activity or expression in untreated cells. Such terms are applied to, for example, levels of expression, and levels of activity.

“Inhibiting Tau” means reducing the level or expression of a Tau mRNA, DNA and/or protein. In certain embodiments, Tau is inhibited in the presence of an antisense compound targeting Tau, including an antisense oligonucleotide targeting Tau, as compared to expression of Tau mRNA, DNA and/or protein levels in the absence of a Tau antisense compound, such as an antisense oligonucleotide.

“Inhibiting the expression or activity” refers to a reduction, blockade of the expression or activity and does not necessarily indicate a total elimination of expression or activity.

“Internucleoside linkage” refers to the chemical bond between nucleosides.

“Intraperitoneal administration” means administration through infusion or injection into the peritoneum.

“Intravenous administration” means administration into a vein.

“Lengthened” antisense oligonucleotides are those that have one or more additional nucleosides relative to an antisense oligonucleotide disclosed herein.

“Linked deoxynucleoside” means a nucleic acid base (A, G, C, T, U) substituted by deoxyribose linked by a phosphate ester to form a nucleotide.

“Linked nucleosides” means adjacent nucleosides linked together by an internucleoside linkage.

“Locked nucleic acid” or “LNA” or “LNA nucleosides” means nucleic acid monomers having a bridge connecting two carbon atoms between the 4′ and 2′ position of the nucleoside sugar unit, thereby forming a bicyclic sugar. Examples of such bicyclic sugar include, but are not limited to A) α-L-Methyleneoxy (4′-CH₂—O-2′) LNA, (B) β-D-methyleneoxy (4′-CH₂—O-2′) LNA, (C) Ethyleneoxy (4′-(CH₂)₂—O-2′) LNA, (D) Aminooxy (4′-CH₂—O—N(R)-2′) LNA and (E) Oxyamino (4′-CH₂—N(R)—O-2′) LNA, as depicted below.

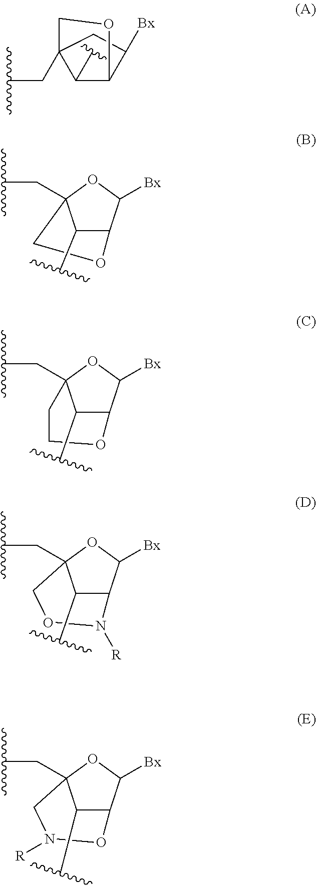

As used herein, LNA compounds include, but are not limited to, compounds having at least one bridge between the 4′ and the 2′ position of the sugar wherein each of the bridges independently comprises 1 or from 2 to 4 linked groups independently selected from —[C(R₁)(R₂)]ₙ—, —C(R₁)═C(R₂)—, —C(R₁)═N—, —C(═NR₁)—, —C(═O)—, —C(═S)—, —O—, —Si(R₁)₂—, —S(═O)ₓ and —N(R₁)—; wherein: x is 0, 1, or 2; n is 1, 2, 3, or 4; each R₁ and R₂ is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, a heterocycle radical, a substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

Examples of 4′-2′ bridging groups encompassed within the definition of LNA include, but are not limited to one of formulae: —[C(R₁)(R₂)]ₙ—, —[C(R₁)(R₂)]ₙ—O—, —C(R₁R₂)—N(R₁)—O— or —C(R₁R₂)—O—N(R₁)—. Furthermore, other bridging groups encompassed with the definition of LNA are 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R₁)-2′ and 4′-CH₂—N(R₁)—O-2′-bridges, wherein each R₁ and R₂ is, independently, H, a protecting group or C₁-C₁₂ alkyl.

Also included within the definition of LNA according to the invention are LNAs in which the 2′-hydroxyl group of the ribosyl sugar ring is connected to the 4′ carbon atom of the sugar ring, thereby forming a methyleneoxy (4′-CH₂—O-2′) bridge to form the bicyclic sugar moiety. The bridge can also be a methylene (—CH₂—) group connecting the 2′ oxygen atom and the 4′ carbon atom, for which the term methyleneoxy (4′-CH₂—O-2′) LNA is used. Furthermore; in the case of the bicyclic sugar moiety having an ethylene bridging group in this position, the term ethyleneoxy (4′-CH₂CH₂—O-2′) LNA is used. α-L-methyleneoxy (4′-CH₂—O-2′), an isomer of methyleneoxy (4′-CH₂—O-2′) LNA is also encompassed within the definition of LNA, as used herein.

“Mismatch” or “non-complementary nucleobase” refers to the case when a nucleobase of a first nucleic acid is not capable of pairing with the corresponding nucleobase of a second or target nucleic acid.

“Modified internucleoside linkage” refers to a substitution or any change from a naturally occurring internucleoside bond (i.e. a phosphodiester internucleoside bond).

“Modified nucleobase” means any nucleobase other than adenine, cytosine, guanine, thymidine, or uracil. An “unmodified nucleobase” means the purine bases adenine (A) and guanine (G), and the pyrimidine bases thymine (T), cytosine (C) and uracil (U).

“Modified nucleoside” means a nucleoside having, independently, a modified sugar moiety and/or modified nucleobase.

“Modified nucleotide” means a nucleotide having, independently, a modified sugar moiety, modified internucleoside linkage, or modified nucleobase.

“Modified oligonucleotide” means an oligonucleotide comprising at least one modified internucleoside linkage, a modified sugar, and/or a modified nucleobase.

“Modified sugar” means substitution and/or any change from a natural sugar moiety.

“Monomer” refers to a single unit of an oligomer. Monomers include, but are not limited to, nucleosides and nucleotides, whether naturally occurring or modified.

“Motif” means the pattern of unmodified and modified nucleosides in an antisense compound.

“Natural sugar moiety” means a sugar moiety found in DNA (2′-H) or RNA (2′-OH).

“Naturally occurring internucleoside linkage” means a 3′ to 5′ phosphodiester linkage.

“Neurodegenerative disorder” means a chronic progressive neuropathy characterized by selective loss of neurons in motor, sensory, or cognitive systems. Neurodegenerative disorders include, but are not limited to, Tau-associated disorders.

“Neurofibrillary inclusion” means interneuronal aggregates largely composed of insoluble hyperphosphorylated tau protein. In certain embodiments, neurofibrillary inclusions may be measured through various means including SPECT perfusion imaging, functional MRI, and PET scans. In certain embodiments, reduction of neurofibrillary inclusions may be inferred by improved scores on cognitive exams such as the Mini-Mental State Exam (MMSE) and the Alzheimer's Disease Assessment Scale Cognitive Behavior Section (ADAS-cog).

“Non-complementary nucleobase” refers to a pair of nucleobases that do not form hydrogen bonds with one another or otherwise support hybridization.

“Nucleic acid” refers to molecules composed of monomeric nucleotides. A nucleic acid includes, but is not limited to, ribonucleic acids (RNA), deoxyribonucleic acids (DNA), single-stranded nucleic acids, double-stranded nucleic acids, small interfering ribonucleic acids (siRNA), and microRNAs (miRNA).

“Nucleobase” means a heterocyclic moiety capable of pairing with a base of another nucleic acid.

“Nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

“Nucleobase sequence” means the order of contiguous nucleobases independent of any sugar, linkage, and/or nucleobase modification.

“Nucleoside” means a nucleobase linked to a sugar.

“Nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo or tricyclo sugar mimetics, e.g., non furanose sugar units. Nucleotide mimetic includes those structures used to replace the nucleoside and the linkage at one or more positions of an oligomeric compound such as for example peptide nucleic acids or morpholinos (morpholinos linked by —N(H)—C(═O)—O— or other non-phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system. “Mimetic” refers to groups that are substituted for a sugar, a nucleobase, and/or internucleoside linkage. Generally, a mimetic is used in place of the sugar or sugar-internucleoside linkage combination, and the nucleobase is maintained for hybridization to a selected target.

In certain embodiments, sugar surrogates comprise rings having more than 5 atoms and more than one heteroatom. For example nucleosides comprising morpholino sugar moieties and their use in oligomeric compounds has been reported (see for example: Braasch et al., Biochemistry, 2002, 41, 4503-4510; and U.S. Pat. Nos. 5,698,685; 5,166,315; 5,185,444; and 5,034,506).

As used here, the term “morpholino” means a sugar surrogate having the following structure:

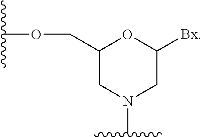

In certain embodiments, morpholinos may be modified, for example by adding or altering various substituent groups from the above morpholino structure. Such sugar surrogates are referred to herein as “modified morpholinos.”

“Nucleotide” means a nucleoside having a phosphate group covalently linked to the sugar portion of the nucleoside.

“Off-target effect” refers to an unwanted or deleterious biological effect associated with modulation of RNA or protein expression of a gene other than the intended target nucleic acid.

“Oligomeric compound” means a polymer of linked monomeric subunits which is capable of hybridizing to at least a region of a nucleic acid molecule.

“Oligonucleoside” means an oligonucleotide in which the internucleoside linkages do not contain a phosphorus atom.

“Oligonucleotide” (also “oligo”) means a polymer of linked nucleosides each of which can be modified or unmodified, independent one from another.

“Parenteral administration” means administration through injection (e.g., bolus injection) or infusion. Parenteral administration includes subcutaneous administration, intravenous administration, intramuscular administration, intraarterial administration, intraperitoneal administration, or intracranial administration, e.g., intrathecal or intracerebroventricular administration.

“Peptide” means a molecule formed by linking at least two amino acids by amide bonds. Without limitation, as used herein, “peptide” refers to polypeptides and proteins.

“Pharmaceutically acceptable carrier” means a medium or diluent that does not interfere with the structure of the oligonucleotide. Certain such carriers enable pharmaceutical compositions to be formulated as, for example, tablets, pills, dragees, capsules, liquids, gels, syrups, slurries, suspension and lozenges for the oral ingestion by a subject.

“Pharmaceutically acceptable derivative” encompasses pharmaceutically acceptable salts, conjugates, prodrugs or isomers of the compounds described herein.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Pharmaceutical agent” means a substance that provides a therapeutic benefit when administered to an individual. For example, in certain embodiments, an antisense oligonucleotide targeted to Tau is a pharmaceutical agent.

“Pharmaceutical composition” means a mixture of substances suitable for administering to a subject. For example, a pharmaceutical composition may comprise an antisense oligonucleotide and a sterile aqueous solution. In certain embodiments, a pharmaceutical composition shows activity in free uptake assay in certain cell lines.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Prevention” or “preventing” refers to delaying or forestalling the onset or development of a condition or disease for a period of time from hours to days, preferably weeks to months.

“Prodrug” means a therapeutic agent that is prepared in an inactive form that is converted to an active form (i.e., drug) within the body or cells thereof by the action of endogenous enzymes or other chemicals and/or conditions.

“Prophylactically effective amount” refers to an amount of a pharmaceutical agent that provides a prophylactic or preventative benefit to an animal.

“Recommended therapy” means a therapeutic regimen recommended by a medical professional for the treatment, amelioration, or prevention of a disease.

“Region” is defined as a portion of the target nucleic acid having at least one identifiable structure, function, or characteristic.

“Ribonucleotide” means a nucleotide having a hydroxy at the 2′ position of the sugar portion of the nucleotide. Ribonucleotides may be modified with any of a variety of substituents.

“Salts” mean a physiologically and pharmaceutically acceptable salts of antisense compounds, i.e., salts that retain the desired biological activity of the parent oligonucleotide and do not impart undesired toxicological effects thereto.

“Scrambled oligo” or “scrambled” or “ISIS 141923” is a 5-10-5 MOE gapmer with no known target having the sequence of SEQ ID NO: 11.

“Segments” are defined as smaller or sub-portions of regions within a target nucleic acid.

“Shortened” or “truncated” versions of antisense oligonucleotides Taught herein have one, two or more nucleosides deleted.

“Side effects” means physiological responses attributable to a treatment other than desired effects. In certain embodiments, side effects include, without limitation, injection site reactions, liver function test abnormalities, renal function abnormalities, liver toxicity, renal toxicity, central nervous system abnormalities, and myopathies. For example, increased aminotransferase levels in serum may indicate liver toxicity or liver function abnormality. For example, increased bilirubin may indicate liver toxicity or liver function abnormality.

“Sites,” as used herein, are defined as unique nucleobase positions within a target nucleic acid.

“Slows progression” means decrease in the development of the said disease.

“Splice modulation site” refers to a site on the Tau pre-mRNA which when hybridized by an antisense oligonucleotide results in altered splicing of the Tau pre-mRNA resulting in an altered splice product. In certain embodiments, such altered splice product is less stable or results in reduced amount of tau protein. In certain embodiments, a splice modulation site comprises a region of Tau pre-mRNA containing an intronic splice silencer. In certain embodiments, a splice modulation site comprises a region of Tau pre-mRNA containing an intronic splice enhancer. In certain embodiments, a splice modulation site comprises a region of Tau pre-mRNA containing serine/arginine-rich splicing factor.

“Specifically hybridizable” refers to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e., under physiological conditions in the case of in vivo assays and therapeutic treatments.

“Stringent hybridization conditions” or “stringent conditions” refer to conditions under which an oligomeric compound will hybridize to its target sequence, but to a minimal number of other sequences.

“Subcutaneous administration” means administration just below the skin.

“Subject” means a human or non-human animal selected for treatment or therapy.

“Target” refers to a protein, the modulation of which is desired.

“Target gene” refers to a gene encoding a target.

“Targeting” means the process of design and selection of an antisense compound that will specifically hybridize to a target nucleic acid and induce a desired effect.

“Target nucleic acid,” “target RNA,” “target RNA transcript” and “nucleic acid target” all mean a nucleic acid capable of being targeted by antisense compounds.

“Target region” means a portion of a target nucleic acid to which one or more antisense compounds is targeted.

“Target segment” means the sequence of nucleotides of a target nucleic acid to which an antisense compound is targeted. “5′ target site” refers to the 5′-most nucleotide of a target segment. “3′ target site” refers to the 3′-most nucleotide of a target segment.

“Tau-associated disease” means any neurological or neurodegenerative disease associated with Tau. Non-limiting examples of Tau-associated disorders include Alzheimer's disease, progressive supranuclear palsy, dementia pugilistica, frontotemporal dementia, parkinsonism linked to chromosome, Lytico-Bodig disease, tangle-predominant dementia, ganglioglioma, gangliocytoma, meningioangiomatosis, subacute sclerosing panencephalitis, lead encephalopathy, tuberous sclerosis, Hallervorden-Spatz disease, Pick's disease, corticobasal ganglionic degeneration, argyrophilic grain disease, supranuclear palsy, corticobasal degeneration, frontotemporal dementia, or frontotemporal lobar degeneration.

“Tauopathy” means disorders characterized by a build-up of Tau protein in the brain.

“Tau-specific inhibitor” includes but is not limited to a “antisense compound” targeted to Tau.

“Therapeutically effective amount” means an amount of a pharmaceutical agent that provides a therapeutic benefit to an individual.

“Treatment” refers to administering a composition to effect an alteration or improvement of the disease or condition.

“Unmodified” nucleobases mean the purine bases adenine (A) and guanine (G), and the pyrimidine bases thymine (T), cytosine (C) and uracil (U).

“Unmodified nucleotide” means a nucleotide composed of naturally occurring nucleobases, sugar moieties, and internucleoside linkages. In certain embodiments, an unmodified nucleotide is an RNA nucleotide (i.e. β-D-ribonucleosides) or a DNA nucleotide (i.e. β-D-deoxyribonucleoside).

“Validated target segment” is defined as at least an 8-nucleobase portion (i.e. 8 consecutive nucleobases) of a target region to which an active oligomeric compound is targeted.

“Wing segment” means a plurality of nucleosides modified to impart to an oligonucleotide properties such as enhanced inhibitory activity, increased binding affinity for a target nucleic acid, or resistance to degradation by in vivo nucleases.

### Certain Embodiments

Certain embodiments provide for methods of administering a Tau antisense compound targeting a Tau nucleic acid for the treatment of a Tau associated disease. In certain embodiments, the Tau nucleic acid is any of the sequences set forth in GENBANK Accession NT_010783.14 truncated from nucleotides 2624000 to U.S. Pat. No. 2,761,000 (incorporated herein as SEQ ID NO: 1); GENBANK Accession No. AK226139.1 (incorporated herein as SEQ ID NO: 2); GENBANK Accession No. NM_001123066.3 (incorporated herein as SEQ ID NO: 3); GENBANK Accession No. NM_001123067.3 (incorporated herein as SEQ ID NO: 4); GENBANK Accession No. NM_001203251.1 (incorporated herein as SEQ ID NO: 5); GENBANK Accession No. NM_001203252.1 (incorporated herein as SEQ ID NO: 6); GENBANK Accession No. NM_005910.5 (incorporated herein as SEQ ID NO: 7); GENBANK Accession No. NM_016834.4 (incorporated herein as SEQ ID NO: 8); GENBANK Accession No. NM_016835.4 (incorporated herein as SEQ ID NO: 9); or GENBANK Accession No. NM 016841.4 (incorporated herein as SEQ ID NO: 10).

A method of treating a Tau associated disease with antisense compounds has been developed. In certain embodiments, neurofibrillary inclusions are reduced. In certain embodiments, neurological function is improved. In certain embodiments, the antisense compounds reduce expression of Tau mRNA and protein. In certain embodiments, the antisense compounds alter the ratio of Tau isoforms. In certain embodiments, the splicing alteration is a decrease in 4R:3R Tau ratio in the central nervous system of the subject. In certain embodiments, the splicing alteration results in a normal 4R:3R Tau ratio. Advantageously, several embodiments provide methods of bypassing the blood brain barrier to specifically target Tau in the central nervous system, administer for an extended period of time, and achieve widespread distribution of therapy throughout the brain and spinal cord where it is most effective.

Certain embodiments provide methods for the treatment, prevention, or amelioration of diseases, disorders, and conditions associated with Tau in an individual in need thereof. Also contemplated are methods for the preparation of a medicament for the treatment, prevention, or amelioration of a disease, disorder, or condition associated with Tau. Tau associated diseases, disorders, and conditions include neurodegenerative diseases. In certain embodiments, the neurodegenerative disease may be any of Alzheimer's Disease, frontotemporal Dementia (FTD), FTDP-17, Progressive Supranuclear Palsy (PSP), Chronic Traumatic Encephalopathy (CTE), Corticobasal Ganglionic Degeneration (CBD), epilepsy, Dravet's Syndrome, dementia pugilistica, parkinsonism linked to chromosome, Lytico-Bodig disease, tangle-predominant dementia, ganglioglioma, gangliocytoma, meningioangiomatosis, subacute sclerosing panencephalitis, lead encephalopathy, tuberous sclerosis, Hallervorden-Spatz disease, Pick's disease, argyrophilic grain disease, supranuclear palsy, corticobasal degeneration, or frontotemporal lobar degeneration.

Described herein are methods comprising administering a Tau antisense compound to an animal for treating a Tau associated disease and thereby reducing neurofibrillary inclusions.

Described herein are methods comprising administering a Tau antisense compound to an animal for treating a Tau associated disease and thereby improving neurological function.

Described herein are methods comprising: (i) identifying an animal having a Tau associated disease; and (ii) administering a Tau antisense compound and thereby reducing neurofibrillary inclusions.

Described herein are methods comprising: (i) identifying an animal having a Tau associated disease; and (ii) administering a Tau antisense compound and thereby improving neurological function.

In certain embodiments, the animal is a human.

In certain embodiments, the antisense compound comprises a single-stranded antisense oligonucleotide complementary to a Tau nucleic acid.

In certain embodiments, the Tau nucleic acid is any of SEQ ID NO: 1-10.

In certain embodiments, the antisense compounds for use in the methods may comprise a single-stranded antisense oligonucleotide comprising a nucleobase sequence at least 80%, at least 85%, at least 90%, at least 95%, at least 96%, at least 97%, at least 98%, or at least 99% complementary to an equal length portion of SEQ ID NOs:1-10. In certain embodiments, the compound may comprise a single-stranded antisense oligonucleotide comprising a nucleobase sequence 100% complementary to an equal length portion of SEQ ID NOs: 1-10.

In certain embodiments, the Tau associated disease is a neurodegenerative disease.

In certain embodiments, the neurodegenerative disease is selected from among Alzheimer's Disease, Fronto-temporal Dementia (FTD), FTDP-17, Progressive Supranuclear Palsy (PSP), Chronic Traumatic Encephalopathy (CTE), Corticobasal Ganglionic Degeneration (CBD), Epilepsy, or Dravet's Syndrome.

In certain embodiments, expression of Tau RNA or expression of Tau protein is reduced.

In certain embodiments, expression of the 4R isoform of Tau RNA or expression of the 4R isoform of Tau protein is reduced.

In certain embodiments, expression of the 3R isoform of Tau RNA or expression of the 3R isoform of Tau protein is increased.

In certain embodiments, expression of the 4R isoform of Tau RNA is reduced and expression of the 3R isoform of Tau RNA is increased.

In certain embodiments, expression of the 4R isoform of Tau protein is reduced and expression of the 3R isoform of Tau protein is increased.

In certain embodiments, the single-stranded antisense oligonucleotide comprises at least one modification.

In certain embodiments, the single-stranded antisense oligonucleotide is specifically hybridizable to a human Tau nucleic acid.

In certain embodiments, the single-stranded antisense oligonucleotide is at least 75%, at least 80%, at least 85%, at least 90%, or at least 95% complementary to an equal length portion of a human Tau nucleic acid.

In certain embodiments, the single-stranded antisense oligonucleotide is 100% complementary to a human Tau nucleic acid.

In certain embodiments, the single-stranded antisense oligonucleotide comprises at least one modified internucleoside linkage.

In certain embodiments, each internucleoside linkage of the single-stranded antisense oligonucleotide is a modified internucleoside linkage.

In certain embodiments, the modified internucleoside linkage is a phosphorothioate internucleoside linkage.

In certain embodiments, the antisense oligonucleotide comprises at least one modified nucleoside.

In certain embodiments, the single-stranded antisense oligonucleotide comprises at least one modified nucleoside having a modified sugar.

In certain embodiments, the single-stranded antisense oligonucleotide comprises at least one modified nucleoside comprising a bicyclic sugar.

In certain embodiments, the bicyclic sugar comprises a 4′ to 2′ bridge selected from among: 4′-(CH2)n-O-2′, wherein n is 1 or 2; and 4′-CH2-O—CH2-2′.

In certain embodiments, the bicyclic sugar comprises a 4′-CH(CH3)-O-2′ bridge.

In certain embodiments, the at least one modified nucleoside having a modified sugar comprises a non-bicyclic 2′-modified sugar moiety.

In certain embodiments, the 2′-modified sugar moiety comprises a 2′-O-methoxyethyl group.

In certain embodiments, the 2′-modified sugar moiety comprises a 2′-O-methyl group.

In certain embodiments, the at least one modified nucleoside having a modified sugar comprises a sugar surrogate.

In certain embodiments, the sugar surrogate is a morpholino.

In certain embodiments, the sugar surrogate is a peptide nucleic acid.

In certain embodiments, each nucleoside is modified.

In certain embodiments, the single-stranded antisense oligonucleotide comprises at least one modified nucleobase.

In certain embodiments, the modified nucleobase is a 5′-methylcytosine.

In certain embodiments, fully 2′-MOE modified antisense oligonucleotides targeted to a tau transcript are administered intrathecally to the CNS. Like the fully modified 2′-MOE oligonucleotides described in WO2010/148249, the fully modified 2′-MOE oligonucleotides described herein are expected to be well tolerated when administered to the CNS.

### Antisense Compounds

Oligomeric compounds include, but are not limited to, oligonucleotides, oligonucleosides, oligonucleotide analogs, oligonucleotide mimetics, antisense compounds, antisense oligonucleotides, and siRNAs. An oligomeric compound may be “antisense” to a target nucleic acid, meaning that is capable of undergoing hybridization to a target nucleic acid through hydrogen bonding.

In certain embodiments, an antisense compound has a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted.

In certain embodiments, an antisense compound targeted to a Tau nucleic acid is 10 to 30 subunits in length. In certain embodiments, an antisense compound targeted to a Tau nucleic acid is 12 to 30 subunits in length. In certain embodiments, an antisense compound targeted to a Tau nucleic acid is 12 to 22 subunits in length. In certain embodiments, an antisense compound targeted to a Tau nucleic acid is 14 to 30 subunits in length. In certain embodiments, an antisense compound targeted to a Tau nucleic acid is 14 to 20 subunits in length. In certain embodiments, an antisense compound targeted to a Tau nucleic acid is 15 to 30 subunits in length. In certain embodiments, an antisense compound targeted to a Tau nucleic acid is 15 to 20 subunits in length. In certain embodiments, an antisense compound targeted to a Tau nucleic acid is 16 to 30 subunits in length. In certain embodiments, an antisense compound targeted to a Tau nucleic acid is 16 to 20 subunits in length. In certain embodiments, an antisense compound targeted to a Tau nucleic acid is 17 to 30 subunits in length. In certain embodiments, an antisense compound targeted to a Tau nucleic acid is 17 to 20 subunits in length. In certain embodiments, an antisense compound targeted to a Tau nucleic acid is 18 to 30 subunits in length. In certain embodiments, an antisense compound targeted to a Tau nucleic acid is 18 to 21 subunits in length. In certain embodiments, an antisense compound targeted to a Tau nucleic acid is 18 to 20 subunits in length. In certain embodiments, an antisense compound targeted to a Tau nucleic acid is 20 to 30 subunits in length. In other words, such antisense compounds are from 12 to 30 linked subunits, 14 to 30 linked subunits, 14 to 20 subunits, 15 to 30 subunits, 15 to 20 subunits, 16 to 30 subunits, 16 to 20 subunits, 17 to 30 subunits, 17 to 20 subunits, 18 to 30 subunits, 18 to 20 subunits, 18 to 21 subunits, 20 to 30 subunits, or 12 to 22 linked subunits, respectively. In certain embodiments, an antisense compound targeted to a Tau nucleic acid is 14 subunits in length. In certain embodiments, an antisense compound targeted to a Tau nucleic acid is 16 subunits in length. In certain embodiments, an antisense compound targeted to a Tau nucleic acid is 17 subunits in length. In certain embodiments, an antisense compound targeted to a Tau nucleic acid is 18 subunits in length. In certain embodiments, an antisense compound targeted to a Tau nucleic acid is 20 subunits in length. In other embodiments, the antisense compound is 8 to 80, 12 to 50, 13 to 30, 13 to 50, 14 to 30, 14 to 50, 15 to 30, 15 to 50, 16 to 30, 16 to 50, 17 to 30, 17 to 50, 18 to 22, 18 to 24, 18 to 30, 18 to 50, 19 to 22, 19 to 30, 19 to 50, or 20 to 30 linked subunits. In certain such embodiments, the antisense compounds are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments the antisense compound is an antisense oligonucleotide, and the linked subunits are nucleosides.

In certain embodiments, an antisense compound targeted to a Tau nucleic acid is a single stranded ribonucleic acid or deoxyribonucleic acid antisense oligonucleotide. Antisense oligonucleotides may target a specific, complementary, coding or non-coding, nucleic acid. Depending on the antisense oligonucleotide used, the binding of the oligonucleotide to its target nucleic acid sequence may or may not activate RNAse H. In some embodiments, the antisense oligonucleotide activates RNAse H, which degrades the target nucleic acid. The antisense oligonucleotides of several embodiments may be any length provided it binds selectively to the intended location. In general, the antisense oligonucleotide may be from 8, 10 or 12 nucleotides in length up to 20, 30, or 50 nucleotides in length.

In certain embodiments antisense oligonucleotides targeted to a Tau nucleic acid may be shortened or truncated. For example, a single subunit may be deleted from the 5′ end (5′ truncation), or alternatively from the 3′ end (3′ truncation). A shortened or truncated antisense compound targeted to a Tau nucleic acid may have two subunits deleted from the 5′ end, or alternatively may have two subunits deleted from the 3′ end, of the antisense compound. Alternatively, the deleted nucleosides may be dispersed throughout the antisense compound, for example, in an antisense compound having one nucleoside deleted from the 5′ end and one nucleoside deleted from the 3′ end.

When a single additional subunit is present in a lengthened antisense compound, the additional subunit may be located at the 5′ or 3′ end of the antisense compound. When two or more additional subunits are present, the added subunits may be adjacent to each other, for example, in an antisense compound having two subunits added to the 5′ end (5′ addition), or alternatively to the 3′ end (3′ addition), of the antisense compound. Alternatively, the added subunits may be dispersed throughout the antisense compound, for example, in an antisense compound having one subunit added to the 5′ end and one subunit added to the 3′ end.

It is possible to increase or decrease the length of an antisense compound, such as an antisense oligonucleotide, and/or introduce mismatch bases without eliminating activity. For example, in Woolf et al. (Proc. Natl Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

Gautschi et al. (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo.

Maher and Dolnick (Nuc. Acid. Res. 16:3341-3358, 1988) tested a series of tandem 14 nucleobase antisense oligonucleotides, and a 28 and 42 nucleobase antisense oligonucleotides comprised of the sequence of two or three of the tandem antisense oligonucleotides, respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

### Antisense Compound Motifs

In certain embodiments, antisense compounds targeted to a Tau nucleic acid have chemically modified subunits arranged in patterns, or motifs, to confer to the antisense compounds properties such as enhanced inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound may optionally serve as a substrate for the cellular endonuclease RNase H, which cleaves the RNA strand of an RNA:DNA duplex.

In certain embodiments, the antisense compounds are uniform sugar-modified oligonucleotides. Antisense compounds having a gapmer motif are considered chimeric antisense compounds. In a gapmer an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer may in some embodiments include β-D-ribonucleosides, β-D-deoxyribonucleosides, 2′-modified nucleosides (such 2′-modified nucleosides may include 2′-MOE and 2′-O—CH₃, among others), and bicyclic sugar modified nucleosides. In certain embodiments, wings may include several modified sugar moieties, including, for example 2′-MOE. In certain embodiments, wings may include several modified and unmodified sugar moieties. In certain embodiments, wings may include various combinations of 2′-MOE nucleosides and 2′-deoxynucleosides.

Each distinct region may comprise uniform sugar moieties, variant, or alternating sugar moieties. The wing-gap-wing motif is frequently described as “X-Y-Z”, where “X” represents the length of the 5′-wing, “Y” represents the length of the gap, and “Z” represents the length of the 3′-wing. “X” and “Z” may comprise uniform, variant, or alternating sugar moieties. In certain embodiments, “X” and “Y” may include one or more 2′-deoxynucleosides. “Y” may comprise 2′-deoxynucleosides. As used herein, a gapmer described as “X-Y-Z” has a configuration such that the gap is positioned immediately adjacent to each of the 5′-wing and the 3′ wing. Thus, no intervening nucleotides exist between the 5′-wing and gap, or the gap and the 3′-wing. Any of the antisense compounds described herein can have a gapmer motif. In certain embodiments, “X” and “Z” are the same; in other embodiments they are different. In certain embodiments, Y is between 8 and 15 nucleosides. X, Y, or Z can be any of 1, 2, 3, 4, 5, 6, 7, 8, 9, 10, 11, 12, 13, 14, 15, 16, 17, 18, 19, 20, 25, 30, or more nucleosides. Thus, gapmers described herein include, but are not limited to, for example, 5-10-5, 5-10-4, 4-10-4, 4-10-3, 3-10-3, 2-10-2, 5-9-5, 5-9-4, 4-9-5, 5-8-5, 5-8-4, 4-8-5, 5-7-5, 4-7-5, 5-7-4, or 4-7-4.

In certain embodiments, antisense compounds targeted to a Tau nucleic acid possess a 5-8-5 gapmer motif.

In certain embodiments, an antisense compound targeted to a Tau nucleic acid has a gap-narrowed motif. In certain embodiments, a gap-narrowed antisense oligonucleotide targeted to a Tau nucleic acid has a gap segment of 9, 8, 7, or 6 2′-deoxynucleotides positioned immediately adjacent to and between wing segments of 5, 4, 3, 2, or 1 chemically modified nucleosides. In certain embodiments, the chemical modification comprises a bicyclic sugar. In certain embodiments, the bicyclic sugar comprises a 4′ to 2′ bridge selected from among: 4′-(CH2)n-O-2′ bridge, wherein n is 1 or 2; and 4′-CH2-O—CH2-2′. In certain embodiments, the bicyclic sugar is comprises a 4′-CH(CH3)-O-2′ bridge. In certain embodiments, the chemical modification comprises a non-bicyclic 2′-modified sugar moiety. In certain embodiments, the non-bicyclic 2′-modified sugar moiety comprises a 2′-O-methylethyl group or a 2′-O-methyl group.

In certain embodiments, an antisense compound targeted to a Tau nucleic acid is uniformly modified. In certain embodiments, the antisense compound comprises 12, 13, 14, 15, 16, 17, 18, 19, 20, 21, 22, 23, 24, or 25 nucleosides. In certain embodiments, each nucleoside is chemically modified. In certain embodiments, the chemical modification comprises a non-bicyclic 2′-modified sugar moiety. In certain embodiments, the 2′-modified sugar moiety comprises a 2′-O-methoxyethyl group. In certain embodiments, the 2′-modified sugar moiety comprises a 2′-O-methyl group.

### Target Nucleic Acids, Target Regions and Nucleotide Sequences

Nucleotide sequences that encode Tau include, without limitation, the following: GENBANK Accession NT_010783.14 truncated from nucleotides 2624000 to U.S. Pat. No. 2,761,000 (incorporated herein as SEQ ID NO: 1); GENBANK Accession No. AK226139.1 (incorporated herein as SEQ ID NO: 2); GENBANK Accession No. NM_001123066.3 (incorporated herein as SEQ ID NO: 3); GENBANK Accession No. NM_001123067.3 (incorporated herein as SEQ ID NO: 4); GENBANK Accession No. NM_001203251.1 (incorporated herein as SEQ ID NO: 5); GENBANK Accession No. NM_001203252.1 (incorporated herein as SEQ ID NO: 6); GENBANK Accession No. NM_005910.5 (incorporated herein as SEQ ID NO: 7); GENBANK Accession No. NM_016834.4 (incorporated herein as SEQ ID NO: 8); GENBANK Accession No. NM_016835.4 (incorporated herein as SEQ ID NO: 9); or GENBANK Accession No. NM_016841.4 (incorporated herein as SEQ ID NO: 10).

It is understood that the sequence set forth in each SEQ ID NO contained herein is independent of any modification to a sugar moiety, an internucleoside linkage, or a nucleobase. As such, antisense compounds defined by a SEQ ID NO may comprise, independently, one or more modifications to a sugar moiety, an internucleoside linkage, or a nucleobase. Antisense compounds described by Isis Number (Isis No) indicate a combination of nucleobase sequence and motif.

In certain embodiments, a target region is a structurally defined region of the target nucleic acid. For example, a target region may encompass a 3′ UTR, a 5′ UTR, an exon, an intron, an exon/intron junction, a coding region, a translation initiation region, translation termination region, or other defined nucleic acid region. The structurally defined regions for Tau can be obtained by accession number from sequence databases such as NCBI and such information is incorporated herein by reference. In certain embodiments, a target region may encompass the sequence from a 5′ target site of one target segment within the target region to a 3′ target site of another target segment within the same target region.

Targeting includes determination of at least one target segment to which an antisense compound hybridizes, such that a desired effect occurs. In certain embodiments, the desired effect is a reduction in mRNA target nucleic acid levels. In certain embodiments, the desired effect is reduction of levels of protein encoded by the target nucleic acid or a phenotypic change associated with the target nucleic acid.

A target region may contain one or more target segments. Multiple target segments within a target region may be overlapping. Alternatively, they may be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceeding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites or 3′ target sites listed herein.

Suitable target segments may be found within a 5′ UTR, a coding region, a 3′ UTR, an intron, an exon, or an exon/intron junction. Target segments containing a start codon or a stop codon are also suitable target segments. A suitable target segment may specifically exclude a certain structurally defined region such as the start codon or stop codon.

The determination of suitable target segments may include a comparison of the sequence of a target nucleic acid to other sequences throughout the genome. For example, the BLAST algorithm may be used to identify regions of similarity amongst different nucleic acids. This comparison can prevent the selection of antisense compound sequences that may hybridize in a non-specific manner to sequences other than a selected target nucleic acid (i.e., non-target or off-target sequences).

There may be variation in activity (e.g., as defined by percent reduction of target nucleic acid levels) of the antisense compounds within an active target region. In certain embodiments, reductions in Tau mRNA levels are indicative of inhibition of Tau expression. Reductions in levels of a Tau protein are also indicative of inhibition of target mRNA expression. In certain embodiments, reductions in the 4R isoform of Tau mRNA levels are indicative of modulation of Tau splicing. Reductions in levels of the 4R isoform of Tau protein are also indicative of modulation of Tau splicing. In certain embodiments, increases in the 3R isoform of Tau mRNA levels are indicative of modulation of Tau splicing. Increases in levels of the 3R isoform of Tau protein are also indicative of modulation of Tau splicing. Reduction in percent of cells staining positive for hyperphosphorylated Tau are indicative of inhibition of Tau expression or modulation of Tau splicing. Improvement in neurological function is indicative of inhibition of Tau expression or modulation of Tau splicing. Improved memory and motor function are indicative of inhibition of Tau expression or modulation of Tau splicing. Reduction of neurofibrillary inclusions is indicative of inhibition of Tau expression or modulation of Tau splicing.

### Hybridization

In some embodiments, hybridization occurs between an antisense compound disclosed herein and a Tau nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art. In certain embodiments, the antisense compounds provided herein are specifically hybridizable with a Tau nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as a Tau nucleic acid).

Non-complementary nucleobases between an antisense compound and a Tau nucleic acid may be tolerated provided that the antisense compound remains able to specifically hybridize to a target nucleic acid. Moreover, an antisense compound may hybridize over one or more segments of a Tau nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a Tau nucleic acid, a target region, target segment, or specified portion thereof. Percent complementarity of an antisense compound with a target nucleic acid can be determined using routine methods.

For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having four noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e. 100% complementary) to a target nucleic acid, or specified portion thereof. For example, an antisense compound may be fully complementary to a Tau nucleic acid, or a target region, or a target segment or target sequence thereof. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound may or may not be fully complementary to the target sequence, depending on whether the remaining 10 nucleobases of the antisense compound are also complementary to the target sequence.

The location of a non-complementary nucleobase may be at the 5′ end or 3′ end of the antisense compound. Alternatively, the non-complementary nucleobase or nucleobases may be at an internal position of the antisense compound. When two or more non-complementary nucleobases are present, they may be contiguous (i.e. linked) or non-contiguous. In one embodiment, a non-complementary nucleobase is located in the wing segment of a gapmer antisense oligonucleotide.

In certain embodiments, antisense compounds that are, or are up to 11, 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a Tau nucleic acid, or specified portion thereof.

In certain embodiments, antisense compounds that are, or are up to 11, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a Tau nucleic acid, or specified portion thereof.

The antisense compounds provided also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds, are complementary to at least an 8 nucleobase portion of a target segment. In certain embodiments, the antisense compounds are complementary to at least a 9 nucleobase portion of a target segment. In certain embodiments, the antisense compounds are complementary to at least a 10 nucleobase portion of a target segment. In certain embodiments, the antisense compounds are complementary to at least an 11 nucleobase portion of a target segment. In certain embodiments, the antisense compounds are complementary to at least a 12 nucleobase portion of a target segment. In certain embodiments, the antisense compounds are complementary to at least a 13 nucleobase portion of a target segment. In certain embodiments, the antisense compounds are complementary to at least a 14 nucleobase portion of a target segment. In certain embodiments, the antisense compounds are complementary to at least a 15 nucleobase portion of a target segment. Also contemplated are antisense compounds that are complementary to at least a 9, 10, 11, 12, 13, 14, 15, 16, 17, 18, 19, 20, or more nucleobase portion of a target segment, or a range defined by any two of these values.

### Identity

The antisense compounds provided herein may also have a defined percent identity to a particular nucleotide sequence, SEQ ID NO, or compound represented by a specific Isis number, or portion thereof. As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases may be adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, are at least 70%, 75%, 80%, 85%, 90%, 95%, 96%, 97%, 98%, 99% or 100% identical to one or more of the antisense compounds or SEQ ID NOs, or a portion thereof, disclosed herein.

In certain embodiments, a portion of the antisense compound is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

In certain embodiments, a portion of the antisense oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to a Tau nucleic acid comprise one or more modified internucleoside linkages. In certain embodiments, the modified internucleoside linkages are interspersed throughout the antisense compound. In certain embodiments, the modified internucleoside linkages are phosphorothioate linkages. In certain embodiments, each internucleoside linkage of an antisense compound is a phosphorothioate internucleoside linkage.

### Modified Sugar Moieties

Oligomeric compounds provided herein may comprise one or more monomers, including a nucleoside or nucleotide, having a modified sugar moiety. For example, the furanosyl sugar ring of a nucleoside or nucleotide can be modified in a number of ways including, but not limited to, addition of a substituent group and bridging of two non-geminal ring atoms to form a Locked Nucleic Acid (LNA).

In certain embodiments, oligomeric compounds comprise one or more monomers having a bicyclic sugar. In certain embodiments, the monomer is an LNA. In certain such embodiments, LNAs include, but are not limited to, (A) α-L-Methyleneoxy (4′-CH₂—O-2′) LNA, (B) β-D-Methyleneoxy (4′-CH₂—O-2′) LNA, (C) Ethyleneoxy (4′-(CH₂)₂—O-2′) LNA, (D) Aminooxy (4′-CH₂—O—N(R)-2′) LNA and (E) Oxyamino (4′-CH₂—N(R)—O-2′) LNA, as depicted below.

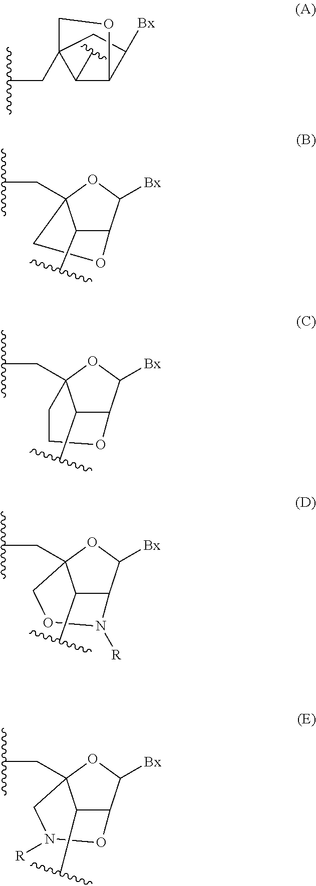

In certain embodiments, LNA compounds include, but are not limited to, compounds having at least one bridge between the 4′ and the 2′ position of the sugar wherein each of the bridges independently comprises 1 or from 2 to 4 linked groups independently selected

from —[C(R₁)(R₂)]ₙ—, —C(R₁)═C(R₂)—, —C(R₁)═N—, —C(═NR₁)—, —C(═O)—, —C(═S)—, —O—, —Si(R₁)₂—, —S(═O)ₓ— and —N(R₁)—;

wherein:

x is 0, 1, or 2;

n is 1, 2, 3, or 4;

each R₁ and R₂ is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, a heterocycle radical, a substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In one embodiment, each of the bridges of the LNA compounds is, independently, —[C(R₁)(R₂)]ₙ—, —[C(R₁)(R₂)]ₙ—O—, —C(R₁R₂)—N(R₁)—O— or —C(R₁R₂)—O—N(R₁)—. In another embodiment, each of said bridges is, independently, 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R₁)-2′ and 4′-CH₂—N(R₁)—O-2′- wherein each R₁ is, independently, H, a protecting group or C₁-C₁₂ alkyl.

Certain LNA's have been prepared and disclosed in the patent literature as well as in scientific literature (see for example: issued U.S. Pat. Nos. 7,053,207; 6,268,490; 6,770,748; 6,794,499; 7,034,133; 6,525,191; 7,696,345; 7,569,575; 7,314,923; 7,217,805; and 7,084,125, hereby incorporated by reference herein in their entirety.

Also provided herein are LNAs in which the 2′-hydroxyl group is connected, to the 4′ carbon atom of the ribosyl sugar ring, thereby forming a methyleneoxy (4′-CH₂—O-2′) bridge to form the bicyclic sugar moiety (reviewed in Elayadi et al., Curr. Opinion Invens. Drugs, 2001, 2, 558-561; Braasch et al., Chem. Biol., 2001, 8 1-7; and Orum et al., Curr. Opinion Mol. Ther., 2001, 3, 239-243; see also U.S. Pat. No. 6,670,461). Furthermore, the bridge can also be a methylene (—CH₂—) group connecting the 2′ oxygen atom to the 4′ carbon atom of the sugar ring, for which the term methyleneoxy (4′-CH₂—O-2′) LNA is used. In the case of the bicyclic sugar moiety having an ethylene bridging group in this position, the term ethyleneoxy (4′-CH₂CH₂—O-2′) LNA is used (Singh et al., Chem. Commun., 1998, 4, 455-456: Morita et al., Bioorganic Medicinal Chemistry, 2003, 11, 2211-2226). Methyleneoxy (4′-CH₂—O-2′) LNA and other bicyclic sugar analogs display very high duplex thermal stabilities with complementary DNA and RNA (Tm=+3 to +10° C.), stability towards 3′-exonucleolytic degradation and good solubility properties. Potent and nontoxic antisense oligonucleotides comprising LNAs have been described (Wahlestedt et al., Proc. Natl. Acad. Sci. U.S.A, 2000, 97, 5633-5638).

An isomer of methyleneoxy (4′-CH₂—O-2′) LNA that has also been discussed is α-L-methyleneoxy (4′-CH₂—O-2′) LNA which has been shown to have superior stability against a 3′-exonuclease. The α-L-methyleneoxy (4′-CH₂—O-2′) LNA's were incorporated into antisense gapmers and chimeras that showed potent antisense activity (Frieden et al., Nucleic Acids Research, 2003, 21, 6365-6372).

The synthesis and preparation of adenine, cytosine, guanine, 5-methyl-cytosine, thymine and uracil LNAs having a methyleneoxy (4′-CH₂—O-2′) bridge, along with their oligomerization, and nucleic acid recognition properties have been described (Koshkin et al., Tetrahedron, 1998, 54, 3607-3630). LNAs and preparation thereof are also described in WO 98/39352 and WO 99/14226, incorporated by reference herein.

Analogs of various LNA nucleosides that have 4′ to 2′ bridging groups such as 4′-CH₂—O-2′ (methyleneoxy) and 4′-CH₂—S-2′ (methylene-thio), have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-LNA, a novel conformationally restricted high-affinity oligonucleotide analog has been described in the art (Singh et al., J. Org. Chem., 1998, 63, 10035-10039). In addition, 2′-amino- and 2′-methylamino-LNA's have been prepared and the thermal stability of their duplexes with complementary RNA and DNA strands has been previously reported.

As used herein, “bicyclic nucleoside” refers to a nucleoside comprising a bridge connecting two carbon atoms of the sugar ring, thereby forming a bicyclic sugar moiety. In certain embodiments, the bridge connects the 2′ carbon and another carbon of the sugar ring.

As used herein, “4′-2′ bicyclic nucleoside” or “4′ to 2′ bicyclic nucleoside” refers to a bicyclic nucleoside comprising a furanose ring comprising a bridge connecting the 2′ carbon atom and the 4′ carbon atom of the sugar ring.

One carbocyclic bicyclic nucleoside having a 4′-(CH₂)₃-2′ bridge and the alkenyl analog, bridge 4′-CH═CH—CH₂-2′, have been described (see, e.g., Freier et al., Nucleic Acids Research, 1997, 25(22), 4429-4443 and Albaek et al., J. Org. Chem., 2006, 71, 7731-7740). The synthesis and preparation of carbocyclic bicyclic nucleosides along with their oligomerization and biochemical studies have also been described (see, e.g., Srivastava et al., J. Am. Chem. Soc. 2007, 129(26), 8362-8379).

Many other bicyclic and tricyclic sugar surrogate ring systems are also known in the art that can be used to modify nucleosides for incorporation into antisense compounds as provided herein (see, e.g., review article: Leumann, J. C, Bioorganic & Medicinal Chemistry, 2002, 10, 841-854). Such ring systems can undergo various additional substitutions to further enhance their activity. Such ring systems can undergo various additional substitutions to enhance activity.

As used herein, “2′-modified sugar” means a furanosyl sugar modified at the 2′ position. In certain embodiments, such modifications include substituents selected from: a halide, including, but not limited to substituted and unsubstituted alkoxy, substituted and unsubstituted thioalkyl, substituted and unsubstituted amino alkyl, substituted and unsubstituted alkyl, substituted and unsubstituted allyl, and substituted and unsubstituted alkynyl. In certain embodiments, 2′ modifications are selected from substituents including, but not limited to: O[(CH₂)—O]ₘCH₃, O(CH₂)—NH₂, O(CH₂)—CH₃, O(CH₂)ₙONH₂, OCH₂C(═O)N(H)CH₃, and O(CH₂)ₙON[(CH₂)ₙCH₃]₂, where n and m are from 1 to about 10. Other 2′-substituent groups can also be selected from: C₁-C₁₂ alkyl; substituted alkyl; alkenyl; alkynyl; alkaryl; aralkyl; O-alkaryl or O-aralkyl; SH; SCH₃; OCN; Cl; Br; CN; CF₃; OCF₃; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving pharmacokinetic properties; and a group for improving the pharmacodynamic properties of an antisense compound, and other substituents having similar properties. In certain embodiments, modified nucleosides comprise a 2′-MOE side chain (see, e.g., Baker et al., J. Biol. Chem., 1997, 272, 11944-12000). Such 2′-MOE substitution have been described as having improved binding affinity compared to unmodified nucleosides and to other modified nucleosides, such as 2′-O-methyl, O-propyl, and O-aminopropyl. Oligonucleotides having the 2′-MOE substituent also have been shown to be antisense inhibitors of gene expression with promising features for in vivo use (see, e.g., Martin, P., Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

As used herein, “2′-modified nucleoside” or “2′-substituted nucleoside” refers to a nucleoside comprising a sugar comprising a substituent at the 2′ position of a furanose ring other than H or OH. 2′ modified nucleosides include, but are not limited to, nucleosides with non-bridging 2′ substituents, such as allyl, amino, azido, thio, O-allyl, O—C₁-C₁₀ alkyl, —OCF₃, O—(CH₂)₂—O—CH₃, 2′-O(CH₂)₂SCH₃, O—(CH₂)₂—O—N(Rₘ)(Rₙ), or O—CH₂—C(═O)—N(Rₘ)(Rₙ), where each Rₘ and Rₙ is, independently, H or substituted or unsubstituted C₁-C₁₀ alkyl. 2′-modified nucleosides may further comprise other modifications, for example, at other positions of the sugar and/or at the nucleobase.

As used herein, “2′-F” refers to modification of the 2′ position of the furanosyl sugar ring to comprise a fluoro group.

As used herein, “2′-OMe” or “2′-OCH₃” or “2′-O-methyl” each refers to modification at the 2′ position of the furanosyl sugar ring to comprise a —OCH₃ group.

As used herein, “oligonucleotide” refers to a compound comprising a plurality of linked nucleosides. In certain embodiments, one or more of the plurality of nucleosides is modified. In certain embodiments, an oligonucleotide comprises one or more ribonucleosides (RNA) and/or deoxyribonucleosides (DNA). Methods for the preparations of modified sugars are well known to those skilled in the art.

In nucleotides having modified sugar moieties, the nucleobase moieties (natural, modified, or a combination thereof) are maintained for hybridization with an appropriate nucleic acid target.

In certain embodiments, antisense compounds comprise one or more nucleosides having modified sugar moieties. In certain embodiments, the modified sugar moiety is 2′-MOE.

### Occupancy

In certain embodiments, antisense compounds are not expected to result in cleavage or the target nucleic acid via RNase H or to result in cleavage or sequestration through the RISC pathway. In certain such embodiments, antisense activity may result from occupancy, wherein the presence of the hybridized antisense compound disrupts the activity of the target nucleic acid. In certain such embodiments, the antisense compound may be uniformly modified or may comprise a mix of modifications and/or modified and unmodified nucleosides.

In certain embodiments, antisense oligonucleotides do not activate RNAse H. In several aspects, antisense oligonucleotides that do not activate RNAse H are complementary to a nucleic acid sequence encoding Tau and disrupts the splicing of the nucleic acid encoding Tau to reduce the 4R:3R Tau ratio.

The antisense oligonucleotide of several embodiments may disrupt the splicing of the nucleic acid encoding Tau to reduce the 4R:3R Tau ratio. The splicing process is a series of reactions, mediated by splicing factors, which is carried out on RNA after transcription but before translation, in which the intron(s) are removed, and the exons joined together sequentially so that the protein may be translated. Each intron is defined by a 5′ splice site, a 3′ splice site, and a branch point situated there between. An antisense oligonucleotide may block these splice elements when the oligonucleotide either fully or partially overlaps the element, or binds to the pre-mRNA at a position sufficiently close to the element to disrupt the binding and function of the splicing factors which would ordinarily mediate the particular splicing reaction which occurs at that element. The antisense oligonucleotide may block a variety of different splice elements to carry out certain embodiments. For instance, the antisense oligonucleotide may block a mutated element, a cryptic element, or a native element; it may block a 5′ splice site, a 3′ splice site, or a branch point.

Methods of making antisense oligonucleotides which do not activate RNase H are known in the art. See, e.g., U.S. Pat. No. 5,149,797 incorporated herein by reference. Such antisense oligonucleotides may contain one or more structural modification which sterically hinders or prevents binding of RNase H to a duplex molecule comprising the oligonucleotide, but does not substantially hinder or disrupt duplex formation. Antisense oligonucleotides that do not activate RNAse H may include oligonucleotides wherein at least one, two or more of the internucleotide bridging phosphate residues are modified phosphates, such as methyl phosphonates, methyl phosphonothioates, phosphoromorpholidates, phosphoropiperazidates and phosphoramidates. For instance, every other one of the internucleotide bridging phosphate residues may be a modified phosphate, contain a 2′ lower alkyl moiety (e.g., C1-C4, linear or branched, saturated or unsaturated alkyl, such as methyl, ethyl, ethenyl, propyl, 1-propenyl, 2-propenyl, and isopropyl) or a combination thereof. In preferred embodiments, the antisense oligonucleotide of the invention that does not activate RNAse H, and disrupts the splicing of the nucleic acid encoding Tau to reduce the 4R:3R Tau ratio is a 2′-O-(2-methoxyethyl) (MOE)-modified antisense oligonucleotide.

Other methods of modifying an oligonucleotide to hinder binding of RNAse H may be found in P. Furdon et al., Nucleic Acids Res. 17, 9193-9204 (1989); S. Agrawal et al., Proc. Natl. Acad. Sci. USA 87, 1401-1405 (1990); C. Baker et al., Nucleic Acids Res. 18, 3537-3543 (1990); B. Sproat et al., Nucleic Acids Res. 17, 3373-3386 (1989); R. Walder and J. Walder, Proc. Natl. Acad. Sci. USA 85, 5011-5015 (1988) the disclosures of all of which are incorporated herein, in their entirety, by reference.

### Compositions and Methods for Formulating Pharmaceutical Compositions

Antisense oligonucleotides may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

An antisense compound targeted to a Tau nucleic acid can be utilized in pharmaceutical compositions by combining the antisense compound with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising an antisense compound targeted to a Tau nucleic acid and a pharmaceutically acceptable diluent. In certain embodiments, the pharmaceutically acceptable diluent is PBS. In certain embodiments, the antisense compound is an antisense oligonucleotide.

Pharmaceutical compositions comprising antisense compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts. A prodrug can include the incorporation of additional nucleosides at one or both ends of an antisense compound which are cleaved by endogenous nucleases within the body, to form the active antisense compound.

In certain embodiments, an antisense oligonucleotide can include a physiologically and pharmaceutically acceptable salts thereof: i.e., salts that retain the desired biological activity of the parent compound and do not impart undesired toxicological effects thereto. Examples of such salts are (a) salts formed with cations such as sodium, potassium, NH4+, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

### Administration

Antisense oligonucleotides of certain embodiments may be administered to a subject by several different means. For instance, oligonucleotides may generally be administered parenterally, intraperitoneally, intravascularly, or intrapulmonarily in dosage unit formulations containing conventional nontoxic pharmaceutically acceptable carriers, adjuvants, and vehicles as desired. In a preferred embodiment, the oligonucleotide may be administered parenterally.

The term parenteral as used herein includes subcutaneous, intravenous, intramuscular, intrathecal, or intrasternal injection, or infusion techniques. Formulation of pharmaceutical compositions is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

Delivery methods are preferably those that are effective to circumvent the blood-brain barrier and are effective to deliver agents to the central nervous system. For example, delivery methods may include the use of nanoparticles. The particles may be of any suitable structure, such as unilamellar or plurilamellar, so long as the antisense oligonucleotide is contained therein.

Positively charged lipids such as N-[1-(2,3-dioleoyloxi)propyl)-N, N,N-trimethyl-amoniummethylsulfate, or “DOTAP,” are particularly preferred for such particles and vesicles. The preparation of such lipid particles is well known in the art. See, e.g., U.S. Pat. No. 4,880,635 to Janoff et al.; U.S. Pat. No. 4,906,477 to Kurono et al.: U.S. Pat. No. 4,911,928 to Wallach; U.S. Pat. No. 4,917,951 to Wallach; U.S. Pat. No. 4,920,016 to Allen et al.: U.S. Pat. No. 4,921,757 to Wheatley et al.; etc.

In one embodiment, the compounds provided herein may be administered in a bolus directly into the central nervous system. The compounds provided herein may be administered to the subject in a bolus once, or multiple times. In some preferred embodiments, the compounds provided herein may be administered once. In other preferred embodiments, the compounds provided herein may be administered multiple times. When administered multiple times, the compounds provided herein may be administered at regular intervals or at intervals that may vary during the treatment of a subject. In some embodiments, the compounds provided herein may be administered multiple times at intervals that may vary during the treatment of a subject. In some embodiments, the compounds provided herein may be administered multiple times at regular intervals.

In another preferred embodiment, the compounds provided herein may be administered by continuous infusion into the central nervous system. Non-limiting examples of methods that may be used to deliver the compounds provided herein into the central nervous system by continuous infusion may include pumps, wafers, gels, foams and fibrin clots. In a preferred embodiment, the compounds provided herein may be delivered into the central nervous system by continuous infusion using an osmotic pump. An osmotic mini pump contains a high-osmolality chamber that surrounds a flexible, yet impermeable, reservoir filled with the targeted delivery composition-containing vehicle. Subsequent to the subcutaneous implantation of this minipump, extracellular fluid enters through an outer semi-permeable membrane into the high-osmolality chamber, thereby compressing the reservoir to release the targeted delivery composition at a controlled, pre-determined rate. The targeted delivery composition, released from the pump, may be directed via a catheter to a stereotaxically placed cannula for infusion into the cerebroventricular space. In certain embodiments, the compounds provided herein may be delivered into the central nervous system by continuous infusion using a pump as described in the Examples.

In another preferred embodiment, the compounds provided herein may be delivered into the central nervous system by intrathecal administration. A catheter may be placed in the intrathecal lumbar space of the animal. The proximal end of the catheter may be attached to a dosing pedestal that may extend through the skin. In further embodiments, the compounds provided herein may be administered as a bolus injection. In other embodiments, the compounds provided herein may be administered as a continuous infusion.

### Conjugated Antisense Compounds

Antisense compounds may be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

Antisense compounds can also be modified to have one or more stabilizing groups that are generally attached to one or both termini of antisense compounds to enhance properties such as, for example, nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense compound to impart nuclease stability include those disclosed in WO 03/004602 published on Jan. 16, 2003.

### Cell Culture and Antisense Compounds Treatment

The effects of antisense compounds on the level, activity or expression of Tau nucleic acids can be tested in vitro in a variety of cell types. Cell types used for such analyses are available from commercial vendors (e.g. American Type Culture Collection, Manassas, Va.; Zen-Bio, Inc., Research Triangle Park, N.C.; Clonetics Corporation, Walkersville, Md.) and are cultured according to the vendor's instructions using commercially available reagents (e.g. Invitrogen Life Technologies, Carlsbad, Calif.). Illustrative cell types include, but are not limited to, SH-SY5Y and A172.

### In Vitro Testing of Antisense Oligonucleotides

Described herein are methods for treatment of cells with antisense oligonucleotides, which can be modified appropriately for treatment with other antisense compounds.

Cells may be treated with antisense oligonucleotides when the cells reach approximately 60-80% confluency in culture.

One reagent commonly used to introduce antisense oligonucleotides into cultured cells includes the cationic lipid transfection reagent LIPOFECTIN (Invitrogen, Carlsbad, Calif.). Antisense oligonucleotides may be mixed with LIPOFECTIN in OPTI-MEM 1 (Invitrogen, Carlsbad, Calif.) to achieve the desired final concentration of antisense oligonucleotide and a LIPOFECTIN concentration that may range from 2 to 12 ug/mL per 100 nM antisense oligonucleotide.

Another reagent used to introduce antisense oligonucleotides into cultured cells includes LIPOFECTAMINE (Invitrogen, Carlsbad, Calif.). Antisense oligonucleotide is mixed with LIPOFECTAMINE in OPTI-MEM 1 reduced serum medium (Invitrogen, Carlsbad, Calif.) to achieve the desired concentration of antisense oligonucleotide and a LIPOFECTAMINE concentration that may range from 2 to 12 ug/mL per 100 nM antisense oligonucleotide.

Another technique used to introduce antisense oligonucleotides into cultured cells includes electroporation.

Cells are treated with antisense oligonucleotides by routine methods. Cells may be harvested 16-24 hours after antisense oligonucleotide treatment, at which time RNA or protein levels of target nucleic acids are measured by methods known in the art and described herein. In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art. Antisense oligonucleotides are typically used at concentrations ranging from 1 nM to 300 nM when transfected with LIPOFECTAMINE. Antisense oligonucleotides are used at higher concentrations ranging from 625 to 20,000 nM when transfected using electroporation.

### RNA Isolation

RNA analysis can be performed on total cellular RNA or poly(A)+ mRNA. Methods of RNA isolation are well known in the art. RNA is prepared using methods well known in the art, for example, using the TRIZOL Reagent (Invitrogen, Carlsbad, Calif.) according to the manufacturer's recommended protocols.

### Analysis of Inhibition of Target Levels or Expression

Inhibition of levels or expression of a Tau nucleic acid can be assayed in a variety of ways known in the art. For example, target nucleic acid levels can be quantitated by, e.g., Northern blot analysis, competitive polymerase chain reaction (PCR), or quantitative real-time PCR. RNA analysis can be performed on total cellular RNA or poly(A)+ mRNA. Methods of RNA isolation are well known in the art. Northern blot analysis is also routine in the art. Quantitative real-time PCR can be conveniently accomplished using the commercially available ABI PRISM 7600, 7700, or 7900 Sequence Detection System, available from PE-Applied Biosystems, Foster City, Calif. and used according to manufacturer's instructions.

### Quantitative Real-Time PCR Analysis of Target RNA Levels

Quantitation of target RNA levels may be accomplished by quantitative real-time PCR using the ABI PRISM 7600, 7700, or 7900 Sequence Detection System (PE-Applied Biosystems, Foster City, Calif.) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents may be obtained from Invitrogen (Carlsbad, Calif.). RT real-time-PCR reactions are carried out by methods well known to those skilled in the art.

Gene (or RNA) target quantities obtained by real time PCR are normalized using either the expression level of a gene whose expression is constant, such as cyclophilin A, or by quantifying total RNA using RIBOGREEN (Invitrogen, Inc. Carlsbad, Calif.). Cyclophilin A expression is quantified by real time PCR, by being run simultaneously with the target, multiplexing, or separately. Total RNA is quantified using RIBOGREEN RNA quantification reagent (Invitrogen, Inc. Eugene, Oreg.). Methods of RNA quantification by RIBOGREEN are Taught in Jones, L. J., et al, (Analytical Biochemistry, 1998, 265, 368-374). A CYTOFLUOR 4000 instrument (PE Applied Biosystems) is used to measure RIBOGREEN fluorescence.

Probes and primers are designed to hybridize to a Tau nucleic acid. Methods for designing real-time PCR probes and primers are well known in the art, and may include the use of software such as PRIMER EXPRESS Software (Applied Biosystems, Foster City, Calif.).

### Quantitative Real-Time PCR Analysis of Target DNA Levels

Quantitation of target DNA levels may be accomplished by quantitative real-time PCR using the ABI PRISM 7600, 7700, or 7900 Sequence Detection System (PE-Applied Biosystems, Foster City, Calif.) according to manufacturer's instructions. Methods of quantitative real-time PCR are well known in the art.

Gene (or DNA) target quantities obtained by real time PCR are normalized using either the expression level of a gene whose expression is constant, such as cyclophilin A, or by quantifying total DNA using RIBOGREEN (Invitrogen, Inc. Carlsbad, Calif.). Cyclophilin A expression is quantified by real time PCR, by being run simultaneously with the target, multiplexing, or separately. Total DNA is quantified using RIBOGREEN RNA quantification reagent (Invitrogen, Inc. Eugene, Oreg.). Methods of DNA quantification by RIBOGREEN are Taught in Jones, L. J., et al, (Analytical Biochemistry, 1998, 265, 368-374). A CYTOFLUOR 4000 instrument (PE Applied Biosystems) is used to measure RIBOGREEN fluorescence.

Probes and primers are designed to hybridize to a Tau nucleic acid. Methods for designing real-time PCR probes and primers are well known in the art, and may include the use of software such as PRIMER EXPRESS Software (Applied Biosystems, Foster City, Calif.).

### Analysis of Protein Levels

Antisense inhibition of Tau nucleic acids can be assessed by measuring Tau protein levels. Protein levels of Tau can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained from a variety of sources, or can be prepared via conventional monoclonal or polyclonal antibody generation methods well known in the art.

### In Vivo Testing of Antisense Compounds

Antisense compounds, for example, antisense oligonucleotides, are tested in animals to assess their ability to inhibit expression of Tau and produce phenotypic changes. Testing may be performed in non-transgenic animals, or in experimental disease models. For administration to animals, antisense oligonucleotides are formulated in a pharmaceutically acceptable diluent, such as phosphate-buffered saline. Administration includes parenteral routes of administration, such as intraperitoneal, intravenous, subcutaneous, intrathecal, and intracerebroventricular. Calculation of antisense oligonucleotide dosage and dosing frequency is within the abilities of those skilled in the art, and depends upon factors such as route of administration and animal body weight. Following a period of treatment with antisense oligonucleotides, RNA is isolated from brain tissue and changes in Tau nucleic acid expression are measured. Changes in Tau DNA levels are also measured. Changes in Tau protein levels are also measured. Changes in Tau splicing are also measured.

### Tau Splicing

Certain embodiments provided herein relate to differential splicing in tau. Accordingly, several embodiments provide methods of treating a tau associated disease by lowering tau or altering the splicing of a nucleic acid encoding tau. Tau is a protein found in multiple tissues, but is particularly abundant in axons of neurons. The primary function of tau is to bind to and stabilize microtubules, which are important structural components of the cytoskeleton involved in mitosis, cytokinesis and vesicular transport. In humans, there are six isoforms of tau that are generated by alternative splicing of exons 2, 3, and 10. Splicing of exons 2 and 3 at the N-terminus of the protein leads to inclusion of zero, one or two 29 amino acid, acidic domains and is termed 0N, 1 N, or 2N tau respectively. Inclusion of exon 10 at the C-terminus leads to inclusion of the microtubule binding domain encoded by exon 10. Since there are 3 microtubule binding domains elsewhere in tau, this tau isoform (with exon 10 included) is termed 4R tau, where R refers to the number of repeats of microtubule binding domains. Tau without exon 10 is termed 3R tau. In healthy subjects, the ratio of 3R:4R tau is developmentally regulated, with fetal tissues expressing exclusively 3R tau and adult human tissues expressing approximately equal levels of 3R/4R tau. Deviations from the normal ratio of 3R:4R tau are characteristic of neurodegenerative syndromes such as FTD tauopathies. In essence, the method decreases the 4R:3R tau ratio in the central nervous system of the subject.

The 4R:3R tau ratio in the central nervous system of the subject may be normal, low or high. As used herein, a “normal 4R:3R tau ratio” in the central nervous system signifies a 4R:3R tau ratio in the central nervous system that is substantially the same as the 4R:3R tau ratio in the central nervous system of a subject from the same species and of approximately the same age not suffering from a neurodegenerative disease. In some embodiments, the method decreases the normal 4R:3R tau ratio in the central nervous system of a subject. In other embodiments, the method decreases a low 4R:3R tau ratio in the central nervous system of a subject.

In certain embodiments, the method decreases a high 4R:3R tau ratio in the central nervous system of a subject. In certain embodiments, the method decreases a high 4R:3R tau ratio caused by a defect in splicing of the nucleic acid encoding tau in the subject. Defects in splicing of the nucleic acid encoding tau in the subject may be caused, for instance, by genetic mutations altering the splicing of the nucleic acid encoding tau and leading to a high 4R:3R tau ratio. A mutation may be either a substitution mutation or a deletion mutation which creates a new, aberrant, splice element. Non-limiting examples of genetic mutations that may alter the splicing of the nucleic acid encoding tau and lead to a high 4R:3R tau ratio may include N279K, P301S, Ll280, L284L, N296H, N296N, Ll296N, P301 S, G303V, E10+11, E10+12, E10+13, E+10+14 and E10+16, and E10+19. Certain embodiments relate to a method of decreasing the 4R:3R tau ratio in the central nervous system of a subject by lowering expression of tau or altering the splicing of a nucleic acid encoding tau administering an antisense compound to the subject.

### Certain Indications

In certain embodiments, provided herein are methods of treating an individual comprising administering one or more pharmaceutical compositions described herein. In certain embodiments, the individual has a neurodegenerative disease. In certain embodiments, the individual is at risk for developing a neurodegenerative disease, including, but not limited to, Alzheimer's Disease, Fronto-temporal Dementia (FTD), FTDP-17, Progressive Supranuclear Palsy (PSP), Chronic Traumatic Encephalopathy (CTE), Corticobasal Ganglionic Degeneration (CBD), Epilepsy, and Dravet's Syndrome. In certain embodiments, the individual has been identified as having a Tau associated disease. In certain embodiments, provided herein are methods for prophylactically reducing Tau expression in an individual. In certain embodiments, provided herein are methods for prophylactically modulating Tau splicing in an individual. Certain embodiments include treating an individual in need thereof by administering to an individual a therapeutically effective amount of an antisense compound targeted to a Tau nucleic acid.

In certain embodiments, administration of a therapeutically effective amount of an antisense compound targeted to a Tau nucleic acid is accompanied by monitoring of Tau levels and Tau isoform in an individual, to determine an individual's response to administration of the antisense compound. An individual's response to administration of the antisense compound may be used by a physician to determine the amount and duration of therapeutic intervention.

In certain embodiments, administration of an antisense compound targeted to a Tau nucleic acid results in reduction of Tau expression by at least 15, 20, 25, 30, 35, 40, 45, 50, 55, 60, 65, 70, 75, 80, 85, 90, 95 or 99%, or a range defined by any two of these values. In certain embodiments, administration of an antisense compound targeted to a Tau nucleic acid results in reduction of the 4R isoform of Tau expression by at least 15, 20, 25, 30, 35, 40, 45, 50, 55, 60, 65, 70, 75, 80, 85, 90, 95 or 99%, or a range defined by any two of these values. In certain embodiments, administration of an antisense compound targeted to a Tau nucleic acid results in reduced memory loss, reduced anxiety, improved motor function in an animal, and/or reduced incidence or severity of seizures. In certain embodiments, administration of a Tau antisense results in reduced memory loss, reduced anxiety, improved motor function; and/or reduced incidence or severity of seizures by at least 15, 20, 25, 30, 35, 40, 45, 50, 55, 60, 65, 70, 75, 80, 85, 90, 95 or 99%, or a range defined by any two of these values.

In certain embodiments, pharmaceutical compositions comprising an antisense compound targeted to Tau are used for the preparation of a medicament for treating a patient suffering or susceptible to a neurodegenerative disease including Alzheimer's Disease, Fronto-temporal Dementia (FTD), FTDP-17, Progressive Supranuclear Palsy (PSP), Chronic Traumatic Encephalopathy (CTE), Corticobasal Ganglionic Degeneration (CBD), Epilepsy, and Dravet's Syndrome.

### Certain Splicing Compounds

In certain embodiments, splicing compounds are useful for treating neurodegenerative syndromes. In certain embodiments, such splicing compounds promote the exclusion of exon 10, resulting in shifting tau isoform from 4R Tau (which is associated with neurodegenerative syndrome) to 3R Tau. In certain embodiments, such splicing compounds are antisense oligonucleotides wherein each nucleoside comprises a high affinity modification. In certain embodiments, the splicing compound is complementary to a human Tau genetic sequence. In certain embodiments, the splicing compound is complementary to SEQ ID NO: 1 (GENBANK Accession No. NT_010783.14 truncated from nucleotides 2624000 to 2761000).

In certain embodiments, splicing compounds promote the exclusion of exon 1, 5, 7, 9, or 11, resulting in introduction of a pre-mature termination codon. In such embodiments, the resulting mRNA may be targeted for degradation by nonsense mediated decay.

In certain embodiments, splicing compounds promote the exclusion or inclusion of exon −1, 1, 2, 4, 5, 7, 9, 11, 12, or 13, resulting in an aberrant mRNA that is recognized and degraded by the cellular machinery.

### EXAMPLES

### Non-Limiting Disclosure and Incorporation by Reference

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1: Design of Antisense Compounds Targeted to Tau

A series of modified oligonucleotides were designed to target the Tau transcript, see Table 1 and Table 2 below. These modified oligonucleotides are 18 nucleosides in length, and each nucleoside of the modified oligonucleotide has a 2′-MOE modification. Each internucleoside linkage throughout the modified oligonucleotide are phosphorothioate internucleoside linkages (P═S). All cytosine residues throughout the modified oligonucleotides are 5-methylcytosines. Each modified oligonucleotide listed in Table 1 and Table 2 below is targeted to GENBANK Accession No NT_165773.2 truncated from nucleotides 15633000 to Ser. No. 15/736,000 (SEQ ID NO: 12). The locations of the target regions on the target Tau transcript are defined in Table A below. Some modified oligonucleotides listed in Tables 1 and 2 have a target region that is completely within an intron. The target regions of such modified oligonucleotides are listed as being near the 3′-end or the 5′-end of the appropriate intron. For example, a modified oligonucleotide with a target region of “5′ Intron 1” is targeted to the 5′-end of Intron 1.

TABLE A
Target Regions on SEQ ID NO: 12

| | SEQ | SEQ |
|---|---|---|
| | ID: 12 | ID: 12 |
| Target | Start | Stop |
| Region | Site | Site |
| Exon-1 | 826 | 997 |
| Intron-1 | 998 | 51757 |
| Exon 1 | 51758 | 51870 |
| Intron 1 | 51871 | 56512 |
| Exon 2 | 56513 | 56599 |
| Intron 2 | 56600 | 59297 |
| Exon 3 | 59298 | 59384 |
| Intron 3 | 59385 | 64248 |
| Exon 4 | 64249 | 64314 |
| Intron 4 | 64315 | 71750 |
| Exon 5 | 71751 | 71800 |
| Intron 5-7 | 71801 | 76015 |
| Exon 7 | 76016 | 76148 |
| Intron 7-9 | 76149 | 79658 |
| Exon 9 | 79659 | 79924 |
| Intron 9 | 79925 | 87546 |
| Exon 10 | 87547 | 87639 |
| Intron 10 | 87640 | 90740 |
| Exon 11 | 90741 | 90822 |
| Intron 11 | 90823 | 91817 |
| Exon 12 | 91818 | 91930 |
| Intron 12 | 91931 | 97361 |
| Exon 13 | 97362 | 101486 |

TABLE 1
Uniform 2′-MOE modified
oligonucleotides targeted to tau

| | | | SEQ | SEQ | |
|---|---|---|---|---|---|
| | | | ID: 12 | ID: 12 | |
| Isis | | | Start | Stop | SEQ |
| No. | ASO seq. | Target Region | Site | Site | ID NO |
| 580255 | TTGTGGCGGCGGCGGCAG | Exon-1 | 928 | 945 | 17 |
| 580256 | GGTGGTTGTGGCGGCGGC | Exon-1 | 933 | 950 | 18 |
| 580257 | GAGAAGGTGGTTGTGGCG | Exon-1 | 938 | 955 | 19 |
| 580258 | CGGAGGAGAAGGTGGTTG | Exon-1 | 943 | 960 | 20 |
| 580259 | GACAGCGGAGGAGAAGGT | Exon-1 | 948 | 965 | 21 |
| 580260 | AAGAGGACAGCGGAGGAG | Exon-1 | 953 | 970 | 22 |
| 580261 | GACAGAAGAGGACAGCGG | Exon-1 | 958 | 975 | 23 |
| 580262 | GCGAGGACAGAAGAGGAC | Exon-1 | 963 | 980 | 24 |
| 580263 | AGAAGGCGAGGACAGAAG | Exon-1 | 968 | 985 | 25 |
| 580264 | TCGACAGAAGGCGAGGAC | Exon-1 | 973 | 990 | 26 |
| 580265 | GATAATCGACAGAAGGCG | Exon-1 | 978 | 995 | 27 |
| 580266 | TACCTGATAATCGACAGA | Exon-1/Intron-1 | 983 | 1000 | 28 |
| 580267 | GCGCTTACCTGATAATCG | Exon-1/Intron-1 | 988 | 1005 | 29 |
| 580268 | CTGCGGCGCTTACCTGAT | Exon-1/Intron-1 | 993 | 1010 | 30 |
| 580269 | CAGAGCTGCGGCGCTTAC | 5′ Intron-1 | 998 | 1015 | 31 |
| 580270 | GATTTCAGAGCTGCGGCG | 5′ Intron-1 | 1003 | 1020 | 32 |
| 580271 | GACTGGATTTCAGAGCTG | 5′ Intron-1 | 1008 | 1025 | 33 |
| 580272 | AATTGGTTACAGGTGGGA | 3′ Intron-1 | 51718 | 51735 | 34 |
| 580273 | AATGGAATTGGTTACAGG | 3′ Intron-1 | 51723 | 51740 | 35 |
| 580274 | AAGACAATGGAATTGGTT | 3′ Intron-1 | 51728 | 51745 | 36 |
| 580275 | AAAAAAAGACAATGGAAT | 3′ Intron-1 | 51733 | 51750 | 37 |
| 580276 | GGAGGAAAAAAAGACAAT | 3′ Intron-1 | 51738 | 51755 | 38 |
| 580277 | AGCCTGGAGGAAAAAAAG | Intron-1/Exon 1 | 51743 | 51760 | 39 |
| 580278 | TTCAAAGCCTGGAGGAAA | Intron-1/Exon 1 | 51748 | 51765 | 40 |
| 580279 | ACTGGTTCAAAGCCTGGA | Intron-1/Exon 1 | 51753 | 51770 | 41 |
| 580280 | GCCATACTGGTTCAAAGC | Exon 1 | 51758 | 51775 | 42 |
| 580281 | GGTCAGCCATACTGGTTC | Exon 1 | 51763 | 51780 | 43 |
| 580282 | GCGAGGGTCAGCCATACT | Exon 1 | 51768 | 51785 | 44 |
| 580283 | TCCTGGCGAGGGTCAGCC | Exon 1 | 51773 | 51790 | 45 |
| 580284 | CAAACTCCTGGCGAGGGT | Exon 1 | 51778 | 51795 | 46 |
| 580285 | TGTGTCAAACTCCTGGCG | Exon 1 | 51783 | 51800 | 47 |
| 580286 | TCCATTGTGTCAAACTCC | Exon 1 | 51788 | 51805 | 48 |
| 580287 | GGTCTTCCATTGTGTCAA | Exon 1 | 51793 | 51810 | 49 |
| 580288 | AGCATGGTCTTCCATTGT | Exon 1 | 51798 | 51815 | 50 |
| 580289 | TCTCCAGCATGGTCTTCC | Exon 1 | 51803 | 51820 | 51 |
| 580290 | TGTAATCTCCAGCATGGT | Exon 1 | 51808 | 51825 | 52 |
| 580291 | CAGAGTGTAATCTCCAGC | Exon 1 | 51813 | 51830 | 53 |
| 580292 | TGGAGCAGAGTGTAATCT | Exon 1 | 51818 | 51835 | 54 |
| 580293 | GGTCTTGGAGCAGAGTGT | Exon 1 | 51823 | 51840 | 55 |
| 580294 | TTCTTGGTCTTGGAGCAG | Exon 1 | 51828 | 51845 | 56 |
| 580295 | TCTCCTTCTTGGTCTTGG | Exon 1 | 51833 | 51850 | 57 |
| 580296 | CCATGTCTCCTTCTTGGT | Exon 1 | 51838 | 51855 | 58 |
| 580297 | ATGGTCCATGTCTCCTTC | Exon 1 | 51843 | 51860 | 59 |
| 580298 | AAGCCATGGTCCATGTCT | Exon 1 | 51848 | 51865 | 60 |
| 580299 | CTTTTAAGCCATGGTCCA | Exon 1 | 51853 | 51870 | 61 |
| 580300 | CTGACCTTTTAAGCCATG | Exon 1/Intron 1 | 51858 | 51875 | 62 |
| 580301 | CCCCACTGACCTTTTAAG | Exon 1/Intron 1 | 51863 | 51880 | 63 |
| 580302 | GTCCACCCCACTGACCTT | Exon 1/Intron 1 | 51868 | 51885 | 64 |
| 580303 | TGTATGTCCACCCCACTG | 5′ Intron 1 | 51873 | 51890 | 65 |
| 580304 | AGTTGTGTATGTCCACCC | 5′ Intron 1 | 51878 | 51895 | 66 |
| 580305 | TGGCCAGTTGTGTATGTC | 5′ Intron 1 | 51883 | 51900 | 67 |
| 580306 | GTGACTGGCCAGTTGTGT | 5′ Intron 1 | 51888 | 51905 | 68 |
| 580307 | CCACTGTGACTGGCCAGT | 5′ Intron 1 | 51893 | 51910 | 69 |
| 580308 | TCAGACCTCAAGAGGAAC | 3′ Intron 1 | 56473 | 56490 | 70 |
| 580309 | AGCCCTCAGACCTCAAGA | 3′ Intron 1 | 56478 | 56495 | 71 |
| 580310 | CAGTGAGCCCTCAGACCT | 3′ Intron 1 | 56483 | 56500 | 72 |
| 580311 | ATATACAGTGAGCCCTCA | 3′ Intron 1 | 56488 | 56505 | 73 |
| 580312 | GGAACATATACAGTGAGC | 3′ Intron 1 | 56493 | 56510 | 74 |
| 580313 | ACTCTGGAACATATACAG | Intron 1/Exon 2 | 56498 | 56515 | 75 |
| 580314 | GGGAGACTCTGGAACATA | Intron 1/Exon 2 | 56503 | 56520 | 76 |
| 580315 | CTCCGCTCCATCATCGGC | Exon 2 | 56533 | 56550 | 77 |
| 580316 | GGTTCCTCCGCTCCATCA | Exon 2 | 56538 | 56555 | 78 |
| 580317 | ACCCTGGTTCCTCCGCTC | Exon 2 | 56543 | 56560 | 79 |
| 580318 | CTCCGACCCTGGTTCCTC | Exon 2 | 56548 | 56565 | 80 |
| 580319 | GAGGTCTCCGACCCTGGT | Exon 2 | 56553 | 56570 | 81 |
| 580320 | CATCGGAGGTCTCCGACC | Exon 2 | 56558 | 56575 | 82 |
| 580321 | CTTAGCATCGGAGGTCTC | Exon 2 | 56563 | 56580 | 83 |
| 580322 | GTGCTCTTAGCATCGGAG | Exon 2 | 56568 | 56585 | 84 |
| 580323 | TTGGAGTGCTCTTAGCAT | Exon 2 | 56573 | 56590 | 85 |
| 580324 | AGCAGTTGGAGTGCTCTT | Exon 2 | 56578 | 56595 | 86 |
| 580325 | CCTTCAGCAGTTGGAGTG | Exon 2/Intron 2 | 56583 | 56600 | 87 |
| 580326 | CCCCACCTTCAGCAGTTG | Exon 2/Intron 2 | 56588 | 56605 | 88 |
| 580327 | GGAGGCCCCACCTTCAGC | Exon 2/Intron 2 | 56593 | 56610 | 89 |
| 580328 | AGTAGGGAGGCCCCACCT | Exon 2/Intron 2 | 56598 | 56615 | 90 |
| 580329 | GTGGGAGTAGGGAGGCCC | 5′ Intron 2 | 56603 | 56620 | 91 |
| 580330 | GAGTGGTGGGAGTAGGGA | 5′ Intron 2 | 56608 | 56625 | 92 |
| 580331 | GAATGGAGTGGTGGGAGT | 5′ Intron 2 | 56613 | 56630 | 93 |
| 580332 | TGAATGAATGGAGTGGTG | 5′ Intron 2 | 56618 | 56635 | 94 |
| 580333 | TAAGCTGAATGAATGGAG | 5′ Intron 2 | 56623 | 56640 | 95 |
| 580334 | GAGGACAGAAGGGAGACT | 3′ Intron 3 | 64209 | 64226 | 96 |
| 580335 | AGGGAGAGGACAGAAGGG | 3′ Intron 3 | 64214 | 64231 | 97 |
| 580336 | AGCAAAGGGAGAGGACAG | 3′ Intron 3 | 64219 | 64236 | 98 |
| 580337 | GAGGCAGCAAAGGGAGAG | 3′ Intron 3 | 64224 | 64241 | 99 |
| 580338 | GGTCCGAGGCAGCAAAGG | 3′ Intron 3 | 64229 | 64246 | 100 |
| 580339 | CGGCTGGTCCGAGGCAGC | Intron 3/Exon 4 | 64234 | 64251 | 101 |
| 580340 | TTCTTCGGCTGGTCCGAG | Intron 3/Exon 4 | 64239 | 64256 | 102 |
| 580341 | CCTGCTTCTTCGGCTGGT | Intron 3/Exon 4 | 64244 | 64261 | 103 |
| 580342 | CGATGCCTGCTTCTTCGG | Exon 4 | 64249 | 64266 | 104 |
| 580343 | GTCTCCGATGCCTGCTTC | Exon 4 | 64254 | 64271 | 105 |
| 580344 | TGGTCCTCCTGGTTCGGG | Exon 4 | 64274 | 64291 | 106 |
| 580345 | CGGCTTGGTCCTCCTGGT | Exon 4 | 64279 | 64296 | 107 |
| 580346 | CCCAGCGGCTTGGTCCTC | Exon 4 | 64284 | 64301 | 108 |
| 580347 | ACATGCCCAGCGGCTTGG | Exon 4 | 64289 | 64306 | 109 |
| 580348 | GAGTCACATGCCCAGCGG | Exon 4 | 64294 | 64311 | 110 |
| 580349 | ACCTTGAGTCACATGCCC | Exon 4/Intron 4 | 64299 | 64316 | 111 |
| 580350 | CACTGACCTTGAGTCACA | Exon 4/Intron 4 | 64304 | 64321 | 112 |
| 580351 | TGGGCCACTGACCTTGAG | Exon 4/Intron 4 | 64309 | 64326 | 113 |
| 580352 | CGATTTGGGCCACTGACC | Exon 4/Intron 4 | 64314 | 64331 | 114 |
| 580353 | AAAGTCGATTTGGGCCAC | 5′ Intron 4 | 64319 | 64336 | 115 |
| 580354 | GTCCCAAAGTCGATTTGG | 5′ Intron 4 | 64324 | 64341 | 116 |
| 580355 | TCTGAGTCCCAAAGTCGA | 5′ Intron 4 | 64329 | 64346 | 117 |
| 580356 | CCCAATCTGAGTCCCAAA | 5′ Intron 4 | 64334 | 64351 | 118 |
| 580357 | CTCCTCCCAATCTGAGTC | 5′ Intron 4 | 64339 | 64356 | 119 |
| 580358 | GTGCACGTGATGCCCCGT | 3′ Intron 4 | 71711 | 71728 | 120 |
| 580359 | AGAGAGTGCACGTGATGC | 3′ Intron 4 | 71716 | 71733 | 121 |
| 580360 | AAAATAGAGAGTGCACGT | 3′ Intron 4 | 71721 | 71738 | 122 |
| 580361 | AATATAAAATAGAGAGTG | 3′ Intron 4 | 71726 | 71743 | 123 |
| 580362 | GGTAAAATATAAAATAGA | 3′ Intron 4 | 71731 | 71748 | 124 |
| 580363 | GAGCTGGTAAAATATAAA | Intron 4/Exon 5 | 71736 | 71753 | 125 |
| 580364 | CACACGAGCTGGTAAAAT | Intron 4/Exon 5 | 71741 | 71758 | 126 |
| 580365 | CTGGCCACACGAGCTGGT | Intron 4/Exon 5 | 71746 | 71763 | 127 |
| 580366 | CTTTGCTGGCCACACGAG | Exon 5 | 71751 | 71768 | 128 |
| 580367 | CCTGTCTTTGCTGGCCAC | Exon 5 | 71756 | 71773 | 129 |
| 580368 | CCTGTCCTGTCTTTGCTG | Exon 5 | 71761 | 71778 | 130 |
| 580369 | CATTTCCTGTCCTGTCTT | Exon 5 | 71766 | 71783 | 131 |
| 580370 | CTCGTCATTTCCTGTCCT | Exon 5 | 71771 | 71788 | 132 |
| 580371 | TTCTTCTCGTCATTTCCT | Exon 5 | 71776 | 71793 | 133 |
| 580372 | TGGCTTTCTTCTCGTCAT | Exon 5 | 71781 | 71798 | 134 |
| 580373 | TACCTTGGCTTTCTTCTC | Exon 5/Intron 5-7 | 71786 | 71803 | 135 |
| 580374 | TAGCTTACCTTGGCTTTC | Exon 5/Intron 5-7 | 71791 | 71808 | 136 |
| 580375 | GTCAGTAGCTTACCTTGG | Exon 5/Intron 5-7 | 71796 | 71813 | 137 |
| 580376 | GGCGGGTCAGTAGCTTAC | 5′ Intron 5-7 | 71801 | 71818 | 138 |
| 580377 | GGACCGGCGGGTCAGTAG | 5′ Intron 5-7 | 71806 | 71823 | 139 |
| 580378 | TTCTAGGACCGGCGGGTC | 5′ Intron 5-7 | 71811 | 71828 | 140 |
| 580379 | GAGCCTTCTAGGACCGGC | 5′ Intron 5-7 | 71816 | 71833 | 141 |
| 580380 | AAGCAGAGCCTTCTAGGA | 5′ Intron 5-7 | 71821 | 71838 | 142 |
| 580381 | GCAGGAAGCAGAGCCTTC | 5′ Intron 5-7 | 71826 | 71843 | 143 |
| 580382 | AGGTTTTCACCACTGGGA | 3′ Intron 5-7 | 75976 | 75993 | 144 |
| 580383 | GTGGAAGGTTTTCACCAC | 3′ Intron 5-7 | 75981 | 75998 | 145 |
| 580384 | TCAGGGTGGAAGGTTTTC | 3′ Intron 5-7 | 75986 | 76003 | 146 |
| 580385 | CCGAATCAGGGTGGAAGG | 3′ Intron 5-7 | 75991 | 76008 | 147 |
| 580386 | GTAAACCGAATCAGGGTG | 3′ Intron 5-7 | 75996 | 76013 | 148 |
| 580387 | GCCCTGTAAACCGAATCA | Intron 5-7/Exon 7 | 76001 | 76018 | 149 |
| 580388 | TCAGCGCCCTGTAAACCG | Intron 5-7/Exon 7 | 76006 | 76023 | 150 |
| 580389 | TGCCATCAGCGCCCTGTA | Intron 5-7/Exon 7 | 76011 | 76028 | 151 |
| 580390 | GGTTTTGCCATCAGCGCC | Exon 7 | 76016 | 76033 | 152 |
| 580391 | GCCCCGGTTTTGCCATCA | Exon 7 | 76021 | 76038 | 153 |
| 580392 | TCTTCGCCCCGGTTTTGC | Exon 7 | 76026 | 76043 | 154 |
| 580393 | GGCGATCTTCGCCCCGGT | Exon 7 | 76031 | 76048 | 155 |
| 580394 | GGTGTGGCGATCTTCGCC | Exon 7 | 76036 | 76053 | 156 |
| 580395 | CCCGAGGTGTGGCGATCT | Exon 7 | 76041 | 76058 | 157 |
| 580396 | TGCTCCCCGAGGTGTGGC | Exon 7 | 76046 | 76063 | 158 |
| 580397 | GAGGCTGCTCCCCGAGGT | Exon 7 | 76051 | 76068 | 159 |
| 580398 | CCGGAGAGGCTGCTCCCC | Exon 7 | 76056 | 76073 | 160 |
| 580399 | CTGGGCCGGAGAGGCTGC | Exon 7 | 76061 | 76078 | 161 |
| 580400 | CCCTTCTGGGCCGGAGAG | Exon 7 | 76066 | 76083 | 162 |
| 580401 | ACGTGCCCTTCTGGGCCG | Exon 7 | 76071 | 76088 | 163 |
| 580402 | GTTGGACGTGCCCTTCTG | Exon 7 | 76076 | 76093 | 164 |
| 580403 | GTGGCGTTGGACGTGCCC | Exon 7 | 76081 | 76098 | 165 |
| 580404 | TCCTGGTGGCGTTGGACG | Exon 7 | 76086 | 76103 | 166 |
| 580405 | CGGGATCCTGGTGGCGTT | Exon 7 | 76091 | 76108 | 167 |
| 580406 | TTGGCCGGGATCCTGGTG | Exon 7 | 76096 | 76113 | 168 |
| 580407 | TGGTCTTGGCCGGGATCC | Exon 7 | 76101 | 76118 | 169 |
| 580408 | GGGCGTGGTCTTGGCCGG | Exon 7 | 76106 | 76123 | 170 |
| 580409 | GGGCTGGGCGTGGTCTTG | Exon 7 | 76111 | 76128 | 171 |
| 580410 | TCTTAGGGCTGGGCGTGG | Exon 7 | 76116 | 76133 | 172 |
| 580411 | AGGAGTCTTAGGGCTGGG | Exon 7 | 76121 | 76138 | 173 |
| 580412 | CCTGGAGGAGTCTTAGGG | Exon 7 | 76126 | 76143 | 174 |
| 580413 | CTGACCCTGGAGGAGTCT | Exon 7 | 76131 | 76148 | 175 |
| 580414 | CTCACCTGACCCTGGAGG | Exon 7/Intron 7-9 | 76136 | 76153 | 176 |
| 580415 | GTAGTCTCACCTGACCCT | Exon 7/Intron 7-9 | 76141 | 76158 | 177 |
| 580416 | AGAGAGTAGTCTCACCTG | Exon 7/Intron 7-9 | 76146 | 76163 | 178 |
| 580417 | ACTCCAGAGAGTAGTCTC | 5′ Intron 7-9 | 76151 | 76168 | 179 |
| 580418 | TTAAAACTCCAGAGAGTA | 5′ Intron 7-9 | 76156 | 76173 | 180 |
| 580419 | CTGGATTAAAACTCCAGA | 5′ Intron 7-9 | 76161 | 76178 | 181 |
| 580420 | AGCTTCTGGATTAAAACT | 5′ Intron 7-9 | 76166 | 76183 | 182 |
| 580421 | CTGGAAGCTTCTGGATTA | 5′ Intron 7-9 | 76171 | 76188 | 183 |
| 580422 | GGGACTGAGGCCACGTGG | 3′ Intron 7-9 | 79619 | 79636 | 184 |
| 580423 | AGGAAGGGACTGAGGCCA | 3′ Intron 7-9 | 79624 | 79641 | 185 |
| 580424 | GAGAGAGGAAGGGACTGA | 3′ Intron 7-9 | 79629 | 79646 | 186 |
| 580425 | GTCGGGAGAGAGGAAGGG | 3′ Intron 7-9 | 79634 | 79651 | 187 |
| 580426 | GGAAAGTCGGGAGAGAGG | 3′ Intron 7-9 | 79639 | 79656 | 188 |
| 580427 | CACCTGGAAAGTCGGGAG | Intron 7-9/Exon 9 | 79644 | 79661 | 189 |
| 580428 | TGGTTCACCTGGAAAGTC | Intron 7-9/Exon 9 | 79649 | 79666 | 190 |
| 580429 | TTTGGTGGTTCACCTGGA | Intron 7-9/Exon 9 | 79654 | 79671 | 191 |
| 580430 | CGGATTTTGGTGGTTCAC | Exon 9 | 79659 | 79676 | 192 |
| 580431 | TTCTCCGGATTTTGGTGG | Exon 9 | 79664 | 79681 | 193 |
| 580432 | CTTCGTTCTCCGGATTTT | Exon 9 | 79669 | 79686 | 194 |
| 580433 | AGCCGCTTCGTTCTCCGG | Exon 9 | 79674 | 79691 | 195 |
| 580434 | GCTGTAGCCGCTTCGTTC | Exon 9 | 79679 | 79696 | 196 |
| 580435 | GGGCTGCTGTAGCCGCTT | Exon 9 | 79684 | 79701 | 197 |
| 580436 | CAGGCGTTCCGGGAGAGC | Exon 9 | 79704 | 79721 | 198 |
| 580437 | ACTGCCAGGCGTTCCGGG | Exon 9 | 79709 | 79726 | 199 |
| 580438 | GAGCGACTGCCAGGCGTT | Exon 9 | 79714 | 79731 | 200 |
| 580439 | TGCGCGAGCGACTGCCAG | Exon 9 | 79719 | 79736 | 201 |
| 580440 | CGGTGTTGGTAGGGATGG | Exon 9 | 79739 | 79756 | 202 |
| 580441 | GTGGGCGGTGTTGGTAGG | Exon 9 | 79744 | 79761 | 203 |
| 580442 | CCCGGGTGGGCGGTGTTG | Exon 9 | 79749 | 79766 | 204 |
| 580443 | GGGCTCCCGGGTGGGCGG | Exon 9 | 79754 | 79771 | 205 |
| 580444 | TTCTTGGGCTCCCGGGTG | Exon 9 | 79759 | 79776 | 206 |
| 580445 | CCACCTTCTTGGGCTCCC | Exon 9 | 79764 | 79781 | 207 |
| 580446 | CACTGCCACCTTCTTGGG | Exon 9 | 79769 | 79786 | 208 |
| 580447 | CGGACCACTGCCACCTTC | Exon 9 | 79774 | 79791 | 209 |
| 580448 | GAGTGCGGACCACTGCCA | Exon 9 | 79779 | 79796 | 210 |
| 580449 | ATGGTGACTTAGGGGGAG | Exon 9 | 79794 | 79811 | 211 |
| 580450 | AGCTGATGGTGACTTAGG | Exon 9 | 79799 | 79816 | 212 |
| 580451 | TTACTAGCTGATGGTGAC | Exon 9 | 79804 | 79821 | 213 |
| 580452 | GGCTCTTACTAGCTGATG | Exon 9 | 79809 | 79826 | 214 |
| 580453 | CAGGCGGCTCTTACTAGC | Exon 9 | 79814 | 79831 | 215 |
| 580454 | GTCTGCAGGCGGCTCTTA | Exon 9 | 79819 | 79836 | 216 |
| 580455 | GGGCAGTCTGCAGGCGGC | Exon 9 | 79824 | 79841 | 217 |
| 580456 | TAGGTCTGGCATGGGCAC | Exon 9 | 79844 | 79861 | 218 |
| 580457 | TTCTTTAGGTCTGGCATG | Exon 9 | 79849 | 79866 | 219 |
| 580458 | TGACATTCTTTAGGTCTG | Exon 9 | 79854 | 79871 | 220 |
| 580459 | CGACCTGACATTCTTTAG | Exon 9 | 79859 | 79876 | 221 |
| 580460 | ATCTTCGACCTGACATTC | Exon 9 | 79864 | 79881 | 222 |
| 580461 | AGCCAATCTTCGACCTGA | Exon 9 | 79869 | 79886 | 223 |
| 580462 | AGTAGAGCCAATCTTCGA | Exon 9 | 79874 | 79891 | 224 |
| 580463 | TTCTCAGTAGAGCCAATC | Exon 9 | 79879 | 79896 | 225 |
| 580464 | TCAGGTTCTCAGTAGAGC | Exon 9 | 79884 | 79901 | 226 |
| 580465 | GTGCTTCAGGTTCTCAGT | Exon 9 | 79889 | 79906 | 227 |
| 580466 | GGCTGGTGCTTCAGGTTC | Exon 9 | 79894 | 79911 | 228 |
| 580467 | CTCCTGGCTGGTGCTTCA | Exon 9 | 79899 | 79916 | 229 |
| 580468 | GCCACCTCCTGGCTGGTG | Exon 9 | 79904 | 79921 | 230 |
| 580469 | ACCTTGCCACCTCCTGGC | Exon 9/Intron 9 | 79909 | 79926 | 231 |
| 580470 | CCCTTACCTTGCCACCTC | Exon 9/Intron 9 | 79914 | 79931 | 232 |
| 580471 | CCACACCCTTACCTTGCC | Exon 9/Intron 9 | 79919 | 79936 | 233 |
| 580472 | ACCAGCCACACCCTTACC | Exon 9/Intron 9 | 79924 | 79941 | 234 |
| 580473 | AGAAGACCAGCCACACCC | 5′ Intron 9 | 79929 | 79946 | 235 |
| 580474 | TTCCCAGAAGACCAGCCA | 5′ Intron 9 | 79934 | 79951 | 236 |
| 580475 | CTGGCTTCCCAGAAGACC | 5′ Intron 9 | 79939 | 79956 | 237 |
| 580476 | GGAGTCTGGCTTCCCAGA | 5′ Intron 9 | 79944 | 79961 | 238 |
| 580477 | AGGTGGGAGTCTGGCTTC | 5′ Intron 9 | 79949 | 79966 | 239 |
| 580478 | GAGAGAGAGAATAAAGGG | 3′ Intron 10 | 90701 | 90718 | 240 |
| 580479 | TGGGCGAGAGAGAGAATA | 3′ Intron 10 | 90706 | 90723 | 241 |
| 580480 | GAGGATGGGCGAGAGAGA | 3′ Intron 10 | 90711 | 90728 | 242 |
| 580481 | TCAAAGAGGATGGGCGAG | 3′ Intron 10 | 90716 | 90733 | 243 |
| 580482 | GCAGGTCAAAGAGGATGG | 3′ Intron 10 | 90721 | 90738 | 244 |
| 580483 | CACCTGCAGGTCAAAGAG | Intron 10/Exon 11 | 90726 | 90743 | 245 |
| 580484 | ATTTGCACCTGCAGGTCA | Intron 10/Exon 11 | 90731 | 90748 | 246 |
| 580485 | AGACTATTTGCACCTGCA | Intron 10/Exon 11 | 90736 | 90753 | 247 |
| 580486 | CTTGTAGACTATTTGCAC | Exon 11 | 90741 | 90758 | 248 |
| 580487 | ACCGGCTTGTAGACTATT | Exon 11 | 90746 | 90763 | 249 |
| 580488 | GGTCCACCGGCTTGTAGA | Exon 11 | 90751 | 90768 | 250 |
| 580489 | GCTCAGGTCCACCGGCTT | Exon 11 | 90756 | 90773 | 251 |
| 580490 | ACTTTGCTCAGGTCCACC | Exon 11 | 90761 | 90778 | 252 |
| 580491 | AGGTCACTTTGCTCAGGT | Exon 11 | 90766 | 90783 | 253 |
| 580492 | CTTGGAGGTCACTTTGCT | Exon 11 | 90771 | 90788 | 254 |
| 580493 | CCACACTTGGAGGTCACT | Exon 11 | 90776 | 90793 | 255 |
| 580494 | ACGAGCCACACTTGGAGG | Exon 11 | 90781 | 90798 | 256 |
| 580495 | CCCTAACGAGCCACACTT | Exon 11 | 90786 | 90803 | 257 |
| 580496 | ATGTTCCCTAACGAGCCA | Exon 11 | 90791 | 90808 | 258 |
| 580497 | GATGGATGTTCCCTAACG | Exon 11 | 90796 | 90813 | 259 |
| 580498 | CTTGTGATGGATGTTCCC | Exon 11 | 90801 | 90818 | 260 |
| 580499 | CCTGGCTTGTGATGGATG | Exon 11/Intron 11 | 90806 | 90823 | 261 |
| 580500 | TGCTACCTGGCTTGTGAT | Exon 11/Intron 11 | 90811 | 90828 | 262 |
| 580501 | TCAAGTGCTACCTGGCTT | Exon 11/Intron 11 | 90816 | 90833 | 263 |
| 580502 | CTTCCTCAAGTGCTACCT | Exon 11/Intron 11 | 90821 | 90838 | 264 |
| 580503 | CCTGTCTTCCTCAAGTGC | 5′ Intron 11 | 90826 | 90843 | 265 |
| 580504 | CAAAGCCTGTCTTCCTCA | 5′ Intron 11 | 90831 | 90848 | 266 |
| 580505 | TGTCCCAAAGCCTGTCTT | 5′ Intron 11 | 90836 | 90853 | 267 |
| 580506 | ACTCCTGTCCCAAAGCCT | 5′ Intron 11 | 90841 | 90858 | 268 |
| 580507 | CCAGCACTCCTGTCCCAA | 5′ Intron 11 | 90846 | 90863 | 269 |
| 580508 | CTCTTGCCCTAGTCTGTG | 3′ Intron 11 | 91778 | 91795 | 270 |
| 580509 | TGAGCCTCTTGCCCTAGT | 3′ Intron 11 | 91783 | 91800 | 271 |
| 580510 | CCACATGAGCCTCTTGCC | 3′ Intron 11 | 91788 | 91805 | 272 |
| 580511 | ACAACCCACATGAGCCTC | 3′ Intron 11 | 91793 | 91810 | 273 |
| 580512 | GGAACACAACCCACATGA | 3′ Intron 11 | 91798 | 91815 | 274 |
| 580513 | CTCCTGGAACACAACCCA | Intron 11/Exon 12 | 91803 | 91820 | 275 |
| 580514 | GCCACCTCCTGGAACACA | Intron 11/Exon 12 | 91808 | 91825 | 276 |
| 580515 | ACCTGGCCACCTCCTGGA | Intron 11/Exon 12 | 91813 | 91830 | 277 |
| 580516 | CTTCCACCTGGCCACCTC | Exon 12 | 91818 | 91835 | 278 |
| 580517 | TTTTACTTCCACCTGGCC | Exon 12 | 91823 | 91840 | 279 |
| 580518 | TCTGATTTTACTTCCACC | Exon 12 | 91828 | 91845 | 280 |
| 580519 | GCTTCTCTGATTTTACTT | Exon 12 | 91833 | 91850 | 281 |
| 580520 | GTCCAGCTTCTCTGATTT | Exon 12 | 91838 | 91855 | 282 |
| 580521 | TTGAAGTCCAGCTTCTCT | Exon 12 | 91843 | 91860 | 283 |
| 580522 | TGTCCTTGAAGTCCAGCT | Exon 12 | 91848 | 91865 | 284 |
| 580523 | GACTCTGTCCTTGAAGTC | Exon 12 | 91853 | 91870 | 285 |
| 580524 | GACTGGACTCTGTCCTTG | Exon 12 | 91858 | 91875 | 286 |
| 580525 | TCTTCGACTGGACTCTGT | Exon 12 | 91863 | 91880 | 287 |
| 580526 | GCCAATCTTCGACTGGAC | Exon 12 | 91868 | 91885 | 288 |
| 580527 | AAGGAGCCAATCTTCGAC | Exon 12 | 91873 | 91890 | 289 |
| 580528 | TATCCAAGGAGCCAATCT | Exon 12 | 91878 | 91895 | 290 |
| 580529 | GATATTATCCAAGGAGCC | Exon 12 | 91883 | 91900 | 291 |
| 580530 | TGGGTGATATTATCCAAG | Exon 12 | 91888 | 91905 | 292 |
| 580531 | GGACGTGGGTGATATTAT | Exon 12 | 91893 | 91910 | 293 |
| 580532 | TCCAGGGACGTGGGTGAT | Exon 12 | 91898 | 91915 | 294 |
| 580533 | CCTCCTCCAGGGACGTGG | Exon 12 | 91903 | 91920 | 295 |
| 580534 | TATTCCCTCCTCCAGGGA | Exon 12 | 91908 | 91925 | 296 |
| 580535 | CTTCTTATTCCCTCCTCC | Exon 12 | 91913 | 91930 | 297 |
| 580536 | CTTACCTTCTTATTCCCT | Exon 12/Intron 12 | 91918 | 91935 | 298 |
| 580537 | ACCCCCTTACCTTCTTAT | Exon 12/Intron 12 | 91923 | 91940 | 299 |
| 580538 | ATTTGACCCCCTTACCTT | Exon 12/Intron 12 | 91928 | 91945 | 300 |
| 580539 | CTCCCATTTGACCCCCTT | 5′ Intron 12 | 91933 | 91950 | 301 |
| 580540 | ATGACCTCCCATTTGACC | 5′ Intron 12 | 91938 | 91955 | 302 |
| 580541 | CCCGTATGACCTCCCATT | 5′ Intron 12 | 91943 | 91960 | 303 |
| 580542 | TTATCCCCGTATGACCTC | 5′ Intron 12 | 91948 | 91965 | 304 |
| 580543 | CCCTCTTATCCCCGTATG | 5′ Intron 12 | 91953 | 91970 | 305 |
| 580544 | TTAATGATAAGGTTCTAT | 3′ Intron 12 | 97322 | 97339 | 306 |
| 580545 | TGAGATTAATGATAAGGT | 3′ Intron 12 | 97327 | 97344 | 307 |
| 580546 | GAGAGTGAGATTAATGAT | 3′ Intron 12 | 97332 | 97349 | 308 |
| 580547 | ATGTAGAGAGTGAGATTA | 3′ Intron 12 | 97337 | 97354 | 309 |
| 580548 | GCAAGATGTAGAGAGTGA | 3′ Intron 12 | 97342 | 97359 | 310 |
| 580549 | AATCTGCAAGATGTAGAG | Intron 12/Exon 13 | 97347 | 97364 | 311 |
| 580550 | GTTTCAATCTGCAAGATG | Intron 12/Exon 13 | 97352 | 97369 | 312 |
| 580551 | TGTGGGTTTCAATCTGCA | Intron 12/Exon 13 | 97357 | 97374 | 313 |
| 580552 | CAGCTTGTGGGTTTCAAT | Exon 13 | 97362 | 97379 | 314 |
| 580553 | AAGGTCAGCTTGTGGGTT | Exon 13 | 97367 | 97384 | 315 |
| 580554 | CCCTGAAGGTCAGCTTGT | Exon 13 | 97372 | 97389 | 316 |
| 580555 | ATTCTCCCTGAAGGTCAG | Exon 13 | 97377 | 97394 | 317 |
| 580556 | TTGGCATTCTCCCTGAAG | Exon 13 | 97382 | 97399 | 318 |
| 580557 | TGGCTTTGGCATTCTCCC | Exon 13 | 97387 | 97404 | 319 |
| 580558 | TGTCTTGGCTTTGGCATT | Exon 13 | 97392 | 97409 | 320 |
| 580559 | TGGTCTGTCTTGGCTTTG | Exon 13 | 97397 | 97414 | 321 |
| 580560 | CTCCATGGTCTGTCTTGG | Exon 13 | 97402 | 97419 | 322 |
| 580561 | TTCTGCTCCATGGTCTGT | Exon 13 | 97407 | 97424 | 323 |
| 580562 | ACAATTTCTGCTCCATGG | Exon 13 | 97412 | 97429 | 324 |

TABLE 2
Uniform 2′-MOE modified
oligonucleotides targeted to tau

| | | | SEQ | SEQ | |
|---|---|---|---|---|---|
| | | | ID: 12 | ID: 12 | |
| Isis | | | Start | Stop | SEQ |
| No. | ASO seq. | Target Region | Site | Site | ID NO |
| 607479 | ATCGACAGAAGGCGAGGA | Exon-1 | 974 | 991 | 325 |
| 607480 | AATCGACAGAAGGCGAGG | Exon-1 | 975 | 992 | 326 |
| 607481 | TAATCGACAGAAGGCGAG | Exon-1 | 976 | 993 | 327 |
| 607482 | ATAATCGACAGAAGGCGA | Exon-1 | 977 | 994 | 328 |
| 607483 | TGATAATCGACAGAAGGC | Exon-1 | 979 | 996 | 329 |
| 607484 | CTGATAATCGACAGAAGG | Exon-1 | 980 | 997 | 330 |
| 607485 | CCTGATAATCGACAGAAG | Exon-1/Intron-1 | 981 | 998 | 331 |
| 607486 | ACCTGATAATCGACAGAA | Exon-1/Intron-1 | 982 | 999 | 332 |
| 607487 | TTACCTGATAATCGACAG | Exon-1/Intron-1 | 984 | 1001 | 333 |
| 607488 | CTTACCTGATAATCGACA | Exon-1/Intron-1 | 985 | 1002 | 334 |
| 607489 | GCTTACCTGATAATCGAC | Exon-1/Intron-1 | 986 | 1003 | 335 |
| 607490 | CGCTTACCTGATAATCGA | Exon-1/Intron-1 | 987 | 1004 | 336 |
| 607491 | GGCGCTTACCTGATAATC | Exon-1/Intron-1 | 989 | 1006 | 337 |
| 607492 | CGGCGCTTACCTGATAAT | Exon-1/Intron-1 | 990 | 1007 | 338 |
| 607493 | GCGGCGCTTACCTGATAA | Exon-1/Intron-1 | 991 | 1008 | 339 |
| 607494 | TGCGGCGCTTACCTGATA | Exon-1/Intron-1 | 992 | 1009 | 340 |
| 607495 | GCTGCGGCGCTTACCTGA | Exon-1/Intron-1 | 994 | 1011 | 341 |
| 607496 | AGCTGCGGCGCTTACCTG | Exon-1/Intron-1 | 995 | 1012 | 342 |
| 607497 | GAGCTGCGGCGCTTACCT | Exon-1/Intron-1 | 996 | 1013 | 343 |
| 607498 | AGAGCTGCGGCGCTTACC | Exon-1/Intron-1 | 997 | 1014 | 344 |

### Example 2: Effects of Uniform 2′-MOE Modified Oligonucleotides on Tau Expression

Modified oligonucleotides were transfected in B16-F10 cells (7500 cells/well) using 2 mg/ml cytofectin in OPTI-MEM for 4 hours and then lysed 24 hours later for RNA isolation. The Modified oligonucleotides were transfected at 50 nM concentration. RNA isolation was performed using Invitrogen RNAeasy 96 well columns with Dnase I digestion on the column Total Tau mRNA was analyzed via qRT-PCR reaction with primer probe set RTS3061 (forward sequence 5′-CGGCACCTCAGCAATGTGT-3′, SEQ ID NO: 13; reverse sequence 5′-TGTGGCAAGCTGTGGTGAGT-3′, SEQ ID NO: 14; probe sequence 5′-TTCCACGGGCAGCATCGACATGX-3′, SEQ ID NO: 15). RNA was normalized to Ribogreen. ISIS No. 424880 is a 5-10-5 MOE gapmer having the sequence: ATCACTGATTTTGAAGTCCC (SEQ ID NO: 16); wherein each internucleoside linkage throughout is a phosphorothioate internucleoside linkages (P═S) and all cytosine residues are 5-methylcytosines. Results are presented as percent reduction of RNA expression, relative to untreated control levels and is denoted as “% Reduction” in the tables below. As illustrated in the tables below, modified oligonucleotides targeted to tau, including tau exon/intron junctions, tau introns, tau exons, and tau splice modulation sites reduce tau mRNA. Modified oligonucleotides that modulate splicing resulting in a frameshift and introduction of a premature stop codon likely work, at least in part, to reduce Tau mRNA via nonsense mediated decay. The tolerability of the modified oligonucleotides can be determined using methods, such as those described in WO2010/148249. Like the fully modified 2′-MOE oligonucleotides described in WO2010/148249, the fully modified 2′-MOE oligonucleotides described herein are expected to be tolerable when administered to the CNS.

TABLE 3
Effect of modified oligonucleotides on Tau expression

| Isis # | % Reduction |
|---|---|
| 580335 | 13 |
| 580336 | 0 |
| 580337 | 17 |
| 580338 | 16 |
| 580339 | 0 |
| 580340 | 0 |
| 580341 | 0 |
| 580342 | 0 |
| 580343 | 8 |
| 580344 | 0 |
| 580345 | 11 |
| 580346 | 5 |
| 580347 | 1 |
| 580348 | 2 |
| 580349 | 0 |
| 580350 | 0 |
| 580351 | 2 |
| 580352 | 11 |
| 580353 | 0 |
| 580354 | 5 |
| 580355 | 0 |
| 580356 | 0 |
| 580357 | 0 |
| 580358 | 0 |
| 580359 | 6 |
| 580360 | 0 |
| 580361 | 22 |
| 580362 | 0 |
| 580363 | 0 |
| 580364 | 0 |
| 580365 | 0 |
| 580366 | 10 |
| 580367 | 17 |
| 580368 | 23 |
| 580369 | 14 |
| 580370 | 17 |
| 580371 | 16 |
| 580372 | 8 |
| 580373 | 8 |
| 580374 | 11 |
| 580375 | 29 |
| 580376 | 9 |
| 580377 | 3 |
| 580378 | 0 |
| 580379 | 0 |
| 580380 | 0 |
| 580381 | 0 |
| 580382 | 12 |
| 580383 | 10 |
| 580384 | 0 |
| 580385 | 32 |
| 580386 | 23 |
| 580387 | 8 |
| 580388 | 13 |
| 580389 | 33 |
| 580390 | 41 |
| 580391 | 10 |
| 580392 | 10 |
| 580393 | 0 |
| 580394 | 19 |
| 580395 | 0 |
| 580396 | 0 |
| 580397 | 14 |
| 580398 | 0 |
| 580399 | 12 |
| 580400 | 0 |
| 580401 | 6 |
| 580402 | 0 |
| 580403 | 0 |
| 580404 | 0 |
| 580405 | 0 |
| 580406 | 0 |
| 580407 | 1 |
| 580408 | 4 |
| 580409 | 8 |
| 580410 | 21 |
| 580411 | 0 |
| 580412 | 7 |
| 580413 | 3 |
| 580414 | 13 |
| 424880 | 84 |
| No Oligo | 0 |

TABLE 4
Effect of modified oligonucleotides on Tau expression

| Isis # | % Reduction |
|---|---|
| 580495 | 0 |
| 580496 | 6 |
| 580497 | 0 |
| 580498 | 20 |
| 580499 | 36 |
| 580500 | 12 |
| 580501 | 3 |
| 580502 | 0 |
| 580503 | 13 |
| 580504 | 7 |
| 580505 | 0 |
| 580506 | 1 |
| 580507 | 8 |
| 580508 | 27 |
| 580509 | 39 |
| 580510 | 19 |
| 580511 | 0 |
| 580512 | 21 |
| 580513 | 8 |
| 580514 | 9 |
| 580515 | 0 |
| 580516 | 15 |
| 580517 | 23 |
| 580518 | 21 |
| 580519 | 17 |
| 580520 | 16 |
| 580521 | 12 |
| 580522 | 18 |
| 580523 | 0 |
| 580524 | 0 |
| 580525 | 12 |
| 580526 | 1 |
| 580527 | 16 |
| 580528 | 25 |
| 580529 | 8 |
| 580530 | 14 |
| 580531 | 3 |
| 580532 | 0 |
| 580533 | 6 |
| 580534 | 0 |
| 580535 | 32 |
| 580536 | 0 |
| 580537 | 8 |
| 580538 | 0 |
| 580539 | 23 |
| 580540 | 12 |
| 580541 | 12 |
| 580542 | 7 |
| 580543 | 3 |
| 580544 | 3 |
| 580545 | 10 |
| 580546 | 0 |
| 580547 | 16 |
| 580548 | 0 |
| 580549 | 0 |
| 580550 | 19 |
| 580551 | 4 |
| 580552 | 7 |
| 580553 | 5 |
| 580554 | 1 |
| 580555 | 19 |
| 580556 | 0 |
| 580557 | 10 |
| 580558 | 8 |
| 580559 | 0 |
| 580560 | 0 |
| 580561 | 15 |
| 580562 | 11 |
| 424880 | 80 |
| No Oligo | 0 |

TABLE 5
Effect of modified oligonucleotides on Tau expression

| ISIS# | % Reduction |
|---|---|
| 580255 | 0 |
| 580256 | 0 |
| 580257 | 0 |
| 580258 | 4 |
| 580259 | 4 |
| 580260 | 9 |
| 580261 | 0 |
| 580262 | 14 |
| 580263 | 16 |
| 580264 | 18 |
| 580265 | 46 |
| 580266 | 50 |
| 580267 | 16 |
| 580268 | 56 |
| 580269 | 20 |
| 580270 | 0 |
| 580271 | 0 |
| 580272 | 13 |
| 580273 | 0 |
| 580274 | 16 |
| 580275 | 16 |
| 580276 | 0 |
| 580277 | 2 |
| 580278 | 10 |
| 580279 | 3 |
| 580280 | 8 |
| 580281 | 25 |
| 580282 | 26 |
| 580283 | 13 |
| 580284 | 11 |
| 580285 | 19 |
| 580286 | 12 |
| 580287 | 11 |
| 580288 | 8 |
| 580289 | 26 |
| 580290 | 21 |
| 580291 | 23 |
| 580292 | 28 |
| 580293 | 26 |
| 580294 | 23 |
| 580295 | 0 |
| 580296 | 19 |
| 580297 | 15 |
| 580298 | 0 |
| 580299 | 29 |
| 580300 | 33 |
| 580301 | 16 |
| 580302 | 42 |
| 580303 | 34 |
| 580304 | 19 |
| 580305 | 20 |
| 580306 | 0 |
| 580307 | 0 |
| 580308 | 15 |
| 580309 | 14 |
| 580310 | 12 |
| 580311 | 2 |
| 580312 | 10 |
| 580313 | 8 |
| 580314 | 0 |
| 580315 | 5 |
| 580316 | 3 |
| 580317 | 0 |
| 580318 | 0 |
| 580319 | 0 |
| 580320 | 0 |
| 580321 | 8 |
| 580322 | 7 |
| 580323 | 0 |
| 580324 | 0 |
| 580325 | 3 |
| 580326 | 13 |
| 580327 | 6 |
| 580328 | 8 |
| 580329 | 6 |
| 580330 | 0 |
| 580331 | 0 |
| 580332 | 0 |
| 580333 | 11 |
| 580334 | 3 |
| 424880 | 82 |
| No Oligo | 0 |

TABLE 6
Effect of modified oligonucleotides on Tau expression

| Isis # | % Reduction |
|---|---|
| 580415 | 21 |
| 580416 | 20 |
| 580417 | 17 |
| 580418 | 8 |
| 580419 | 14 |
| 580420 | 5 |
| 580421 | 4 |
| 580422 | 12 |
| 580423 | 15 |
| 580424 | 7 |
| 580425 | 21 |
| 580426 | 19 |
| 580427 | 5 |
| 580428 | 14 |
| 580429 | 0 |
| 580430 | 38 |
| 580431 | 26 |
| 580432 | 17 |
| 580433 | 3 |
| 580434 | 15 |
| 580435 | 0 |
| 580436 | 0 |
| 580437 | 0 |
| 580438 | 23 |
| 580439 | 7 |
| 580440 | 12 |
| 580441 | 3 |
| 580442 | 0 |
| 580443 | 12 |
| 580444 | 4 |
| 580445 | 8 |
| 580446 | 0 |
| 580447 | 16 |
| 580448 | 11 |
| 580449 | 13 |
| 580450 | 19 |
| 580451 | 15 |
| 580452 | 15 |
| 580453 | 24 |
| 580454 | 8 |
| 580455 | 2 |
| 580456 | 22 |
| 580457 | 19 |
| 580458 | 23 |
| 580459 | 21 |
| 580460 | 20 |
| 580461 | 19 |
| 580462 | 34 |
| 580463 | 36 |
| 580464 | 13 |
| 580465 | 33 |
| 580466 | 15 |
| 580467 | 22 |
| 580468 | 0 |
| 580469 | 20 |
| 580470 | 7 |
| 580471 | 4 |
| 580472 | 16 |
| 580473 | 4 |
| 580474 | 0 |
| 580475 | 11 |
| 580476 | 0 |
| 580477 | 1 |
| 580478 | 7 |
| 580479 | 12 |
| 580480 | 16 |
| 580481 | 9 |
| 580482 | 31 |
| 580483 | 0 |
| 580484 | 0 |
| 580485 | 42 |
| 580486 | 40 |
| 580487 | 41 |
| 580488 | 40 |
| 580489 | 22 |
| 580490 | 34 |
| 580491 | 44 |
| 580492 | 44 |
| 580493 | 46 |
| 580494 | 45 |
| 424880 | 81 |
| No Oligo | 0 |

TABLE 7
Effect of modified oligonucleotides on Tau expression

| Isis # | % Reduction |
|---|---|
| 607479 | 39 |
| 607480 | 37 |
| 607481 | 34 |
| 607482 | 36 |
| 607483 | 50 |
| 607484 | 65 |
| 607485 | 54 |
| 607486 | 48 |
| 607487 | 47 |
| 607488 | 29 |
| 607489 | 15 |
| 607490 | 1 |
| 607491 | 48 |
| 607492 | 50 |
| 607493 | 66 |
| 607494 | 62 |
| 607495 | 43 |
| 607496 | 38 |
| 607497 | 40 |
| 607498 | 38 |
| 424880 | 85 |
| No Oligo | 0 |

1. An antisense compound comprising a modified oligonucleotide consisting of 10-30 linked nucleosides and having a nucleobase sequence complementary to an intron/exon junction or an exon/intron junction or a splice modulation site of a Tau transcript.

2. An antisense compound comprising a modified oligonucleotide consisting of 8 to 80 linked nucleosides having a nucleobase sequence comprising any one of SEQ ID NOs: 17-344.

3. An antisense compound comprising a modified oligonucleotide having a nucleobase sequence consisting of any one of SEQ ID NOs: 17-344.

4. An antisense compound comprising a modified oligonucleotide consisting of 10 to 30 linked nucleosides and having a nucleobase sequence comprising at least 8 contiguous nucleobases of any of the nucleobase sequences of SEQ ID NOs: 17-344.

5. An antisense compound comprising a modified oligonucleotide consisting of 10 to 30 linked nucleosides and having a nucleobase sequence comprising at least 9 contiguous nucleobases of any of the nucleobase sequences of SEQ ID NOs: 17-344.

6. An antisense compound comprising a modified oligonucleotide consisting of 10 to 30 linked nucleosides and having a nucleobase sequence comprising at least 10 contiguous nucleobases of any of the nucleobase sequences of SEQ ID NOs: 17-344.

7. An antisense compound comprising a modified oligonucleotide consisting of 10 to 30 linked nucleosides and having a nucleobase sequence comprising at least 11 contiguous nucleobases of any of the nucleobase sequences of SEQ ID NOs: 17-344.

8. An antisense compound comprising a modified oligonucleotide consisting of 10 to 30 linked nucleosides and having a nucleobase sequence comprising at least 12 contiguous nucleobases of any of the nucleobase sequences of SEQ ID NOs: 17-344.

9. An antisense compound comprising a modified oligonucleotide consisting of 10 to 30 linked nucleosides and having a nucleobase sequence comprising the nucleobase sequence of any one of SEQ ID NOs: 17-344.

10. An antisense compound comprising a modified oligonucleotide consisting of the nucleobase sequence of any one of SEQ ID NOs: 17-344.

11. The antisense compound of any of claims 1-10, wherein the antisense compound is single-stranded.

12. The antisense compound of any of claims 1-10, wherein the antisense compound is double-stranded.

13. The antisense compound of any of claims 1-12, wherein the antisense compound comprises at least one conjugate.

14. The antisense compound of any of claims 1-13, wherein the modified oligonucleotide comprises at least one modified nucleoside.

15. The antisense compound of claim 14, wherein the modified oligonucleotide comprises at least one modified nucleoside comprising a modified sugar.

16. The antisense compound of claim 15, wherein the at least one modified sugar is selected from among a bicyclic sugar, a non-bicyclic 2′-modified sugar, and a sugar surrogate.

17. The antisense compound of claim 16, wherein at least one modified nucleoside is a 2′-MOE modified nucleoside.

18. The antisense compound of claim 16, wherein at least one modified nucleoside is a morpholino nucleoside.

19. The antisense compound of any of claims 14-18, wherein essentially each nucleoside of the modified oligonucleotide is modified.

20. The antisense compound of any of claims 14-18, wherein each nucleoside of the modified oligonucleotide is modified.

21. The antisense compound of any of claims 14-20, wherein each modified nucleoside has the same modification.

22. The antisense compound of any of claims 14-20, wherein at least two modified nucleoside have different modifications from one another.

23. The antisense compound of any of claims 1-22, wherein the modified oligonucleotide comprises at least one modified internucleoside linkage.

24. The antisense compound of claim 23, wherein each internucleoside linkage of the modified oligonucleotide is a modified internucleoside linkage.

25. The antisense compound of claim 23 or 24, wherein the modified internucleoside linkage is a phosphorothioate internucleoside linkage.

26. The antisense compound of any of claims 1-25, wherein the modified oligonucleotide consists of 15, 16, 17, 18, 19, 20, 21, 22, 23, 24, or 25 linked nucleosides.

27. The antisense compound of any of claims 1-26, wherein the modified oligonucleotide is at least 75%, at least 80%, at least 85%, at least 90%, or at least 95% complementary to an equal length portion of a human Tau nucleic acid.

28. The antisense compound of any of claims 1-26, wherein the modified oligonucleotide is 100% complementary to an equal length portion of a human Tau nucleic acid.

29. The antisense compound of any of claims 1-28, wherein the modified oligonucleotide is complementary to the junction of exon −1/intron −1 of the Tau transcript.

30. The antisense compound of any of claims 1-28, wherein the modified oligonucleotide is complementary to the junction of intron −1/exon 1 of the Tau transcript.

31. The antisense compound of any of claims 1-28, wherein the modified oligonucleotide is complementary to the junction of exon 1/intron 1 of the Tau transcript.

32. The antisense compound of any of claims 1-28, wherein the modified oligonucleotide is complementary to the junction of intron 1/exon 2 of the Tau transcript.

33. The antisense compound of any of claims 1-28, wherein the modified oligonucleotide is complementary to the junction of exon 2/intron 2 of the Tau transcript.

34. The antisense compound of any of claims 1-28, wherein the modified oligonucleotide is complementary to the junction of intron 2/exon 3 of the Tau transcript.

35. The antisense compound of any of claims 1-28, wherein the modified oligonucleotide is complementary to the junction of exon 3/intron 3 of the Tau transcript.

36. The antisense compound of any of claims 1-28, wherein the modified oligonucleotide is complementary to the junction of intron 3/exon 4 of the Tau transcript.

37. The antisense compound of any of claims 1-28, wherein the modified oligonucleotide is complementary to the junction of exon 4/intron 4 of the Tau transcript.

38. The antisense compound of any of claims 1-28, wherein the modified oligonucleotide is complementary to the junction of intron 4/exon 5 of the Tau transcript.

39. The antisense compound of any of claims 1-28, wherein the modified oligonucleotide is complementary to the junction of exon 5/intron 5 of the Tau transcript.

40. The antisense compound of any of claims 1-28, wherein the modified oligonucleotide is complementary to the junction of intron 5/exon 6 of the Tau transcript.

41. The antisense compound of any of claims 1-28, wherein the modified oligonucleotide is complementary to the junction of exon 6/intron 6 of the Tau transcript.

42. The antisense compound of any of claims 1-28, wherein the modified oligonucleotide is complementary to the junction of intron 6/exon 7 of the Tau transcript.

43. The antisense compound of any of claims 1-28, wherein the modified oligonucleotide is complementary to the junction of exon 7/intron 7 of the Tau transcript.

44. The antisense compound of any of claims 1-28, wherein the modified oligonucleotide is complementary to the junction of intron 7/exon 8 of the Tau transcript.

45. The antisense compound of any of claims 1-28, wherein the modified oligonucleotide is complementary to the junction of exon 8/intron 8 of the Tau transcript.

46. The antisense compound of any of claims 1-28, wherein the modified oligonucleotide is complementary to the junction of intron 8/exon 9 of the Tau transcript.

47. The antisense compound of any of claims 1-28, wherein the modified oligonucleotide is complementary to the junction of exon 9/intron 9 of the Tau transcript.

48. The antisense compound of any of claims 1-28, wherein the modified oligonucleotide is complementary to the junction of intron 9/exon 10 of the Tau transcript.

49. The antisense compound of any of claims 1-28, wherein the modified oligonucleotide is complementary to the junction of exon 10/intron 10 of the Tau transcript.

50. The antisense compound of any of claims 1-28, wherein the modified oligonucleotide is complementary to the junction of intron 10/exon 11 of the Tau transcript.

51. The antisense compound of any of claims 1-28, wherein the modified oligonucleotide is complementary to the junction of exon 11/intron 11 of the Tau transcript.

52. The antisense compound of any of claims 1-28, wherein the modified oligonucleotide is complementary to the junction of intron 11/exon 12 of the Tau transcript.

53. The antisense compound of any of claims 1-28, wherein the modified oligonucleotide is complementary to the junction of exon 12/intron 12 of the Tau transcript.

54. The antisense compound of any of claims 1-28, wherein the modified oligonucleotide is complementary to the junction of intron 12/exon 13 of the Tau transcript.

55. The antisense compound of any of claims 1-28, wherein the modified oligonucleotide is complementary to the junction of exon 13/intron 13 of the Tau transcript.

56. The antisense compound of any of claims 1-28, wherein the modified oligonucleotide is complementary to a splice modulation site within intron −1, 1, 2, 3, 4, 5, 6, 7, 8, 9, 10, 11, 12, or 13 of the Tau transcript.

57. The antisense compound of any of claims 1-28, wherein the modified oligonucleotide is complementary to a splice modulation site within exon −1, 1, 2, 3, 4, 5, 6, 7, 8, 9, 10, 11, 12, or 13 of the Tau transcript.

58. The antisense compound of any of claims 1-57, wherein the Tau transcript is a mouse Tau transcript.

59. The antisense compound of any of claims 1-57, wherein the Tau transcript is a human Tau transcript.

60. The antisense compound of any of claims 1-58, wherein the modified oligonucleotide has a nucleobase sequence comprising at least 8, 10, 12, 14, 16, 18, 20, or 22 contiguous nucleobases of a nucleobase sequence selected from among of any of the oligonucleotides described in the present disclosure.

61. A method comprising contacting a cell with an antisense compound according to any of claims 1-60.

62. The method of claim 61, wherein the cell is in vitro.

63. The method of claim 61, wherein the cell is in an animal.

64. A method for reducing the amount or activity of a Tau transcript in a cell comprising contacting the cell with an antisense compound of any of claims 1-60 and thereby reducing the amount or activity of the Tau transcript in the cell.

65. The method of claim 64, wherein the amount of Tau transcript is reduced.

66. A method of treating a Tau disorder in an animal comprising administering to the animal an antisense compound according to any of claims 1-60.

67. The method of claim 66, wherein the Tau disorder is selected from among: Tauopathy, Alzheimer's Disease, Fronto-temporal Dementia (FTD), FTDP-17, Progressive Supranuclear Palsy (PSP), Chronic Traumatic Encephalopathy (CTE), Corticobasal Ganglionic Degeneration (CBD), Epilepsy, or Dravet's Syndrome.

68. The method of claim 66 or 67, wherein the animal is a mouse.

69. The method of claim 66 or 67, wherein the animal is a human.

70. A method of reducing the amount or activity of a Tau transcript in a cell comprising contacting the cell with an antisense compound, wherein the antisense compound comprises a fully modified oligonucleotide, wherein each nucleoside of the fully modified oligonucleotide comprises the same sugar modification.

71. The method of claim 70, wherein the sugar modification is 2′-MOE.

72. The method of claim 71, wherein the sugar modification is morpholino or 2′-OMethyl.

73. The method of any of claims 70-72, wherein the modified oligonucleotide comprises a phosphorothioate internucleoside linkage.

74. The method of claim 73, wherein each internucleoside linkage of the modified oligonucleotide is a phosphorothioate internucleoside linkage.

75. The method of claim 73, wherein each internucleoside linkage of the modified oligonucleotide is, independently, a phosphate internucleoside linkage or a phosphorothioate internucleoside linkage.

76. The method of any of claims 70-75, wherein the modified oligonucleotide has a sequence selected from among SEQ ID NO: 37, 206, 207, 209, 227, 228, 237, 278, 279, 287, 313, 314, 315, and 324.

77. The method of any of claims 70-76, wherein the cell is in an animal.

78. The method of claim 77, wherein the cell in the central nervous system of the animal.

79. The method of any of claims 77-78, wherein the antisense compound is administered intrathecally to the animal.

80. A method of reducing the amount or activity of a transcript in the central nervous system of an animal comprising intrathecal administration of an antisense compound to the animal, wherein the antisense compound comprises a fully modified oligonucleotide, and wherein the amount or activity of the transcript is reduced via nonsense mediated decay.

81. The method of claim 80, wherein each nucleoside of the fully modified oligonucleotide comprises the same sugar modification.

82. The method of claim 81, wherein the sugar modification is 2′-MOE, 2′-OMethyl, or morpholino.

83. The method of claim 82, wherein the sugar modification is 2′-MOE.

84. The method of any of claims 80-83, wherein the modified oligonucleotide comprises a phosphorothioate internucleoside linkage.

85. The method of claim 84, wherein each internucleoside linkage of the modified oligonucleotide is a phosphorothioate internucleoside linkage.

86. The method of claim 84, wherein each internucleoside linkage of the modified oligonucleotide is, independently, a phosphate internucleoside linkage or a phosphorothioate internucleoside linkage.

87. The method of claim 80, wherein the antisense compound is the antisense compound of any of claims 1-60.
